(19) United States
(12) Patent Application Publication
(10) Pub. No.: US 2023/0235064 A1
(43) Pub. Date: Jul. 27, 2023

(54) BISPECIFIC IMMUNE CELL ENGAGERS WITH BINDING SPECIFICITY FOR HLA-G AND ANOTHER ANTIGEN

(71) Applicant: TIZONA THERAPEUTICS, South San Francisco, CA (US)

(72) Inventors: Courtney Beers, South San Francisco, CA (US); Ling-Hon Matthew Chu, South San Francisco, CA (US); Doug Hodges, South San Francisco, CA (US); Christina M. Kochel, South San Francisco, CA (US)

(21) Appl. No.: 18/009,686

(22) PCT Filed: Jun. 10, 2021
(86) PCT No.: PCT/US2021/036838

§ 371 Date: Dec. 9, 2022

Related U.S. Application Data
(60) Provisional application No. 63/037,985, filed on Jun. 11, 2020.

(51) Int. Cl. C07K 16/28

(52) U.S. Cl. CPC C07K 16/2833; C07K 16/2827; C07K 16/2896; C07K 16/283; C07K 16/2809; C07K 2317/565; C07K 2317/31; C07K 2317/732

(57) ABSTRACT

Provided herein are bispecific immune cell engagers with binding specificity for HLA-G and an additional antigen, including pharmaceutical compositions, diagnostic compositions, and kits.

### FIELD

Provided herein are bispecific immune cell engagers with binding specificity for HLA-G and an additional antigen, including pharmaceutical compositions, diagnostic compositions, and kits.

### BACKGROUND

A bispecific antibody is a protein that can bind to two different antigens. Bispecific antibodies are widely used in cancer immunotherapy, where bispecific antibodies that function as immune cell engagers are engineered to simultaneously bind a cytotoxic cell and a target like a tumor cell to be killed. For example, the link between T cells and tumor cells causes T cells to exert cytotoxic activity on tumor cells by producing proteins like perforin and granzymes. These proteins enter tumor cells and initiate a cell's apoptosis process.

Bispecific antibodies can direct a host's immune system against cancer cells. For example, a bispecific antibody can be made that binds to CD3 in a T cell and a second antigen on a tumor cell. Bridging T cells and tumor cells and killing tumor cells as a result can induce dramatic regression of malignancies. This can even lead to complete remission in some cases.

Bispecific T-cell engagers (BiTEs) are a class of bispecific antibodies that are particularly useful as anti-cancer compounds. BiTEs and other bispecific immune cell engagers are usually fusion proteins consisting of two different antibodies or amino acid sequences, such as, for example, two different single-chain variable fragments (scFvs). One of the scFvs binds to an activating receptor on immune cells, for example CD3 on T cells, and the other targets a tumor cell via a specific molecule. Bispecific T-cell engagers can thus be used to eliminate target-expressing tumor cells by activated T cells.

Human leukocyte antigen-G (HLA-G) is an immune regulatory molecule that belongs to the non-classical HLA-class I family of receptors and is encoded by the HLA-G gene. HLA-G is a heterodimer consisting of a heavy chain and a light chain (beta-2 microglobulin). There are membrane bound and soluble forms of HLA-G.

HLA-G was first identified in placenta samples. HLA-G is normally expressed at the maternal-fetal interface and other immune-privileged sites. HLA-G may play a role in immune tolerance in pregnancy, being expressed in the placenta by extravillous trophoblast cells, while the classical MHC class I genes (HLA-A and HLA-B) are not.

HLA-G has been shown to be immune-suppressive. By binding receptors expressed on various myeloid and lymphoid cells, HLA-G may directly inhibit the functions of NK cells, cytotoxic T-lymphocytes, B cells, neutrophils, monocytes, macrophages, and dendritic cells. HLA-G also inhibits T and NK cell proliferation and cytolytic activities. HLA-G suppresses phagocytosis and induces the generation or expansion of regulatory T cells.

HLA-G mediates immune function through at least three ITIM-containing inhibitory receptors, ILT2, ILT4, and KIR2DL4. On lymphoid cells, for example, HLA-G mediates function through ILT2. On myeloid cells, HLA-G mediates function through ILT2 and ILT4. On decidual NK cells, HLA-G mediates immune function through KIR2DL4 and ILT2.

HLA-G is an immune checkpoint target. HLA-G can directly inhibit immune cell function through receptor binding and/or trogocytosis and impairment of chemotaxis. HLA-G can lend tumor cells a higher invasive and metastatic potential. HLA-G promotes evasion of tumor immune surveillance and enhances metastasis and the progression of malignancies. During tumor progression, HLA-G has other effects, such as inhibition of immune cell cytolysis, induction of immune cell apoptosis, and/or the generation of regulatory cells through receptor binding and/or trogocytosis.

HLA-G is an ideal antigen for a bispecific antibody. HLA-G expression is upregulated on a broad spectrum of tumors and is associated with poor prognosis and disease progression. Serum HLA-G levels are elevated in, for example, without limitation, breast, lung, colorectal cancer (CRC), gastric, esophageal, neuroblastoma, cervical, and hematological cancers. HLA-G has also been found to be correlated with clinical parameters in advanced disease, such as tumor metastasis, poor prognosis, immune escape, and tumor invasiveness.

A bispecific antibody that binds an immune cell, such as a T cell, and HLA-G would be useful. For example, a bispecific T-cell engager that can bind both CD3 and HLA-G would be particularly useful.

### SUMMARY

A first aspect provides a bispecific antigen binding construct comprising a binding domain capable of binding to an HLA-G epitope and an additional binding domain capable of binding to a second epitope. In some embodiments, the second epitope comprises or consists of a CD3ε epitope. In some embodiments, the CD3ε epitope comprises or consists of an amino acid sequence set forth in SEQ ID NO: 629.

In some embodiments, the additional binding domain capable of binding to a CD3ε epitope comprises or consists of a heavy chain variable region (VH) and a light chain variable region (VL), with VH and/or VL comprising 1, 2, 3, 4, 5, or 6 of the following:
a) a VHCDR1 having the sequence set forth in any one of SEQ ID NOS: 346-349 or 354-357,
b) a VHCDR2 having the sequence set forth in any one of SEQ ID NOS: 362-365 or 371-375,
c) a VHCDR3 having the sequence set forth in any one of SEQ ID NOS: 379-382,
d) a VLCDR1 having the sequence set forth in any one of SEQ ID NOS: 388-392,
e) a VLCDR2 having the sequence set forth in any one of SEQ ID NOS: 396-400, and
f) a VLCDR3 having the sequence set forth in any one of SEQ ID NOS: 404-408.

In some embodiments, the additional binding domain comprises or consists of an NK cell engager, dendritic cell engager, monocyte, or macrophage engager. In some embodiments, the NK cell engager comprises or consists an antibody for a CD16 epitope. In some embodiments, the NK cell engager comprises or consists of an antibody for NKp46. In some embodiments, the NK cell engager comprises or consists of an antibody for NKp30. In some embodiments, the monocyte or macrophage engager comprises or consists of an antibody for a CD16 epitope.

In some embodiments, the HLA-G epitope comprises or consists of an amino acid sequence set forth in SEQ ID NO: 342. In some embodiments, the binding domain capable of binding to an HLA-G epitope comprises or consists of a heavy chain variable region (VH) and a light chain variable region (VL), with VH and/or VL comprising 1, 2, 3, 4, 5, or 6 of the following:
a) a VHCDR1 having the sequence set forth in any one of SEQ ID NOS: 1-14 or 18-34,
b) a VHCDR2 having the sequence set forth in any one of SEQ ID NOS: 38-50 or 54-71,
c) a VHCDR3 having the sequence set forth in any one of SEQ ID NOS: 76-101,
d) a VLCDR1 having the sequence set forth in any one of SEQ ID NOS: 105-124,
e) a VLCDR2 having the sequence set forth in any one of SEQ ID NOS: 128-145, and
f) a VLCDR3 having the sequence set forth in any one of SEQ ID NOS: 149-166.

In some embodiments, the binding domain capable of binding to an HLA-G epitope comprises a heavy chain variable region (VH) and a light chain variable region (VL), with VH comprising, consisting of, or consisting essentially of a VH having the sequence set forth in SEQ ID NOS: 170-200 and with the VL comprising, consisting of, or consisting essentially of a VL having the sequence set forth in SEQ ID NO: 204-228 and the additional binding domain capable of binding to a CD3ε epitope comprises a heavy chain variable region (VH) and a light chain variable region (VL), with VH comprising, consisting of, or consisting essentially of a VH having the sequence set forth in SEQ ID NOS: 413-418 and with the VL comprising, consisting of, or consisting essentially of a VL having the sequence set forth in SEQ ID NOS: 422-427.

Some embodiments provide a pharmaceutical composition comprising or consisting of any of the bispecific antigen binding constructs provided herein. In some embodiments, the pharmaceutical composition further comprises an effective amount of one or more of:
a) an anti-PD-L1 antibody or small molecule inhibitor;
b) an anti-PD-1 antibody or small molecule inhibitor;
c) an anti-CD38 antibody or small molecule inhibitor;
d) an anti-CD39 antibody or small molecule inhibitor;
e) an anti-CD73 antibody or small molecule inhibitor;
f) an anti-A2A receptor antibody or small molecule inhibitor;
g) an anti-A2B receptor antibody or small molecule inhibitor;
h) an anti-A2A/A2B dual receptor antibody or small molecule inhibitor;
i) an anti-CD47 antibody or small molecule inhibitor;
j) an anti-CTLA-4 antibody or small molecule inhibitor;
k) an anti-LAG-3 antibody or small molecule inhibitor;
l) an anti-TIM-3 antibody or small molecule inhibitor;
m) an anti-TIGIT antibody or small molecule inhibitor;
n) an anti-VISTA antibody or small molecule inhibitor;
o) an anti-CD94 antibody or small molecule inhibitor;
p) a small molecule inhibitor;
q) an oncolytic virus;
r) a chemotherapy;
s) ADCC capable therapies using effector competent antibodies such as anti-CD19, anti-CD20, anti-EGFR, anti-Her2, anti-SLAMF7, anti-CD52, anti-BCMA, anti-GD2, and/or anti-CCR4.
   In some embodiments, the pharmaceutical composition further comprises one or both of:
a) an antibody to an immune inhibitory receptor or ligand and/or
b) an antibody to an immune stimulatory receptor or ligand.

A second aspect provides one or more nucleic acids encoding any of the bispecific antigen binding constructs provided herein. In some embodiments, the one or more nucleic acids comprises one or more vectors. Some embodiments provide a host transformed with the one or more vectors.

Some embodiments provide a method for the production of one or more bispecific antigen binding construct comprising the steps of expressing any of the one or more nucleic acids provided herein in a prokaryotic or eukaryotic host cell and recovering the one or more bispecific antigen binding construct from the cell or the cell culture supernatant.

A third aspect provides a method for treating a subject with cancer comprising administering a therapeutically effective amount of any of the bispecific antigen binding constructs or pharmaceutical compositions provided herein to the subject.

In some embodiments, the cancer is a solid cancer. In some embodiments, the cancer is a hematological cancer. In some embodiments, the cancer is selected from the group consisting of a hematopeietic cancer, hepatocellular carcinoma, leukemia, colorectal cancer (CRC), breast cancer, gastric cancer, esophageal cancer, endometrial cancer, prostate cancer, bladder cancer, thyroid cancer, liver cancer, pancreatic cancer, triple negative breast cancer, cervical cancer, ovarian cancer, uterine cancer, vaginal cancer, vulvar cancer, lung cancer, head and neck cancer, melanoma, renal cell carcinoma, cutaneous squamous cell carcinoma, Hodgkin's lymphoma, a metastasis of the brain, a metastasis of the lung, a metastasis of the liver, and/or a metastasis of the bone, or an unresectable or metastatic solid tumor with DNA mismatch repair deficiencies or a microsatellite instability-high state. In some embodiments, the cancer is a cancer that expresses HLA-G.

In some embodiments, the method further comprises one or more of the following:
a) administering chemotherapy to the subject;
b) administering radiation therapy to the subject; and/or
c) administering one or more additional therapeutic agents to the subject.
   In some embodiments, the one or more additional therapeutic agents comprise one or more immunomodulatory agents. For example, these agents could be ones that antagonize or block inhibitory signals and interactions. The agents could provide activation signals or costimulation to immune cells.

In some embodiments, the one or more immunomodulatory agents comprise an antagonist to an inhibitory receptor of an immune cell. In some embodiments, the inhibitory receptor is at least one of LILRB1, LILRB2, LILRB4, KIR2DL4, CTLA-4, PD-1, PD-L1, PD-L2, LAG-3, Tim3, TIGIT, B7-H3, B7-H4, neuritin, BTLA, CECAM-1, CECAM-5, VISTA, LAIR1, CD160, 2B4, TGF-B receptor, NKG2A, and/or a Killer-cell immunoglobulin-like receptor (KIR). In some embodiments, the one or more immunomodulatory agents comprise an agonist of a co-stimulatory receptor of an immune cell. In some embodiments, the co-stimulatory receptor is at least one of OX40, CD2, CD27, ICAM-1, LFA-1, ICOS (CD278), 4-1BB (CD137), GITR, CD28, CD30, CD40, BAFFR, HVEM, CD7, LIGHT, NKG2C, SLAMF7, NKp30, NKp46, NKp80, CD160, and/or CD83.

In some embodiments, the one or more immunomodulatory agents is one or more cytokines. In some embodiments, the one or more cytokines is at least one of G-CSF, GM-CSF, IFN-alpha, IFN-beta, IFN-gamma, FLt3 ligand, IL-1, IL-2, IL-5, IL-7, IL-10, IL-12, IL-15, IL-18, IL-21, and/or IL-27.

In some embodiments, the one or more immunomodulatory agents is one or more oncolytic viruses. In some embodiments, the one or more oncolytic viruses is a Herpes simplex virus, a Vesicular stomatitis virus, an adenovirus, a Newcastle disease virus, a vaccinia virus, and/or a maraba virus.

### BRIEF DESCRIPTION OF THE DRAWINGS

FIG. 1 shows evaluation of an HLA-G×CD3 bispecific antibody binding to cancer cell lines with engineered (FIG. 1A) or endogenous (FIG. 1B) HLA-G expression. The parental anti-HLA-G monoclonal antibody was included for comparison. Control cell lines lacking HLA-G expression are depicted in grey.

FIG. 2 provides the binding profiles for an HLA-G×CD3 bispecific antibody and the parental anti-CD3 monoclonal antibody to CD4⁺ and CD8⁺ T cells from isolated human peripheral blood mononuclear cells. Representative binding profiles from two individual donors are shown. Isotype controls are depicted with open symbols.

FIG. 3 shows the activity of an HLA-G×CD3 bispecific antibody to induce T cell-dependent cellular cytotoxicity of cancer cell lines with engineered (FIG. 3A) or endogenous (FIG. 3B) HLA-G expression.

### DETAILED DESCRIPTION

Provided herein are bispecific immune cell engagers with binding specificity for HLA-G and another antigen, including pharmaceutical compositions, diagnostic compositions, and kits. In particular, heteromultimeric antibodies e.g., full length antibodies or antigen binding fragments thereof, that specifically bind to HLA-G and another antigen, such as CD3, are provided. Compositions comprising such heteromultimeric antibodies, methods for producing and purifying such heterodimeric antibodies, and their use in diagnostics and therapeutics are also provided.

### I. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value ±10%, ±5%, or ±1%. In certain embodiments, the term “about” indicates the designated value ±one standard deviation of that value.

The term “combinations thereof” includes every possible combination of elements to which the term refers.

The terms “HLA-G,” “MHC-G,” and “major histocompatibility complex, class I, G” as well as any terms known to one skilled in the art are used interchangeably herein. HLA-G belongs to the HLA class I heavy chain paralogues. The class I HLA-G molecule is a heterodimer consisting of a heavy chain and a light chain (beta-2 microglobulin). The heavy chain is anchored in the membrane. HLA-G is expressed on fetal derived placental cells. The heavy chain is approximately 45 kDa and its gene contains 8 exons. Exon one encodes the leader peptide, exons 2 and 3 encode the alpha1 and alpha2 domain, which both bind the peptide, exon 4 encodes the alpha3 domain, exon 5 encodes the transmembrane region, and exon 6 encodes the cytoplasmic tail.

Unless specified otherwise, the terms include any variants, isoforms, and species homologs of human HLA-G that are naturally expressed by cells, or that are expressed by cells transfected with an HLA-G gene.

The terms “CD3” or “cluster of differentiation 3” as well as any terms known to one skilled in the art are used interchangeably herein. Unless specified otherwise, the terms include any variants, isoforms, and species homologs of human CD3 that are naturally expressed by cells, or that are expressed by cells transfected with a CD3 gene. In some embodiments, CD3 proteins include murine CD3. In some embodiments, CD3 proteins include cynomolgus CD3.

The terms “CD16,” “cluster of differentiation 16,” and “FcγRIII” as well as any terms known to one skilled in the art are used interchangeably herein. Unless specified otherwise, the terms include any variants, isoforms, and species homologs of human CD16 that are naturally expressed by cells, or that are expressed by cells transfected with a CD16 gene. In some embodiments, CD16 proteins include murine CD16. In some embodiments, CD16 proteins include cynomolgus CD16.

The terms “NKp46,” “NCR1,” “natural cytotoxicity triggering receptor 1,” “CD335,” “NKP46,” “NK-p46,” and “LY94” as well as any terms known to one skilled in the art are used interchangeably herein. Unless specified otherwise, the terms include any variants, isoforms, and species homologs of human NKp46 that are naturally expressed by cells, or that are expressed by cells transfected with a NCR1 gene. In some embodiments, NKp46 proteins include murine NKp46. In some embodiments, NKp46 proteins include cynomolgus NKp46.

The terms “NKp30,” “natural cytotoxicity triggering receptor 3,” “CD337,” “cluster of differentiation 337,” as w ell as any terms known to one skilled in the art are used interchangably herein. Unless specified otherwise, the terms include any variants, isoforms, and species homologs of human NKp30 that are naturally expressed by cells, or that are expressed by cells transfected with a NCR1 gene. In some embodiments, NKp30 proteins include murine NKp30. In some embodiments, NKp30 proteins include cynomolgus NKp30.

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, Pa. Briefly, each heavy chain typically comprises a heavy chain variable region (V_{H}) and a heavy chain constant region (C_{H}). The heavy chain constant region typically comprises three domains, CH1, CH2, and CH3. Each light chain typically comprises a light chain variable region (VL) and a light chain constant region. The light chain constant region typically comprises one domain, abbreviated CL.

The term “antibody” describes a type of immunoglobulin molecule and is used herein in its broadest sense. An antibody specifically includes intact antibodies (e.g., intact immunoglobulins) and antibody fragments.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and confer antigen specificity and binding affinity to the antibody. See Kabat et al., Sequences of Proteins of Immunological Interest 5th ed. (1991) Public Health Service, National Institutes of Health, Bethesda, Md., incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme), each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

Unless otherwise specified, the numbering scheme used for identification of a particular CDR herein is the Kabat numbering scheme. Variant and equivalent antibodies with a Chothia numbering scheme are intended to be within the scope of the invention.

TABLE 1
Residues in CDRs according to Kabat
and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments. In some embodiments, an antibody or antigen binding fragment that binds HLA-G and an antibody or antigen binding fragment that binds an additional binding domain, such as CD3, includes antibody fragments of binding domain that binds HLA-G and a binding domain that binds an additional binding domain.

“Fv” fragments comprise a non-covalently linked dimer of one heavy chain variable domain and one light chain variable domain.

“Fab” fragments comprise, in addition to the heavy and light chain variable domains, the constant domain of the light chain and the first constant domain (Cm) of the heavy chain. Fab fragments may be generated, for example, by papain digestion of a full-length antibody.

“F(ab′)₂” fragments contain two Fab′ fragments joined, near the hinge region, by disulfide bonds. F(ab′)₂ fragments may be generated, for example, by pepsin digestion of an intact antibody. The F(ab′) fragments can be dissociated, for example, by treatment with β-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety. “scFv-Fc” fragments comprise an scFv attached to an Fc domain. For example, an Fc domain may be attached to the C-terminal of the scFv. The Fc domain may follow the V_{H} or V_{L} depending on the orientation of the variable domains in the scFv (i.e., V_{H}-V_{L} or V_{L}-V_{H}). Any suitable Fc domain known in the art or described herein may be used.

A “minibody” comprises an antibody which features a smaller molecular weight than that of a traditional larger antibody.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

The term “bispecific antigen binding construct” or “bispecific antibody” or any related term known or used by one skilled in the art describes a type of immunoglobulin that can bind at least two different antigens. For example, without limitation, a bispecific antigen binding construct may bind HLA-G and CD3.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, llama, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” is one that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. An isolated antibody includes an antibody in situ within recombinant cells, since at least one component of the antibody's natural environment is not present. In some embodiments, an isolated antibody is prepared by at least one purification step.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Affinity can be determined, for example, using surface plasmon resonance (SPR) technology, such as a Biacore® instrument.

With regard to the binding of an antibody to a target molecule, the terms “binding” or “binds to” a particular antigen (e.g., a polypeptide target) or an epitope on a particular antigen mean binding that is measurably different from a non-selective interaction. Binding can be measured, for example, by determining binding of a molecule compared to binding of a control molecule. Binding can also be determined by competition with a control molecule that is similar to the target, such as an excess of non-labeled target. In that case, binding is indicated if the binding of the labeled target to a probe is competitively inhibited by the excess non-labeled target.

The term “k_{d}” (sec⁻¹), as used herein, refers to the dissociation rate constant of a particular antibody-antigen interaction. This value is also referred to as the k_{off} value.

The term “kₐ” (M⁻¹×sec⁻¹), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=k_{d}/kₐ.

The term “K_{A}” (M⁻¹), as used herein, refers to the association equilibrium constant of a particular antibody-antigen interaction. K_{A}=kₐ/k_{d}.

Percent “identity” between a polypeptide sequence and a reference sequence is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, or CLUSTAL OMEGA software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution of one or more amino acids with one or more chemically or functionally similar amino acids. Conservative substitution tables providing similar amino acids are well known in the art. Polypeptide sequences having such substitutions are known as “conservatively modified variants.” Such conservatively modified variants are in addition to and do not exclude polymorphic variants, interspecies homologs, and alleles. By way of example, the following groups of amino acids are considered conservative substitutions for one another.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

| | |
|---|---|
| Alcohol Group-Containing Residues | S and T |
| Aliphatic Residues | I, L, V, and M |
| Cycloalkenyl-associated Residues | F, H, W, and Y |
| Hydrophobic Residues | A, C, F, G, H, I, L, M, T, V, W, |
| | and Y |
| Negatively Charged Residues | D and E |
| Polar Residues | C, D, E, H, K, N, Q, R, S, and T |
| Positively Charged Residues | H, K, and R |
| Small Residues | A, C, D, G, N, P, S, T, and V |
| Very Small Residues | A, G, and S |
| Residues Involved in Turn Formation | A, C, D, E, G, H, K, N, Q, R, S, |
| | P, and T |
| Flexible Residues | Q, T, K, S, G, P, D, E, and R |

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, N.Y. An antibody generated by making one or more conservative substitutions of amino acid residues in a parent antibody is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

“Treating” or “treatment” of any cancer refers, in certain embodiments, to ameliorating a cancer that exists in a subject. In another embodiment, “treating” or “treatment” includes ameliorating at least one physical parameter, which may be indiscernible by the subject. In yet another embodiment, “treating” or “treatment” includes modulating the cancer, either physically (e.g., stabilization of a discernible symptom) or physiologically (e.g., stabilization of a physical parameter) or both.

As used herein, the term “therapeutically effective amount” or “effective amount” refers to an amount of an antibody or composition that when administered to a subject is effective to treat a cancer. In some embodiments, a therapeutically effective comprises or consists of exemplary doses of each antibody. In some embodiments, a therapeutically effective amount comprises or consists of determining an amount used to achieve a response according to a clinical endpoint. In some embodiments, the clinical endpoint comprises Objective Response Rate (ORR), Progression Free Survival (PFS), and/or Response Evaluation Criteria in Solid Tumors (“RECIST”).

As used herein, the term “subject” means a mammal or a human. In some embodiments subjects include, but are not limited to, monkeys, dogs, cats, mice, rats, cows, horses, camels, avians, goats, and sheep.

A first aspect provides a bispecific antigen binding construct comprising a binding domain capable of binding to an HLA-G epitope and an additional binding domain capable of binding to a second epitope.

In some embodiments, the binding domain comprises a light chain. In some aspects, the light chain is a kappa light chain. In some embodiments, the light chain is a lambda light chain.

In some embodiments, the binding domain comprises a heavy chain. In some embodiments, the heavy chain is an IgA. In some embodiments, the heavy chain is an IgD. In some embodiments, the heavy chain is an IgE. In some embodiments, the heavy chain is an IgG. In some embodiments, the heavy chain is an IgM. In some embodiments, the heavy chain is an IgG1. In some embodiments, the heavy chain is an IgG2. In some embodiments, the heavy chain is an IgG3. In some embodiments, the heavy chain is an IgG4. In some embodiments, the heavy chain is an IgA1. In some embodiments, the heavy chain is an IgA2.

In some embodiments, the binding domain is an antibody fragment. In some embodiments, the antibody fragment is an Fv fragment. In some embodiments, the antibody fragment is a Fab fragment. In some embodiments, the antibody fragment is a F(ab′)2 fragment. In some embodiments, the antibody fragment is a Fab′ fragment. In some embodiments, the antibody fragment is an scFv (sFv) fragment. In some embodiments, the antibody fragment is an scFv-Fc fragment. In some embodiments, the antibody fragment is a minibody. In some embodiments, the antibody fragment is a single domain antibody.

In some embodiments, the binding domain is a chimeric antibody. In some embodiments, the binding domain is a humanized antibody. In some embodiments, the binding domain is a human antibody.

In some embodiments, the binding domain is an affinity matured antibody. In some embodiments, the binding domain is an affinity matured antibody derived from an illustrative sequence provided in this disclosure.

### Binding Domains Capable of Binding to HLA-G

In some embodiments, the HLA-G epitope comprises or consists of an amino acid sequence set forth in SEQ ID NO: 342.

### V_{H} Sequences Comprising Illustrative CDRs

In some embodiments, the binding domain capable of binding to an HLA-G epitope comprises a V_{H} sequence comprising one or more CDR-H sequences comprising, consisting of, or consisting essentially of one or more illustrative CDR-H sequences provided in this disclosure, and variants thereof.

### V_{H} Sequences Comprising Illustrative Kabat CDRs

In some embodiments, the binding domain capable of binding to an HLA-G epitope comprises a V_{H} sequence comprising one or more Kabat CDR-H sequences comprising, consisting of, or consisting essentially of one or more illustrative Kabat CDR-H sequences provided in this disclosure, and variants thereof.

### Kabat CDR-H3

In some embodiments, the binding domain capable of binding to an HLA-G epitope comprises a V_{H} sequence comprising a Kabat CDR-H3 sequence comprising, consisting of, or consisting essentially of a sequence selected from SEQ ID NOS: 76-101. In some embodiments, the binding domain capable of binding to an HLA-G epitope comprises a V_{H} sequence comprising a Kabat CDR-H3 sequence comprising, consisting of, or consisting essentially of SEQ ID NO: 76. In some embodiments, the binding domain capable of binding to an HLA-G epitope comprises a V_{H} sequence comprising a Kabat CDR-H3 sequence comprising, consisting of, or consisting essentially of SEQ ID NO: 77. In some embodiments, the binding domain capable of binding to an HLA-G epitope comprises a V_{H} sequence comprising a Kabat CDR-H3 sequence comprising, consisting of, or consisting essentially of SEQ ID NO: 78. In some embodiments, the binding domain capable of binding to an HLA-G epitope comprises a V_{H} sequence comprising a Kabat CDR-H3 sequence comprising, consisting of, or consisting essentially of SEQ ID NO: 79. In some embodiments, the binding domain capable of binding to an HLA-G epitope comprises a V_{H} sequence comprising a Kabat CDR-H3 sequence comprising, consisting of, or consisting essentially of SEQ ID NO: 80. In some embodiments, the binding domain capable of binding to an HLA-G epitope comprises a V_{H} sequence comprising a Kabat CDR-H3 sequence comprising, consisting of, or consisting essentially of SEQ ID NO: 81. In some embodiments, the binding domain capable of binding to an HLA-G epitope comprises a V_{H} sequence comprising a Kabat CDR-H3 sequence comprising, consisting of, or consisting essentially of SEQ ID NO: 82. In some embodiments, the binding domain capable of binding to an HLA-G epitope comprises a V_{H} sequence comprising a Kabat CDR-H3 sequence comprising, consisting of, or consisting essentially of SEQ ID NO: 83. In some embodiments, the binding domain capable of binding to an HLA-G epitope comprises a V_{H} sequence comprising a Kabat CDR-H3 sequence comprising, consisting of, or consisting essentially of SEQ ID NO: 84. In some embodiments, the binding domain capable of binding to an HLA-G epitope comprises a V_{H} sequence comprising a Kabat CDR-H3 sequence comprising, consisting of, or consisting essentially of SEQ ID NO: 85. In some embodiments, the binding domain capable of binding to an HLA-G epitope comprises a V_{H} sequence comprising a Kabat CDR-H3 sequence comprising, consisting of, or consisting essentially of SEQ ID NO: 86. In some embodiments, the binding domain capable of binding to an HLA-G epitope comprises a V_{H} sequence comprising a Kabat CDR-H3 sequence comprising, consisting of, or consisting essentially of SEQ ID NO: 87. In some embodiments, the binding domain capable of binding to an HLA-G epitope comprises a V_{H} sequence comprising a Kabat CDR-H3 sequence comprising, consisting of, or consisting essentially of SEQ ID NO: 88. In some embodiments, the binding domain capable of binding to an HLA-G epitope comprises a V_{H} sequence comprising a Kabat CDR-H3 sequence comprising, consisting of, or consisting essentially of SEQ ID NO: 89. In some embodiments, the binding domain capable of binding to an HLA-G epitope comprises a V_{H} sequence comprising a Kabat CDR-H3 sequence comprising, consisting of, or consisting essentially of SEQ ID NO: 90. In some embodiments, the binding domain capable of binding to an HLA-G epitope comprises a V_{H} sequence comprising a Kabat CDR-H3 sequence comprising, consisting of, or consisting essentially of SEQ ID NO: 91. In some embodiments, the binding domain capable of binding to an HLA-G epitope comprises a V_{H} sequence comprising a Kabat CDR-H3 sequence comprising, consisting of, or consisting essentially of SEQ ID NO: 92. In some embodiments, the binding domain capable of binding to an HLA-G epitope comprises a V_{H} sequence comprising a Kabat CDR-H3 sequence comprising, consisting of, or consisting essentially of SEQ ID NO: 94. In some embodiments, the binding domain capable of binding to an HLA-G epitope comprises a V_{H} sequence comprising a Kabat CDR-H3 sequence comprising, consisting of, or consisting essentially of SEQ ID NO: 95. In some embodiments, the binding domain capable of binding to an HLA-G epitope comprises a V_{H} sequence comprising a Kabat CDR-H3 sequence comprising, consisting of, or consisting essentially of SEQ ID NO: 96. In some embodiments, the binding domain capable of binding to an HLA-G epitope comprises a V_{H} sequence comprising a Kabat CDR-H3 sequence comprising, consisting of, or consisting essentially of SEQ ID NO: 97. In some embodiments, the binding domain capable of binding to an HLA-G epitope comprises a V_{H} sequence comprising a Kabat CDR-H3 sequence comprising, consisting of, or consisting essentially of SEQ ID NO: 98. In some embodiments, the binding domain capable of binding to an HLA-G epitope comprises a V_{H} sequence comprising a Kabat CDR-H3 sequence comprising, consisting of, or consisting essentially of SEQ ID NO: 99. In some embodiments, the binding domain capable of binding to an HLA-G epitope comprises a V_{H} sequence comprising a Kabat CDR-H3 sequence comprising, consisting of, or consisting essentially of SEQ ID NO: 100. In some embodiments, the binding domain capable of binding to an HLA-G epitope comprises a V_{H} sequence comprising a Kabat CDR-H3 sequence comprising, consisting of, or consisting essentially of SEQ ID NO: 101.

In some embodiments, the CDR-H3 sequence comprises, consists of, or consists essentially of a variant of an illustrative CDR-H3 sequence provided in this disclosure. In some embodiments, the CDR-H3 sequence comprises, consists of, or consists essentially of a sequence having at least 70%, 75%, 80%, 85%, 90%, or 95% identity with any of the illustrative CDR-H3 sequences provided in this disclosure. In some embodiments, the CDR-H3 sequence comprises, consists of, or consists essentially of any of the illustrative CDR-H3 sequences provided in this disclosure, with 1, 2, or 3 amino acid substitutions. In some embodiments, the amino acid substitutions are conservative amino acid substitutions.

### Kabat CDR-H2

In some embodiments, the binding domain capable of binding to an HLA-G epitope comprises a V_{H} sequence comprising a Kabat CDR-H2 sequence comprising, consisting of, or consisting essentially of a sequence selected from SEQ ID NOS: 54-71. In some embodiments, the binding domain capable of binding to an HLA-G epitope comprises a V_{H} sequence comprising a Kabat CDR-H2 sequence comprising, consisting of, or consisting essentially of SEQ ID NO: 54. In some embodiments, the binding domain capable of binding to an HLA-G epitope comprises a V_{H} sequence comprising a Kabat CDR-H2 sequence comprising, consisting of, or consisting essentially of SEQ ID NO: 55. In some embodiments, the binding domain capable of binding to an HLA-G epitope comprises a V_{H} sequence comprising a Kabat CDR-H2 sequence comprising, consisting of, or consisting essentially of SEQ ID NO: 56. In some embodiments, the binding domain capable of binding to an HLA-G epitope comprises a V_{H} sequence comprising a Kabat CDR-H2 sequence comprising, consisting of, or consisting essentially of SEQ ID NO: 57. In some embodiments, the binding domain capable of binding to an HLA-G epitope comprises a V_{H} sequence comprising a Kabat CDR-H2 sequence comprising, consisting of, or consisting essentially of SEQ ID NO: 58. In some embodiments, the binding domain capable of binding to an HLA-G epitope comprises a V_{H} sequence comprising a Kabat CDR-H2 sequence comprising, consisting of, or consisting essentially of SEQ ID NO: 59. In some embodiments, the binding domain capable of binding to an HLA-G epitope comprises a V_{H} sequence comprising a Kabat CDR-H2 sequence comprising, consisting of, or consisting essentially of SEQ ID NO: 60. In some embodiments, the binding domain capable of binding to an HLA-G epitope comprises a V_{H} sequence comprising a Kabat CDR-H2 sequence comprising, consisting of, or consisting essentially of SEQ ID NO: 61. In some embodiments, the binding domain capable of binding to an HLA-G epitope comprises a V_{H} sequence comprising a Kabat CDR-H2 sequence comprising, consisting of, or consisting essentially of SEQ ID NO: 62. In some embodiments, the binding domain capable of binding to an HLA-G epitope comprises a V_{H} sequence comprising a Kabat CDR-H2 sequence comprising, consisting of, or consisting essentially of SEQ ID NO: 63. In some embodiments, the binding domain capable of binding to an HLA-G epitope comprises a V_{H} sequence comprising a Kabat CDR-H2 sequence comprising, consisting of, or consisting essentially of SEQ ID NO: 64. In some embodiments, the binding domain capable of binding to an HLA-G epitope comprises a V_{H} sequence comprising a Kabat CDR-H2 sequence comprising, consisting of, or consisting essentially of SEQ ID NO: 65. In some embodiments, the binding domain capable of binding to an HLA-G epitope comprises a V_{H} sequence comprising a Kabat CDR-H2 sequence comprising, consisting of, or consisting essentially of SEQ ID NO: 66. In some embodiments, the binding domain capable of binding to an HLA-G epitope comprises a V_{H} sequence comprising a Kabat CDR-H2 sequence comprising, consisting of, or consisting essentially of SEQ ID NO: 67. In some embodiments, the binding domain capable of binding to an HLA-G epitope comprises a V_{H} sequence comprising a Kabat CDR-H2 sequence comprising, consisting of, or consisting essentially of SEQ ID NO: 68. In some embodiments, the binding domain capable of binding to an HLA-G epitope comprises a V_{H} sequence comprising a Kabat CDR-H2 sequence comprising, consisting of, or consisting essentially of SEQ ID NO: 69. In some embodiments, the binding domain capable of binding to an HLA-G epitope comprises a V_{H} sequence comprising a Kabat CDR-H2 sequence comprising, consisting of, or consisting essentially of SEQ ID NO: 70. In some embodiments, the binding domain capable of binding to an HLA-G epitope comprises a V_{H} sequence comprising a Kabat CDR-H2 sequence comprising, consisting of, or consisting essentially of SEQ ID NO: 71.

### Kabat CDR-H1

In some embodiments, the binding domain capable of binding to an HLA-G epitope comprises a V_{H} sequence comprising a Kabat CDR-H1 sequence comprising, consisting of, or consisting essentially of a sequence selected from SEQ ID NOS: 18-34. In some embodiments, the binding domain capable of binding to an HLA-G epitope comprises a V_{H} sequence comprising a Kabat CDR-H1 sequence comprising, consisting of, or consisting essentially of SEQ ID NO: 18. In some embodiments, the binding domain capable of binding to an HLA-G epitope comprises a V_{H} sequence comprising a Kabat CDR-H1 sequence comprising, consisting of, or consisting essentially of SEQ ID NO: 19. In some embodiments, the binding domain capable of binding to an HLA-G epitope comprises a V_{H} sequence comprising a Kabat CDR-H1 sequence comprising, consisting of, or consisting essentially of SEQ ID NO: 20. In some embodiments, the binding domain capable of binding to an HLA-G epitope comprises a V_{H} sequence comprising a Kabat CDR-H1 sequence comprising, consisting of, or consisting essentially of SEQ ID NO: 21. In some embodiments, the binding domain capable of binding to an HLA-G epitope comprises a V_{H} sequence comprising a Kabat CDR-H1 sequence comprising, consisting of, or consisting essentially of SEQ ID NO: 22. In some embodiments, the binding domain capable of binding to an HLA-G epitope comprises a V_{H} sequence comprising a Kabat CDR-H1 sequence comprising, consisting of, or consisting essentially of SEQ ID NO: 23. In some embodiments, the binding domain capable of binding to an HLA-G epitope comprises a V_{H} sequence comprising a Kabat CDR-H1 sequence comprising, consisting of, or consisting essentially of SEQ ID NO: 24. In some embodiments, the binding domain capable of binding to an HLA-G epitope comprises a V_{H} sequence comprising a Kabat CDR-H1 sequence comprising, consisting of, or consisting essentially of SEQ ID NO: 25. In some embodiments, the binding domain capable of binding to an HLA-G epitope comprises a V_{H} sequence comprising a Kabat CDR-H1 sequence comprising, consisting of, or consisting essentially of SEQ ID NO: 26. In some embodiments, the binding domain capable of binding to an HLA-G epitope comprises a V_{H} sequence comprising a Kabat CDR-H1 sequence comprising, consisting of, or consisting essentially of SEQ ID NO: 27. In some embodiments, the binding domain capable of binding to an HLA-G epitope comprises a V_{H} sequence comprising a Kabat CDR-H1 sequence comprising, consisting of, or consisting essentially of SEQ ID NO: 28. In some embodiments, the binding domain capable of binding to an HLA-G epitope comprises a V_{H} sequence comprising a Kabat CDR-H1 sequence comprising, consisting of, or consisting essentially of SEQ ID NO: 29. In some embodiments, the binding domain capable of binding to an HLA-G epitope comprises a V_{H} sequence comprising a Kabat CDR-H1 sequence comprising, consisting of, or consisting essentially of SEQ ID NO: 30. In some embodiments, the binding domain capable of binding to an HLA-G epitope comprises a V_{H} sequence comprising a Kabat CDR-H1 sequence comprising, consisting of, or consisting essentially of SEQ ID NO: 31. In some embodiments, the binding domain capable of binding to an HLA-G epitope comprises a V_{H} sequence comprising a Kabat CDR-H1 sequence comprising, consisting of, or consisting essentially of SEQ ID NO: 32. In some embodiments, the binding domain capable of binding to an HLA-G epitope comprises a V_{H} sequence comprising a Kabat CDR-H1 sequence comprising, consisting of, or consisting essentially of SEQ ID NO: 33. In some embodiments, the binding domain capable of binding to an HLA-G epitope comprises a V_{H} sequence comprising a Kabat CDR-H1 sequence comprising, consisting of, or consisting essentially of SEQ ID NO: 34.

### Kabat CDR-H3+Kabat CDR-H2

In some embodiments, the binding domain capable of binding to an HLA-G epitope comprises a V_{H} sequence comprising a Kabat CDR-H3 sequence comprising, consisting of, or consisting essentially of a sequence selected from SEQ ID NOS: 76-101, and a Kabat CDR-H2 sequence comprising, consisting of, or consisting essentially of a sequence selected from SEQ ID NOS: 54-71. In some embodiments, the Kabat CDR-H3 sequence and the Kabat CDR-H2 sequence are both from a single illustrative V_{H} sequence provided in this disclosure. For example, in some embodiments, the Kabat CDR-H3 and Kabat CDR-H2 are both from a single illustrative V_{H} sequence selected from SEQ ID NOS: 170-200.

Kabat CDR-H3+Kabat CDR-H1

In some embodiments, the binding domain capable of binding to an HLA-G epitope comprises a V_{H} sequence comprising a Kabat CDR-H3 sequence comprising, consisting of, or consisting essentially of a sequence selected from SEQ ID NOS: 76-101, and a Kabat CDR-H1 sequence comprising, consisting of, or consisting essentially of a sequence selected from SEQ ID NOS: 18-34. In some embodiments, the Kabat CDR-H3 sequence and the Kabat CDR-H1 sequence are both from a single illustrative V_{H} sequence provided in this disclosure. For example, in some embodiments, the Kabat CDR-H3 and Kabat CDR-H1 are both from a single illustrative V_{H} sequence selected from SEQ ID NOS: 170-200.

### Kabat CDR-H1+Kabat CDR-H2

In some embodiments, the binding domain capable of binding to an HLA-G epitope comprises a V_{H} sequence comprising a Kabat CDR-H1 sequence comprising, consisting of, or consisting essentially of a sequence selected from SEQ ID NOS: 18-34 and a Kabat CDR-H2 sequence comprising, consisting of, or consisting essentially of a sequence selected from SEQ ID NOS: 54-71. In some embodiments, the Kabat CDR-H1 sequence and the Kabat CDR-H2 sequence are both from a single illustrative V_{H} sequence provided in this disclosure. For example, in some embodiments, the Kabat CDR-H1 and Kabat CDR-H2 are both from a single illustrative V_{H} sequence selected from SEQ ID NOS: 170-200.

### Kabat CDR-H1+Kabat CDR-H2+Kabat CDR-H3

In some embodiments, the binding domain capable of binding to an HLA-G epitope comprises a V_{H} sequence comprising a Kabat CDR-H1 sequence comprising, consisting of, or consisting essentially of a sequence selected from SEQ ID NOS: 18-34, a Kabat CDR-H2 sequence comprising, consisting of, or consisting essentially of a sequence selected from SEQ ID NOS: 54-71, and a Kabat CDR-H3 sequence comprising, consisting of, or consisting essentially of a sequence selected from SEQ ID NOS: 76-101. In some embodiments, the Kabat CDR-H1 sequence, Kabat CDR-H2 sequence, and Kabat CDR-H3 sequence are all from a single illustrative V_{H} sequence provided in this disclosure. For example, in some embodiments, the Kabat CDR-H1, Kabat CDR-H2, and Kabat CDR-H3 are all from a single illustrative V_{H} sequence selected from SEQ ID NOS: 170-200.

In some embodiments, the binding domain capable of binding to an HLA-G epitope comprises a V_{H} sequence comprising a Kabat CDR-H1 sequence comprising SEQ ID NO: 18, a Kabat CDR-H2 sequence comprising SEQ ID NO: 54, and a Kabat CDR-H3 sequence comprising SEQ ID NO: 76. In some embodiments, the binding domain capable of binding to an HLA-G epitope comprises a V_{H} sequence comprising a Kabat CDR-H1 sequence comprising SEQ ID NO: 19, a Kabat CDR-H2 sequence comprising SEQ ID NO: 55, and a Kabat CDR-H3 sequence comprising SEQ ID NO: 77. In some embodiments, the binding domain capable of binding to an HLA-G epitope comprises a V_{H} sequence comprising a Kabat CDR-H1 sequence comprising SEQ ID NO: 20, a Kabat CDR-H2 sequence comprising SEQ ID NO: 56, and a Kabat CDR-H3 sequence comprising SEQ ID NO: 78. In some embodiments, the binding domain capable of binding to an HLA-G epitope comprises a V_{H} sequence comprising a Kabat CDR-H1 sequence comprising SEQ ID NO: 21, a Kabat CDR-H2 sequence comprising SEQ ID NO: 57, and a Kabat CDR-H3 sequence comprising SEQ ID NO: 79. In some embodiments, the binding domain capable of binding to an HLA-G epitope comprises a V_{H} sequence comprising a Kabat CDR-H1 sequence comprising SEQ ID NO: 22, a Kabat CDR-H2 sequence comprising SEQ ID NO: 58, and a Kabat CDR-H3 sequence comprising SEQ ID NO: 80. In some embodiments, the binding domain capable of binding to an HLA-G epitope comprises a V_{H} sequence comprising a Kabat CDR-H1 sequence comprising SEQ ID NO: 21, a Kabat CDR-H2 sequence comprising SEQ ID NO: 57, and a Kabat CDR-H3 sequence comprising SEQ ID NO: 76. In some embodiments, the binding domain capable of binding to an HLA-G epitope comprises a V_{H} sequence comprising a Kabat CDR-H1 sequence comprising SEQ ID NO: 23, a Kabat CDR-H2 sequence comprising SEQ ID NO: 59, and a Kabat CDR-H3 sequence comprising SEQ ID NO: 76. In some embodiments, the binding domain capable of binding to an HLA-G epitope comprises a V_{H} sequence comprising a Kabat CDR-H1 sequence comprising SEQ ID NO: 23, a Kabat CDR-H2 sequence comprising SEQ ID NO: 60, and a Kabat CDR-H3 sequence comprising SEQ ID NO: 81. In some embodiments, the binding domain capable of binding to an HLA-G epitope comprises a V_{H} sequence comprising a Kabat CDR-H1 sequence comprising SEQ ID NO: 23, a Kabat CDR-H2 sequence comprising SEQ ID NO: 59, and a Kabat CDR-H3 sequence comprising SEQ ID NO: 82. In some embodiments, the binding domain capable of binding to an HLA-G epitope comprises a V_{H} sequence comprising a Kabat CDR-H1 sequence comprising SEQ ID NO: 23, a Kabat CDR-H2 sequence comprising SEQ ID NO: 59, and a Kabat CDR-H3 sequence comprising SEQ ID NO: 76. In some embodiments, the binding domain capable of binding to an HLA-G epitope comprises a V_{H} sequence comprising a Kabat CDR-H1 sequence comprising SEQ ID NO: 23, a Kabat CDR-H2 sequence comprising SEQ ID NO: 59, and a Kabat CDR-H3 sequence comprising SEQ ID NO: 81. In some embodiments, the binding domain capable of binding to an HLA-G epitope comprises a V_{H} sequence comprising a Kabat CDR-H1 sequence comprising SEQ ID NO: 21, a Kabat CDR-H2 sequence comprising SEQ ID NO: 57, and a Kabat CDR-H3 sequence comprising SEQ ID NO: 83. In some embodiments, the binding domain capable of binding to an HLA-G epitope comprises a V_{H} sequence comprising a Kabat CDR-H1 sequence comprising SEQ ID NO: 21, a Kabat CDR-H2 sequence comprising SEQ ID NO: 57, and a Kabat CDR-H3 sequence comprising SEQ ID NO: 84. In some embodiments, the binding domain capable of binding to an HLA-G epitope comprises a V_{H} sequence comprising a Kabat CDR-H1 sequence comprising SEQ ID NO: 24, a Kabat CDR-H2 sequence comprising SEQ ID NO: 61, and a Kabat CDR-H3 sequence comprising SEQ ID NO: 85. In some embodiments, the binding domain capable of binding to an HLA-G epitope comprises a V_{H} sequence comprising a Kabat CDR-H1 sequence comprising SEQ ID NO: 24, a Kabat CDR-H2 sequence comprising SEQ ID NO: 61, and a Kabat CDR-H3 sequence comprising SEQ ID NO: 86. In some embodiments, the binding domain capable of binding to an HLA-G epitope comprises a V_{H} sequence comprising a Kabat CDR-H1 sequence comprising SEQ ID NO: 25, a Kabat CDR-H2 sequence comprising SEQ ID NO: 62, and a Kabat CDR-H3 sequence comprising SEQ ID NO: 87. In some embodiments, the binding domain capable of binding to an HLA-G epitope comprises a V_{H} sequence comprising a Kabat CDR-H1 sequence comprising SEQ ID NO: 26, a Kabat CDR-H2 sequence comprising SEQ ID NO: 63, and a Kabat CDR-H3 sequence comprising SEQ ID NO: 88. In some embodiments, the binding domain capable of binding to an HLA-G epitope comprises a V_{H} sequence comprising a Kabat CDR-H1 sequence comprising SEQ ID NO: 26, a Kabat CDR-H2 sequence comprising SEQ ID NO: 63, and a Kabat CDR-H3 sequence comprising SEQ ID NO: 89. In some embodiments, the binding domain capable of binding to an HLA-G epitope comprises a V_{H} sequence comprising a Kabat CDR-H1 sequence comprising SEQ ID NO: 25, a Kabat CDR-H2 sequence comprising SEQ ID NO: 63, and a Kabat CDR-H3 sequence comprising SEQ ID NO: 90. In some embodiments, the binding domain capable of binding to an HLA-G epitope comprises a V_{H} sequence comprising a Kabat CDR-H1 sequence comprising SEQ ID NO: 27, a Kabat CDR-H2 sequence comprising SEQ ID NO: 64, and a Kabat CDR-H3 sequence comprising SEQ ID NO: 90. In some embodiments, the binding domain capable of binding to an HLA-G epitope comprises a V_{H} sequence comprising a Kabat CDR-H1 sequence comprising SEQ ID NO: 28, a Kabat CDR-H2 sequence comprising SEQ ID NO: 62, and a Kabat CDR-H3 sequence comprising SEQ ID NO: 91. In some embodiments, the binding domain capable of binding to an HLA-G epitope comprises a V_{H} sequence comprising a Kabat CDR-H1 sequence comprising SEQ ID NO: 29, a Kabat CDR-H2 sequence comprising SEQ ID NO: 64, and a Kabat CDR-H3 sequence comprising SEQ ID NO: 92. In some embodiments, the binding domain capable of binding to an HLA-G epitope comprises a V_{H} sequence comprising a Kabat CDR-H1 sequence comprising SEQ ID NO: 25, a Kabat CDR-H2 sequence comprising SEQ ID NO: 65, and a Kabat CDR-H3 sequence comprising SEQ ID NO: 93. In some embodiments, the binding domain capable of binding to an HLA-G epitope comprises a V_{H} sequence comprising a Kabat CDR-H1 sequence comprising SEQ ID NO: 30, a Kabat CDR-H2 sequence comprising SEQ ID NO: 66, and a Kabat CDR-H3 sequence comprising SEQ ID NO: 94. In some embodiments, the binding domain capable of binding to an HLA-G epitope comprises a V_{H} sequence comprising a Kabat CDR-H1 sequence comprising SEQ ID NO: 31, a Kabat CDR-H2 sequence comprising SEQ ID NO: 67, and a Kabat CDR-H3 sequence comprising SEQ ID NO: 95. In some embodiments, the binding domain capable of binding to an HLA-G epitope comprises a V_{H} sequence comprising a Kabat CDR-H1 sequence comprising SEQ ID NO: 32, a Kabat CDR-H2 sequence comprising SEQ ID NO: 68, and a Kabat CDR-H3 sequence comprising SEQ ID NO: 96. In some embodiments, the binding domain capable of binding to an HLA-G epitope comprises a V_{H} sequence comprising a Kabat CDR-H1 sequence comprising SEQ ID NO: 33, a Kabat CDR-H2 sequence comprising SEQ ID NO: 69, and a Kabat CDR-H3 sequence comprising SEQ ID NO: 97. In some embodiments, the binding domain capable of binding to an HLA-G epitope comprises a V_{H} sequence comprising a Kabat CDR-H1 sequence comprising SEQ ID NO: 34, a Kabat CDR-H2 sequence comprising SEQ ID NO: 70, and a Kabat CDR-H3 sequence comprising SEQ ID NO: 98. In some embodiments, the binding domain capable of binding to an HLA-G epitope comprises a V_{H} sequence comprising a Kabat CDR-H1 sequence comprising SEQ ID NO: 18, a Kabat CDR-H2 sequence comprising SEQ ID NO: 54, and a Kabat CDR-H3 sequence comprising SEQ ID NO: 99. In some embodiments, the binding domain capable of binding to an HLA-G epitope comprises a V_{H} sequence comprising a Kabat CDR-H1 sequence comprising SEQ ID NO: 31, a Kabat CDR-H2 sequence comprising SEQ ID NO: 71, and a Kabat CDR-H3 sequence comprising SEQ ID NO: 100. In some embodiments, the binding domain capable of binding to an HLA-G epitope comprises a V_{H} sequence comprising a Kabat CDR-H1 sequence comprising SEQ ID NO: 24, a Kabat CDR-H2 sequence comprising SEQ ID NO: 61, and a Kabat CDR-H3 sequence comprising SEQ ID NO: 101.

### Variants of V_{H} Sequences Comprising Illustrative Kabat CDRs

In some embodiments, the V_{H} sequences provided herein comprise a variant of an illustrative Kabat CDR-H3, CDR-H2, and/or CDR-H1 sequence provided in this disclosure.

In some embodiments, the Kabat CDR-H3 sequence comprises, consists of, or consists essentially of a variant of an illustrative Kabat CDR-H3 sequence provided in this disclosure. In some embodiments, the Kabat CDR-H3 sequence comprises, consists of, or consists essentially of a sequence having at least 70%, 75%, 80%, 85%, 90%, or 95% identity with any of the illustrative Kabat CDR-H3 sequences provided in this disclosure. In some embodiments, the Kabat CDR-H3 sequence comprises, consists of, or consists essentially of any of the illustrative Kabat CDR-H3 sequences provided in this disclosure, with 1, 2, or 3 amino acid substitutions. In some embodiments, the amino acid substitutions are conservative amino acid substitutions.

In some embodiments, the Kabat CDR-H2 sequence comprises, consists of, or consists essentially of a variant of an illustrative Kabat CDR-H2 sequence provided in this disclosure. In some embodiments, the Kabat CDR-H2 sequence comprises, consists of, or consists essentially of a sequence having at least 70%, 75%, 80%, 85%, 90%, or 95% identity with any of the illustrative Kabat CDR-H2 sequences provided in this disclosure. In some embodiments, the Kabat CDR-H2 sequence comprises, consists of, or consists essentially of any of the illustrative Kabat CDR-H2 sequences provided in this disclosure, with 1, 2, or 3 amino acid substitutions. In some embodiments, the amino acid substitutions are conservative amino acid substitutions.

In some embodiments, the Kabat CDR-H1 sequence comprises, consists of, or consists essentially of a variant of an illustrative Kabat CDR-H1 sequence provided in this disclosure. In some embodiments, the Kabat CDR-H1 sequence comprises, consists of, or consists essentially of a sequence having at least 70%, 75%, 80%, 85%, 90%, or 95% identity with any of the illustrative Kabat CDR-H1 sequences provided in this disclosure. In some embodiments, the Kabat CDR-H1 sequence comprises, consists of, or consists essentially of any of the illustrative Kabat CDR-H1 sequences provided in this disclosure, with 1, 2, or 3 amino acid substitutions. In some embodiments, the amino acid substitutions are conservative amino acid substitutions.

### V_{H} Sequences Comprising Illustrative Chothia CDRs

In some embodiments, the binding domain capable of binding to an HLA-G epitope comprises a V_{H} sequence comprising one or more Chothia CDR-H sequences comprising, consisting of, or consisting essentially of one or more illustrative Chothia CDR-H sequences provided in this disclosure, and variants thereof.

### Chothia CDR-H3

In some embodiments, the binding domain capable of binding to an HLA-G epitope comprises a V_{H} sequence comprising a Chothia CDR-H3 sequence comprising, consisting of, or consisting essentially of a sequence selected from SEQ ID NOS: 76-101. In some embodiments, the binding domain capable of binding to an HLA-G epitope comprises a V_{H} sequence comprising a Chothia CDR-H3 sequence comprising, consisting of, or consisting essentially of SEQ ID NO: 76. In some embodiments, the binding domain capable of binding to an HLA-G epitope comprises a V_{H} sequence comprising a Chothia CDR-H3 sequence comprising, consisting of, or consisting essentially of SEQ ID NO: 77. In some embodiments, the binding domain capable of binding to an HLA-G epitope comprises a V_{H} sequence comprising a Chothia CDR-H3 sequence comprising, consisting of, or consisting essentially of SEQ ID NO: 78. In some embodiments, the binding domain capable of binding to an HLA-G epitope comprises a V_{H} sequence comprising a Chothia CDR-H3 sequence comprising, consisting of, or consisting essentially of SEQ ID NO: 79. In some embodiments, the binding domain capable of binding to an HLA-G epitope comprises a V_{H} sequence comprising a Chothia CDR-H3 sequence comprising, consisting of, or consisting essentially of SEQ ID NO: 80. In some embodiments, the binding domain capable of binding to an HLA-G epitope comprises a V_{H} sequence comprising a Chothia CDR-H3 sequence comprising, consisting of, or consisting essentially of SEQ ID NO: 81. In some embodiments, the binding domain capable of binding to an HLA-G epitope comprises a V_{H} sequence comprising a Chothia CDR-H3 sequence comprising, consisting of, or consisting essentially of SEQ ID NO: 82. In some embodiments, the binding domain capable of binding to an HLA-G epitope comprises a V_{H} sequence comprising a Chothia CDR-H3 sequence comprising, consisting of, or consisting essentially of SEQ ID NO: 83. In some embodiments, the binding domain capable of binding to an HLA-G epitope comprises a V_{H} sequence comprising a Chothia CDR-H3 sequence comprising, consisting of, or consisting essentially of SEQ ID NO: 84. In some embodiments, the binding domain capable of binding to an HLA-G epitope comprises a V_{H} sequence comprising a Chothia CDR-H3 sequence comprising, consisting of, or consisting essentially of SEQ ID NO: 85. In some embodiments, the binding domain capable of binding to an HLA-G epitope comprises a V_{H} sequence comprising a Chothia CDR-H3 sequence comprising, consisting of, or consisting essentially of SEQ ID NO: 86. In some embodiments, the binding domain capable of binding to an HLA-G epitope comprises a V_{H} sequence comprising a Chothia CDR-H3 sequence comprising, consisting of, or consisting essentially of SEQ ID NO: 87. In some embodiments, the binding domain capable of binding to an HLA-G epitope comprises a V_{H} sequence comprising a Chothia CDR-H3 sequence comprising, consisting of, or consisting essentially of SEQ ID NO: 88. In some embodiments, the binding domain capable of binding to an HLA-G epitope comprises a V_{H} sequence comprising a Chothia CDR-H3 sequence comprising, consisting of, or consisting essentially of SEQ ID NO: 89. In some embodiments, the binding domain capable of binding to an HLA-G epitope comprises a V_{H} sequence comprising a Chothia CDR-H3 sequence comprising, consisting of, or consisting essentially of SEQ ID NO: 90. In some embodiments, the binding domain capable of binding to an HLA-G epitope comprises a V_{H} sequence comprising a Chothia CDR-H3 sequence comprising, consisting of, or consisting essentially of SEQ ID NO: 91. In some embodiments, the binding domain capable of binding to an HLA-G epitope comprises a V_{H} sequence comprising a Chothia CDR-H3 sequence comprising, consisting of, or consisting essentially of SEQ ID NO: 92. In some embodiments, the binding domain capable of binding to an HLA-G epitope comprises a V_{H} sequence comprising a Chothia CDR-H3 sequence comprising, consisting of, or consisting essentially of SEQ ID NO: 93. In some embodiments, the binding domain capable of binding to an HLA-G epitope comprises a V_{H} sequence comprising a Chothia CDR-H3 sequence comprising, consisting of, or consisting essentially of SEQ ID NO: 94. In some embodiments, the binding domain capable of binding to an HLA-G epitope comprises a V_{H} sequence comprising a Chothia CDR-H3 sequence comprising, consisting of, or consisting essentially of SEQ ID NO: 95. In some embodiments, the binding domain capable of binding to an HLA-G epitope comprises a V_{H} sequence comprising a Chothia CDR-H3 sequence comprising, consisting of, or consisting essentially of SEQ ID NO: 96. In some embodiments, the binding domain capable of binding to an HLA-G epitope comprises a V_{H} sequence comprising a Chothia CDR-H3 sequence comprising, consisting of, or consisting essentially of SEQ ID NO: 97. In some embodiments, the binding domain capable of binding to an HLA-G epitope comprises a V_{H} sequence comprising a Chothia CDR-H3 sequence comprising, consisting of, or consisting essentially of SEQ ID NO: 98. In some embodiments, the binding domain capable of binding to an HLA-G epitope comprises a V_{H} sequence comprising a Chothia CDR-H3 sequence comprising, consisting of, or consisting essentially of SEQ ID NO: 99. In some embodiments, the binding domain capable of binding to an HLA-G epitope comprises a V_{H} sequence comprising a Chothia CDR-H3 sequence comprising, consisting of, or consisting essentially of SEQ ID NO: 100. In some embodiments, the binding domain capable of binding to an HLA-G epitope comprises a V_{H} sequence comprising a Chothia CDR-H3 sequence comprising, consisting of, or consisting essentially of SEQ ID NO: 101.

### Chothia CDR-H2

In some embodiments, the binding domain capable of binding to an HLA-G epitope comprises a V_{H} sequence comprising a Chothia CDR-H2 sequence comprising, consisting of, or consisting essentially of a sequence selected from SEQ ID NOS: 38-50. In some embodiments, the binding domain capable of binding to an HLA-G epitope comprises a V_{H} sequence comprising a Chothia CDR-H2 sequence comprising, consisting of, or consisting essentially of SEQ ID NO: 38. In some embodiments, the binding domain capable of binding to an HLA-G epitope comprises a V_{H} sequence comprising a Chothia CDR-H2 sequence comprising, consisting of, or consisting essentially of SEQ ID NO: 39. In some embodiments, the binding domain capable of binding to an HLA-G epitope comprises a V_{H} sequence comprising a Chothia CDR-H2 sequence comprising, consisting of, or consisting essentially of SEQ ID NO: 40. In some embodiments, the binding domain capable of binding to an HLA-G epitope comprises a V_{H} sequence comprising a Chothia CDR-H2 sequence comprising, consisting of, or consisting essentially of SEQ ID NO: 41. In some embodiments, the binding domain capable of binding to an HLA-G epitope comprises a V_{H} sequence comprising a Chothia CDR-H2 sequence comprising, consisting of, or consisting essentially of SEQ ID NO: 42. In some embodiments, the binding domain capable of binding to an HLA-G epitope comprises a V_{H} sequence comprising a Chothia CDR-H2 sequence comprising, consisting of, or consisting essentially of SEQ ID NO: 43. In some embodiments, the binding domain capable of binding to an HLA-G epitope comprises a V_{H} sequence comprising a Chothia CDR-H2 sequence comprising, consisting of, or consisting essentially of SEQ ID NO: 44. In some embodiments, the binding domain capable of binding to an HLA-G epitope comprises a V_{H} sequence comprising a Chothia CDR-H2 sequence comprising, consisting of, or consisting essentially of SEQ ID NO: 45. In some embodiments, the binding domain capable of binding to an HLA-G epitope comprises a V_{H} sequence comprising a Chothia CDR-H2 sequence comprising, consisting of, or consisting essentially of SEQ ID NO: 46. In some embodiments, the binding domain capable of binding to an HLA-G epitope comprises a V_{H} sequence comprising a Chothia CDR-H2 sequence comprising, consisting of, or consisting essentially of SEQ ID NO: 47. In some embodiments, the binding domain capable of binding to an HLA-G epitope comprises a V_{H} sequence comprising a Chothia CDR-H2 sequence comprising, consisting of, or consisting essentially of SEQ ID NO: 48. In some embodiments, the binding domain capable of binding to an HLA-G epitope comprises a V_{H} sequence comprising a Chothia CDR-H2 sequence comprising, consisting of, or consisting essentially of SEQ ID NO: 49. In some embodiments, the binding domain capable of binding to an HLA-G epitope comprises a V_{H} sequence comprising a Chothia CDR-H2 sequence comprising, consisting of, or consisting essentially of SEQ ID NO: 50.

### Chothia CDR-H1

In some embodiments, the binding domain capable of binding to an HLA-G epitope comprises a V_{H} sequence comprising a Chothia CDR-H1 sequence comprising, consisting of, or consisting essentially of a sequence selected from SEQ ID NOS: 1-14. In some embodiments, the binding domain capable of binding to an HLA-G epitope comprises a V_{H} sequence comprising a Chothia CDR-H1 sequence comprising, consisting of, or consisting essentially of SEQ ID NO: 1. In some embodiments, the binding domain capable of binding to an HLA-G epitope comprises a V_{H} sequence comprising a Chothia CDR-H1 sequence comprising, consisting of, or consisting essentially of SEQ ID NO: 2. In some embodiments, the binding domain capable of binding to an HLA-G epitope comprises a V_{H} sequence comprising a Chothia CDR-H1 sequence comprising, consisting of, or consisting essentially of SEQ ID NO: 3. In some embodiments, the binding domain capable of binding to an HLA-G epitope comprises a V_{H} sequence comprising a Chothia CDR-H1 sequence comprising, consisting of, or consisting essentially of SEQ ID NO: 4. In some embodiments, the binding domain capable of binding to an HLA-G epitope comprises a V_{H} sequence comprising a Chothia CDR-H1 sequence comprising, consisting of, or consisting essentially of SEQ ID NO: 5. In some embodiments, the binding domain capable of binding to an HLA-G epitope comprises a V_{H} sequence comprising a Chothia CDR-H1 sequence comprising, consisting of, or consisting essentially of SEQ ID NO: 6. In some embodiments, the binding domain capable of binding to an HLA-G epitope comprises a V_{H} sequence comprising a Chothia CDR-H1 sequence comprising, consisting of, or consisting essentially of SEQ ID NO: 7. In some embodiments, the binding domain capable of binding to an HLA-G epitope comprises a V_{H} sequence comprising a Chothia CDR-H1 sequence comprising, consisting of, or consisting essentially of SEQ ID NO: 8. In some embodiments, the binding domain capable of binding to an HLA-G epitope comprises a V_{H} sequence comprising a Chothia CDR-H1 sequence comprising, consisting of, or consisting essentially of SEQ ID NO: 9. In some embodiments, the binding domain capable of binding to an HLA-G epitope comprises a V_{H} sequence comprising a Chothia CDR-H1 sequence comprising, consisting of, or consisting essentially of SEQ ID NO: 10. In some embodiments, the binding domain capable of binding to an HLA-G epitope comprises a V_{H} sequence comprising a Chothia CDR-H1 sequence comprising, consisting of, or consisting essentially of SEQ ID NO: 11. In some embodiments, the binding domain capable of binding to an HLA-G epitope comprises a V_{H} sequence comprising a Chothia CDR-H1 sequence comprising, consisting of, or consisting essentially of SEQ ID NO: 12. In some embodiments, the binding domain capable of binding to an HLA-G epitope comprises a V_{H} sequence comprising a Chothia CDR-H1 sequence comprising, consisting of, or consisting essentially of SEQ ID NO: 13. In some embodiments, the binding domain capable of binding to an HLA-G epitope comprises a V_{H} sequence comprising a Chothia CDR-H1 sequence comprising, consisting of, or consisting essentially of SEQ ID NO: 14.

### Chothia CDR-H3+Chothia CDR-H2

In some embodiments, the binding domain capable of binding to an HLA-G epitope comprises a V_{H} sequence comprising a Chothia CDR-H3 sequence comprising, consisting of, or consisting essentially of a sequence selected from SEQ ID NOS: 76-101, and a Chothia CDR-H2 sequence comprising, consisting of, or consisting essentially of a sequence selected from SEQ ID NOS: 38-50. In some embodiments, the Chothia CDR-H3 sequence and the Chothia CDR-H2 sequence are both from a single illustrative V_{H} sequence provided in this disclosure. For example, in some embodiments, the Chothia CDR-H3 and Chothia CDR-H2 are both from a single illustrative V_{H} sequence selected from SEQ ID NOS: 170-200.

### Chothia CDR-H3+Chothia CDR-H1

In some embodiments, the binding domain capable of binding to an HLA-G epitope comprises a V_{H} sequence comprising a Chothia CDR-H3 sequence comprising, consisting of, or consisting essentially of a sequence selected from SEQ ID NOS: 76-101, and a Chothia CDR-H1 sequence comprising, consisting of, or consisting essentially of a sequence selected from SEQ ID NOS: 1-14. In some embodiments, the Chothia CDR-H3 sequence and the Chothia CDR-H1 sequence are both from a single illustrative V_{H} sequence provided in this disclosure. For example, in some embodiments, the Chothia CDR-H3 and Chothia CDR-H1 are both from a single illustrative V_{H} sequence selected from SEQ ID NOS: 170-200.

### Chothia CDR-H1+Chothia CDR-H2

In some embodiments, the binding domain capable of binding to an HLA-G epitope comprises a V_{H} sequence comprising a Chothia CDR-H1 sequence comprising, consisting of, or consisting essentially of a sequence selected from SEQ ID NOS: 1-14 and a Chothia CDR-H2 sequence comprising, consisting of, or consisting essentially of a sequence selected from SEQ ID NOS: 38-50. In some embodiments, the Chothia CDR-H1 sequence and the Chothia CDR-H2 sequence are both from a single illustrative V_{H} sequence provided in this disclosure. For example, in some embodiments, the Chothia CDR-H1 and Chothia CDR-H2 are both from a single illustrative V_{H} sequence selected from SEQ ID NOS: 170-200.

### Chothia CDR-H1+Chothia CDR-H2+Chothia CDR-H3

In some embodiments, the binding domain capable of binding to an HLA-G epitope comprises a V_{H} sequence comprising a Chothia CDR-H1 sequence comprising, consisting of, or consisting essentially of a sequence selected from SEQ ID NOS: 1-14, a Chothia CDR-H2 sequence comprising, consisting of, or consisting essentially of a sequence selected from SEQ ID NOS: 38-50, and a Chothia CDR-H3 sequence comprising, consisting of, or consisting essentially of a sequence selected from SEQ ID NOS: 76-101. In some embodiments, the Chothia CDR-H1 sequence, Chothia CDR-H2 sequence, and Chothia CDR-H3 sequence are all from a single illustrative V_{H} sequence provided in this disclosure. For example, in some embodiments, the Chothia CDR-H1, Chothia CDR-H2, and Chothia CDR-H3 are all from a single illustrative V_{H} sequence selected from SEQ ID NOS: 170-200.

In some embodiments, the binding domain capable of binding to an HLA-G epitope comprises a V_{H} sequence comprising a Chothia CDR-H1 sequence comprising SEQ ID NO: 1, a Chothia CDR-H2 sequence comprising SEQ ID NO: 38, and a Chothia CDR-H3 sequence comprising SEQ ID NO: 76. In some embodiments, the binding domain capable of binding to an HLA-G epitope comprises a V_{H} sequence comprising a Chothia CDR-H1 sequence comprising SEQ ID NO: 2, a Chothia CDR-H2 sequence comprising SEQ ID NO: 39, and a Chothia CDR-H3 sequence comprising SEQ ID NO: 77. In some embodiments, the binding domain capable of binding to an HLA-G epitope comprises a V_{H} sequence comprising a Chothia CDR-H1 sequence comprising SEQ ID NO: 2, a Chothia CDR-H2 sequence comprising SEQ ID NO: 40, and a Chothia CDR-H3 sequence comprising SEQ ID NO: 78. In some embodiments, the binding domain capable of binding to an HLA-G epitope comprises a V_{H} sequence comprising a Chothia CDR-H1 sequence comprising SEQ ID NO: 3, a Chothia CDR-H2 sequence comprising SEQ ID NO: 41, and a Chothia CDR-H3 sequence comprising SEQ ID NO: 79. In some embodiments, the binding domain capable of binding to an HLA-G epitope comprises a V_{H} sequence comprising a Chothia CDR-H1 sequence comprising SEQ ID NO: 4, a Chothia CDR-H2 sequence comprising SEQ ID NO: 41, and a Chothia CDR-H3 sequence comprising SEQ ID NO: 80. In some embodiments, the binding domain capable of binding to an HLA-G epitope comprises a V_{H} sequence comprising a Chothia CDR-H1 sequence comprising SEQ ID NO: 3, a Chothia CDR-H2 sequence comprising SEQ ID NO: 41, and a Chothia CDR-H3 sequence comprising SEQ ID NO: 76. In some embodiments, the binding domain capable of binding to an HLA-G epitope comprises a V_{H} sequence comprising a Chothia CDR-H1 sequence comprising SEQ ID NO: 2, a Chothia CDR-H2 sequence comprising SEQ ID NO: 42, and a Chothia CDR-H3 sequence comprising SEQ ID NO: 76. In some embodiments, the binding domain capable of binding to an HLA-G epitope comprises a V_{H} sequence comprising a Chothia CDR-H1 sequence comprising SEQ ID NO: 2, a Chothia CDR-H2 sequence comprising SEQ ID NO: 43, and a Chothia CDR-H3 sequence comprising SEQ ID NO: 81. In some embodiments, the binding domain capable of binding to an HLA-G epitope comprises a V_{H} sequence comprising a Chothia CDR-H1 sequence comprising SEQ ID NO: 2, a Chothia CDR-H2 sequence comprising SEQ ID NO: 42, and a Chothia CDR-H3 sequence comprising SEQ ID NO: 82. In some embodiments, the binding domain capable of binding to an HLA-G epitope comprises a V_{H} sequence comprising a Chothia CDR-H1 sequence comprising SEQ ID NO: 5, a Chothia CDR-H2 sequence comprising SEQ ID NO: 42, and a Chothia CDR-H3 sequence comprising SEQ ID NO: 76. In some embodiments, the binding domain capable of binding to an HLA-G epitope comprises a V_{H} sequence comprising a Chothia CDR-H1 sequence comprising SEQ ID NO: 2, a Chothia CDR-H2 sequence comprising SEQ ID NO: 42, and a Chothia CDR-H3 sequence comprising SEQ ID NO: 81. In some embodiments, the binding domain capable of binding to an HLA-G epitope comprises a V_{H} sequence comprising a Chothia CDR-H1 sequence comprising SEQ ID NO: 3, a Chothia CDR-H2 sequence comprising SEQ ID NO: 41, and a Chothia CDR-H3 sequence comprising SEQ ID NO: 83. In some embodiments, the binding domain capable of binding to an HLA-G epitope comprises a V_{H} sequence comprising a Chothia CDR-H1 sequence comprising SEQ ID NO: 3, a Chothia CDR-H2 sequence comprising SEQ ID NO: 41, and a Chothia CDR-H3 sequence comprising SEQ ID NO: 84. In some embodiments, the binding domain capable of binding to an HLA-G epitope comprises a V_{H} sequence comprising a Chothia CDR-H1 sequence comprising SEQ ID NO: 6, a Chothia CDR-H2 sequence comprising SEQ ID NO: 44, and a Chothia CDR-H3 sequence comprising SEQ ID NO: 85. In some embodiments, the binding domain capable of binding to an HLA-G epitope comprises a V_{H} sequence comprising a Chothia CDR-H1 sequence comprising SEQ ID NO: 6, a Chothia CDR-H2 sequence comprising SEQ ID NO: 44, and a Chothia CDR-H3 sequence comprising SEQ ID NO: 86. In some embodiments, the binding domain capable of binding to an HLA-G epitope comprises a V_{H} sequence comprising a Chothia CDR-H1 sequence comprising SEQ ID NO: 7, a Chothia CDR-H2 sequence comprising SEQ ID NO: 45, and a Chothia CDR-H3 sequence comprising SEQ ID NO: 87. In some embodiments, the binding domain capable of binding to an HLA-G epitope comprises a V_{H} sequence comprising a Chothia CDR-H1 sequence comprising SEQ ID NO: 8, a Chothia CDR-H2 sequence comprising SEQ ID NO: 44, and a Chothia CDR-H3 sequence comprising SEQ ID NO: 88. In some embodiments, the binding domain capable of binding to an HLA-G epitope comprises a V_{H} sequence comprising a Chothia CDR-H1 sequence comprising SEQ ID NO: 8, a Chothia CDR-H2 sequence comprising SEQ ID NO: 44, and a Chothia CDR-H3 sequence comprising SEQ ID NO: 89. In some embodiments, the binding domain capable of binding to an HLA-G epitope comprises a V_{H} sequence comprising a Chothia CDR-H1 sequence comprising SEQ ID NO: 7, a Chothia CDR-H2 sequence comprising SEQ ID NO: 44, and a Chothia CDR-H3 sequence comprising SEQ ID NO: 90. In some embodiments, the binding domain capable of binding to an HLA-G epitope comprises a V_{H} sequence comprising a Chothia CDR-H1 sequence comprising SEQ ID NO: 9, a Chothia CDR-H2 sequence comprising SEQ ID NO: 44, and a Chothia CDR-H3 sequence comprising SEQ ID NO: 90. In some embodiments, the binding domain capable of binding to an HLA-G epitope comprises a V_{H} sequence comprising a Chothia CDR-H1 sequence comprising SEQ ID NO: 6, a Chothia CDR-H2 sequence comprising SEQ ID NO: 45, and a Chothia CDR-H3 sequence comprising SEQ ID NO: 91. In some embodiments, the binding domain capable of binding to an HLA-G epitope comprises a V_{H} sequence comprising a Chothia CDR-H1 sequence comprising SEQ ID NO: 8, a Chothia CDR-H2 sequence comprising SEQ ID NO: 44, and a Chothia CDR-H3 sequence comprising SEQ ID NO: 92. In some embodiments, the binding domain capable of binding to an HLA-G epitope comprises a V_{H} sequence comprising a Chothia CDR-H1 sequence comprising SEQ ID NO: 7, a Chothia CDR-H2 sequence comprising SEQ ID NO: 44, and a Chothia CDR-H3 sequence comprising SEQ ID NO: 93. In some embodiments, the binding domain capable of binding to an HLA-G epitope comprises a V_{H} sequence comprising a Chothia CDR-H1 sequence comprising SEQ ID NO: 10, a Chothia CDR-H2 sequence comprising SEQ ID NO: 46, and a Chothia CDR-H3 sequence comprising SEQ ID NO: 94. In some embodiments, the binding domain capable of binding to an HLA-G epitope comprises a V_{H} sequence comprising a Chothia CDR-H1 sequence comprising SEQ ID NO: 11, a Chothia CDR-H2 sequence comprising SEQ ID NO: 47, and a Chothia CDR-H3 sequence comprising SEQ ID NO: 95. In some embodiments, the binding domain capable of binding to an HLA-G epitope comprises a V_{H} sequence comprising a Chothia CDR-H1 sequence comprising SEQ ID NO: 12, a Chothia CDR-H2 sequence comprising SEQ ID NO: 48, and a Chothia CDR-H3 sequence comprising SEQ ID NO: 96. In some embodiments, the binding domain capable of binding to an HLA-G epitope comprises a V_{H} sequence comprising a Chothia CDR-H1 sequence comprising SEQ ID NO: 13, a Chothia CDR-H2 sequence comprising SEQ ID NO: 44, and a Chothia CDR-H3 sequence comprising SEQ ID NO: 97. In some embodiments, the binding domain capable of binding to an HLA-G epitope comprises a V_{H} sequence comprising a Chothia CDR-H1 sequence comprising SEQ ID NO: 14, a Chothia CDR-H2 sequence comprising SEQ ID NO: 49, and a Chothia CDR-H3 sequence comprising SEQ ID NO: 98. In some embodiments, the binding domain capable of binding to an HLA-G epitope comprises a V_{H} sequence comprising a Chothia CDR-H1 sequence comprising SEQ ID NO: 1, a Chothia CDR-H2 sequence comprising SEQ ID NO: 38, and a Chothia CDR-H3 sequence comprising SEQ ID NO: 99. In some embodiments, the binding domain capable of binding to an HLA-G epitope comprises a V_{H} sequence comprising a Chothia CDR-H1 sequence comprising SEQ ID NO: 11, a Chothia CDR-H2 sequence comprising SEQ ID NO: 50, and a Chothia CDR-H3 sequence comprising SEQ ID NO: 100. In some embodiments, the binding domain capable of binding to an HLA-G epitope comprises a V_{H} sequence comprising a Chothia CDR-H1 sequence comprising SEQ ID NO: 6, a Chothia CDR-H2 sequence comprising SEQ ID NO: 44, and a Chothia CDR-H3 sequence comprising SEQ ID NO: 101.

### Variants of V_{H} Sequences Comprising Illustrative Chothia CDRs

In some embodiments, the V_{H} sequences provided herein comprise a variant of an illustrative Chothia CDR-H3, CDR-H2, and/or CDR-H1 sequence provided in this disclosure.

In some embodiments, the Chothia CDR-H3 sequence comprises, consists of, or consists essentially of a variant of an illustrative Chothia CDR-H3 sequence provided in this disclosure. In some embodiments, the Chothia CDR-H3 sequence comprises, consists of, or consists essentially of a sequence having at least 70%, 75%, 80%, 85%, 90%, or 95% identity with any of the illustrative Chothia CDR-H3 sequences provided in this disclosure. In some embodiments, the Chothia CDR-H3 sequence comprises, consists of, or consists essentially of any of the illustrative Chothia CDR-H3 sequences provided in this disclosure, with 1, 2, or 3 amino acid substitutions. In some embodiments, the amino acid substitutions are conservative amino acid substitutions.

In some embodiments, the Chothia CDR-H2 sequence comprises, consists of, or consists essentially of a variant of an illustrative Chothia CDR-H2 sequence provided in this disclosure. In some embodiments, the Chothia CDR-H2 sequence comprises, consists of, or consists essentially of a sequence having at least 70%, 75%, 80%, 85%, 90%, or 95% identity with any of the illustrative Chothia CDR-H2 sequences provided in this disclosure. In some embodiments, the Chothia CDR-H2 sequence comprises, consists of, or consists essentially of any of the illustrative Chothia CDR-H2 sequences provided in this disclosure, with 1, 2, or 3 amino acid substitutions. In some embodiments, the amino acid substitutions are conservative amino acid substitutions.

In some embodiments, the Chothia CDR-H1 sequence comprises, consists of, or consists essentially of a variant of an illustrative Chothia CDR-H1 sequence provided in this disclosure. In some embodiments, the Chothia CDR-H1 sequence comprises, consists of, or consists essentially of a sequence having at least 70%, 75%, 80%, 85%, 90%, or 95% identity with any of the illustrative Chothia CDR-H1 sequences provided in this disclosure. In some embodiments, the Chothia CDR-H1 sequence comprises, consists of, or consists essentially of any of the illustrative Chothia CDR-H1 sequences provided in this disclosure, with 1, 2, or 3 amino acid substitutions. In some embodiments, the amino acid substitutions are conservative amino acid substitutions.

### V_{H} Sequences

In some embodiments, the binding domain capable of binding to an HLA-G epitope comprises a V_{H} sequence comprising, consisting of, or consisting essentially of a sequence selected from SEQ ID NOS: 170-200. In some embodiments, the binding domain capable of binding to an HLA-G epitope comprises a V_{H} sequence comprising, consisting of, or consisting essentially of SEQ ID NO: 170. In some embodiments, the binding domain capable of binding to an HLA-G epitope comprises a V_{H} sequence comprising, consisting of, or consisting essentially of SEQ ID NO: 171. In some embodiments, the binding domain capable of binding to an HLA-G epitope comprises a V_{H} sequence comprising, consisting of, or consisting essentially of SEQ ID NO: 172. In some embodiments, the binding domain capable of binding to an HLA-G epitope comprises a V_{H} sequence comprising, consisting of, or consisting essentially of SEQ ID NO: 173. In some embodiments, the binding domain capable of binding to an HLA-G epitope comprises a V_{H} sequence comprising, consisting of, or consisting essentially of SEQ ID NO: 174. In some embodiments, the binding domain capable of binding to an HLA-G epitope comprises a V_{H} sequence comprising, consisting of, or consisting essentially of SEQ ID NO: 175. In some embodiments, the binding domain capable of binding to an HLA-G epitope comprises a V_{H} sequence comprising, consisting of, or consisting essentially of SEQ ID NO: 176. In some embodiments, the binding domain capable of binding to an HLA-G epitope comprises a V_{H} sequence comprising, consisting of, or consisting essentially of SEQ ID NO: 177. In some embodiments, the binding domain capable of binding to an HLA-G epitope comprises a V_{H} sequence comprising, consisting of, or consisting essentially of SEQ ID NO: 178. In some embodiments, the binding domain capable of binding to an HLA-G epitope comprises a V_{H} sequence comprising, consisting of, or consisting essentially of SEQ ID NO: 179. In some embodiments, the binding domain capable of binding to an HLA-G epitope comprises a V_{H} sequence comprising, consisting of, or consisting essentially of SEQ ID NO: 180. In some embodiments, the binding domain capable of binding to an HLA-G epitope comprises a V_{H} sequence comprising, consisting of, or consisting essentially of SEQ ID NO: 181. In some embodiments, the binding domain capable of binding to an HLA-G epitope comprises a V_{H} sequence comprising, consisting of, or consisting essentially of SEQ ID NO: 182. In some embodiments, the binding domain capable of binding to an HLA-G epitope comprises a V_{H} sequence comprising, consisting of, or consisting essentially of SEQ ID NO: 183. In some embodiments, the binding domain capable of binding to an HLA-G epitope comprises a V_{H} sequence comprising, consisting of, or consisting essentially of SEQ ID NO: 184. In some embodiments, the binding domain capable of binding to an HLA-G epitope comprises a V_{H} sequence comprising, consisting of, or consisting essentially of SEQ ID NO: 185. In some embodiments, the binding domain capable of binding to an HLA-G epitope comprises a V_{H} sequence comprising, consisting of, or consisting essentially of SEQ ID NO: 186. In some embodiments, the binding domain capable of binding to an HLA-G epitope comprises a V_{H} sequence comprising, consisting of, or consisting essentially of SEQ ID NO: 187. In some embodiments, the binding domain capable of binding to an HLA-G epitope comprises a V_{H} sequence comprising, consisting of, or consisting essentially of SEQ ID NO: 188. In some embodiments, the binding domain capable of binding to an HLA-G epitope comprises a V_{H} sequence comprising, consisting of, or consisting essentially of SEQ ID NO: 189. In some embodiments, the binding domain capable of binding to an HLA-G epitope comprises a V_{H} sequence comprising, consisting of, or consisting essentially of SEQ ID NO: 190. In some embodiments, the binding domain capable of binding to an HLA-G epitope comprises a V_{H} sequence comprising, consisting of, or consisting essentially of SEQ ID NO: 191. In some embodiments, the binding domain capable of binding to an HLA-G epitope comprises a V_{H} sequence comprising, consisting of, or consisting essentially of SEQ ID NO: 192. In some embodiments, the binding domain capable of binding to an HLA-G epitope comprises a V_{H} sequence comprising, consisting of, or consisting essentially of SEQ ID NO: 193. In some embodiments, the binding domain capable of binding to an HLA-G epitope comprises a V_{H} sequence comprising, consisting of, or consisting essentially of SEQ ID NO: 194. In some embodiments, the binding domain capable of binding to an HLA-G epitope comprises a V_{H} sequence comprising, consisting of, or consisting essentially of SEQ ID NO: 195. In some embodiments, the binding domain capable of binding to an HLA-G epitope comprises a V_{H} sequence comprising, consisting of, or consisting essentially of SEQ ID NO: 196. In some embodiments, the binding domain capable of binding to an HLA-G epitope comprises a V_{H} sequence comprising, consisting of, or consisting essentially of SEQ ID NO: 197. In some embodiments, the binding domain capable of binding to an HLA-G epitope comprises a V_{H} sequence comprising, consisting of, or consisting essentially of SEQ ID NO: 198. In some embodiments, the binding domain capable of binding to an HLA-G epitope comprises a V_{H} sequence comprising, consisting of, or consisting essentially of SEQ ID NO: 199. In some embodiments, the binding domain capable of binding to an HLA-G epitope comprises a V_{H} sequence comprising, consisting of, or consisting essentially of SEQ ID NO: 200.

In some embodiments, the binding domain capable of binding to an HLA-G epitope comprises three heavy chain CDRs each comprising, consisting of, or consisting essentially of a CDR sequence of a V_{H} having the sequence set forth in one of SEQ ID NOS: 170-200.

### Variants of V_{H} Sequences

In some embodiments, the V_{H} sequences provided herein comprise, consist of, or consist essentially of a variant of an illustrative V_{H} sequence provided in this disclosure.

In some embodiments, the V_{H} sequence comprises, consists of, or consists essentially of a variant of an illustrative V_{H} sequence provided in this disclosure. In some embodiments, the V_{H} sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

### CDR-L3 Sequences

In some embodiments, the binding domain capable of binding to an HLA-G epitope comprises a CDR-L3 sequence comprising, consisting of, or consisting essentially of a sequence selected from SEQ ID NOS: 149-166. In some embodiments, the binding domain capable of binding to an HLA-G epitope comprises a CDR-L3 sequence comprising, consisting of, or consisting essentially of SEQ ID NO: 149. In some embodiments, the binding domain capable of binding to an HLA-G epitope comprises a CDR-L3 sequence comprising, consisting of, or consisting essentially of SEQ ID NO: 150. In some embodiments, the binding domain capable of binding to an HLA-G epitope comprises a CDR-L3 sequence comprising, consisting of, or consisting essentially of SEQ ID NO: 151. In some embodiments, the binding domain capable of binding to an HLA-G epitope comprises a CDR-L3 sequence comprising, consisting of, or consisting essentially of SEQ ID NO: 152. In some embodiments, the binding domain capable of binding to an HLA-G epitope comprises a CDR-L3 sequence comprising, consisting of, or consisting essentially of SEQ ID NO: 153. In some embodiments, the binding domain capable of binding to an HLA-G epitope comprises a CDR-L3 sequence comprising, consisting of, or consisting essentially of SEQ ID NO: 154. In some embodiments, the binding domain capable of binding to an HLA-G epitope comprises a CDR-L3 sequence comprising, consisting of, or consisting essentially of SEQ ID NO: 155. In some embodiments, the binding domain capable of binding to an HLA-G epitope comprises a CDR-L3 sequence comprising, consisting of, or consisting essentially of SEQ ID NO: 156. In some embodiments, the binding domain capable of binding to an HLA-G epitope comprises a CDR-L3 sequence comprising, consisting of, or consisting essentially of SEQ ID NO: 157. In some embodiments, the binding domain capable of binding to an HLA-G epitope comprises a CDR-L3 sequence comprising, consisting of, or consisting essentially of SEQ ID NO: 158. In some embodiments, the binding domain capable of binding to an HLA-G epitope comprises a CDR-L3 sequence comprising, consisting of, or consisting essentially of SEQ ID NO: 159. In some embodiments, the binding domain capable of binding to an HLA-G epitope comprises a CDR-L3 sequence comprising, consisting of, or consisting essentially of SEQ ID NO: 160. In some embodiments, the binding domain capable of binding to an HLA-G epitope comprises a CDR-L3 sequence comprising, consisting of, or consisting essentially of SEQ ID NO: 161. In some embodiments, the binding domain capable of binding to an HLA-G epitope comprises a CDR-L3 sequence comprising, consisting of, or consisting essentially of SEQ ID NO: 162. In some embodiments, the binding domain capable of binding to an HLA-G epitope comprises a CDR-L3 sequence comprising, consisting of, or consisting essentially of SEQ ID NO: 163. In some embodiments, the binding domain capable of binding to an HLA-G epitope comprises a CDR-L3 sequence comprising, consisting of, or consisting essentially of SEQ ID NO: 164. In some embodiments, the binding domain capable of binding to an HLA-G epitope comprises a CDR-L3 sequence comprising, consisting of, or consisting essentially of SEQ ID NO: 165. In some embodiments, the binding domain capable of binding to an HLA-G epitope comprises a CDR-L3 sequence comprising, consisting of, or consisting essentially of SEQ ID NO: 166.

In some embodiments, the CDR-L3 sequence comprises, consists of, or consists essentially of a variant of an illustrative CDR-L3 sequence provided in this disclosure. In some embodiments, the CDR-L3 sequence comprises, consists of, or consists essentially of a sequence having at least 70%, 75%, 80%, 85%, 90%, or 95% identity with any of the illustrative CDR-L3 sequences provided in this disclosure. In some embodiments, the CDR-L3 sequence comprises, consists of, or consists essentially of any of the illustrative CDR-L3 sequences provided in this disclosure, with 1, 2, or 3 amino acid substitutions. In some embodiments, the amino acid substitutions are conservative amino acid substitutions.

### V_{L} Sequences Comprising Illustrative CDRs

In some embodiments, the binding domain capable of binding to an HLA-G epitope comprises a V_{L} sequence comprising one or more CDR-L sequences comprising, consisting of, or consisting essentially of one or more illustrative CDR-L sequences provided in this disclosure, and variants thereof.

### CDR-L3

In some embodiments, the binding domain capable of binding to an HLA-G epitope comprises a V_{L} sequence comprising a CDR-L3 sequence comprising, consisting of, or consisting essentially of a sequence selected from SEQ ID NOS: 149-166. In some embodiments, the binding domain capable of binding to an HLA-G epitope comprises a V_{L} sequence comprising a CDR-L3 sequence comprising, consisting of, or consisting essentially of SEQ ID NO: 149. In some embodiments, the binding domain capable of binding to an HLA-G epitope comprises a V_{L} sequence comprising a CDR-L3 sequence comprising, consisting of, or consisting essentially of SEQ ID NO: 150. In some embodiments, the binding domain capable of binding to an HLA-G epitope comprises a V_{L} sequence comprising a CDR-L3 sequence comprising, consisting of, or consisting essentially of SEQ ID NO: 151. In some embodiments, the binding domain capable of binding to an HLA-G epitope comprises a V_{L} sequence comprising a CDR-L3 sequence comprising, consisting of, or consisting essentially of SEQ ID NO: 152. In some embodiments, the binding domain capable of binding to an HLA-G epitope comprises a V_{L} sequence comprising a CDR-L3 sequence comprising, consisting of, or consisting essentially of SEQ ID NO: 153. In some embodiments, the binding domain capable of binding to an HLA-G epitope comprises a V_{L} sequence comprising a CDR-L3 sequence comprising, consisting of, or consisting essentially of SEQ ID NO: 154. In some embodiments, the binding domain capable of binding to an HLA-G epitope comprises a V_{L} sequence comprising a CDR-L3 sequence comprising, consisting of, or consisting essentially of SEQ ID NO: 155. In some embodiments, the binding domain capable of binding to an HLA-G epitope comprises a V_{L} sequence comprising a CDR-L3 sequence comprising, consisting of, or consisting essentially of SEQ ID NO: 156. In some embodiments, the binding domain capable of binding to an HLA-G epitope comprises a V_{L} sequence comprising a CDR-L3 sequence comprising, consisting of, or consisting essentially of SEQ ID NO: 157. In some embodiments, the binding domain capable of binding to an HLA-G epitope comprises a V_{L} sequence comprising a CDR-L3 sequence comprising, consisting of, or consisting essentially of SEQ ID NO: 158. In some embodiments, the binding domain capable of binding to an HLA-G epitope comprises a V_{L} sequence comprising a CDR-L3 sequence comprising, consisting of, or consisting essentially of SEQ ID NO: 159. In some embodiments, the binding domain capable of binding to an HLA-G epitope comprises a V_{L} sequence comprising a CDR-L3 sequence comprising, consisting of, or consisting essentially of SEQ ID NO: 160. In some embodiments, the binding domain capable of binding to an HLA-G epitope comprises a V_{L} sequence comprising a CDR-L3 sequence comprising, consisting of, or consisting essentially of SEQ ID NO: 161. In some embodiments, the binding domain capable of binding to an HLA-G epitope comprises a V_{L} sequence comprising a CDR-L3 sequence comprising, consisting of, or consisting essentially of SEQ ID NO: 162. In some embodiments, the binding domain capable of binding to an HLA-G epitope comprises a V_{L} sequence comprising a CDR-L3 sequence comprising, consisting of, or consisting essentially of SEQ ID NO: 163. In some embodiments, the binding domain capable of binding to an HLA-G epitope comprises a V_{L} sequence comprising a CDR-L3 sequence comprising, consisting of, or consisting essentially of SEQ ID NO: 164. In some embodiments, the binding domain capable of binding to an HLA-G epitope comprises a V_{L} sequence comprising a CDR-L3 sequence comprising, consisting of, or consisting essentially of SEQ ID NO: 165. In some embodiments, the binding domain capable of binding to an HLA-G epitope comprises a V_{L} sequence comprising a CDR-L3 sequence comprising, consisting of, or consisting essentially of SEQ ID NO: 166.

### CDR-L2

In some embodiments, the binding domain capable of binding to an HLA-G epitope comprises a V_{L} sequence comprising a CDR-L2 sequence comprising, consisting of, or consisting essentially of a sequence selected from SEQ ID NOS: 128-145. In some embodiments, the binding domain capable of binding to an HLA-G epitope comprises a V_{L} sequence comprising a CDR-L2 sequence comprising, consisting of, or consisting essentially of SEQ ID NO: 128. In some embodiments, the binding domain capable of binding to an HLA-G epitope comprises a V_{L} sequence comprising a CDR-L2 sequence comprising, consisting of, or consisting essentially of SEQ ID NO: 129. In some embodiments, the binding domain capable of binding to an HLA-G epitope comprises a V_{L} sequence comprising a CDR-L2 sequence comprising, consisting of, or consisting essentially of SEQ ID NO: 130. In some embodiments, the binding domain capable of binding to an HLA-G epitope comprises a V_{L} sequence comprising a CDR-L2 sequence comprising, consisting of, or consisting essentially of SEQ ID NO: 131. In some embodiments, the binding domain capable of binding to an HLA-G epitope comprises a V_{L} sequence comprising a CDR-L2 sequence comprising, consisting of, or consisting essentially of SEQ ID NO: 132. In some embodiments, the binding domain capable of binding to an HLA-G epitope comprises a V_{L} sequence comprising a CDR-L2 sequence comprising, consisting of, or consisting essentially of SEQ ID NO: 133. In some embodiments, the binding domain capable of binding to an HLA-G epitope comprises a V_{L} sequence comprising a CDR-L2 sequence comprising, consisting of, or consisting essentially of SEQ ID NO: 134. In some embodiments, the binding domain capable of binding to an HLA-G epitope comprises a V_{L} sequence comprising a CDR-L2 sequence comprising, consisting of, or consisting essentially of SEQ ID NO: 135. In some embodiments, the binding domain capable of binding to an HLA-G epitope comprises a V_{L} sequence comprising a CDR-L2 sequence comprising, consisting of, or consisting essentially of SEQ ID NO: 136. In some embodiments, the binding domain capable of binding to an HLA-G epitope comprises a V_{L} sequence comprising a CDR-L2 sequence comprising, consisting of, or consisting essentially of SEQ ID NO: 137. In some embodiments, the binding domain capable of binding to an HLA-G epitope comprises a V_{L} sequence comprising a CDR-L2 sequence comprising, consisting of, or consisting essentially of SEQ ID NO: 138. In some embodiments, the binding domain capable of binding to an HLA-G epitope comprises a V_{L} sequence comprising a CDR-L2 sequence comprising, consisting of, or consisting essentially of SEQ ID NO: 139. In some embodiments, the binding domain capable of binding to an HLA-G epitope comprises a V_{L} sequence comprising a CDR-L2 sequence comprising, consisting of, or consisting essentially of SEQ ID NO: 140. In some embodiments, the binding domain capable of binding to an HLA-G epitope comprises a V_{L} sequence comprising a CDR-L2 sequence comprising, consisting of, or consisting essentially of SEQ ID NO: 141. In some embodiments, the binding domain capable of binding to an HLA-G epitope comprises a V_{L} sequence comprising a CDR-L2 sequence comprising, consisting of, or consisting essentially of SEQ ID NO: 142. In some embodiments, the binding domain capable of binding to an HLA-G epitope comprises a V_{L} sequence comprising a CDR-L2 sequence comprising, consisting of, or consisting essentially of SEQ ID NO: 143. In some embodiments, the binding domain capable of binding to an HLA-G epitope comprises a V_{L} sequence comprising a CDR-L2 sequence comprising, consisting of, or consisting essentially of SEQ ID NO: 144. In some embodiments, the binding domain capable of binding to an HLA-G epitope comprises a V_{L} sequence comprising a CDR-L2 sequence comprising, consisting of, or consisting essentially of SEQ ID NO: 145.

### CDR-L1

In some embodiments, the binding domain capable of binding to an HLA-G epitope comprises a V_{L} sequence comprising a CDR-L1 sequence comprising, consisting of, or consisting essentially of a sequence selected from SEQ ID NOS: 105-124. In some embodiments, the binding domain capable of binding to an HLA-G epitope comprises a V_{L} sequence comprising a CDR-L1 sequence comprising, consisting of, or consisting essentially of SEQ ID NO: 105. In some embodiments, the binding domain capable of binding to an HLA-G epitope comprises a V_{L} sequence comprising a CDR-L1 sequence comprising, consisting of, or consisting essentially of SEQ ID NO: 106. In some embodiments, the binding domain capable of binding to an HLA-G epitope comprises a V_{L} sequence comprising a CDR-L1 sequence comprising, consisting of, or consisting essentially of SEQ ID NO: 107. In some embodiments, the binding domain capable of binding to an HLA-G epitope comprises a V_{L} sequence comprising a CDR-L1 sequence comprising, consisting of, or consisting essentially of SEQ ID NO: 108. In some embodiments, the binding domain capable of binding to an HLA-G epitope comprises a V_{L} sequence comprising a CDR-L1 sequence comprising, consisting of, or consisting essentially of SEQ ID NO: 109. In some embodiments, the binding domain capable of binding to an HLA-G epitope comprises a V_{L} sequence comprising a CDR-L1 sequence comprising, consisting of, or consisting essentially of SEQ ID NO: 110. In some embodiments, the binding domain capable of binding to an HLA-G epitope comprises a V_{L} sequence comprising a CDR-L1 sequence comprising, consisting of, or consisting essentially of SEQ ID NO: 111. In some embodiments, the binding domain capable of binding to an HLA-G epitope comprises a V_{L} sequence comprising a CDR-L1 sequence comprising, consisting of, or consisting essentially of SEQ ID NO: 112. In some embodiments, the binding domain capable of binding to an HLA-G epitope comprises a V_{L} sequence comprising a CDR-L1 sequence comprising, consisting of, or consisting essentially of SEQ ID NO: 113. In some embodiments, the binding domain capable of binding to an HLA-G epitope comprises a V_{L} sequence comprising a CDR-L1 sequence comprising, consisting of, or consisting essentially of SEQ ID NO: 114. In some embodiments, the binding domain capable of binding to an HLA-G epitope comprises a V_{L} sequence comprising a CDR-L1 sequence comprising, consisting of, or consisting essentially of SEQ ID NO: 115. In some embodiments, the binding domain capable of binding to an HLA-G epitope comprises a V_{L} sequence comprising a CDR-L1 sequence comprising, consisting of, or consisting essentially of SEQ ID NO: 116. In some embodiments, the binding domain capable of binding to an HLA-G epitope comprises a V_{L} sequence comprising a CDR-L1 sequence comprising, consisting of, or consisting essentially of SEQ ID NO: 117. In some embodiments, the binding domain capable of binding to an HLA-G epitope comprises a V_{L} sequence comprising a CDR-L1 sequence comprising, consisting of, or consisting essentially of SEQ ID NO: 118. In some embodiments, the binding domain capable of binding to an HLA-G epitope comprises a V_{L} sequence comprising a CDR-L1 sequence comprising, consisting of, or consisting essentially of SEQ ID NO: 119. In some embodiments, the binding domain capable of binding to an HLA-G epitope comprises a V_{L} sequence comprising a CDR-L1 sequence comprising, consisting of, or consisting essentially of SEQ ID NO: 120. In some embodiments, the binding domain capable of binding to an HLA-G epitope comprises a V_{L} sequence comprising a CDR-L1 sequence comprising, consisting of, or consisting essentially of SEQ ID NO: 121. In some embodiments, the binding domain capable of binding to an HLA-G epitope comprises a V_{L} sequence comprising a CDR-L1 sequence comprising, consisting of, or consisting essentially of SEQ ID NO: 122. In some embodiments, the binding domain capable of binding to an HLA-G epitope comprises a V_{L} sequence comprising a CDR-L1 sequence comprising, consisting of, or consisting essentially of SEQ ID NO: 123. In some embodiments, the binding domain capable of binding to an HLA-G epitope comprises a V_{L} sequence comprising a CDR-L1 sequence comprising, consisting of, or consisting essentially of SEQ ID NO: 124.

### CDR-L3+CDR-L2

In some embodiments, the binding domain capable of binding to an HLA-G epitope comprises a V_{L} sequence comprising a CDR-L3 sequence comprising, consisting of, or consisting essentially of a sequence selected from SEQ ID NOS: 149-166 and a CDR-L2 sequence comprising, consisting of, or consisting essentially of a sequence selected from SEQ ID NOS: 128-145. In some embodiments, the CDR-L3 sequence and the CDR-L2 sequence are both from a single illustrative V_{L} sequence provided in this disclosure. For example, in some embodiments, the CDR-L3 and CDR-L2 are both from a single illustrative V_{L} sequence selected from SEQ ID NOS: 204-228.

### CDR-L3+CDR-L1

In some embodiments, the binding domain capable of binding to an HLA-G epitope comprises a V_{L} sequence comprising a CDR-L3 sequence comprising, consisting of, or consisting essentially of a sequence selected from SEQ ID NOS: 149-166 and a CDR-L1 sequence comprising, consisting of, or consisting essentially of a sequence selected from SEQ ID NOS: 105-124. In some embodiments, the CDR-L3 sequence and the CDR-L1 sequence are both from a single illustrative V_{L} sequence provided in this disclosure. For example, in some embodiments, the CDR-L3 and CDR-L1 are both from a single illustrative V_{L} sequence selected from SEQ ID NOS: 204-228.

### CDR-L1+CDR-L2

In some embodiments, the binding domain capable of binding to an HLA-G epitope comprises a V_{L} sequence comprising a CDR-L1 sequence comprising, consisting of, or consisting essentially of a sequence selected from SEQ ID NOS: 105-124 and a CDR-L2 sequence comprising, consisting of, or consisting essentially of a sequence selected from SEQ ID NOS: 128-145. In some embodiments, the CDR-L1 sequence and the CDR-L2 sequence are both from a single illustrative V_{L} sequence provided in this disclosure. For example, in some embodiments, the CDR-L1 and CDR-L2 are both from a single illustrative V_{L} sequence selected from SEQ ID NOS: 204-228.

### CDR-L1+CDR-L2+CDR-L3

In some embodiments, the binding domain capable of binding to an HLA-G epitope comprises a V_{L} sequence comprising a CDR-L1 sequence comprising, consisting of, or consisting essentially of a sequence selected from SEQ ID NOS: 105-124, a CDR-L2 sequence comprising, consisting of, or consisting essentially of a sequence selected from SEQ ID NOS: 128-145, and a CDR-L3 sequence comprising, consisting of, or consisting essentially of a sequence selected from SEQ ID NOS: 149-166. In some embodiments, the CDR-L1 sequence, CDR-L2 sequence, and CDR-L3 sequence are all from a single illustrative V_{L} sequence provided in this disclosure. For example, in some embodiments, the CDR-L1, CDR-L2, and CDR-L3 are all from a single illustrative V_{L} sequence selected from SEQ ID NOS: 204-228.

In some embodiments, the binding domain capable of binding to an HLA-G epitope comprises a VL sequence comprising a CDR-L1 sequence comprising SEQ ID NO: 105, a CDR-L2 sequence comprising SEQ ID NO: 128, and a CDR-L3 sequence SEQ ID NO: 149. In some embodiments, the binding domain capable of binding to an HLA-G epitope comprises a VL sequence comprising a CDR-L1 sequence comprising SEQ ID NO: 106, a CDR-L2 sequence comprising SEQ ID NO: 129, and a CDR-L3 sequence SEQ ID NO: 150. In some embodiments, the binding domain capable of binding to an HLA-G epitope comprises a VL sequence comprising a CDR-L1 sequence comprising SEQ ID NO: 107, a CDR-L2 sequence comprising SEQ ID NO: 128, and a CDR-L3 sequence SEQ ID NO: 151. In some embodiments, the binding domain capable of binding to an HLA-G epitope comprises a VL sequence comprising a CDR-L1 sequence comprising SEQ ID NO: 108, a CDR-L2 sequence comprising SEQ ID NO: 130, and a CDR-L3 sequence SEQ ID NO: 151. In some embodiments, the binding domain capable of binding to an HLA-G epitope comprises a VL sequence comprising a CDR-L1 sequence comprising SEQ ID NO: 109, a CDR-L2 sequence comprising SEQ ID NO: 131, and a CDR-L3 sequence SEQ ID NO: 152. In some embodiments, the binding domain capable of binding to an HLA-G epitope comprises a VL sequence comprising a CDR-L1 sequence comprising SEQ ID NO: 107, a CDR-L2 sequence comprising SEQ ID NO: 132, and a CDR-L3 sequence SEQ ID NO: 153. In some embodiments, the binding domain capable of binding to an HLA-G epitope comprises a VL sequence comprising a CDR-L1 sequence comprising SEQ ID NO: 110, a CDR-L2 sequence comprising SEQ ID NO: 132, and a CDR-L3 sequence SEQ ID NO: 151. In some embodiments, the binding domain capable of binding to an HLA-G epitope comprises a VL sequence comprising a CDR-L1 sequence comprising SEQ ID NO: 111, a CDR-L2 sequence comprising SEQ ID NO: 133, and a CDR-L3 sequence SEQ ID NO: 151. In some embodiments, the binding domain capable of binding to an HLA-G epitope comprises a VL sequence comprising a CDR-L1 sequence comprising SEQ ID NO: 105, a CDR-L2 sequence comprising SEQ ID NO: 128, and a CDR-L3 sequence SEQ ID NO: 154. In some embodiments, the binding domain capable of binding to an HLA-G epitope comprises a VL sequence comprising a CDR-L1 sequence comprising SEQ ID NO: 112, a CDR-L2 sequence comprising SEQ ID NO: 134, and a CDR-L3 sequence SEQ ID NO: 155. In some embodiments, the binding domain capable of binding to an HLA-G epitope comprises a VL sequence comprising a CDR-L1 sequence comprising SEQ ID NO: 113, a CDR-L2 sequence comprising SEQ ID NO: 135, and a CDR-L3 sequence SEQ ID NO: 156. In some embodiments, the binding domain capable of binding to an HLA-G epitope comprises a VL sequence comprising a CDR-L1 sequence comprising SEQ ID NO: 114, a CDR-L2 sequence comprising SEQ ID NO: 136, and a CDR-L3 sequence SEQ ID NO: 157. In some embodiments, the binding domain capable of binding to an HLA-G epitope comprises a VL sequence comprising a CDR-L1 sequence comprising SEQ ID NO: 115, a CDR-L2 sequence comprising SEQ ID NO: 135, and a CDR-L3 sequence SEQ ID NO: 157. In some embodiments, the binding domain capable of binding to an HLA-G epitope comprises a VL sequence comprising a CDR-L1 sequence comprising SEQ ID NO: 116, a CDR-L2 sequence comprising SEQ ID NO: 128, and a CDR-L3 sequence SEQ ID NO: 155. In some embodiments, the binding domain capable of binding to an HLA-G epitope comprises a VL sequence comprising a CDR-L1 sequence comprising SEQ ID NO: 117, a CDR-L2 sequence comprising SEQ ID NO: 137, and a CDR-L3 sequence SEQ ID NO: 155. In some embodiments, the binding domain capable of binding to an HLA-G epitope comprises a VL sequence comprising a CDR-L1 sequence comprising SEQ ID NO: 118, a CDR-L2 sequence comprising SEQ ID NO: 137, and a CDR-L3 sequence SEQ ID NO: 158. In some embodiments, the binding domain capable of binding to an HLA-G epitope comprises a VL sequence comprising a CDR-L1 sequence comprising SEQ ID NO: 118, a CDR-L2 sequence comprising SEQ ID NO: 138, and a CDR-L3 sequence SEQ ID NO: 155. In some embodiments, the binding domain capable of binding to an HLA-G epitope comprises a VL sequence comprising a CDR-L1 sequence comprising SEQ ID NO: 119, a CDR-L2 sequence comprising SEQ ID NO: 139, and a CDR-L3 sequence SEQ ID NO: 159. In some embodiments, the binding domain capable of binding to an HLA-G epitope comprises a VL sequence comprising a CDR-L1 sequence comprising SEQ ID NO: 120, a CDR-L2 sequence comprising SEQ ID NO: 140, and a CDR-L3 sequence SEQ ID NO: 160. In some embodiments, the binding domain capable of binding to an HLA-G epitope comprises a VL sequence comprising a CDR-L1 sequence comprising SEQ ID NO: 121, a CDR-L2 sequence comprising SEQ ID NO: 141, and a CDR-L3 sequence SEQ ID NO: 161. In some embodiments, the binding domain capable of binding to an HLA-G epitope comprises a VL sequence comprising a CDR-L1 sequence comprising SEQ ID NO: 122, a CDR-L2 sequence comprising SEQ ID NO: 142, and a CDR-L3 sequence SEQ ID NO: 162. In some embodiments, the binding domain capable of binding to an HLA-G epitope comprises a VL sequence comprising a CDR-L1 sequence comprising SEQ ID NO: 105, a CDR-L2 sequence comprising SEQ ID NO: 143, and a CDR-L3 sequence SEQ ID NO: 163. In some embodiments, the binding domain capable of binding to an HLA-G epitope comprises a VL sequence comprising a CDR-L1 sequence comprising SEQ ID NO: 105, a CDR-L2 sequence comprising SEQ ID NO: 143, and a CDR-L3 sequence SEQ ID NO: 164. In some embodiments, the binding domain capable of binding to an HLA-G epitope comprises a VL sequence comprising a CDR-L1 sequence comprising SEQ ID NO: 123, a CDR-L2 sequence comprising SEQ ID NO: 144, and a CDR-L3 sequence SEQ ID NO: 165. In some embodiments, the binding domain capable of binding to an HLA-G epitope comprises a VL sequence comprising a CDR-L1 sequence comprising SEQ ID NO: 124, a CDR-L2 sequence comprising SEQ ID NO: 145, and a CDR-L3 sequence SEQ ID NO: 166.

### Variants of V_{L} Sequences Comprising Illustrative CDR-Ls

In some embodiments, the V_{L} sequences provided herein comprise a variant of an illustrative CDR-L3, CDR-L2, and/or CDR-L1 sequence provided in this disclosure.

In some embodiments, the CDR-L3 sequence comprises, consists of, or consists essentially of a variant of an illustrative CDR-L3 sequence provided in this disclosure. In some embodiments, the CDR-L3 sequence comprises, consists of, or consists essentially of a sequence having at least 70%, 75%, 80%, 85%, 90%, or 95% identity with any of the illustrative CDR-L3 sequences provided in this disclosure. In some embodiments, the CDR-L3 sequence comprises, consists of, or consists essentially of any of the illustrative CDR-L3 sequences provided in this disclosure, with 1, 2, or 3 amino acid substitutions. In some embodiments, the amino acid substitutions are conservative amino acid substitutions.

In some embodiments, the CDR-L2 sequence comprises, consists of, or consists essentially of a variant of an illustrative CDR-L2 sequence provided in this disclosure. In some embodiments, the CDR-L2 sequence comprises, consists of, or consists essentially of a sequence having at least 70%, 75%, 80%, 85%, 90%, or 95% identity with any of the illustrative CDR-L2 sequences provided in this disclosure. In some embodiments, the CDR-L2 sequence comprises, consists of, or consists essentially of any of the illustrative CDR-L2 sequences provided in this disclosure, with 1, 2, or 3 amino acid substitutions. In some embodiments, the amino acid substitutions are conservative amino acid substitutions.

In some embodiments, the CDR-L1 sequence comprises, consists of, or consists essentially of a variant of an illustrative CDR-L1 sequence provided in this disclosure. In some embodiments, the CDR-L1 sequence comprises, consists of, or consists essentially of a sequence having at least 70%, 75%, 80%, 85%, 90%, or 95% identity with any of the illustrative CDR-L1 sequences provided in this disclosure. In some embodiments, the CDR-L1 sequence comprises, consists of, or consists essentially of any of the illustrative CDR-L1 sequences provided in this disclosure, with 1, 2, or 3 amino acid substitutions. In some embodiments, the amino acid substitutions are conservative amino acid substitutions.

### V_{L} Sequences

In some embodiments, the binding domain capable of binding to an HLA-G epitope comprises a V_{L} sequence comprising, consisting of, or consisting essentially of a sequence selected from SEQ ID NOS: 204-228. In some embodiments, the binding domain capable of binding to an HLA-G epitope comprises a V_{L} sequence comprising, consisting of, or consisting essentially of SEQ ID NO: 204. In some embodiments, the binding domain capable of binding to an HLA-G epitope comprises a V_{L} sequence comprising, consisting of, or consisting essentially of SEQ ID NO: 205. In some embodiments, the binding domain capable of binding to an HLA-G epitope comprises a V_{L} sequence comprising, consisting of, or consisting essentially of SEQ ID NO: 206. In some embodiments, the binding domain capable of binding to an HLA-G epitope comprises a V_{L} sequence comprising, consisting of, or consisting essentially of SEQ ID NO: 207. In some embodiments, the binding domain capable of binding to an HLA-G epitope comprises a V_{L} sequence comprising, consisting of, or consisting essentially of SEQ ID NO: 208. In some embodiments, the binding domain capable of binding to an HLA-G epitope comprises a V_{L} sequence comprising, consisting of, or consisting essentially of SEQ ID NO: 209. In some embodiments, the binding domain capable of binding to an HLA-G epitope comprises a V_{L} sequence comprising, consisting of, or consisting essentially of SEQ ID NO: 210. In some embodiments, the binding domain capable of binding to an HLA-G epitope comprises a V_{L} sequence comprising, consisting of, or consisting essentially of SEQ ID NO: 211. In some embodiments, the binding domain capable of binding to an HLA-G epitope comprises a V_{L} sequence comprising, consisting of, or consisting essentially of SEQ ID NO: 212. In some embodiments, the binding domain capable of binding to an HLA-G epitope comprises a V_{L} sequence comprising, consisting of, or consisting essentially of SEQ ID NO: 213. In some embodiments, the binding domain capable of binding to an HLA-G epitope comprises a V_{L} sequence comprising, consisting of, or consisting essentially of SEQ ID NO: 214. In some embodiments, the binding domain capable of binding to an HLA-G epitope comprises a V_{L} sequence comprising, consisting of, or consisting essentially of SEQ ID NO: 215. In some embodiments, the binding domain capable of binding to an HLA-G epitope comprises a V_{L} sequence comprising, consisting of, or consisting essentially of SEQ ID NO: 216. In some embodiments, the binding domain capable of binding to an HLA-G epitope comprises a V_{L} sequence comprising, consisting of, or consisting essentially of SEQ ID NO: 217. In some embodiments, the binding domain capable of binding to an HLA-G epitope comprises a V_{L} sequence comprising, consisting of, or consisting essentially of SEQ ID NO: 218. In some embodiments, the binding domain capable of binding to an HLA-G epitope comprises a V_{L} sequence comprising, consisting of, or consisting essentially of SEQ ID NO: 219. In some embodiments, the binding domain capable of binding to an HLA-G epitope comprises a V_{L} sequence comprising, consisting of, or consisting essentially of SEQ ID NO: 220. In some embodiments, the binding domain capable of binding to an HLA-G epitope comprises a V_{L} sequence comprising, consisting of, or consisting essentially of SEQ ID NO: 221. In some embodiments, the binding domain capable of binding to an HLA-G epitope comprises a V_{L} sequence comprising, consisting of, or consisting essentially of SEQ ID NO: 222. In some embodiments, the binding domain capable of binding to an HLA-G epitope comprises a V_{L} sequence comprising, consisting of, or consisting essentially of SEQ ID NO: 223. In some embodiments, the binding domain capable of binding to an HLA-G epitope comprises a V_{L} sequence comprising, consisting of, or consisting essentially of SEQ ID NO: 224. In some embodiments, the binding domain capable of binding to an HLA-G epitope comprises a V_{L} sequence comprising, consisting of, or consisting essentially of SEQ ID NO: 225. In some embodiments, the binding domain capable of binding to an HLA-G epitope comprises a V_{L} sequence comprising, consisting of, or consisting essentially of SEQ ID NO: 226. In some embodiments, the binding domain capable of binding to an HLA-G epitope comprises a V_{L} sequence comprising, consisting of, or consisting essentially of SEQ ID NO: 227. In some embodiments, the binding domain capable of binding to an HLA-G epitope comprises a V_{L} sequence comprising, consisting of, or consisting essentially of SEQ ID NO: 228.

In some embodiments, the binding domain capable of binding to an HLA-G epitope comprises three light chain CDRs, each comprising, consisting of, or consisting essentially of a CDR sequence of a VL having the sequence set forth in one of SEQ ID NO: 204-228.

### Variants of V_{L} Sequences

In some embodiments, the V_{L} sequences provided herein comprise, consist of, or consist essentially of a variant of an illustrative V_{L} sequence provided in this disclosure.

In some embodiments, the V_{L} sequence comprises, consists of, or consists essentially of a variant of an illustrative V_{L} sequence provided in this disclosure. In some embodiments, the V_{L} sequence comprises, consists of, or consists essentially of a sequence having at least 85%, 90%, 95%, 96%, 97%, 98%, 99%, or 99.05% identity with any of the illustrative V_{L} sequences provided in this disclosure.

In some embodiments, the V_{L} sequence comprises, consists of, or consists essentially of any of the illustrative V_{L} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

### Pairs

### CDR-H3-CDR-L3 Pairs

In some embodiments, the binding domain capable of binding to an HLA-G epitope comprises a CDR-H3 sequence and a CDR-L3 sequence. In some embodiments, the CDR-H3 sequence is part of a V_{H} and the CDR-L3 sequence is part of a V_{L}.

In some embodiments, the CDR-H3 sequence is a CDR-H3 sequence comprising, consisting of, or consisting essentially of SEQ ID NOS: 76-101, and the CDR-L3 sequence is a CDR-L3 sequence comprising, consisting of, or consisting essentially of SEQ ID NOS: 149-166.

In some embodiments, the CDR-H3 sequence is SEQ ID NO: 76 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 77 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: 160. In some embodiments, the CDR-L3 sequence is SEQ ID NO: 161. In some embodiments, the CDR-L3 sequence is SEQ ID NO: 162. In some embodiments, the CDR-L3 sequence is SEQ ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 78 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 79 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: 160. In some embodiments, the CDR-L3 sequence is SEQ ID NO: 161. In some embodiments, the CDR-L3 sequence is SEQ ID NO: 162. In some embodiments, the CDR-L3 sequence is SEQ ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 80 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 81 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: 160. In some embodiments, the CDR-L3 sequence is SEQ ID NO: 161. In some embodiments, the CDR-L3 sequence is SEQ ID NO: 162. In some embodiments, the CDR-L3 sequence is SEQ ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 82 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 83 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: 160. In some embodiments, the CDR-L3 sequence is SEQ ID NO: 161. In some embodiments, the CDR-L3 sequence is SEQ ID NO: 162. In some embodiments, the CDR-L3 sequence is SEQ ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 84 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 85 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: 160. In some embodiments, the CDR-L3 sequence is SEQ ID NO: 161. In some embodiments, the CDR-L3 sequence is SEQ ID NO: 162. In some embodiments, the CDR-L3 sequence is SEQ ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 86 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 87 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: 160. In some embodiments, the CDR-L3 sequence is SEQ ID NO: 161. In some embodiments, the CDR-L3 sequence is SEQ ID NO: 162. In some embodiments, the CDR-L3 sequence is SEQ ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 88 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 89 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: 160. In some embodiments, the CDR-L3 sequence is SEQ ID NO: 161. In some embodiments, the CDR-L3 sequence is SEQ ID NO: 162. In some embodiments, the CDR-L3 sequence is SEQ ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 90 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 91 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: 160. In some embodiments, the CDR-L3 sequence is SEQ ID NO: 161. In some embodiments, the CDR-L3 sequence is SEQ ID NO: 162. In some embodiments, the CDR-L3 sequence is SEQ ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 92 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 93 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: 160. In some embodiments, the CDR-L3 sequence is SEQ ID NO: 161. In some embodiments, the CDR-L3 sequence is SEQ ID NO: 162. In some embodiments, the CDR-L3 sequence is SEQ ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 94 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 95 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: 160. In some embodiments, the CDR-L3 sequence is SEQ ID NO: 161. In some embodiments, the CDR-L3 sequence is SEQ ID NO: 162. In some embodiments, the CDR-L3 sequence is SEQ ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 96 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 97 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: 160. In some embodiments, the CDR-L3 sequence is SEQ ID NO: 161. In some embodiments, the CDR-L3 sequence is SEQ ID NO: 162. In some embodiments, the CDR-L3 sequence is SEQ ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 98 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 99 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: 160. In some embodiments, the CDR-L3 sequence is SEQ ID NO: 161. In some embodiments, the CDR-L3 sequence is SEQ ID NO: 162. In some embodiments, the CDR-L3 sequence is SEQ ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 100 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: 160. In some embodiments, the CDR-L3 sequence is SEQ ID NO: 161. In some embodiments, the CDR-L3 sequence is SEQ ID NO: 162. In some embodiments, the CDR-L3 sequence is SEQ ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

In some embodiments, the CDR-H3 sequence is SEQ ID NO: 101 and the CDR-L3 sequence is selected from SEQ ID NOS: 149-166. In some embodiments, the CDR-L3 sequence is SEQ ID NO: 149. In some embodiments, the CDR-L3 sequence is SEQ ID NO: 150. In some embodiments, the CDR-L3 sequence is SEQ ID NO: 151. In some embodiments, the CDR-L3 sequence is SEQ ID NO: 152. In some embodiments, the CDR-L3 sequence is SEQ ID NO: 153. In some embodiments, the CDR-L3 sequence is SEQ ID NO: 154. In some embodiments, the CDR-L3 sequence is SEQ ID NO: 155. In some embodiments, the CDR-L3 sequence is SEQ ID NO: 156. In some embodiments, the CDR-L3 sequence is SEQ ID NO: 157. In some embodiments, the CDR-L3 sequence is SEQ ID NO: 158. In some embodiments, the CDR-L3 sequence is SEQ ID NO: 159. In some embodiments, the CDR-L3 sequence is SEQ ID NO: 160. In some embodiments, the CDR-L3 sequence is SEQ ID NO: 161. In some embodiments, the CDR-L3 sequence is SEQ ID NO: 162. In some embodiments, the CDR-L3 sequence is SEQ ID NO: 163. In some embodiments, the CDR-L3 sequence is SEQ ID NO: 164. In some embodiments, the CDR-L3 sequence is SEQ ID NO: 165. In some embodiments, the CDR-L3 sequence is SEQ ID NO: 166.

### 1.1.1.1. Variants of CDR-H3-CDR-L3 Pairs

In some embodiments, the CDR-H3-CDR-L3 pairs provided herein comprise a variant of an illustrative CDR-H3 and/or CDR-L1 sequence provided in this disclosure.

In some embodiments, the CDR-H3 sequence comprises, consists of, or consists essentially of a variant of an illustrative CDR-H3 sequence provided in this disclosure. In some embodiments, the CDR-H3 sequence comprises, consists of, or consists essentially of a sequence having at least 70%, 75%, 80%, 85%, 90%, or 95% identity with any of the illustrative CDR-H3 sequences provided in this disclosure. In some embodiments, the CDR-H3 sequence comprises, consists of, or consists essentially of any of the illustrative CDR-H3 sequences provided in this disclosure, with 1, 2, or 3 amino acid substitutions. In some embodiments, the amino acid substitutions are conservative amino acid substitutions.

In some embodiments, the CDR-L3 sequence comprises, consists of, or consists essentially of a variant of an illustrative CDR-L3 sequence provided in this disclosure. In some embodiments, the CDR-L3 sequence comprises, consists of, or consists essentially of a sequence having at least 70%, 75%, 80%, 85%, 90%, or 95% identity with any of the illustrative CDR-L3 sequences provided in this disclosure. In some embodiments, the CDR-L3 sequence comprises, consists of, or consists essentially of any of the illustrative CDR-L3 sequences provided in this disclosure, with 1, 2, or 3 amino acid substitutions. In some embodiments, the amino acid substitutions are conservative amino acid substitutions.

V_{H}-V_{L} Pairs

In some embodiments, the binding domain capable of binding to an HLA-G epitope comprises a V_{H} sequence and/or a V_{L} sequence.

In some embodiments, the V_{H} sequence is a V_{H} sequence comprising, consisting of, or consisting essentially of SEQ ID NOS: 170-200 and the V_{L} sequence is a V_{L} sequence comprising, consisting of, or consisting essentially of SEQ ID NOS: 204-228.

In some embodiments, the V_{H} sequence is SEQ ID NO: 170 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 171 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 172 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 173 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 174 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 175 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 176 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 177 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 178 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 179 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 180 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 181 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 182 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 183 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 184 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 185 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 186 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 187 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 188 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 189 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 190 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 191 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 192 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 193 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 194 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 195 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 196 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 197 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 198 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 199 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the V_{H} sequence is SEQ ID NO: 200 and the V_{L} sequence is selected from SEQ ID NOS: 204-228. In some embodiments, the V_{L} sequence is SEQ ID NO: 204. In some embodiments, the V_{L} sequence is SEQ ID NO: 205. In some embodiments, the V_{L} sequence is SEQ ID NO: 206. In some embodiments, the V_{L} sequence is SEQ ID NO: 207. In some embodiments, the V_{L} sequence is SEQ ID NO: 208. In some embodiments, the V_{L} sequence is SEQ ID NO: 209. In some embodiments, the V_{L} sequence is SEQ ID NO: 210. In some embodiments, the V_{L} sequence is SEQ ID NO: 211. In some embodiments, the V_{L} sequence is SEQ ID NO: 212. In some embodiments, the V_{L} sequence is SEQ ID NO: 213. In some embodiments, the V_{L} sequence is SEQ ID NO: 214. In some embodiments, the V_{L} sequence is SEQ ID NO: 215. In some embodiments, the V_{L} sequence is SEQ ID NO: 216. In some embodiments, the V_{L} sequence is SEQ ID NO: 217. In some embodiments, the V_{L} sequence is SEQ ID NO: 218. In some embodiments, the V_{L} sequence is SEQ ID NO: 219. In some embodiments, the V_{L} sequence is SEQ ID NO: 220. In some embodiments, the V_{L} sequence is SEQ ID NO: 221. In some embodiments, the V_{L} sequence is SEQ ID NO: 222. In some embodiments, the V_{L} sequence is SEQ ID NO: 223. In some embodiments, the V_{L} sequence is SEQ ID NO: 224. In some embodiments, the V_{L} sequence is SEQ ID NO: 225. In some embodiments, the V_{L} sequence is SEQ ID NO: 226. In some embodiments, the V_{L} sequence is SEQ ID NO: 227. In some embodiments, the V_{L} sequence is SEQ ID NO: 228.

In some embodiments, the binding domain capable of binding to an HLA-G epitope comprises three heavy chain CDRs and three light chain CDRs, each comprising, consisting of, or consisting essentially of a CDR sequence of a VH-VL pair set forth above. In some embodiments, the binding domain capable of binding to an HLA-G epitope comprises three heavy chain CDRs and three light chain CDRs, each comprising, consisting of, or consisting essentially of a CDR sequence of a VH-VL pair set forth in Table S.

### CDR-H1+CDR-H2+CDR-H3+CDR-L1+CDR-L2+CDR-L3

In some embodiments, the binding domain capable of binding to an HLA-G epitope comprises a V_{H} sequence comprising a Kabat CDR-H1 sequence comprising SEQ ID NO: 18, a Kabat CDR-H2 sequence comprising SEQ ID NO: 54, and a Kabat CDR-H3 sequence comprising SEQ ID NO: 76 and a VL sequence comprising a CDR-L1 sequence comprising SEQ ID NO: 105, a CDR-L2 sequence comprising SEQ ID NO: 128, and a CDR-L3 sequence SEQ ID NO: 149. In some embodiments, the binding domain capable of binding to an HLA-G epitope comprises a V_{H} sequence comprising a Kabat CDR-H1 sequence comprising SEQ ID NO: 19, a Kabat CDR-H2 sequence comprising SEQ ID NO: 55, and a Kabat CDR-H3 sequence comprising SEQ ID NO: 77 and a VL sequence comprising a CDR-L1 sequence comprising SEQ ID NO: 105, a CDR-L2 sequence comprising SEQ ID NO: 128, and a CDR-L3 sequence SEQ ID NO: 149. In some embodiments, the binding domain capable of binding to an HLA-G epitope comprises a V_{H} sequence comprising a Kabat CDR-H1 sequence comprising SEQ ID NO: 20, a Kabat CDR-H2 sequence comprising SEQ ID NO: 56, and a Kabat CDR-H3 sequence comprising SEQ ID NO: 78 and a VL sequence comprising a CDR-L1 sequence comprising SEQ ID NO: 105, a CDR-L2 sequence comprising SEQ ID NO: 128, and a CDR-L3 sequence SEQ ID NO: 149. In some embodiments, the binding domain capable of binding to an HLA-G epitope comprises a V_{H} sequence comprising a Kabat CDR-H1 sequence comprising SEQ ID NO: 21, a Kabat CDR-H2 sequence comprising SEQ ID NO: 57, and a Kabat CDR-H3 sequence comprising SEQ ID NO: 79 and a VL sequence comprising a CDR-L1 sequence comprising SEQ ID NO: 105, a CDR-L2 sequence comprising SEQ ID NO: 128, and a CDR-L3 sequence SEQ ID NO: 149. In some embodiments, the binding domain capable of binding to an HLA-G epitope comprises a V_{H} sequence comprising a Kabat CDR-H1 sequence comprising SEQ ID NO: 22, a Kabat CDR-H2 sequence comprising SEQ ID NO: 58, and a Kabat CDR-H3 sequence comprising SEQ ID NO: 80 and a VL sequence comprising a CDR-L1 sequence comprising SEQ ID NO: 105, a CDR-L2 sequence comprising SEQ ID NO: 128, and a CDR-L3 sequence SEQ ID NO: 149. In some embodiments, the binding domain capable of binding to an HLA-G epitope comprises a V_{H} sequence comprising a Kabat CDR-H1 sequence comprising SEQ ID NO: 21, a Kabat CDR-H2 sequence comprising SEQ ID NO: 57, and a Kabat CDR-H3 sequence comprising SEQ ID NO: 76 and a VL sequence comprising a CDR-L1 sequence comprising SEQ ID NO: 106, a CDR-L2 sequence comprising SEQ ID NO: 129, and a CDR-L3 sequence SEQ ID NO: 150. In some embodiments, the binding domain capable of binding to an HLA-G epitope comprises a V_{H} sequence comprising a Kabat CDR-H1 sequence comprising SEQ ID NO: 23, a Kabat CDR-H2 sequence comprising SEQ ID NO: 59, and a Kabat CDR-H3 sequence comprising SEQ ID NO: 76 and a VL sequence comprising a CDR-L1 sequence comprising SEQ ID NO: 107, a CDR-L2 sequence comprising SEQ ID NO: 128, and a CDR-L3 sequence SEQ ID NO: 151. In some embodiments, the binding domain capable of binding to an HLA-G epitope comprises a V_{H} sequence comprising a Kabat CDR-H1 sequence comprising SEQ ID NO: 23, a Kabat CDR-H2 sequence comprising SEQ ID NO: 60, and a Kabat CDR-H3 sequence comprising SEQ ID NO: 81 and a VL sequence comprising a CDR-L1 sequence comprising SEQ ID NO: 108, a CDR-L2 sequence comprising SEQ ID NO: 130, and a CDR-L3 sequence SEQ ID NO: 151. In some embodiments, the binding domain capable of binding to an HLA-G epitope comprises a V_{H} sequence comprising a Kabat CDR-H1 sequence comprising SEQ ID NO: 23, a Kabat CDR-H2 sequence comprising SEQ ID NO: 59, and a Kabat CDR-H3 sequence comprising SEQ ID NO: 82 and a VL sequence comprising a CDR-L1 sequence comprising SEQ ID NO: 109, a CDR-L2 sequence comprising SEQ ID NO: 131, and a CDR-L3 sequence SEQ ID NO: 152. In some embodiments, the binding domain capable of binding to an HLA-G epitope comprises a V_{H} sequence comprising a Kabat CDR-H1 sequence comprising SEQ ID NO: 23, a Kabat CDR-H2 sequence comprising SEQ ID NO: 59, and a Kabat CDR-H3 sequence comprising SEQ ID NO: 76 and a VL sequence comprising a CDR-L1 sequence comprising SEQ ID NO: 107, a CDR-L2 sequence comprising SEQ ID NO: 132, and a CDR-L3 sequence SEQ ID NO:153. In some embodiments, the binding domain capable of binding to an HLA-G epitope comprises a V_{H} sequence comprising a Kabat CDR-H1 sequence comprising SEQ ID NO: 23, a Kabat CDR-H2 sequence comprising SEQ ID NO: 59, and a Kabat CDR-H3 sequence comprising SEQ ID NO: 81 and a VL sequence comprising a CDR-L1 sequence comprising SEQ ID NO: 110, a CDR-L2 sequence comprising SEQ ID NO: 132, and a CDR-L3 sequence SEQ ID NO: 151. In some embodiments, the binding domain capable of binding to an HLA-G epitope comprises a V_{H} sequence comprising a Kabat CDR-H1 sequence comprising SEQ ID NO: 21, a Kabat CDR-H2 sequence comprising SEQ ID NO: 57, and a Kabat CDR-H3 sequence comprising SEQ ID NO: 83 and a VL sequence comprising a CDR-L1 sequence comprising SEQ ID NO: 111, a CDR-L2 sequence comprising SEQ ID NO: 133, and a CDR-L3 sequence SEQ ID NO: 151. In some embodiments, the binding domain capable of binding to an HLA-G epitope comprises a V_{H} sequence comprising a Kabat CDR-H1 sequence comprising SEQ ID NO: 21, a Kabat CDR-H2 sequence comprising SEQ ID NO: 57, and a Kabat CDR-H3 sequence comprising SEQ ID NO: 84 and a VL sequence comprising a CDR-L1 sequence comprising SEQ ID NO: 105, a CDR-L2 sequence comprising SEQ ID NO: 128, and a CDR-L3 sequence SEQ ID NO: 149. In some embodiments, the binding domain capable of binding to an HLA-G epitope comprises a V_{H} sequence comprising a Kabat CDR-H1 sequence comprising SEQ ID NO: 24, a Kabat CDR-H2 sequence comprising SEQ ID NO: 61, and a Kabat CDR-H3 sequence comprising SEQ ID NO: 85 and a VL sequence comprising a CDR-L1 sequence comprising SEQ ID NO: 105, a CDR-L2 sequence comprising SEQ ID NO: 128, and a CDR-L3 sequence SEQ ID NO: 154. In some embodiments, the binding domain capable of binding to an HLA-G epitope comprises a V_{H} sequence comprising a Kabat CDR-H1 sequence comprising SEQ ID NO: 24, a Kabat CDR-H2 sequence comprising SEQ ID NO: 61, and a Kabat CDR-H3 sequence comprising SEQ ID NO: 86 and a VL sequence comprising a CDR-L1 sequence comprising SEQ ID NO: 105, a CDR-L2 sequence comprising SEQ ID NO: 128, and a CDR-L3 sequence SEQ ID NO: 154. In some embodiments, the binding domain capable of binding to an HLA-G epitope comprises a V_{H} sequence comprising a Kabat CDR-H1 sequence comprising SEQ ID NO: 25, a Kabat CDR-H2 sequence comprising SEQ ID NO: 62, and a Kabat CDR-H3 sequence comprising SEQ ID NO: 87 and a VL sequence comprising a CDR-L1 sequence comprising SEQ ID NO: 112, a CDR-L2 sequence comprising SEQ ID NO: 134, and a CDR-L3 sequence SEQ ID NO: 155. In some embodiments, the binding domain capable of binding to an HLA-G epitope comprises a V_{H} sequence comprising a Kabat CDR-H1 sequence comprising SEQ ID NO: 26, a Kabat CDR-H2 sequence comprising SEQ ID NO: 63, and a Kabat CDR-H3 sequence comprising SEQ ID NO: 88 and a VL sequence comprising a CDR-L1 sequence comprising SEQ ID NO: 113, a CDR-L2 sequence comprising SEQ ID NO: 135, and a CDR-L3 sequence SEQ ID NO: 156. In some embodiments, the binding domain capable of binding to an HLA-G epitope comprises a V_{H} sequence comprising a Kabat CDR-H1 sequence comprising SEQ ID NO: 26, a Kabat CDR-H2 sequence comprising SEQ ID NO: 63, and a Kabat CDR-H3 sequence comprising SEQ ID NO: 89 and a VL sequence comprising a CDR-L1 sequence comprising SEQ ID NO: 114, a CDR-L2 sequence comprising SEQ ID NO: 136, and a CDR-L3 sequence SEQ ID NO: 157. In some embodiments, the binding domain capable of binding to an HLA-G epitope comprises a V_{H} sequence comprising a Kabat CDR-H1 sequence comprising SEQ ID NO: 25, a Kabat CDR-H2 sequence comprising SEQ ID NO: 63, and a Kabat CDR-H3 sequence comprising SEQ ID NO: 90 and a VL sequence comprising a CDR-L1 sequence comprising SEQ ID NO: 115, a CDR-L2 sequence comprising SEQ ID NO: 135, and a CDR-L3 sequence SEQ ID NO: 157. In some embodiments, the binding domain capable of binding to an HLA-G epitope comprises a V_{H} sequence comprising a Kabat CDR-H1 sequence comprising SEQ ID NO: 27, a Kabat CDR-H2 sequence comprising SEQ ID NO: 64, and a Kabat CDR-H3 sequence comprising SEQ ID NO: 90 and a VL sequence comprising a CDR-L1 sequence comprising SEQ ID NO: 116, a CDR-L2 sequence comprising SEQ ID NO: 128, and a CDR-L3 sequence SEQ ID NO: 155. In some embodiments, the binding domain capable of binding to an HLA-G epitope comprises a V_{H} sequence comprising a Kabat CDR-H1 sequence comprising SEQ ID NO: 28, a Kabat CDR-H2 sequence comprising SEQ ID NO: 62, and a Kabat CDR-H3 sequence comprising SEQ ID NO: 91 and a VL sequence comprising a CDR-L1 sequence comprising SEQ ID NO: 117, a CDR-L2 sequence comprising SEQ ID NO: 137, and a CDR-L3 sequence SEQ ID NO: 155. In some embodiments, the binding domain capable of binding to an HLA-G epitope comprises a V_{H} sequence comprising a Kabat CDR-H1 sequence comprising SEQ ID NO: 29, a Kabat CDR-H2 sequence comprising SEQ ID NO: 64, and a Kabat CDR-H3 sequence comprising SEQ ID NO: 92 and a VL sequence comprising a CDR-L1 sequence comprising SEQ ID NO: 118, a CDR-L2 sequence comprising SEQ ID NO: 137, and a CDR-L3 sequence SEQ ID NO: 158. In some embodiments, the binding domain capable of binding to an HLA-G epitope comprises a V_{H} sequence comprising a Kabat CDR-H1 sequence comprising SEQ ID NO: 25, a Kabat CDR-H2 sequence comprising SEQ ID NO: 65, and a Kabat CDR-H3 sequence comprising SEQ ID NO: 93 and a VL sequence comprising a CDR-L1 sequence comprising SEQ ID NO: 118, a CDR-L2 sequence comprising SEQ ID NO: 138, and a CDR-L3 sequence SEQ ID NO: 155. In some embodiments, the binding domain capable of binding to an HLA-G epitope comprises a V_{H} sequence comprising a Kabat CDR-H1 sequence comprising SEQ ID NO: 30, a Kabat CDR-H2 sequence comprising SEQ ID NO: 66, and a Kabat CDR-H3 sequence comprising SEQ ID NO: 94 and a VL sequence comprising a CDR-L1 sequence comprising SEQ ID NO: 119, a CDR-L2 sequence comprising SEQ ID NO: 139, and a CDR-L3 sequence SEQ ID NO: 159. In some embodiments, the binding domain capable of binding to an HLA-G epitope comprises a V_{H} sequence comprising a Kabat CDR-H1 sequence comprising SEQ ID NO: 31, a Kabat CDR-H2 sequence comprising SEQ ID NO: 67, and a Kabat CDR-H3 sequence comprising SEQ ID NO: 95 and a VL sequence comprising a CDR-L1 sequence comprising SEQ ID NO: 120, a CDR-L2 sequence comprising SEQ ID NO: 140, and a CDR-L3 sequence SEQ ID NO: 160. In some embodiments, the binding domain capable of binding to an HLA-G epitope comprises a V_{H} sequence comprising a Kabat CDR-H1 sequence comprising SEQ ID NO: 32, a Kabat CDR-H2 sequence comprising SEQ ID NO: 68, and a Kabat CDR-H3 sequence comprising SEQ ID NO: 96 and a VL sequence comprising a CDR-L1 sequence comprising SEQ ID NO: 121, a CDR-L2 sequence comprising SEQ ID NO: 141, and a CDR-L3 sequence SEQ ID NO: 161. In some embodiments, the binding domain capable of binding to an HLA-G epitope comprises a V_{H} sequence comprising a Kabat CDR-H1 sequence comprising SEQ ID NO: 33, a Kabat CDR-H2 sequence comprising SEQ ID NO: 69, and a Kabat CDR-H3 sequence comprising SEQ ID NO: 97 and a VL sequence comprising a CDR-L1 sequence comprising SEQ ID NO: 122, a CDR-L2 sequence comprising SEQ ID NO: 142, and a CDR-L3 sequence SEQ ID NO: 162. In some embodiments, the binding domain capable of binding to an HLA-G epitope comprises a V_{H} sequence comprising a Kabat CDR-H1 sequence comprising SEQ ID NO: 34, a Kabat CDR-H2 sequence comprising SEQ ID NO: 70, and a Kabat CDR-H3 sequence comprising SEQ ID NO: 98 and a VL sequence comprising a CDR-L1 sequence comprising SEQ ID NO: 105, a CDR-L2 sequence comprising SEQ ID NO: 143, and a CDR-L3 sequence SEQ ID NO: 163. In some embodiments, the binding domain capable of binding to an HLA-G epitope comprises a V_{H} sequence comprising a Kabat CDR-H1 sequence comprising SEQ ID NO: 18, a Kabat CDR-H2 sequence comprising SEQ ID NO: 54, and a Kabat CDR-H3 sequence comprising SEQ ID NO: 99 and a VL sequence comprising a CDR-L1 sequence comprising SEQ ID NO: 105, a CDR-L2 sequence comprising SEQ ID NO: 143, and a CDR-L3 sequence SEQ ID NO: 164. In some embodiments, the binding domain capable of binding to an HLA-G epitope comprises a V_{H} sequence comprising a Kabat CDR-H1 sequence comprising SEQ ID NO: 31, a Kabat CDR-H2 sequence comprising SEQ ID NO: 71, and a Kabat CDR-H3 sequence comprising SEQ ID NO: 100 and a VL sequence comprising a CDR-L1 sequence comprising SEQ ID NO: 123, a CDR-L2 sequence comprising SEQ ID NO: 144, and a CDR-L3 sequence SEQ ID NO: 165. In some embodiments, the binding domain capable of binding to an HLA-G epitope comprises a V_{H} sequence comprising a Kabat CDR-H1 sequence comprising SEQ ID NO: 24, a Kabat CDR-H2 sequence comprising SEQ ID NO: 61, and a Kabat CDR-H3 sequence comprising SEQ ID NO: 101 and a VL sequence comprising a CDR-L1 sequence comprising SEQ ID NO: 124, a CDR-L2 sequence comprising SEQ ID NO: 145, and a CDR-L3 sequence SEQ ID NO: 166.

In some embodiments, the binding domain capable of binding to an HLA-G epitope comprises a V_{H} sequence comprising a Chothia CDR-H1 sequence comprising SEQ ID NO: 1, a Chothia CDR-H2 sequence comprising SEQ ID NO: 38, and a Chothia CDR-H3 sequence comprising SEQ ID NO: 76 and a VL sequence comprising a CDR-L1 sequence comprising SEQ ID NO: 105, a CDR-L2 sequence comprising SEQ ID NO: 128, and a CDR-L3 sequence SEQ ID NO: 149. In some embodiments, the binding domain capable of binding to an HLA-G epitope comprises a V_{H} sequence comprising a Chothia CDR-H1 sequence comprising SEQ ID NO: 2, a Chothia CDR-H2 sequence comprising SEQ ID NO: 39, and a Chothia CDR-H3 sequence comprising SEQ ID NO: 77 and a VL sequence comprising a CDR-L1 sequence comprising SEQ ID NO: 105, a CDR-L2 sequence comprising SEQ ID NO: 128, and a CDR-L3 sequence SEQ ID NO: 149. In some embodiments, the binding domain capable of binding to an HLA-G epitope comprises a V_{H} sequence comprising a Chothia CDR-H1 sequence comprising SEQ ID NO: 2, a Chothia CDR-H2 sequence comprising SEQ ID NO: 40, and a Chothia CDR-H3 sequence comprising SEQ ID NO: 78 and a VL sequence comprising a CDR-L1 sequence comprising SEQ ID NO: 105, a CDR-L2 sequence comprising SEQ ID NO: 128, and a CDR-L3 sequence SEQ ID NO: 149. In some embodiments, the binding domain capable of binding to an HLA-G epitope comprises a V_{H} sequence comprising a Chothia CDR-H1 sequence comprising SEQ ID NO: 3, a Chothia CDR-H2 sequence comprising SEQ ID NO: 41, and a Chothia CDR-H3 sequence comprising SEQ ID NO: 79 and a VL sequence comprising a CDR-L1 sequence comprising SEQ ID NO: 105, a CDR-L2 sequence comprising SEQ ID NO: 128, and a CDR-L3 sequence SEQ ID NO: 149. In some embodiments, the binding domain capable of binding to an HLA-G epitope comprises a V_{H} sequence comprising a Chothia CDR-H1 sequence comprising SEQ ID NO: 4, a Chothia CDR-H2 sequence comprising SEQ ID NO: 41, and a Chothia CDR-H3 sequence comprising SEQ ID NO: 80 and a VL sequence comprising a CDR-L1 sequence comprising SEQ ID NO: 105, a CDR-L2 sequence comprising SEQ ID NO: 128, and a CDR-L3 sequence SEQ ID NO: 149. In some embodiments, the binding domain capable of binding to an HLA-G epitope comprises a V_{H} sequence comprising a Chothia CDR-H1 sequence comprising SEQ ID NO: 3, a Chothia CDR-H2 sequence comprising SEQ ID NO: 41, and a Chothia CDR-H3 sequence comprising SEQ ID NO: 76 and a VL sequence comprising a CDR-L1 sequence comprising SEQ ID NO: 106, a CDR-L2 sequence comprising SEQ ID NO: 129, and a CDR-L3 sequence SEQ ID NO: 150. In some embodiments, the binding domain capable of binding to an HLA-G epitope comprises a V_{H} sequence comprising a Chothia CDR-H1 sequence comprising SEQ ID NO: 2, a Chothia CDR-H2 sequence comprising SEQ ID NO: 42, and a Chothia CDR-H3 sequence comprising SEQ ID NO: 76 and a VL sequence comprising a CDR-L1 sequence comprising SEQ ID NO: 107, a CDR-L2 sequence comprising SEQ ID NO: 128, and a CDR-L3 sequence SEQ ID NO: 151. In some embodiments, the binding domain capable of binding to an HLA-G epitope comprises a V_{H} sequence comprising a Chothia CDR-H1 sequence comprising SEQ ID NO: 2, a Chothia CDR-H2 sequence comprising SEQ ID NO: 43, and a Chothia CDR-H3 sequence comprising SEQ ID NO: 81 and a VL sequence comprising a CDR-L1 sequence comprising SEQ ID NO: 108, a CDR-L2 sequence comprising SEQ ID NO: 130, and a CDR-L3 sequence SEQ ID NO: 151. In some embodiments, the binding domain capable of binding to an HLA-G epitope comprises a V_{H} sequence comprising a Chothia CDR-H1 sequence comprising SEQ ID NO: 2, a Chothia CDR-H2 sequence comprising SEQ ID NO: 42, and a Chothia CDR-H3 sequence comprising SEQ ID NO: 82 and a VL sequence comprising a CDR-L1 sequence comprising SEQ ID NO: 109, a CDR-L2 sequence comprising SEQ ID NO: 131, and a CDR-L3 sequence SEQ ID NO: 152. In some embodiments, the binding domain capable of binding to an HLA-G epitope comprises a V_{H} sequence comprising a Chothia CDR-H1 sequence comprising SEQ ID NO: 5, a Chothia CDR-H2 sequence comprising SEQ ID NO: 42, and a Chothia CDR-H3 sequence comprising SEQ ID NO: 76 and a VL sequence comprising a CDR-L1 sequence comprising SEQ ID NO: 107, a CDR-L2 sequence comprising SEQ ID NO: 132, and a CDR-L3 sequence SEQ ID NO: 153. In some embodiments, the binding domain capable of binding to an HLA-G epitope comprises a V_{H} sequence comprising a Chothia CDR-H1 sequence comprising SEQ ID NO: 2, a Chothia CDR-H2 sequence comprising SEQ ID NO: 42, and a Chothia CDR-H3 sequence comprising SEQ ID NO: 81 and a VL sequence comprising a CDR-L1 sequence comprising SEQ ID NO: 110, a CDR-L2 sequence comprising SEQ ID NO: 132, and a CDR-L3 sequence SEQ ID NO: 151. In some embodiments, the binding domain capable of binding to an HLA-G epitope comprises a V_{H} sequence comprising a Chothia CDR-H1 sequence comprising SEQ ID NO: 3, a Chothia CDR-H2 sequence comprising SEQ ID NO: 41, and a Chothia CDR-H3 sequence comprising SEQ ID NO: 83 and a VL sequence comprising a CDR-L1 sequence comprising SEQ ID NO: 111, a CDR-L2 sequence comprising SEQ ID NO: 133, and a CDR-L3 sequence SEQ ID NO: 151. In some embodiments, the binding domain capable of binding to an HLA-G epitope comprises a V_{H} sequence comprising a Chothia CDR-H1 sequence comprising SEQ ID NO: 3, a Chothia CDR-H2 sequence comprising SEQ ID NO: 41, and a Chothia CDR-H3 sequence comprising SEQ ID NO: 84 and a VL sequence comprising a CDR-L1 sequence comprising SEQ ID NO: 105, a CDR-L2 sequence comprising SEQ ID NO: 128, and a CDR-L3 sequence SEQ ID NO: 149. In some embodiments, the binding domain capable of binding to an HLA-G epitope comprises a V_{H} sequence comprising a Chothia CDR-H1 sequence comprising SEQ ID NO: 6, a Chothia CDR-H2 sequence comprising SEQ ID NO: 44, and a Chothia CDR-H3 sequence comprising SEQ ID NO: 85 and a VL sequence comprising a CDR-L1 sequence comprising SEQ ID NO: 105, a CDR-L2 sequence comprising SEQ ID NO: 128, and a CDR-L3 sequence SEQ ID NO: 154. In some embodiments, the binding domain capable of binding to an HLA-G epitope comprises a V_{H} sequence comprising a Chothia CDR-H1 sequence comprising SEQ ID NO: 6, a Chothia CDR-H2 sequence comprising SEQ ID NO: 44, and a Chothia CDR-H3 sequence comprising SEQ ID NO: 86 and a VL sequence comprising a CDR-L1 sequence comprising SEQ ID NO: 105, a CDR-L2 sequence comprising SEQ ID NO: 128, and a CDR-L3 sequence SEQ ID NO: 154. In some embodiments, the binding domain capable of binding to an HLA-G epitope comprises a V_{H} sequence comprising a Chothia CDR-H1 sequence comprising SEQ ID NO: 7, a Chothia CDR-H2 sequence comprising SEQ ID NO: 45, and a Chothia CDR-H3 sequence comprising SEQ ID NO: 87 and a VL sequence comprising a CDR-L1 sequence comprising SEQ ID NO: 112, a CDR-L2 sequence comprising SEQ ID NO: 134, and a CDR-L3 sequence SEQ ID NO: 155. In some embodiments, the binding domain capable of binding to an HLA-G epitope comprises a V_{H} sequence comprising a Chothia CDR-H1 sequence comprising SEQ ID NO: 8, a Chothia CDR-H2 sequence comprising SEQ ID NO: 44, and a Chothia CDR-H3 sequence comprising SEQ ID NO: 88 and a VL sequence comprising a CDR-L1 sequence comprising SEQ ID NO: 113, a CDR-L2 sequence comprising SEQ ID NO: 135, and a CDR-L3 sequence SEQ ID NO: 156. In some embodiments, the binding domain capable of binding to an HLA-G epitope comprises a V_{H} sequence comprising a Chothia CDR-H1 sequence comprising SEQ ID NO: 8, a Chothia CDR-H2 sequence comprising SEQ ID NO: 44, and a Chothia CDR-H3 sequence comprising SEQ ID NO: 89 and a VL sequence comprising a CDR-L1 sequence comprising SEQ ID NO: 114, a CDR-L2 sequence comprising SEQ ID NO: 136, and a CDR-L3 sequence SEQ ID NO: 157. In some embodiments, the binding domain capable of binding to an HLA-G epitope comprises a V_{H} sequence comprising a Chothia CDR-H1 sequence comprising SEQ ID NO: 7, a Chothia CDR-H2 sequence comprising SEQ ID NO: 44, and a Chothia CDR-H3 sequence comprising SEQ ID NO: 90 and a VL sequence comprising a CDR-L1 sequence comprising SEQ ID NO: 115, a CDR-L2 sequence comprising SEQ ID NO: 135, and a CDR-L3 sequence SEQ ID NO: 157. In some embodiments, the binding domain capable of binding to an HLA-G epitope comprises a V_{H} sequence comprising a Chothia CDR-H1 sequence comprising SEQ ID NO: 9, a Chothia CDR-H2 sequence comprising SEQ ID NO: 44, and a Chothia CDR-H3 sequence comprising SEQ ID NO: 90 and a VL sequence comprising a CDR-L1 sequence comprising SEQ ID NO: 116, a CDR-L2 sequence comprising SEQ ID NO: 128, and a CDR-L3 sequence SEQ ID NO: 155. In some embodiments, the binding domain capable of binding to an HLA-G epitope comprises a V_{H} sequence comprising a Chothia CDR-H1 sequence comprising SEQ ID NO: 6, a Chothia CDR-H2 sequence comprising SEQ ID NO: 45, and a Chothia CDR-H3 sequence comprising SEQ ID NO: 91 and a VL sequence comprising a CDR-L1 sequence comprising SEQ ID NO: 117, a CDR-L2 sequence comprising SEQ ID NO: 137, and a CDR-L3 sequence SEQ ID NO: 155. In some embodiments, the binding domain capable of binding to an HLA-G epitope comprises a V_{H} sequence comprising a Chothia CDR-H1 sequence comprising SEQ ID NO: 8, a Chothia CDR-H2 sequence comprising SEQ ID NO: 44, and a Chothia CDR-H3 sequence comprising SEQ ID NO: 92 and a VL sequence comprising a CDR-L1 sequence comprising SEQ ID NO: 118, a CDR-L2 sequence comprising SEQ ID NO: 137, and a CDR-L3 sequence SEQ ID NO: 158. In some embodiments, the binding domain capable of binding to an HLA-G epitope comprises a V_{H} sequence comprising a Chothia CDR-H1 sequence comprising SEQ ID NO: 7, a Chothia CDR-H2 sequence comprising SEQ ID NO: 44, and a Chothia CDR-H3 sequence comprising SEQ ID NO: 93 and a VL sequence comprising a CDR-L1 sequence comprising SEQ ID NO: 118, a CDR-L2 sequence comprising SEQ ID NO: 138, and a CDR-L3 sequence SEQ ID NO: 155. In some embodiments, the binding domain capable of binding to an HLA-G epitope comprises a V_{H} sequence comprising a Chothia CDR-H1 sequence comprising SEQ ID NO: 10, a Chothia CDR-H2 sequence comprising SEQ ID NO: 46, and a Chothia CDR-H3 sequence comprising SEQ ID NO: 94 and a VL sequence comprising a CDR-L1 sequence comprising SEQ ID NO: 119, a CDR-L2 sequence comprising SEQ ID NO: 139, and a CDR-L3 sequence SEQ ID NO: 159. In some embodiments, the binding domain capable of binding to an HLA-G epitope comprises a V_{H} sequence comprising a Chothia CDR-H1 sequence comprising SEQ ID NO: 11, a Chothia CDR-H2 sequence comprising SEQ ID NO: 47, and a Chothia CDR-H3 sequence comprising SEQ ID NO: 95 and a VL sequence comprising a CDR-L1 sequence comprising SEQ ID NO: 120, a CDR-L2 sequence comprising SEQ ID NO: 140, and a CDR-L3 sequence SEQ ID NO: 160. In some embodiments, the binding domain capable of binding to an HLA-G epitope comprises a V_{H} sequence comprising a Chothia CDR-H1 sequence comprising SEQ ID NO: 12, a Chothia CDR-H2 sequence comprising SEQ ID NO: 48, and a Chothia CDR-H3 sequence comprising SEQ ID NO: 96 and a VL sequence comprising a CDR-L1 sequence comprising SEQ ID NO: 121, a CDR-L2 sequence comprising SEQ ID NO: 141, and a CDR-L3 sequence SEQ ID NO: 161. In some embodiments, the binding domain capable of binding to an HLA-G epitope comprises a V_{H} sequence comprising a Chothia CDR-H1 sequence comprising SEQ ID NO: 13, a Chothia CDR-H2 sequence comprising SEQ ID NO: 44, and a Chothia CDR-H3 sequence comprising SEQ ID NO: 97 and a VL sequence comprising a CDR-L1 sequence comprising SEQ ID NO: 122, a CDR-L2 sequence comprising SEQ ID NO: 142, and a CDR-L3 sequence SEQ ID NO: 162. In some embodiments, the binding domain capable of binding to an HLA-G epitope comprises a V_{H} sequence comprising a Chothia CDR-H1 sequence comprising SEQ ID NO: 14, a Chothia CDR-H2 sequence comprising SEQ ID NO: 49, and a Chothia CDR-H3 sequence comprising SEQ ID NO: 98 and a VL sequence comprising a CDR-L1 sequence comprising SEQ ID NO: 105, a CDR-L2 sequence comprising SEQ ID NO: 143, and a CDR-L3 sequence SEQ ID NO: 163. In some embodiments, the binding domain capable of binding to an HLA-G epitope comprises a V_{H} sequence comprising a Chothia CDR-H1 sequence comprising SEQ ID NO: 1, a Chothia CDR-H2 sequence comprising SEQ ID NO: 38, and a Chothia CDR-H3 sequence comprising SEQ ID NO: 99 and a VL sequence comprising a CDR-L1 sequence comprising SEQ ID NO: 105, a CDR-L2 sequence comprising SEQ ID NO: 143, and a CDR-L3 sequence SEQ ID NO: 164. In some embodiments, the binding domain capable of binding to an HLA-G epitope comprises a V_{H} sequence comprising a Chothia CDR-H1 sequence comprising SEQ ID NO: 11, a Chothia CDR-H2 sequence comprising SEQ ID NO: 50, and a Chothia CDR-H3 sequence comprising SEQ ID NO: 100 and a VL sequence comprising a CDR-L1 sequence comprising SEQ ID NO: 123, a CDR-L2 sequence comprising SEQ ID NO: 144, and a CDR-L3 sequence SEQ ID NO: 165. In some embodiments, the binding domain capable of binding to an HLA-G epitope comprises a V_{H} sequence comprising a Chothia CDR-H1 sequence comprising SEQ ID NO: 6, a Chothia CDR-H2 sequence comprising SEQ ID NO: 44, and a Chothia CDR-H3 sequence comprising SEQ ID NO: 101 and a VL sequence comprising a CDR-L1 sequence comprising SEQ ID NO: 124, a CDR-L2 sequence comprising SEQ ID NO: 145, and a CDR-L3 sequence SEQ ID NO: 166.

In some embodiments, the binding domain capable of binding to an HLA-G epitope comprises three heavy chain CDRs and three light chain CDRs, each comprising, consisting of, or consisting essentially of a CDR sequence of a VH-VL pair, with the VH having the sequence set forth in one of SEQ ID NOS: 170-200 and with the VL having the sequence set forth in one of SEQ ID NOS: 204-228.

Variants of V_{H}-V_{L} Pairs

In some embodiments, the V_{H}-V_{L} pairs provided herein comprise a variant of an illustrative V_{H} and/or V_{L} sequence provided in this disclosure.

In some embodiments, the V_{H} sequence comprises, consists of, or consists essentially of a variant of an illustrative V_{H} sequence provided in this disclosure. In some embodiments, the V_{H} sequence comprises, consists of, or consists essentially of a sequence having at least 85%, 90%, 95%, 96%, 97%, 98%, 99%, or 99.1% identity with any of the illustrative V_{H} sequences provided in this disclosure.

In some embodiments, the V_{H} sequence comprises, consists of, or consists essentially of any of the illustrative V_{H} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

In some embodiments, the V_{L} sequence comprises, consists of, or consists essentially of a variant of an illustrative V_{L} sequence provided in this disclosure. In some embodiments, the V_{L} sequence comprises, consists of, or consists essentially of a sequence having at least 85%, 90%, 95%, 96%, 97%, 98%, 99%, or 99.05% identity with any of the illustrative V_{L} sequences provided in this disclosure.

In some embodiments, the V_{L} sequence comprises, consists of, or consists essentially of any of the illustrative V_{L} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

### HC+LC

In some embodiments, the binding domain capable of binding to an HLA-G epitope comprises or consists of one or more heavy chains consisting of an HC sequence and one or more light chains consisting of an LC sequence. In some embodiments, the binding domain capable of binding to an HLA-G epitope comprises or consists of two identical heavy chains consisting of an HC sequence and two identical light chains consisting of an LC sequence.

In some embodiments, the HC sequence is an HC sequence comprising, consisting of, or consisting essentially of a sequence selected from SEQ ID NOS: 232-262 and the LC sequence is an LC sequence comprising, consisting of, or consisting essentially of SEQ ID NOS: 300-330. In some embodiments, the HC sequence is an HC sequence consisting of a sequence selected from SEQ ID NOS: 232-262 and the LC sequence is an LC sequence consisting of a sequence selected from SEQ ID NOS: 300-330. In some embodiments, the HC sequence is an HC sequence comprising, consisting of, or consisting essentially of a sequence selected from SEQ ID NOS: 266-296 and the LC sequence is an LC sequence comprising, consisting of, or consisting essentially of SEQ ID NOS: 300-330. In some embodiments, the HC sequence is an HC sequence consisting of a sequence selected from SEQ ID NOS: 266-296 and the LC sequence is an LC sequence consisting of a sequence selected from SEQ ID NOS: 300-330.

In some embodiments, the HC sequence is an HC sequence consisting of SEQ ID NO: 232 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 233 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 234 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 235 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 236 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 237 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 238 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 239 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 240 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 241 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 242 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 243 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 244 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 245 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 246 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 247 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 248 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 249 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 250 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 251 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 252 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 253 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 254 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 255 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 256 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 257 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 258 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 259 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 260 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 261 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 262 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 266 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 267 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 268 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 269 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 270 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 271 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 272 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 273 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 274 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 275 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 276 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 277 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 278 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 279 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 280 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 281 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 282 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 283 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 284 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 285 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 286 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 287 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 288 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 289 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 290 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 291 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 292 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 293 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 294 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 295 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 296 and the LC sequence is an LC sequence consisting of a sequence selected from SEQ ID NOS: 300-330. In some embodiments, the LC sequence is SEQ ID NO: 300. In some embodiments, the LC sequence is SEQ ID NO: 301. In some embodiments, the LC sequence is SEQ ID NO: 302. In some embodiments, the LC sequence is SEQ ID NO: 303. In some embodiments, the LC sequence is SEQ ID NO: 304. In some embodiments, the LC sequence is SEQ ID NO: 305. In some embodiments, the LC sequence is SEQ ID NO: 306. In some embodiments, the LC sequence is SEQ ID NO: 307. In some embodiments, the LC sequence is SEQ ID NO: 308. In some embodiments, the LC sequence is SEQ ID NO: 309. In some embodiments, the LC sequence is SEQ ID NO: 310. In some embodiments, the LC sequence is SEQ ID NO: 311. In some embodiments, the LC sequence is SEQ ID NO: 312. In some embodiments, the LC sequence is SEQ ID NO: 313. In some embodiments, the LC sequence is SEQ ID NO: 314. In some embodiments, the LC sequence is SEQ ID NO: 315. In some embodiments, the LC sequence is SEQ ID NO: 316. In some embodiments, the LC sequence is SEQ ID NO: 317. In some embodiments, the LC sequence is SEQ ID NO: 318. In some embodiments, the LC sequence is SEQ ID NO: 319. In some embodiments, the LC sequence is SEQ ID NO: 320. In some embodiments, the LC sequence is SEQ ID NO: 321. In some embodiments, the LC sequence is SEQ ID NO: 322. In some embodiments, the LC sequence is SEQ ID NO: 323. In some embodiments, the LC sequence is SEQ ID NO: 324. In some embodiments, the LC sequence is SEQ ID NO: 325. In some embodiments, the LC sequence is SEQ ID NO: 326. In some embodiments, the LC sequence is SEQ ID NO: 327. In some embodiments, the LC sequence is SEQ ID NO: 328. In some embodiments, the LC sequence is SEQ ID NO: 329. In some embodiments, the LC sequence is SEQ ID NO: 330.

In some embodiments, the HC sequence is an HC sequence consisting of SEQ ID NO: 232 and the LC sequence is an LC sequence consisting of sequence SEQ ID NO: 300. In some embodiments, the HC sequence is an HC sequence consisting of SEQ ID NO: 233 and the LC sequence is an LC sequence consisting of sequence SEQ ID NO: 301. In some embodiments, the HC sequence is an HC sequence consisting of SEQ ID NO: 234 and the LC sequence is an LC sequence consisting of sequence SEQ ID NO: 302. In some embodiments, the HC sequence is an HC sequence consisting of SEQ ID NO: 235 and the LC sequence is an LC sequence consisting of sequence SEQ ID NO: 303. In some embodiments, the HC sequence is an HC sequence consisting of SEQ ID NO: 236 and the LC sequence is an LC sequence consisting of sequence SEQ ID NO: 304. In some embodiments, the HC sequence is an HC sequence consisting of SEQ ID NO: 237 and the LC sequence is an LC sequence consisting of sequence SEQ ID NO: 305. In some embodiments, the HC sequence is an HC sequence consisting of SEQ ID NO: 238 and the LC sequence is an LC sequence consisting of sequence SEQ ID NO: 306. In some embodiments, the HC sequence is an HC sequence consisting of SEQ ID NO: 239 and the LC sequence is an LC sequence consisting of sequence SEQ ID NO: 307. In some embodiments, the HC sequence is an HC sequence consisting of SEQ ID NO: 240 and the LC sequence is an LC sequence consisting of sequence SEQ ID NO: 308. In some embodiments, the HC sequence is an HC sequence consisting of SEQ ID NO: 241 and the LC sequence is an LC sequence consisting of sequence SEQ ID NO: 309. In some embodiments, the HC sequence is an HC sequence consisting of SEQ ID NO: 242 and the LC sequence is an LC sequence consisting of sequence SEQ ID NO: 310. In some embodiments, the HC sequence is an HC sequence consisting of SEQ ID NO: 243 and the LC sequence is an LC sequence consisting of sequence SEQ ID NO: 311. In some embodiments, the HC sequence is an HC sequence consisting of SEQ ID NO: 244 and the LC sequence is an LC sequence consisting of sequence SEQ ID NO: 312. In some embodiments, the HC sequence is an HC sequence consisting of SEQ ID NO: 245 and the LC sequence is an LC sequence consisting of sequence SEQ ID NO: 313. In some embodiments, the HC sequence is an HC sequence consisting of SEQ ID NO: 246 and the LC sequence is an LC sequence consisting of sequence SEQ ID NO: 314. In some embodiments, the HC sequence is an HC sequence consisting of SEQ ID NO: 247 and the LC sequence is an LC sequence consisting of sequence SEQ ID NO: 315. In some embodiments, the HC sequence is an HC sequence consisting of SEQ ID NO: 248 and the LC sequence is an LC sequence consisting of sequence SEQ ID NO: 316. In some embodiments, the HC sequence is an HC sequence consisting of SEQ ID NO: 249 and the LC sequence is an LC sequence consisting of sequence SEQ ID NO: 317. In some embodiments, the HC sequence is an HC sequence consisting of SEQ ID NO: 250 and the LC sequence is an LC sequence consisting of sequence SEQ ID NO: 318. In some embodiments, the HC sequence is an HC sequence consisting of SEQ ID NO: 251 and the LC sequence is an LC sequence consisting of sequence SEQ ID NO: 319. In some embodiments, the HC sequence is an HC sequence consisting of SEQ ID NO: 252 and the LC sequence is an LC sequence consisting of sequence SEQ ID NO: 320. In some embodiments, the HC sequence is an HC sequence consisting of SEQ ID NO: 253 and the LC sequence is an LC sequence consisting of sequence SEQ ID NO: 321. In some embodiments, the HC sequence is an HC sequence consisting of SEQ ID NO: 254 and the LC sequence is an LC sequence consisting of sequence SEQ ID NO: 322. In some embodiments, the HC sequence is an HC sequence consisting of SEQ ID NO: 255 and the LC sequence is an LC sequence consisting of sequence SEQ ID NO: 323. In some embodiments, the HC sequence is an HC sequence consisting of SEQ ID NO: 256 and the LC sequence is an LC sequence consisting of sequence SEQ ID NO: 324. In some embodiments, the HC sequence is an HC sequence consisting of SEQ ID NO: 257 and the LC sequence is an LC sequence consisting of sequence SEQ ID NO: 325. In some embodiments, the HC sequence is an HC sequence consisting of SEQ ID NO: 258 and the LC sequence is an LC sequence consisting of sequence SEQ ID NO: 326. In some embodiments, the HC sequence is an HC sequence consisting of SEQ ID NO: 259 and the LC sequence is an LC sequence consisting of sequence SEQ ID NO: 327. In some embodiments, the HC sequence is an HC sequence consisting of SEQ ID NO: 260 and the LC sequence is an LC sequence consisting of sequence SEQ ID NO: 328. In some embodiments, the HC sequence is an HC sequence consisting of SEQ ID NO: 261 and the LC sequence is an LC sequence consisting of sequence SEQ ID NO: 329. In some embodiments, the HC sequence is an HC sequence consisting of SEQ ID NO: 262 and the LC sequence is an LC sequence consisting of sequence SEQ ID NO: 330.

In some embodiments, the HC sequence is an HC sequence consisting of SEQ ID NO: 266 and the LC sequence is an LC sequence consisting of a sequence SEQ ID NO: 300. In some embodiments, the HC sequence is an HC sequence consisting of SEQ ID NO: 267 and the LC sequence is an LC sequence consisting of sequence SEQ ID NO: 301. In some embodiments, the HC sequence is an HC sequence consisting of SEQ ID NO: 268 and the LC sequence is an LC sequence consisting of sequence SEQ ID NO: 302. In some embodiments, the HC sequence is an HC sequence consisting of SEQ ID NO: 269 and the LC sequence is an LC sequence consisting of sequence SEQ ID NO: 303. In some embodiments, the HC sequence is an HC sequence consisting of SEQ ID NO: 270 and the LC sequence is an LC sequence consisting of sequence SEQ ID NO: 304. In some embodiments, the HC sequence is an HC sequence consisting of SEQ ID NO: 271 and the LC sequence is an LC sequence consisting of sequence SEQ ID NO: 305. In some embodiments, the HC sequence is an HC sequence consisting of SEQ ID NO: 272 and the LC sequence is an LC sequence consisting of sequence SEQ ID NO: 306. In some embodiments, the HC sequence is an HC sequence consisting of SEQ ID NO: 273 and the LC sequence is an LC sequence consisting of sequence SEQ ID NO: 307. In some embodiments, the HC sequence is an HC sequence consisting of SEQ ID NO: 274 and the LC sequence is an LC sequence consisting of sequence SEQ ID NO: 308. In some embodiments, the HC sequence is an HC sequence consisting of SEQ ID NO: 275 and the LC sequence is an LC sequence consisting of sequence SEQ ID NO: 309. In some embodiments, the HC sequence is an HC sequence consisting of SEQ ID NO: 276 and the LC sequence is an LC sequence consisting of sequence SEQ ID NO: 310. In some embodiments, the HC sequence is an HC sequence consisting of SEQ ID NO: 277 and the LC sequence is an LC sequence consisting of sequence SEQ ID NO: 311. In some embodiments, the HC sequence is an HC sequence consisting of SEQ ID NO: 278 and the LC sequence is an LC sequence consisting of sequence SEQ ID NO: 312. In some embodiments, the HC sequence is an HC sequence consisting of SEQ ID NO: 279 and the LC sequence is an LC sequence consisting of sequence SEQ ID NO: 313. In some embodiments, the HC sequence is an HC sequence consisting of SEQ ID NO: 280 and the LC sequence is an LC sequence consisting of sequence SEQ ID NO: 314. In some embodiments, the HC sequence is an HC sequence consisting of SEQ ID NO: 281 and the LC sequence is an LC sequence consisting of sequence SEQ ID NO: 315. In some embodiments, the HC sequence is an HC sequence consisting of SEQ ID NO: 282 and the LC sequence is an LC sequence consisting of sequence SEQ ID NO: 316. In some embodiments, the HC sequence is an HC sequence consisting of SEQ ID NO: 283 and the LC sequence is an LC sequence consisting of sequence SEQ ID NO: 317. In some embodiments, the HC sequence is an HC sequence consisting of SEQ ID NO: 284 and the LC sequence is an LC sequence consisting of sequence SEQ ID NO: 318. In some embodiments, the HC sequence is an HC sequence consisting of SEQ ID NO: 285 and the LC sequence is an LC sequence consisting of sequence SEQ ID NO: 319. In some embodiments, the HC sequence is an HC sequence consisting of SEQ ID NO: 286 and the LC sequence is an LC sequence consisting of sequence SEQ ID NO: 320. In some embodiments, the HC sequence is an HC sequence consisting of SEQ ID NO: 287 and the LC sequence is an LC sequence consisting of sequence SEQ ID NO: 321. In some embodiments, the HC sequence is an HC sequence consisting of SEQ ID NO: 288 and the LC sequence is an LC sequence consisting of sequence SEQ ID NO: 322. In some embodiments, the HC sequence is an HC sequence consisting of SEQ ID NO: 289 and the LC sequence is an LC sequence consisting of sequence SEQ ID NO: 323. In some embodiments, the HC sequence is an HC sequence consisting of SEQ ID NO: 290 and the LC sequence is an LC sequence consisting of sequence SEQ ID NO: 324. In some embodiments, the HC sequence is an HC sequence consisting of SEQ ID NO: 291 and the LC sequence is an LC sequence consisting of sequence SEQ ID NO: 325. In some embodiments, the HC sequence is an HC sequence consisting of SEQ ID NO: 292 and the LC sequence is an LC sequence consisting of sequence SEQ ID NO: 326. In some embodiments, the HC sequence is an HC sequence consisting of SEQ ID NO: 293 and the LC sequence is an LC sequence consisting of sequence SEQ ID NO: 327. In some embodiments, the HC sequence is an HC sequence consisting of SEQ ID NO: 294 and the LC sequence is an LC sequence consisting of sequence SEQ ID NO: 328. In some embodiments, the HC sequence is an HC sequence consisting of SEQ ID NO: 295 and the LC sequence is an LC sequence consisting of sequence SEQ ID NO: 329. In some embodiments, the HC sequence is an HC sequence consisting of SEQ ID NO: 296 and the LC sequence is an LC sequence consisting of sequence SEQ ID NO: 330.

In some embodiments, the binding domain capable of binding to an HLA-G epitope comprises three heavy chain CDRs and three light chain CDRs, each comprising, consisting of, or consisting essentially of a CDR sequence of a HC-LC pair set forth above. In some embodiments, the binding domain capable of binding to an HLA-G epitope comprises three heavy chain CDRs and three light chain CDRs, each comprising, consisting of, or consisting essentially of a CDR sequence of a HC-LC pair set forth in Table S.

### Binding Domains Capable of Binding to CD3

In some embodiments, the second epitope comprises a CD3ε epitope. In some embodiments, the CD3ε epitope comprises or consists of an amino acid sequence set forth in SEQ ID NO: 629.

CD3 is a protein complex and T cell co-receptor that is involved in activating the cytotoxic T cell (CD8⁺ T cells), T helper cells (CD4⁺ T cells) and natural killer T cells (NKT cells).” CD3 is composed of four distinct chains. In mammals, the complex contains a CD3γ chain, a CD3δ chain, and two CD3ε chains. These chains associate with the T-cell receptor (TCR) and the ζ-chain (zeta-chain) to generate an activation signal in T lymphocytes. The TCR, ζ-chain, and CD3 molecules together constitute the TCR complex.

The CD3γ, CD3δ, and CD3ε chains are highly related cell-surface proteins of the immunoglobulin superfamily containing a single extracellular immunoglobulin domain. The intracellular tails of the CD3γ, CD3ε, and CD3δ molecules each contain a single conserved motif known as an immunoreceptor tyrosine-based activation motif or ITAM for short, which is essential for the signaling capacity of the TCR. The intracellular tail of CD3ζ contains 3 ITAM motifs. Phosphorylation of the ITAM on CD3 renders the CD3 chain capable of binding an enzyme called ZAP70 (zeta associated protein), a kinase that is important in the signaling cascade of the T cell. In some embodiments, CD3 proteins include murine CD3. In some embodiments, CD3 proteins include cynomolgus CD3.

### CDR-H3 Sequences

In some embodiments, the additional binding domain capable of binding to a CD3ε epitope comprises a CDR-H3 sequence comprising, consisting of, or consisting essentially of a sequence selected from SEQ ID NOS: 379-382. In some embodiments, the additional binding domain capable of binding to a CD3ε epitope comprises a CDR-H3 sequence comprising, consisting of, or consisting essentially of SEQ ID NO: 379.

In some embodiments, the CDR-H3 sequence comprises, consists of, or consists essentially of a variant of an illustrative CDR-H3 sequence provided in this disclosure. In some embodiments, the CDR-H3 sequence comprises, consists of, or consists essentially of a sequence having at least 70%, 75%, 80%, 85%, 90%, or 95% identity with any of the illustrative CDR-H3 sequences provided in this disclosure. In some embodiments, the CDR-H3 sequence comprises, consists of, or consists essentially of any of the illustrative CDR-H3 sequences provided in this disclosure, with 1, 2, or 3 amino acid substitutions. In some embodiments, the amino acid substitutions are conservative amino acid substitutions.

### V_{H} Sequences Comprising Illustrative CDRs

In some embodiments, the additional binding domain capable of binding to a CD3ε epitope comprises a V_{H} sequence comprising one or more CDR-H sequences comprising, consisting of, or consisting essentially of one or more illustrative CDR-H sequences provided in this disclosure, and variants thereof.

### V_{H} Sequences Comprising Illustrative Kabat CDRs

In some embodiments, the additional binding domain capable of binding to a CD3ε epitope comprises a V_{H} sequence comprising one or more Kabat CDR-H sequences comprising, consisting of, or consisting essentially of one or more illustrative Kabat CDR-H sequences provided in this disclosure, and variants thereof.

### Kabat CDR-H3

In some embodiments, the additional binding domain capable of binding to a CD3ε epitope comprises a V_{H} sequence comprising a Kabat CDR-H3 sequence comprising, consisting of, or consisting essentially of a sequence selected from SEQ ID NOS: 379-382. In some embodiments, the additional binding domain capable of binding to a CD3ε epitope comprises a V_{H} sequence comprising a Kabat CDR-H3 sequence comprising, consisting of, or consisting essentially of SEQ ID NO: 379.

### Kabat CDR-H2

In some embodiments, the additional binding domain capable of binding to a CD3ε epitope comprises a V_{H} sequence comprising a Kabat CDR-H2 sequence comprising, consisting of, or consisting essentially of a sequence selected from SEQ ID NOS: 371-375. In some embodiments, the additional binding domain capable of binding to a CD3ε epitope comprises a V_{H} sequence comprising a Kabat CDR-H2 sequence comprising, consisting of, or consisting essentially of SEQ ID NO: 371.

### Kabat CDR-H1

In some embodiments, the additional binding domain capable of binding to a CD3ε epitope comprises a V_{H} sequence comprising a Kabat CDR-H1 sequence comprising, consisting of, or consisting essentially of a sequence selected from SEQ ID NOS: 354-357. In some embodiments, the additional binding domain capable of binding to a CD3ε epitope comprises a V_{H} sequence comprising a Kabat CDR-H1 sequence comprising, consisting of, or consisting essentially of SEQ ID NO: 354.

### Kabat CDR-H3+Kabat CDR-H2

In some embodiments, the additional binding domain capable of binding to a CD3ε epitope comprises a V_{H} sequence comprising a Kabat CDR-H3 sequence comprising, consisting of, or consisting essentially of a sequence selected from SEQ ID NOS: 379-382, and a Kabat CDR-H2 sequence comprising, consisting of, or consisting essentially of a sequence selected from SEQ ID NOS: 371-375. In some embodiments, the Kabat CDR-H3 sequence and the Kabat CDR-H2 sequence are both from a single illustrative V_{H} sequence provided in this disclosure. For example, in some embodiments, the Kabat CDR-H3 and Kabat CDR-H2 are both from a single illustrative V_{H} sequence selected from SEQ ID NOS: 413-418.

### Kabat CDR-H3+Kabat CDR-H1

In some embodiments, the additional binding domain capable of binding to a CD3ε epitope comprises a V_{H} sequence comprising a Kabat CDR-H3 sequence comprising, consisting of, or consisting essentially of a sequence selected from SEQ ID NOS: 379-382, and a Kabat CDR-H1 sequence comprising, consisting of, or consisting essentially of a sequence selected from SEQ ID NOS: 354-357. In some embodiments, the Kabat CDR-H3 sequence and the Kabat CDR-H1 sequence are both from a single illustrative V_{H} sequence provided in this disclosure. For example, in some embodiments, the Kabat CDR-H3 and Kabat CDR-H1 are both from a single illustrative V_{H} sequence selected from SEQ ID NOS: 413-418.

### Kabat CDR-H1+Kabat CDR-H2

In some embodiments, the additional binding domain capable of binding to a CD3ε epitope comprises a V_{H} sequence comprising a Kabat CDR-H1 sequence comprising, consisting of, or consisting essentially of a sequence selected from SEQ ID NOS: 354-357 and a Kabat CDR-H2 sequence comprising, consisting of, or consisting essentially of a sequence selected from SEQ ID NOS: 371-375. In some embodiments, the Kabat CDR-H1 sequence and the Kabat CDR-H2 sequence are both from a single illustrative V_{H} sequence provided in this disclosure. For example, in some embodiments, the Kabat CDR-H1 and Kabat CDR-H2 are both from a single illustrative V_{H} sequence selected from SEQ ID NOS: 413-418.

### Kabat CDR-H1+Kabat CDR-H2+Kabat CDR-H3

In some embodiments, the additional binding domain capable of binding to a CD3ε epitope comprises a V_{H} sequence comprising a Kabat CDR-H1 sequence comprising, consisting of, or consisting essentially of a sequence selected from SEQ ID NOS: 354-357, a Kabat CDR-H2 sequence comprising, consisting of, or consisting essentially of a sequence selected from SEQ ID NOS: 371-375, and a Kabat CDR-H3 sequence comprising, consisting of, or consisting essentially of a sequence selected from SEQ ID NOS: 379-382. In some embodiments, the Kabat CDR-H1 sequence, Kabat CDR-H2 sequence, and Kabat CDR-H3 sequence are all from a single illustrative V_{H} sequence provided in this disclosure. For example, in some embodiments, the Kabat CDR-H1, Kabat CDR-H2, and Kabat CDR-H3 are all from a single illustrative V_{H} sequence selected from SEQ ID NOS: 413-418.

In some embodiments, the additional binding domain capable of binding to a CD3ε epitope comprises a V_{H} sequence comprising a Kabat CDR-H1 sequence comprising SEQ ID NO: 354, a Kabat CDR-H2 sequence comprising SEQ ID NO: 371, and a Kabat CDR-H3 sequence comprising SEQ ID NO: 379.

### Variants of V_{H} Sequences Comprising Illustrative Kabat CDRs

In some embodiments, the V_{H} sequences provided herein comprise a variant of an illustrative Kabat CDR-H3, CDR-H2, and/or CDR-H1 sequence provided in this disclosure.

In some embodiments, the Kabat CDR-H3 sequence comprises, consists of, or consists essentially of a variant of an illustrative Kabat CDR-H3 sequence provided in this disclosure. In some embodiments, the Kabat CDR-H3 sequence comprises, consists of, or consists essentially of a sequence having at least 70%, 75%, 80%, 85%, 90%, or 95% identity with any of the illustrative Kabat CDR-H3 sequences provided in this disclosure. In some embodiments, the Kabat CDR-H3 sequence comprises, consists of, or consists essentially of any of the illustrative Kabat CDR-H3 sequences provided in this disclosure, with 1, 2, or 3 amino acid substitutions. In some embodiments, the amino acid substitutions are conservative amino acid substitutions.

In some embodiments, the Kabat CDR-H2 sequence comprises, consists of, or consists essentially of a variant of an illustrative Kabat CDR-H2 sequence provided in this disclosure. In some embodiments, the Kabat CDR-H2 sequence comprises, consists of, or consists essentially of a sequence having at least 70%, 75%, 80%, 85%, 90%, or 95% identity with any of the illustrative Kabat CDR-H2 sequences provided in this disclosure. In some embodiments, the Kabat CDR-H2 sequence comprises, consists of, or consists essentially of any of the illustrative Kabat CDR-H2 sequences provided in this disclosure, with 1, 2, or 3 amino acid substitutions. In some embodiments, the amino acid substitutions are conservative amino acid substitutions.

In some embodiments, the Kabat CDR-H1 sequence comprises, consists of, or consists essentially of a variant of an illustrative Kabat CDR-H1 sequence provided in this disclosure. In some embodiments, the Kabat CDR-H1 sequence comprises, consists of, or consists essentially of a sequence having at least 70%, 75%, 80%, 85%, 90%, or 95% identity with any of the illustrative Kabat CDR-H1 sequences provided in this disclosure. In some embodiments, the Kabat CDR-H1 sequence comprises, consists of, or consists essentially of any of the illustrative Kabat CDR-H1 sequences provided in this disclosure, with 1, 2, or 3 amino acid substitutions. In some embodiments, the amino acid substitutions are conservative amino acid substitutions.

### V_{H} Sequences Comprising Illustrative Chothia CDRs

In some embodiments, the additional binding domain capable of binding to a CD3ε epitope comprises a V_{H} sequence comprising one or more Chothia CDR-H sequences comprising, consisting of, or consisting essentially of one or more illustrative Chothia CDR-H sequences provided in this disclosure, and variants thereof.

### Chothia CDR-H3

In some embodiments, the additional binding domain capable of binding to a CD3ε epitope comprises a V_{H} sequence comprising a Chothia CDR-H3 sequence comprising, consisting of, or consisting essentially of a sequence selected from SEQ ID NOS: 379-382. In some embodiments, the additional binding domain capable of binding to a CD3ε epitope comprises a V_{H} sequence comprising a Chothia CDR-H3 sequence comprising, consisting of, or consisting essentially of SEQ ID NO: 413-418. In some embodiments, the additional binding domain capable of binding to a CD3ε epitope comprises a V_{H} sequence comprising a Chothia CDR-H3 sequence comprising, consisting of, or consisting essentially of SEQ ID NO: 379.

### Chothia CDR-H2

In some embodiments, the additional binding domain capable of binding to a CD3ε epitope comprises a V_{H} sequence comprising a Chothia CDR-H2 sequence comprising, consisting of, or consisting essentially of a sequence selected from SEQ ID NOS: 362-365. In some embodiments, the additional binding domain capable of binding to a CD3ε epitope comprises a V_{H} sequence comprising a Chothia CDR-H2 sequence comprising, consisting of, or consisting essentially of SEQ ID NO: 413-418. In some embodiments, the additional binding domain capable of binding to a CD3ε epitope comprises a V_{H} sequence comprising a Kabat CDR-H2 sequence comprising, consisting of, or consisting essentially of SEQ ID NO: 362.

### Chothia CDR-H1

In some embodiments, the additional binding domain capable of binding to a CD3ε epitope comprises a V_{H} sequence comprising a Chothia CDR-H1 sequence comprising, consisting of, or consisting essentially of a sequence selected from SEQ ID NOS: 346-349. In some embodiments, the additional binding domain capable of binding to a CD3ε epitope comprises a V_{H} sequence comprising a Chothia CDR-H1 sequence comprising, consisting of, or consisting essentially of SEQ ID NO: 413-418. In some embodiments, the additional binding domain capable of binding to a CD3ε epitope comprises a V_{H} sequence comprising a Chothia CDR-H2 sequence comprising, consisting of, or consisting essentially of SEQ ID NO: 346.

### Chothia CDR-H3+Chothia CDR-H2

In some embodiments, the additional binding domain capable of binding to a CD3ε epitope comprises a V_{H} sequence comprising a Chothia CDR-H3 sequence comprising, consisting of, or consisting essentially of a sequence selected from SEQ ID NOS: 379-382, and a Chothia CDR-H2 sequence comprising, consisting of, or consisting essentially of a sequence selected from SEQ ID NOS: 362-365. In some embodiments, the Chothia CDR-H3 sequence and the Chothia CDR-H2 sequence are both from a single illustrative V_{H} sequence provided in this disclosure. For example, in some embodiments, the Chothia CDR-H3 and Chothia CDR-H2 are both from a single illustrative V_{H} sequence selected from SEQ ID NOS: 413-418.

### Chothia CDR-H3+Chothia CDR-H1

In some embodiments, the additional binding domain capable of binding to a CD3ε epitope comprises a V_{H} sequence comprising a Chothia CDR-H3 sequence comprising, consisting of, or consisting essentially of a sequence selected from SEQ ID NOS: 379-382, and a Chothia CDR-H1 sequence comprising, consisting of, or consisting essentially of a sequence selected from SEQ ID NOS: 346-349. In some embodiments, the Chothia CDR-H3 sequence and the Chothia CDR-H1 sequence are both from a single illustrative V_{H} sequence provided in this disclosure. For example, in some embodiments, the Chothia CDR-H3 and Chothia CDR-H1 are both from a single illustrative V_{H} sequence selected from SEQ ID NOS: 413-418.

### Chothia CDR-H1+Chothia CDR-H2

In some embodiments, the additional binding domain capable of binding to a CD3ε epitope comprises a V_{H} sequence comprising a Chothia CDR-H1 sequence comprising, consisting of, or consisting essentially of a sequence selected from SEQ ID NOS: 346-349 and a Chothia CDR-H2 sequence comprising, consisting of, or consisting essentially of a sequence selected from SEQ ID NOS: 362-365. In some embodiments, the Chothia CDR-H1 sequence and the Chothia CDR-H2 sequence are both from a single illustrative V_{H} sequence provided in this disclosure. For example, in some embodiments, the Chothia CDR-H1 and Chothia CDR-H2 are both from a single illustrative V_{H} sequence selected from SEQ ID NOS: 413-418.

### Chothia CDR-H1+Chothia CDR-H2+Chothia CDR-H3

In some embodiments, the additional binding domain capable of binding to a CD3ε epitope comprises a V_{H} sequence comprising a Chothia CDR-H1 sequence comprising, consisting of, or consisting essentially of a sequence selected from SEQ ID NOS: 346-349, a Chothia CDR-H2 sequence comprising, consisting of, or consisting essentially of a sequence selected from SEQ ID NOS: 362-365, and a Chothia CDR-H3 sequence comprising, consisting of, or consisting essentially of a sequence selected from SEQ ID NOS: 379-382. In some embodiments, the Chothia CDR-H1 sequence, Chothia CDR-H2 sequence, and Chothia CDR-H3 sequence are all from a single illustrative V_{H} sequence provided in this disclosure. For example, in some embodiments, the Chothia CDR-H1, Chothia CDR-H2, and Chothia CDR-H3 are all from a single illustrative V_{H} sequence selected from SEQ ID NOS: 413-418.

In some embodiments, the additional binding domain capable of binding to a CD3ε epitope comprises a V_{H} sequence comprising a Chothia CDR-H1 sequence comprising SEQ ID NO: 346, a Chothia CDR-H2 sequence comprising SEQ ID NO: 362, and a Chothia CDR-H3 sequence comprising SEQ ID NO: 379.

### Variants of V_{H} Sequences Comprising Illustrative Chothia CDRs

In some embodiments, the V_{H} sequences provided herein comprise a variant of an illustrative Chothia CDR-H3, CDR-H2, and/or CDR-H1 sequence provided in this disclosure.

In some embodiments, the Chothia CDR-H3 sequence comprises, consists of, or consists essentially of a variant of an illustrative Chothia CDR-H3 sequence provided in this disclosure. In some embodiments, the Chothia CDR-H3 sequence comprises, consists of, or consists essentially of a sequence having at least 70%, 75%, 80%, 85%, 90%, or 95% identity with any of the illustrative Chothia CDR-H3 sequences provided in this disclosure. In some embodiments, the Chothia CDR-H3 sequence comprises, consists of, or consists essentially of any of the illustrative Chothia CDR-H3 sequences provided in this disclosure, with 1, 2, or 3 amino acid substitutions. In some embodiments, the amino acid substitutions are conservative amino acid substitutions.

In some embodiments, the Chothia CDR-H2 sequence comprises, consists of, or consists essentially of a variant of an illustrative Chothia CDR-H2 sequence provided in this disclosure. In some embodiments, the Chothia CDR-H2 sequence comprises, consists of, or consists essentially of a sequence having at least 70%, 75%, 80%, 85%, 90%, or 95% identity with any of the illustrative Chothia CDR-H2 sequences provided in this disclosure. In some embodiments, the Chothia CDR-H2 sequence comprises, consists of, or consists essentially of any of the illustrative Chothia CDR-H2 sequences provided in this disclosure, with 1, 2, or 3 amino acid substitutions. In some embodiments, the amino acid substitutions are conservative amino acid substitutions.

In some embodiments, the Chothia CDR-H1 sequence comprises, consists of, or consists essentially of a variant of an illustrative Chothia CDR-H1 sequence provided in this disclosure. In some embodiments, the Chothia CDR-H1 sequence comprises, consists of, or consists essentially of a sequence having at least 70%, 75%, 80%, 85%, 90%, or 95% identity with any of the illustrative Chothia CDR-H1 sequences provided in this disclosure. In some embodiments, the Chothia CDR-H1 sequence comprises, consists of, or consists essentially of any of the illustrative Chothia CDR-H1 sequences provided in this disclosure, with 1, 2, or 3 amino acid substitutions. In some embodiments, the amino acid substitutions are conservative amino acid substitutions.

### V_{H} Sequences

In some embodiments, the additional binding domain capable of binding to a CD3ε epitope comprises a V_{H} sequence comprising, consisting of, or consisting essentially of a sequence selected from SEQ ID NOS: 413-418. In some embodiments, the additional binding domain capable of binding to a CD3ε epitope comprises a V_{H} sequence comprising, consisting of, or consisting essentially of SEQ ID NO: 413. In some embodiments, the additional binding domain capable of binding to a CD3ε epitope comprises a V_{H} sequence comprising, consisting of, or consisting essentially of SEQ ID NO: 414. In some embodiments, the additional binding domain capable of binding to a CD3ε epitope comprises a V_{H} sequence comprising, consisting of, or consisting essentially of SEQ ID NO: 415. In some embodiments, the additional binding domain capable of binding to a CD3ε epitope comprises a V_{H} sequence comprising, consisting of, or consisting essentially of SEQ ID NO: 416. In some embodiments, the additional binding domain capable of binding to a CD3ε epitope comprises a V_{H} sequence comprising, consisting of, or consisting essentially of SEQ ID NO: 417. In some embodiments, the additional binding domain capable of binding to a CD3ε epitope comprises a V_{H} sequence comprising, consisting of, or consisting essentially of SEQ ID NO: 418.

In some embodiments, the binding domain capable of binding to a CD3ε epitope comprises three heavy chain CDRs each comprising, consisting of, or consisting essentially of a CDR sequence of a VH having the sequence set forth in one of SEQ ID NOS: 413-418.

### Variants of V_{H} Sequences

In some embodiments, the V_{H} sequences provided herein comprise, consist of, or consist essentially of a variant of an illustrative V_{H} sequence provided in this disclosure.

In some embodiments, the V_{H} sequence comprises, consists of, or consists essentially of a variant of an illustrative V_{H} sequence provided in this disclosure. In some embodiments, the V_{H} sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

### CDR-L3 Sequences

In some embodiments, the additional binding domain capable of binding to a CD3ε epitope comprises a CDR-L3 sequence comprising, consisting of, or consisting essentially of a sequence selected from SEQ ID NOS: 404-408. In some embodiments, the additional binding domain capable of binding to a CD3ε epitope comprises a CDR-L3 sequence comprising, consisting of, or consisting essentially of SEQ ID NO: 404.

In some embodiments, the CDR-L3 sequence comprises, consists of, or consists essentially of a variant of an illustrative CDR-L3 sequence provided in this disclosure. In some embodiments, the CDR-L3 sequence comprises, consists of, or consists essentially of a sequence having at least 70%, 75%, 80%, 85%, 90%, or 95% identity with any of the illustrative CDR-L3 sequences provided in this disclosure. In some embodiments, the CDR-L3 sequence comprises, consists of, or consists essentially of any of the illustrative CDR-L3 sequences provided in this disclosure, with 1, 2, or 3 amino acid substitutions. In some embodiments, the amino acid substitutions are conservative amino acid substitutions.

### V_{L} Sequences Comprising Illustrative CDRs

In some embodiments, the additional binding domain capable of binding to a CD3ε epitope comprises a V_{L} sequence comprising one or more CDR-L sequences comprising, consisting of, or consisting essentially of one or more illustrative CDR-L sequences provided in this disclosure, and variants thereof.

### CDR-L3

In some embodiments, the additional binding domain capable of binding to a CD3ε epitope comprises a V_{L} sequence comprising a CDR-L3 sequence comprising, consisting of, or consisting essentially of a sequence selected from SEQ ID NOS: 404-408. In some embodiments, the additional binding domain capable of binding to a CD3ε epitope comprises a V_{L} sequence comprising a CDR-L3 sequence comprising, consisting of, or consisting essentially of SEQ ID NO: 404.

### CDR-L2

In some embodiments, the additional binding domain capable of binding to a CD3ε epitope comprises a V_{L} sequence comprising a CDR-L2 sequence comprising, consisting of, or consisting essentially of a sequence selected from SEQ ID NOS: 396-400. In some embodiments, the additional binding domain capable of binding to a CD3ε epitope comprises a V_{L} sequence comprising a CDR-L2 sequence comprising, consisting of, or consisting essentially of SEQ ID NO: 396.

### CDR-L1

In some embodiments, the additional binding domain capable of binding to a CD3ε epitope comprises a V_{L} sequence comprising a CDR-L1 sequence comprising, consisting of, or consisting essentially of a sequence selected from SEQ ID NOS: 388-392. In some embodiments, the additional binding domain capable of binding to a CD3ε epitope comprises a V_{L} sequence comprising a CDR-L1 sequence comprising, consisting of, or consisting essentially of SEQ ID NO: 388.

### CDR-L3+CDR-L2

In some embodiments, the additional binding domain capable of binding to a CD3ε epitope comprises a V_{L} sequence comprising a CDR-L3 sequence comprising, consisting of, or consisting essentially of a sequence selected from SEQ ID NOS: 404-408 and a CDR-L2 sequence comprising, consisting of, or consisting essentially of a sequence selected from SEQ ID NOS: 396-400. In some embodiments, the CDR-L3 sequence and the CDR-L2 sequence are both from a single illustrative V_{L} sequence provided in this disclosure. For example, in some embodiments, the CDR-L3 and CDR-L2 are both from a single illustrative V_{L} sequence selected from SEQ ID NOS: 422-427.

### CDR-L3+CDR-L1

In some embodiments, the additional binding domain capable of binding to a CD3ε epitope comprises a V_{L} sequence comprising a CDR-L3 sequence comprising, consisting of, or consisting essentially of a sequence selected from SEQ ID NOS: 404-408 and a CDR-L1 sequence comprising, consisting of, or consisting essentially of a sequence selected from SEQ ID NOS: 388-392. In some embodiments, the CDR-L3 sequence and the CDR-L1 sequence are both from a single illustrative V_{L} sequence provided in this disclosure. For example, in some embodiments, the CDR-L3 and CDR-L1 are both from a single illustrative V_{L} sequence selected from SEQ ID NOS: 422-427.

### CDR-L1+CDR-L2

In some embodiments, the additional binding domain capable of binding to a CD3ε epitope comprises a V_{L} sequence comprising a CDR-L1 sequence comprising, consisting of, or consisting essentially of a sequence selected from SEQ ID NOS: 388-392 and a CDR-L2 sequence comprising, consisting of, or consisting essentially of a sequence selected from SEQ ID NOS: 396-400. In some embodiments, the CDR-L1 sequence and the CDR-L2 sequence are both from a single illustrative V_{L} sequence provided in this disclosure. For example, in some embodiments, the CDR-L1 and CDR-L2 are both from a single illustrative V_{L} sequence selected from SEQ ID NOS: 422-427.

### CDR-L1+CDR-L2+CDR-L3

In some embodiments, the additional binding domain capable of binding to a CD3ε epitope comprises a V_{L} sequence comprising a CDR-L1 sequence comprising, consisting of, or consisting essentially of a sequence selected from SEQ ID NOS: 388-392, a CDR-L2 sequence comprising, consisting of, or consisting essentially of a sequence selected from SEQ ID NOS: 396-400, and a CDR-L3 sequence comprising, consisting of, or consisting essentially of a sequence selected from SEQ ID NOS: 404-408. In some embodiments, the CDR-L1 sequence, CDR-L2 sequence, and CDR-L3 sequence are all from a single illustrative V_{L} sequence provided in this disclosure. For example, in some embodiments, the CDR-L1, CDR-L2, and CDR-L3 are all from a single illustrative V_{L} sequence selected from SEQ ID NOS: 422-427.

In some embodiments, the additional binding domain capable of binding to a CD3ε epitope comprises a VL sequence comprising a CDR-L1 sequence comprising SEQ ID NO: 388, a CDR-L2 sequence comprising SEQ ID NO: 396, and a CDR-L3 sequence SEQ ID NO: 404.

### Variants of V_{L} Sequences Comprising Illustrative CDR-Ls

In some embodiments, the V_{L} sequences provided herein comprise a variant of an illustrative CDR-L3, CDR-L2, and/or CDR-L1 sequence provided in this disclosure.

In some embodiments, the CDR-L3 sequence comprises, consists of, or consists essentially of a variant of an illustrative CDR-L3 sequence provided in this disclosure. In some embodiments, the CDR-L3 sequence comprises, consists of, or consists essentially of a sequence having at least 70%, 75%, 80%, 85%, 90%, or 95% identity with any of the illustrative CDR-L3 sequences provided in this disclosure. In some embodiments, the CDR-L3 sequence comprises, consists of, or consists essentially of any of the illustrative CDR-L3 sequences provided in this disclosure, with 1, 2, or 3 amino acid substitutions. In some embodiments, the amino acid substitutions are conservative amino acid substitutions.

In some embodiments, the CDR-L2 sequence comprises, consists of, or consists essentially of a variant of an illustrative CDR-L2 sequence provided in this disclosure. In some embodiments, the CDR-L2 sequence comprises, consists of, or consists essentially of a sequence having at least 70%, 75%, 80%, 85%, 90%, or 95% identity with any of the illustrative CDR-L2 sequences provided in this disclosure. In some embodiments, the CDR-L2 sequence comprises, consists of, or consists essentially of any of the illustrative CDR-L2 sequences provided in this disclosure, with 1, 2, or 3 amino acid substitutions. In some embodiments, the amino acid substitutions are conservative amino acid substitutions.

In some embodiments, the CDR-L1 sequence comprises, consists of, or consists essentially of a variant of an illustrative CDR-L1 sequence provided in this disclosure. In some embodiments, the CDR-L1 sequence comprises, consists of, or consists essentially of a sequence having at least 70%, 75%, 80%, 85%, 90%, or 95% identity with any of the illustrative CDR-L1 sequences provided in this disclosure. In some embodiments, the CDR-L1 sequence comprises, consists of, or consists essentially of any of the illustrative CDR-L1 sequences provided in this disclosure, with 1, 2, or 3 amino acid substitutions. In some embodiments, the amino acid substitutions are conservative amino acid substitutions.

### V_{L} Sequences

In some embodiments, the additional binding domain capable of binding to a CD3ε epitope comprises a V_{L} sequence comprising, consisting of, or consisting essentially of a sequence selected from SEQ ID NOS: 422-427. In some embodiments, the additional binding domain capable of binding to a CD3ε epitope comprises a V_{L} sequence comprising, consisting of, or consisting essentially of SEQ ID NO: 422. In some embodiments, the additional binding domain capable of binding to a CD3ε epitope comprises a V_{L} sequence comprising, consisting of, or consisting essentially of SEQ ID NO: 423. In some embodiments, the additional binding domain capable of binding to a CD3ε epitope comprises a V_{L} sequence comprising, consisting of, or consisting essentially of SEQ ID NO: 424. In some embodiments, the additional binding domain capable of binding to a CD3ε epitope comprises a V_{L} sequence comprising, consisting of, or consisting essentially of SEQ ID NO: 425. In some embodiments, the additional binding domain capable of binding to a CD3ε epitope comprises a V_{L} sequence comprising, consisting of, or consisting essentially of SEQ ID NO: 426. In some embodiments, the additional binding domain capable of binding to a CD3ε epitope comprises a V_{L} sequence comprising, consisting of, or consisting essentially of SEQ ID NO: 427.

In some embodiments, the binding domain capable of binding to a CD3ε epitope comprises three light chain CDRs, each comprising, consisting of, or consisting essentially of a CDR sequence of a VL having the sequence set forth in one of SEQ ID NO: 422-427.

### Variants of V_{L} Sequences

In some embodiments, the V_{L} sequences provided herein comprise, consist of, or consist essentially of a variant of an illustrative V_{L} sequence provided in this disclosure.

In some embodiments, the V_{L} sequence comprises, consists of, or consists essentially of a variant of an illustrative V_{L} sequence provided in this disclosure. In some embodiments, the V_{L} sequence comprises, consists of, or consists essentially of a sequence having at least 85%, 90%, 95%, 96%, 97%, 98%, 99%, or 99.05% identity with any of the illustrative V_{L} sequences provided in this disclosure.

In some embodiments, the V_{L} sequence comprises, consists of, or consists essentially of any of the illustrative V_{L} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

### Pairs

### CDR-H3-CDR-L3 Pairs

In some embodiments, the additional binding domain capable of binding to a CD3ε epitope comprises a CDR-H3 sequence and a CDR-L3 sequence. In some embodiments, the CDR-H3 sequence is part of a V_{H} and the CDR-L3 sequence is part of a V_{L}.

In some embodiments, the CDR-H3 sequence is a CDR-H3 sequence comprising, consisting of, or consisting essentially of SEQ ID NOS: 379-382, and the CDR-L3 sequence is a CDR-L3 sequence comprising, consisting of, or consisting essentially of SEQ ID NOS: 404-408.

In some embodiments, the CDR-H3 sequence is SEQ ID NO: 379 and the CDR-L3 sequence is selected from SEQ ID NOS: 404-408. In some embodiments, the CDR-L3 sequence is SEQ ID NO: 404. In some embodiments, the CDR-L3 sequence is SEQ ID NO: 405. In some embodiments, the CDR-L3 sequence is SEQ ID NO: 406. In some embodiments, the CDR-L3 sequence is SEQ ID NO: 407. In some embodiments, the CDR-L3 sequence is SEQ ID NO: 408.

### Variants of CDR-H3-CDR-L3 Pairs

In some embodiments, the CDR-H3-CDR-L3 pairs provided herein comprise a variant of an illustrative CDR-H3 and/or CDR-L1 sequence provided in this disclosure.

In some embodiments, the CDR-H3 sequence comprises, consists of, or consists essentially of a variant of an illustrative CDR-H3 sequence provided in this disclosure. In some embodiments, the CDR-H3 sequence comprises, consists of, or consists essentially of a sequence having at least 70%, 75%, 80%, 85%, 90%, or 95% identity with any of the illustrative CDR-H3 sequences provided in this disclosure. In some embodiments, the CDR-H3 sequence comprises, consists of, or consists essentially of any of the illustrative CDR-H3 sequences provided in this disclosure, with 1, 2, or 3 amino acid substitutions. In some embodiments, the amino acid substitutions are conservative amino acid substitutions.

In some embodiments, the CDR-L3 sequence comprises, consists of, or consists essentially of a variant of an illustrative CDR-L3 sequence provided in this disclosure. In some embodiments, the CDR-L3 sequence comprises, consists of, or consists essentially of a sequence having at least 70%, 75%, 80%, 85%, 90%, or 95% identity with any of the illustrative CDR-L3 sequences provided in this disclosure. In some embodiments, the CDR-L3 sequence comprises, consists of, or consists essentially of any of the illustrative CDR-L3 sequences provided in this disclosure, with 1, 2, or 3 amino acid substitutions. In some embodiments, the amino acid substitutions are conservative amino acid substitutions.

V_{H}-V_{L} Pairs

In some embodiments, the additional binding domain capable of binding to a CD3ε epitope comprises a V_{H} sequence and a V_{L} sequence.

In some embodiments, the V_{H} sequence is a V_{H} sequence comprising, consisting of, or consisting essentially of SEQ ID NOS: 413-418 and the V_{L} sequence is a V_{L} sequence comprising, consisting of, or consisting essentially of SEQ ID NOS: 422-427.

In some embodiments, the V_{H} sequence is SEQ ID NO: 413 and the V_{L} sequence is selected from SEQ ID NOS: 422-427. In some embodiments, the V_{L} sequence is SEQ ID NO: 422. In some embodiments, the V_{L} sequence is SEQ ID NO: 423. In some embodiments, the V_{L} sequence is SEQ ID NO: 424. In some embodiments, the V_{L} sequence is SEQ ID NO: 425. In some embodiments, the V_{L} sequence is SEQ ID NO: 426. In some embodiments, the V_{L} sequence is SEQ ID NO: 427.

In some embodiments, the V_{H} sequence is SEQ ID NO: 414 and the V_{L} sequence is selected from SEQ ID NOS: 422-427. In some embodiments, the V_{L} sequence is SEQ ID NO: 422. In some embodiments, the V_{L} sequence is SEQ ID NO: 423. In some embodiments, the V_{L} sequence is SEQ ID NO: 424. In some embodiments, the V_{L} sequence is SEQ ID NO: 425. In some embodiments, the V_{L} sequence is SEQ ID NO: 426. In some embodiments, the V_{L} sequence is SEQ ID NO: 427.

In some embodiments, the V_{H} sequence is SEQ ID NO: 415 and the V_{L} sequence is selected from SEQ ID NOS: 422-427. In some embodiments, the V_{L} sequence is SEQ ID NO: 422. In some embodiments, the V_{L} sequence is SEQ ID NO: 423. In some embodiments, the V_{L} sequence is SEQ ID NO: 424. In some embodiments, the V_{L} sequence is SEQ ID NO: 425. In some embodiments, the V_{L} sequence is SEQ ID NO: 426. In some embodiments, the V_{L} sequence is SEQ ID NO: 427.

In some embodiments, the V_{H} sequence is SEQ ID NO: 416 and the V_{L} sequence is selected from SEQ ID NOS: 422-427. In some embodiments, the V_{L} sequence is SEQ ID NO: 422. In some embodiments, the V_{L} sequence is SEQ ID NO: 423. In some embodiments, the V_{L} sequence is SEQ ID NO: 424. In some embodiments, the V_{L} sequence is SEQ ID NO: 425. In some embodiments, the V_{L} sequence is SEQ ID NO: 426. In some embodiments, the V_{L} sequence is SEQ ID NO: 427.

In some embodiments, the V_{H} sequence is SEQ ID NO: 417 and the V_{L} sequence is selected from SEQ ID NOS: 422-427. In some embodiments, the V_{L} sequence is SEQ ID NO: 422. In some embodiments, the V_{L} sequence is SEQ ID NO: 423. In some embodiments, the V_{L} sequence is SEQ ID NO: 424. In some embodiments, the V_{L} sequence is SEQ ID NO: 425. In some embodiments, the V_{L} sequence is SEQ ID NO: 426. In some embodiments, the V_{L} sequence is SEQ ID NO: 427.

In some embodiments, the V_{H} sequence is SEQ ID NO: 418 and the V_{L} sequence is selected from SEQ ID NOS: 422-427. In some embodiments, the V_{L} sequence is SEQ ID NO: 422. In some embodiments, the V_{L} sequence is SEQ ID NO: 423. In some embodiments, the V_{L} sequence is SEQ ID NO: 424. In some embodiments, the V_{L} sequence is SEQ ID NO: 425. In some embodiments, the V_{L} sequence is SEQ ID NO: 426. In some embodiments, the V_{L} sequence is SEQ ID NO: 427.

In some embodiments, the binding domain capable of binding to a CD3ε epitope comprises three heavy chain CDRs and three light chain CDRs, each comprising, consisting of, or consisting essentially of a CDR sequence of a VH-VL pair set forth above. In some embodiments, the binding domain capable of binding to an HLA-G epitope comprises three heavy chain CDRs and three light chain CDRs, each comprising, consisting of, or consisting essentially of a CDR sequence of a VH-VL pair set forth in Table S.

### CDR-H1+CDR-H2+CDR-H3+CDR-L1+CDR-L2+CDR-L3

In some embodiments, the additional binding domain capable of binding to a CD3ε epitope comprises a V_{H} sequence comprising a Kabat CDR-H1 sequence comprising SEQ ID NO: 354, a Kabat CDR-H2 sequence comprising SEQ ID NO: 371, and a Kabat CDR-H3 sequence comprising SEQ ID NO: 379 and a VL sequence comprising a CDR-L1 sequence comprising SEQ ID NO: 388, a CDR-L2 sequence comprising SEQ ID NO: 396, and a CDR-L3 sequence SEQ ID NO: 404.

In some embodiments, the additional binding domain capable of binding to a CD3ε epitope comprises a V_{H} sequence comprising a Chothia CDR-H1 sequence comprising SEQ ID NO: 346, a Chothia CDR-H2 sequence comprising SEQ ID NO: 362, and a Chothia CDR-H3 sequence comprising SEQ ID NO: 379 and a VL sequence comprising a CDR-L1 sequence comprising SEQ ID NO: 388, a CDR-L2 sequence comprising SEQ ID NO: 396, and a CDR-L3 sequence SEQ ID NO: 404.

Variants of V_{H}-V_{L} Pairs

In some embodiments, the V_{H}-V_{L} pairs provided herein comprise a variant of an illustrative V_{H} and/or V_{L} sequence provided in this disclosure.

In some embodiments, the V_{H} sequence comprises, consists of, or consists essentially of a variant of an illustrative V_{H} sequence provided in this disclosure. In some embodiments, the V_{H} sequence comprises, consists of, or consists essentially of a sequence having at least 85%, 90%, 95%, 96%, 97%, 98%, 99%, or 99.1% identity with any of the illustrative V_{H} sequences provided in this disclosure.

In some embodiments, the V_{H} sequence comprises, consists of, or consists essentially of any of the illustrative V_{H} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

In some embodiments, the V_{L} sequence comprises, consists of, or consists essentially of a variant of an illustrative V_{L} sequence provided in this disclosure. In some embodiments, the V_{L} sequence comprises, consists of, or consists essentially of a sequence having at least 85%, 90%, 95%, 96%, 97%, 98%, 99%, or 99.05% identity with any of the illustrative V_{L} sequences provided in this disclosure.

In some embodiments, the V_{L} sequence comprises, consists of, or consists essentially of any of the illustrative V_{L} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

Additional Binding Domains Capable of Binding CD16, NKp46, and/or NKp30

### CD16

In some embodiments, the additional binding domain comprises an NK cell engager, dendritic cell engager, monocyte, or macrophage engager. In some embodiments, the NK cell engager comprises an antibody for a CD16 epitope. In some embodiments, the monocyte or macrophage engager comprises an antibody for a CD16 epitope. In some embodiments, the epitope comprises one or more amino acids on SEQ ID NOS: 630-631.

In some embodiments, the epitope comprises one or more amino acids on a polymorphism of SEQ ID NO: 630. In some embodiments, the polymorphism comprises a polypeptide sequence comprising or consisting of SEQ ID NO: 630, wherein the amino acid at position 176 is a V. In some embodiments, the polymorphism comprises a polypeptide sequence comprising or consisting of SEQ ID NO: 630, wherein the amino acid at position 176 is an F. In some embodiments, the polymorphism comprises or consists of a polypeptide sequence having an F or having a V at the bold and underlined position or SEQ ID NO: 630 set forth in Table S, herein.

CD16 is a cluster of differentiation molecule found on the surface of natural killer cells, neutrophils, monocytes, dendritic cells, and macrophages. CD16 has been identified as Fc receptors FcγRIIIa (CD16a) and FcγRIIIb (CD16b), which participate in signal transduction. The most well-researched membrane receptor implicated in triggering lysis by NK cells, CD16 is a molecule of the immunoglobulin superfamily (IgSF) involved in antibody-dependent cellular cytotoxicity (ADCC). CD16 can be used to isolate populations of specific immune cells through fluorescent-activated cell sorting (FACS) or magnetic-activated cell sorting, using antibodies directed towards CD16.

In humans, CD16 exists in two different forms: FcγRIIIa (CD16a) and FcγRIIIb (CD16b), which have 96% sequence similarity in the extracellular immunoglobulin binding regions. While FcγRIIIa is expressed on mast cells, macrophages, and natural killer cells as a transmembrane receptor, FcγRIIIb is only expressed on neutrophils. In addition, FcγRIIIb is the only Fc receptor anchored to the cell membrane by a glycosyl-phosphatidylinositol (GPI) linker, and also plays a significant role in triggering calcium mobilization and neutrophil degranulation. FcγRIIIa and FcγRIIIb together are able to activate degranulation, phagocytosis, and oxidative burst, which allows neutrophils to clear opsonized pathogens.

CD16 is required for ADCC processes carried out by human monocytes. In humans, monocytes expressing CD16 have a variety of ADCC capabilities in the presence of specific antibodies and can kill primary leukemic cells, cancer cell lines, and cells infected with hepatitis B virus. In addition, CD16 is able to mediate the direct killing of some virally infected and cancer cells without antibodies.

After binding to ligands such as the conserved section of IgG antibodies, CD16 on human NK cells induce gene transcription of surface activation molecules such as IL-2R (CD25) and inflammatory cytokines such as IFN-gamma and TNF. CD16-induced expression of cytokine mRNA in NK cells is mediated by the nuclear factor of activated T cells (NFATp), a cyclosporin A (CsA)-sensitive factor that regulates the transcription of various cytokines. The upregulated expression of specific cytokine genes occurs via a CsA-sensitive and calcium-dependent mechanism.

The crystal structures of FcεRIa, FcγRIIa, FcγRIIb, and FcγRIII have been experimentally determined. These structures revealed a conserved immunoglobulin-like (Ig-like) structure. In addition, the structures demonstrated a common feature in all known Ig superfamily Fc receptors: the acute hinge angle between the N- and C-terminal Ig domains. Specifically, the structure of CD16 (FcγRIIIb) consists of two immunoglobulin-like domains, with an interdomain hinge angle of around 50°. The receptor's Fc binding region also carries a net positive charge, which complements the negatively charged receptor binding regions on Fc.

### CDR-H3 Sequences

In some embodiments, the additional binding domain capable of binding to a CD16 epitope comprises a CDR-H3 sequence comprising, consisting of, or consisting essentially of a sequence selected from SEQ ID NOS: 465-472.

In some embodiments, the CDR-H3 sequence comprises, consists of, or consists essentially of a variant of an illustrative CDR-H3 sequence provided in this disclosure. In some embodiments, the CDR-H3 sequence comprises, consists of, or consists essentially of a sequence having at least 70%, 75%, 80%, 85%, 90%, or 95% identity with any of the illustrative CDR-H3 sequences provided in this disclosure. In some embodiments, the CDR-H3 sequence comprises, consists of, or consists essentially of any of the illustrative CDR-H3 sequences provided in this disclosure, with 1, 2, or 3 amino acid substitutions. In some embodiments, the amino acid substitutions are conservative amino acid substitutions.

### V_{H} Sequences Comprising Illustrative CDRs

In some embodiments, the additional binding domain capable of binding to a CD16 epitope comprises a V_{H} sequence comprising one or more CDR-H sequences comprising, consisting of, or consisting essentially of one or more illustrative CDR-H sequences provided in this disclosure, and variants thereof.

### V_{H} Sequences Comprising Illustrative Kabat CDRs

In some embodiments, the additional binding domain capable of binding to a CD16 epitope comprises a V_{H} sequence comprising one or more Kabat CDR-H sequences comprising, consisting of, or consisting essentially of one or more illustrative Kabat CDR-H sequences provided in this disclosure, and variants thereof.

### Kabat CDR-H3

In some embodiments, the additional binding domain capable of binding to a CD16 epitope comprises a V_{H} sequence comprising a Kabat CDR-H3 sequence comprising, consisting of, or consisting essentially of a sequence selected from SEQ ID NOS: 465-472.

### Kabat CDR-H2

In some embodiments, the additional binding domain capable of binding to a CD16 epitope comprises a V_{H} sequence comprising a Kabat CDR-H2 sequence comprising, consisting of, or consisting essentially of a sequence selected from SEQ ID NOS: 458-461.

### Kabat CDR-H1

In some embodiments, the additional binding domain capable of binding to a CD16 epitope comprises a V_{H} sequence comprising a Kabat CDR-H1 sequence comprising, consisting of, or consisting essentially of a sequence selected from SEQ ID NOS: 441-444.

### Kabat CDR-H3+Kabat CDR-H2

In some embodiments, the additional binding domain capable of binding to a CD16 epitope comprises a V_{H} sequence comprising a Kabat CDR-H3 sequence comprising, consisting of, or consisting essentially of a sequence selected from SEQ ID NOS: 465-472, and a Kabat CDR-H2 sequence comprising, consisting of, or consisting essentially of a sequence selected from SEQ ID NOS: 458-461. In some embodiments, the Kabat CDR-H3 sequence and the Kabat CDR-H2 sequence are both from a single illustrative V_{H} sequence provided in this disclosure. For example, in some embodiments, the Kabat CDR-H3 and Kabat CDR-H2 are both from a single illustrative V_{H} sequence selected from SEQ ID NOS: 501-513.

### Kabat CDR-H3+Kabat CDR-H1

In some embodiments, the additional binding domain capable of binding to a CD16 epitope comprises a V_{H} sequence comprising a Kabat CDR-H3 sequence comprising, consisting of, or consisting essentially of a sequence selected from SEQ ID NOS: 465-472, and a Kabat CDR-H1 sequence comprising, consisting of, or consisting essentially of a sequence selected from SEQ ID NOS: 441-444. In some embodiments, the Kabat CDR-H3 sequence and the Kabat CDR-H1 sequence are both from a single illustrative V_{H} sequence provided in this disclosure. For example, in some embodiments, the Kabat CDR-H3 and Kabat CDR-H1 are both from a single illustrative V_{H} sequence selected from SEQ ID NOS: 501-513.

### Kabat CDR-H1+Kabat CDR-H2

In some embodiments, the additional binding domain capable of binding to a CD16 epitope comprises a V_{H} sequence comprising a Kabat CDR-H1 sequence comprising, consisting of, or consisting essentially of a sequence selected from SEQ ID NOS: 441-444 and a Kabat CDR-H2 sequence comprising, consisting of, or consisting essentially of a sequence selected from SEQ ID NOS: 458-461. In some embodiments, the Kabat CDR-H1 sequence and the Kabat CDR-H2 sequence are both from a single illustrative V_{H} sequence provided in this disclosure. For example, in some embodiments, the Kabat CDR-H1 and Kabat CDR-H2 are both from a single illustrative V_{H} sequence selected from SEQ ID NOS: 501-513.

### Kabat CDR-H1+Kabat CDR-H2+Kabat CDR-H3

In some embodiments, the additional binding domain capable of binding to a CD16 epitope comprises a V_{H} sequence comprising a Kabat CDR-H1 sequence comprising, consisting of, or consisting essentially of a sequence selected from SEQ ID NOS: 441-444, a Kabat CDR-H2 sequence comprising, consisting of, or consisting essentially of a sequence selected from SEQ ID NOS: 458-461, and a Kabat CDR-H3 sequence comprising, consisting of, or consisting essentially of a sequence selected from SEQ ID NOS: 465-472. In some embodiments, the Kabat CDR-H1 sequence, Kabat CDR-H2 sequence, and Kabat CDR-H3 sequence are all from a single illustrative V_{H} sequence provided in this disclosure. For example, in some embodiments, the Kabat CDR-H1, Kabat CDR-H2, and Kabat CDR-H3 are all from a single illustrative V_{H} sequence selected from SEQ ID NOS: 501-513.

### Variants of V_{H} Sequences Comprising Illustrative Kabat CDRs

In some embodiments, the V_{H} sequences provided herein comprise a variant of an illustrative Kabat CDR-H3, CDR-H2, and/or CDR-H1 sequence provided in this disclosure.

In some embodiments, the Kabat CDR-H3 sequence comprises, consists of, or consists essentially of a variant of an illustrative Kabat CDR-H3 sequence provided in this disclosure. In some embodiments, the Kabat CDR-H3 sequence comprises, consists of, or consists essentially of a sequence having at least 70%, 75%, 80%, 85%, 90%, or 95% identity with any of the illustrative Kabat CDR-H3 sequences provided in this disclosure. In some embodiments, the Kabat CDR-H3 sequence comprises, consists of, or consists essentially of any of the illustrative Kabat CDR-H3 sequences provided in this disclosure, with 1, 2, or 3 amino acid substitutions. In some embodiments, the amino acid substitutions are conservative amino acid substitutions.

In some embodiments, the Kabat CDR-H2 sequence comprises, consists of, or consists essentially of a variant of an illustrative Kabat CDR-H2 sequence provided in this disclosure. In some embodiments, the Kabat CDR-H2 sequence comprises, consists of, or consists essentially of a sequence having at least 70%, 75%, 80%, 85%, 90%, or 95% identity with any of the illustrative Kabat CDR-H2 sequences provided in this disclosure. In some embodiments, the Kabat CDR-H2 sequence comprises, consists of, or consists essentially of any of the illustrative Kabat CDR-H2 sequences provided in this disclosure, with 1, 2, or 3 amino acid substitutions. In some embodiments, the amino acid substitutions are conservative amino acid substitutions.

In some embodiments, the Kabat CDR-H1 sequence comprises, consists of, or consists essentially of a variant of an illustrative Kabat CDR-H1 sequence provided in this disclosure. In some embodiments, the Kabat CDR-H1 sequence comprises, consists of, or consists essentially of a sequence having at least 70%, 75%, 80%, 85%, 90%, or 95% identity with any of the illustrative Kabat CDR-H1 sequences provided in this disclosure. In some embodiments, the Kabat CDR-H1 sequence comprises, consists of, or consists essentially of any of the illustrative Kabat CDR-H1 sequences provided in this disclosure, with 1, 2, or 3 amino acid substitutions. In some embodiments, the amino acid substitutions are conservative amino acid substitutions.

### V_{H} Sequences Comprising Illustrative Chothia CDRs

In some embodiments, the additional binding domain capable of binding to a CD16 epitope comprises a V_{H} sequence comprising one or more Chothia CDR-H sequences comprising, consisting of, or consisting essentially of one or more illustrative Chothia CDR-H sequences provided in this disclosure, and variants thereof.

### Chothia CDR-H3

In some embodiments, the additional binding domain capable of binding to a CD16 epitope comprises a V_{H} sequence comprising a Chothia CDR-H3 sequence comprising, consisting of, or consisting essentially of a sequence selected from SEQ ID NOS: 465-472.

### Chothia CDR-H2

In some embodiments, the additional binding domain capable of binding to a CD16 epitope comprises a V_{H} sequence comprising a Chothia CDR-H2 sequence comprising, consisting of, or consisting essentially of a sequence selected from SEQ ID NOS: 448-454.

### Chothia CDR-H1

In some embodiments, the additional binding domain capable of binding to a CD16 epitope comprises a V_{H} sequence comprising a Chothia CDR-H1 sequence comprising, consisting of, or consisting essentially of a sequence selected from SEQ ID NOS: 431-437.

### Chothia CDR-H3+Chothia CDR-H2

In some embodiments, the additional binding domain capable of binding to a CD16 epitope comprises a V_{H} sequence comprising a Chothia CDR-H3 sequence comprising, consisting of, or consisting essentially of a sequence selected from SEQ ID NOS: 465-472, and a Chothia CDR-H2 sequence comprising, consisting of, or consisting essentially of a sequence selected from SEQ ID NOS: 448-454. In some embodiments, the Chothia CDR-H3 sequence and the Chothia CDR-H2 sequence are both from a single illustrative V_{H} sequence provided in this disclosure. For example, in some embodiments, the Chothia CDR-H3 and Chothia CDR-H2 are both from a single illustrative V_{H} sequence selected from SEQ ID NOS: 501-513.

### Chothia CDR-H3+Chothia CDR-H1

In some embodiments, the additional binding domain capable of binding to a CD16 epitope comprises a V_{H} sequence comprising a Chothia CDR-H3 sequence comprising, consisting of, or consisting essentially of a sequence selected from SEQ ID NOS: 465-472, and a Chothia CDR-H1 sequence comprising, consisting of, or consisting essentially of a sequence selected from SEQ ID NOS: 431-437. In some embodiments, the Chothia CDR-H3 sequence and the Chothia CDR-H1 sequence are both from a single illustrative V_{H} sequence provided in this disclosure. For example, in some embodiments, the Chothia CDR-H3 and Chothia CDR-H1 are both from a single illustrative V_{H} sequence selected from SEQ ID NOS: 501-513.

### Chothia CDR-H1+Chothia CDR-H2

In some embodiments, the additional binding domain capable of binding to a CD16 epitope comprises a V_{H} sequence comprising a Chothia CDR-H1 sequence comprising, consisting of, or consisting essentially of a sequence selected from SEQ ID NOS: 431-437 and a Chothia CDR-H2 sequence comprising, consisting of, or consisting essentially of a sequence selected from SEQ ID NOS: 448-454. In some embodiments, the Chothia CDR-H1 sequence and the Chothia CDR-H2 sequence are both from a single illustrative V_{H} sequence provided in this disclosure. For example, in some embodiments, the Chothia CDR-H1 and Chothia CDR-H2 are both from a single illustrative V_{H} sequence selected from SEQ ID NOS: 501-513.

### Chothia CDR-H1+Chothia CDR-H2+Chothia CDR-H3

In some embodiments, the additional binding domain capable of binding to a CD16 epitope comprises a V_{H} sequence comprising a Chothia CDR-H1 sequence comprising, consisting of, or consisting essentially of a sequence selected from SEQ ID NOS: 431-437, a Chothia CDR-H2 sequence comprising, consisting of, or consisting essentially of a sequence selected from SEQ ID NOS: 448-454, and a Chothia CDR-H3 sequence comprising, consisting of, or consisting essentially of a sequence selected from SEQ ID NOS: 465-472. In some embodiments, the Chothia CDR-H1 sequence, Chothia CDR-H2 sequence, and Chothia CDR-H3 sequence are all from a single illustrative V_{H} sequence provided in this disclosure. For example, in some embodiments, the Chothia CDR-H1, Chothia CDR-H2, and Chothia CDR-H3 are all from a single illustrative V_{H} sequence selected from SEQ ID NOS: 501-513.

### Variants of V_{H} Sequences Comprising Illustrative Chothia CDRs

In some embodiments, the V_{H} sequences provided herein comprise a variant of an illustrative Chothia CDR-H3, CDR-H2, and/or CDR-H1 sequence provided in this disclosure.

In some embodiments, the Chothia CDR-H3 sequence comprises, consists of, or consists essentially of a variant of an illustrative Chothia CDR-H3 sequence provided in this disclosure. In some embodiments, the Chothia CDR-H3 sequence comprises, consists of, or consists essentially of a sequence having at least 70%, 75%, 80%, 85%, 90%, or 95% identity with any of the illustrative Chothia CDR-H3 sequences provided in this disclosure. In some embodiments, the Chothia CDR-H3 sequence comprises, consists of, or consists essentially of any of the illustrative Chothia CDR-H3 sequences provided in this disclosure, with 1, 2, or 3 amino acid substitutions. In some embodiments, the amino acid substitutions are conservative amino acid substitutions.

In some embodiments, the Chothia CDR-H2 sequence comprises, consists of, or consists essentially of a variant of an illustrative Chothia CDR-H2 sequence provided in this disclosure. In some embodiments, the Chothia CDR-H2 sequence comprises, consists of, or consists essentially of a sequence having at least 70%, 75%, 80%, 85%, 90%, or 95% identity with any of the illustrative Chothia CDR-H2 sequences provided in this disclosure. In some embodiments, the Chothia CDR-H2 sequence comprises, consists of, or consists essentially of any of the illustrative Chothia CDR-H2 sequences provided in this disclosure, with 1, 2, or 3 amino acid substitutions. In some embodiments, the amino acid substitutions are conservative amino acid substitutions.

In some embodiments, the Chothia CDR-H1 sequence comprises, consists of, or consists essentially of a variant of an illustrative Chothia CDR-H1 sequence provided in this disclosure. In some embodiments, the Chothia CDR-H1 sequence comprises, consists of, or consists essentially of a sequence having at least 70%, 75%, 80%, 85%, 90%, or 95% identity with any of the illustrative Chothia CDR-H1 sequences provided in this disclosure. In some embodiments, the Chothia CDR-H1 sequence comprises, consists of, or consists essentially of any of the illustrative Chothia CDR-H1 sequences provided in this disclosure, with 1, 2, or 3 amino acid substitutions. In some embodiments, the amino acid substitutions are conservative amino acid substitutions.

### V_{H} Sequences

In some embodiments, the additional binding domain capable of binding to a CD16 epitope comprises a V_{H} sequence comprising, consisting of, or consisting essentially of a sequence selected from SEQ ID NOS: 501-513. In some embodiments, the additional binding domain capable of binding to a CD16 epitope comprises a V_{H} sequence comprising, consisting of, or consisting essentially SEQ ID NO: 501. In some embodiments, the additional binding domain capable of binding to a CD16 epitope comprises a V_{H} sequence comprising, consisting of, or consisting essentially SEQ ID NO: 502. In some embodiments, the additional binding domain capable of binding to a CD16 epitope comprises a V_{H} sequence comprising, consisting of, or consisting essentially SEQ ID NO: 503. In some embodiments, the additional binding domain capable of binding to a CD16 epitope comprises a V_{H} sequence comprising, consisting of, or consisting essentially SEQ ID NO: 504. In some embodiments, the additional binding domain capable of binding to a CD16 epitope comprises a V_{H} sequence comprising, consisting of, or consisting essentially SEQ ID NO: 505. In some embodiments, the additional binding domain capable of binding to a CD16 epitope comprises a V_{H} sequence comprising, consisting of, or consisting essentially SEQ ID NO: 506. In some embodiments, the additional binding domain capable of binding to a CD16 epitope comprises a V_{H} sequence comprising, consisting of, or consisting essentially SEQ ID NO: 507. In some embodiments, the additional binding domain capable of binding to a CD16 epitope comprises a V_{H} sequence comprising, consisting of, or consisting essentially SEQ ID NO: 508. In some embodiments, the additional binding domain capable of binding to a CD16 epitope comprises a V_{H} sequence comprising, consisting of, or consisting essentially SEQ ID NO: 509. In some embodiments, the additional binding domain capable of binding to a CD16 epitope comprises a V_{H} sequence comprising, consisting of, or consisting essentially SEQ ID NO: 510. In some embodiments, the additional binding domain capable of binding to a CD16 epitope comprises a V_{H} sequence comprising, consisting of, or consisting essentially SEQ ID NO: 511. In some embodiments, the additional binding domain capable of binding to a CD16 epitope comprises a V_{H} sequence comprising, consisting of, or consisting essentially SEQ ID NO: 512. In some embodiments, the additional binding domain capable of binding to a CD16 epitope comprises a V_{H} sequence comprising, consisting of, or consisting essentially SEQ ID NO: 513.

In some embodiments, the binding domain capable of binding to a CD16 epitope comprises three heavy chain CDRs each comprising, consisting of, or consisting essentially of a CDR sequence of a V_{H} having the sequence set forth in one of SEQ ID NOS: 501-513.

### Variants of V_{H} Sequences

In some embodiments, the V_{H} sequences provided herein comprise, consist of, or consist essentially of a variant of an illustrative V_{H} sequence provided in this disclosure.

In some embodiments, the V_{H} sequence comprises, consists of, or consists essentially of a variant of an illustrative V_{H} sequence provided in this disclosure. In some embodiments, the V_{H} sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

### CDR-L3 Sequences

In some embodiments, the additional binding domain capable of binding to a CD16 epitope comprises a CDR-L3 sequence comprising, consisting of, or consisting essentially of a sequence selected from SEQ ID NOS: 492-497.

In some embodiments, the CDR-L3 sequence comprises, consists of, or consists essentially of a variant of an illustrative CDR-L3 sequence provided in this disclosure. In some embodiments, the CDR-L3 sequence comprises, consists of, or consists essentially of a sequence having at least 70%, 75%, 80%, 85%, 90%, or 95% identity with any of the illustrative CDR-L3 sequences provided in this disclosure. In some embodiments, the CDR-L3 sequence comprises, consists of, or consists essentially of any of the illustrative CDR-L3 sequences provided in this disclosure, with 1, 2, or 3 amino acid substitutions. In some embodiments, the amino acid substitutions are conservative amino acid substitutions.

### V_{L} Sequences Comprising Illustrative CDRs

In some embodiments, the additional binding domain capable of binding to a CD16 epitope comprises a V_{L} sequence comprising one or more CDR-L sequences comprising, consisting of, or consisting essentially of one or more illustrative CDR-L sequences provided in this disclosure, and variants thereof.

### CDR-L3

In some embodiments, the additional binding domain capable of binding to a CD16 epitope comprises a V_{L} sequence comprising a CDR-L3 sequence comprising, consisting of, or consisting essentially of a sequence selected from SEQ ID NOS: 492-497.

### CDR-L2

In some embodiments, the additional binding domain capable of binding to a CD16 epitope comprises a V_{L} sequence comprising a CDR-L2 sequence comprising, consisting of, or consisting essentially of a sequence selected from SEQ ID NOS: 484-488.

### CDR-L1

In some embodiments, the additional binding domain capable of binding to a CD16 epitope comprises a V_{L} sequence comprising a CDR-L1 sequence comprising, consisting of, or consisting essentially of a sequence selected from SEQ ID NOS: 476-480.

### CDR-L3+CDR-L2

In some embodiments, the additional binding domain capable of binding to a CD16 epitope comprises a V_{L} sequence comprising a CDR-L3 sequence comprising, consisting of, or consisting essentially of a sequence selected from SEQ ID NOS: 492-497 and a CDR-L2 sequence comprising, consisting of, or consisting essentially of a sequence selected from SEQ ID NOS: 484-488. In some embodiments, the CDR-L3 sequence and the CDR-L2 sequence are both from a single illustrative V_{L} sequence provided in this disclosure. For example, in some embodiments, the CDR-L3 and CDR-L2 are both from a single illustrative V_{L} sequence selected from SEQ ID NOS: 517-524.

### CDR-L3+CDR-L1

In some embodiments, the additional binding domain capable of binding to a CD16 epitope comprises a V_{L} sequence comprising a CDR-L3 sequence comprising, consisting of, or consisting essentially of a sequence selected from SEQ ID NOS: 492-497 and a CDR-L1 sequence comprising, consisting of, or consisting essentially of a sequence selected from SEQ ID NOS: 476-480. In some embodiments, the CDR-L3 sequence and the CDR-L1 sequence are both from a single illustrative V_{L} sequence provided in this disclosure. For example, in some embodiments, the CDR-L3 and CDR-L1 are both from a single illustrative V_{L} sequence selected from SEQ ID NOS: 517-524.

### CDR-L1+CDR-L2

In some embodiments, the additional binding domain capable of binding to a CD16 epitope comprises a V_{L} sequence comprising a CDR-L1 sequence comprising, consisting of, or consisting essentially of a sequence selected from SEQ ID NOS: 476-480 and a CDR-L2 sequence comprising, consisting of, or consisting essentially of a sequence selected from SEQ ID NOS: 484-488. In some embodiments, the CDR-L1 sequence and the CDR-L2 sequence are both from a single illustrative V_{L} sequence provided in this disclosure. For example, in some embodiments, the CDR-L1 and CDR-L2 are both from a single illustrative V_{L} sequence selected from SEQ ID NOS: 517-524.

### CDR-L1+CDR-L2+CDR-L3

In some embodiments, the additional binding domain capable of binding to a CD16 epitope comprises a V_{L} sequence comprising a CDR-L1 sequence comprising, consisting of, or consisting essentially of a sequence selected from SEQ ID NOS: 476-480, a CDR-L2 sequence comprising, consisting of, or consisting essentially of a sequence selected from SEQ ID NOS: 484-488, and a CDR-L3 sequence comprising, consisting of, or consisting essentially of a sequence selected from SEQ ID NOS: 492-497. In some embodiments, the CDR-L1 sequence, CDR-L2 sequence, and CDR-L3 sequence are all from a single illustrative V_{L} sequence provided in this disclosure. For example, in some embodiments, the CDR-L1, CDR-L2, and CDR-L3 are all from a single illustrative V_{L} sequence selected from SEQ ID NOS: 517-524.

### Variants of V_{L} Sequences Comprising Illustrative CDR-Ls

In some embodiments, the V_{L} sequences provided herein comprise a variant of an illustrative CDR-L3, CDR-L2, and/or CDR-L1 sequence provided in this disclosure.

In some embodiments, the CDR-L3 sequence comprises, consists of, or consists essentially of a variant of an illustrative CDR-L3 sequence provided in this disclosure. In some embodiments, the CDR-L3 sequence comprises, consists of, or consists essentially of a sequence having at least 70%, 75%, 80%, 85%, 90%, or 95% identity with any of the illustrative CDR-L3 sequences provided in this disclosure. In some embodiments, the CDR-L3 sequence comprises, consists of, or consists essentially of any of the illustrative CDR-L3 sequences provided in this disclosure, with 1, 2, or 3 amino acid substitutions. In some embodiments, the amino acid substitutions are conservative amino acid substitutions.

In some embodiments, the CDR-L2 sequence comprises, consists of, or consists essentially of a variant of an illustrative CDR-L2 sequence provided in this disclosure. In some embodiments, the CDR-L2 sequence comprises, consists of, or consists essentially of a sequence having at least 70%, 75%, 80%, 85%, 90%, or 95% identity with any of the illustrative CDR-L2 sequences provided in this disclosure. In some embodiments, the CDR-L2 sequence comprises, consists of, or consists essentially of any of the illustrative CDR-L2 sequences provided in this disclosure, with 1, 2, or 3 amino acid substitutions. In some embodiments, the amino acid substitutions are conservative amino acid substitutions.

In some embodiments, the CDR-L1 sequence comprises, consists of, or consists essentially of a variant of an illustrative CDR-L1 sequence provided in this disclosure. In some embodiments, the CDR-L1 sequence comprises, consists of, or consists essentially of a sequence having at least 70%, 75%, 80%, 85%, 90%, or 95% identity with any of the illustrative CDR-L1 sequences provided in this disclosure. In some embodiments, the CDR-L1 sequence comprises, consists of, or consists essentially of any of the illustrative CDR-L1 sequences provided in this disclosure, with 1, 2, or 3 amino acid substitutions. In some embodiments, the amino acid substitutions are conservative amino acid substitutions.

### V_{L} Sequences

In some embodiments, the additional binding domain capable of binding to a CD16 epitope comprises a V_{L} sequence comprising, consisting of, or consisting essentially of a sequence selected from SEQ ID NOS: 517-524. In some embodiments, the additional binding domain capable of binding to a CD16 epitope comprises a V_{L} sequence comprising, consisting of, or consisting essentially of SEQ ID NO: 517. In some embodiments, the additional binding domain capable of binding to a CD16 epitope comprises a V_{L} sequence comprising, consisting of, or consisting essentially of SEQ ID NO: 518. In some embodiments, the additional binding domain capable of binding to a CD16 epitope comprises a V_{L} sequence comprising, consisting of, or consisting essentially of SEQ ID NO: 519. In some embodiments, the additional binding domain capable of binding to a CD16 epitope comprises a V_{L} sequence comprising, consisting of, or consisting essentially of SEQ ID NO: 520. In some embodiments, the additional binding domain capable of binding to a CD16 epitope comprises a V_{L} sequence comprising, consisting of, or consisting essentially of SEQ ID NO: 521. In some embodiments, the additional binding domain capable of binding to a CD16 epitope comprises a V_{L} sequence comprising, consisting of, or consisting essentially of SEQ ID NO: 522. In some embodiments, the additional binding domain capable of binding to a CD16 epitope comprises a V_{L} sequence comprising, consisting of, or consisting essentially of SEQ ID NO: 523. In some embodiments, the additional binding domain capable of binding to a CD16 epitope comprises a V_{L} sequence comprising, consisting of, or consisting essentially of SEQ ID NO: 524.

In some embodiments, the binding domain capable of binding to a CD16 epitope comprises three light chain CDRs each comprising, consisting of, or consisting essentially of a CDR sequence of a VL having the sequence set forth in one of SEQ ID NOS: 517-524.

### Variants of V_{L} Sequences

In some embodiments, the V_{L} sequences provided herein comprise, consist of, or consist essentially of a variant of an illustrative V_{L} sequence provided in this disclosure.

In some embodiments, the V_{L} sequence comprises, consists of, or consists essentially of a variant of an illustrative V_{L} sequence provided in this disclosure. In some embodiments, the V_{L} sequence comprises, consists of, or consists essentially of a sequence having at least 85%, 90%, 95%, 96%, 97%, 98%, 99%, or 99.05% identity with any of the illustrative V_{L} sequences provided in this disclosure.

In some embodiments, the V_{L} sequence comprises, consists of, or consists essentially of any of the illustrative V_{L} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

### Pairs

### CDR-H3-CDR-L3 Pairs

In some embodiments, the additional binding domain capable of binding to a CD16 epitope comprises a CDR-H3 sequence and a CDR-L3 sequence. In some embodiments, the CDR-H3 sequence is part of a V_{H} and the CDR-L3 sequence is part of a V_{L}.

In some embodiments, the CDR-H3 sequence is a CDR-H3 sequence comprising, consisting of, or consisting essentially of SEQ ID NOS: 465-472, and the CDR-L3 sequence is a CDR-L3 sequence comprising, consisting of, or consisting essentially of SEQ ID NOS: 492-497.

### Variants of CDR-H3-CDR-L3 Pairs

In some embodiments, the CDR-H3-CDR-L3 pairs provided herein comprise a variant of an illustrative CDR-H3 and/or CDR-L1 sequence provided in this disclosure.

In some embodiments, the CDR-H3 sequence comprises, consists of, or consists essentially of a variant of an illustrative CDR-H3 sequence provided in this disclosure. In some embodiments, the CDR-H3 sequence comprises, consists of, or consists essentially of a sequence having at least 70%, 75%, 80%, 85%, 90%, or 95% identity with any of the illustrative CDR-H3 sequences provided in this disclosure. In some embodiments, the CDR-H3 sequence comprises, consists of, or consists essentially of any of the illustrative CDR-H3 sequences provided in this disclosure, with 1, 2, or 3 amino acid substitutions. In some embodiments, the amino acid substitutions are conservative amino acid substitutions.

In some embodiments, the CDR-L3 sequence comprises, consists of, or consists essentially of a variant of an illustrative CDR-L3 sequence provided in this disclosure. In some embodiments, the CDR-L3 sequence comprises, consists of, or consists essentially of a sequence having at least 70%, 75%, 80%, 85%, 90%, or 95% identity with any of the illustrative CDR-L3 sequences provided in this disclosure. In some embodiments, the CDR-L3 sequence comprises, consists of, or consists essentially of any of the illustrative CDR-L3 sequences provided in this disclosure, with 1, 2, or 3 amino acid substitutions. In some embodiments, the amino acid substitutions are conservative amino acid substitutions.

V_{H}-V_{L} Pairs

In some embodiments, the additional binding domain capable of binding to a CD16 epitope comprises a V_{H} sequence and a V_{L} sequence.

In some embodiments, the V_{H} sequence is a V_{H} sequence comprising, consisting of, or consisting essentially of SEQ ID NOS: 501-513 and the V_{L} sequence is a V_{L} sequence comprising, consisting of, or consisting essentially of SEQ ID NOS: 517-524.

Variants of V_{H}-V_{L} Pairs

In some embodiments, the V_{H}-V_{L} pairs provided herein comprise a variant of an illustrative V_{H} and/or V_{L} sequence provided in this disclosure.

In some embodiments, the V_{H} sequence comprises, consists of, or consists essentially of a variant of an illustrative V_{H} sequence provided in this disclosure. In some embodiments, the V_{H} sequence comprises, consists of, or consists essentially of a sequence having at least 85%, 90%, 95%, 96%, 97%, 98%, 99%, or 99.1% identity with any of the illustrative V_{H} sequences provided in this disclosure.

In some embodiments, the V_{H} sequence comprises, consists of, or consists essentially of any of the illustrative V_{H} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

In some embodiments, the V_{L} sequence comprises, consists of, or consists essentially of a variant of an illustrative V_{L} sequence provided in this disclosure. In some embodiments, the V_{L} sequence comprises, consists of, or consists essentially of a sequence having at least 85%, 90%, 95%, 96%, 97%, 98%, 99%, or 99.05% identity with any of the illustrative V_{L} sequences provided in this disclosure.

In some embodiments, the V_{L} sequence comprises, consists of, or consists essentially of any of the illustrative V_{L} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

### NKp46

In some embodiments, the NK cell engager comprises an antibody for an NKp46 epitope. In some embodiments, the NKp46 epitopes comprises at least one of SEQ ID NOS: 632-637. In some embodiments, the additional binding domain capable of binding to an NKp46 epitope comprises a sequence comprising, consisting of, or consisting essentially of SEQ ID NO: 544.

NKp46 is a protein that in humans is encoded by the NCR1 gene. NKp46 is a major NK cell-activating receptor that is involved in the elimination of target cells. NK cells form different types of synapses that result in distinct functional outcomes: cytotoxic, inhibitory, and regulatory. NKp46 is a member of the natural cytotoxicity receptor (NCR) family.

Engagement of the NKp46 receptor on NK cells results in increased cellular activation, manifesting as increased cytokine production and release of cytolytic granules. The receptor can confer tumor cell recognition by NK cells and can specifically bind viral hemagglutinins. Normal NK cells in humans and in rodents uniformly express NKp46, which is upregulated during NK cell maturation following commitment to the NK cell lineage.

NKp46 is often considered a highly selective if not specific marker of NK cells in basic immunology. NKp46 is a highly specific marker of NK cells and is of potential utility in the diagnosis of NK cell and some T-cell-derived neoplasms.

### V_{H} Sequences Comprising Illustrative CDRs

In some embodiments, the additional binding domain capable of binding to an NKp46 epitope comprises a V_{H} sequence comprising one or more CDR-H sequences comprising, consisting of, or consisting essentially of one or more illustrative CDR-H sequences provided in this disclosure, and variants thereof.

In some embodiments, the CDR-H3 sequence comprises, consists of, or consists essentially of a variant of an illustrative CDR-H3 sequence provided in this disclosure. In some embodiments, the CDR-H3 sequence comprises, consists of, or consists essentially of a sequence having at least 70%, 75%, 80%, 85%, 90%, or 95% identity with any of the illustrative CDR-H3 sequences provided in this disclosure. In some embodiments, the CDR-H3 sequence comprises, consists of, or consists essentially of any of the illustrative CDR-H3 sequences provided in this disclosure, with 1, 2, or 3 amino acid substitutions. In some embodiments, the amino acid substitutions are conservative amino acid substitutions.

### V_{H} Sequences Comprising Illustrative Kabat CDRs

In some embodiments, the additional binding domain capable of binding to an NKp46 epitope comprises a V_{H} sequence comprising one or more Kabat CDR-H sequences comprising, consisting of, or consisting essentially of one or more illustrative Kabat CDR-H sequences provided in this disclosure, and variants thereof.

### Kabat CDR-H3

In some embodiments, the additional binding domain capable of binding to an NKp46 epitope comprises a V_{H} sequence comprising a Kabat CDR-H3 sequence comprising, consisting of, or consisting essentially of SEQ ID NO: 544.

### Kabat CDR-H2

In some embodiments, the additional binding domain capable of binding to an NKp46 epitope comprises a V_{H} sequence comprising a Kabat CDR-H2 sequence comprising, consisting of, or consisting essentially of SEQ ID NO: 540.

### Kabat CDR-H1

In some embodiments, the additional binding domain capable of binding to an NKp46 epitope comprises a V_{H} sequence comprising a Kabat CDR-H1 sequence comprising, consisting of, or consisting essentially of SEQ ID NO: 532.

### Kabat CDR-H3+Kabat CDR-H2

In some embodiments, the additional binding domain capable of binding to an NKp46 epitope comprises a V_{H} sequence comprising a Kabat CDR-H3 sequence comprising, consisting of, or consisting essentially of SEQ ID NO: 544, and a Kabat CDR-H2 sequence comprising, consisting of, or consisting essentially of SEQ ID NO: 540. In some embodiments, the Kabat CDR-H3 sequence and the Kabat CDR-H2 sequence are both from a single illustrative V_{H} sequence provided in this disclosure. For example, in some embodiments, the Kabat CDR-H3 and Kabat CDR-H2 are both from a single illustrative V_{H} sequence having SEQ ID NO: 560.

### Kabat CDR-H3+Kabat CDR-H1

In some embodiments, the additional binding domain capable of binding to an NKp46 epitope comprises a V_{H} sequence comprising a Kabat CDR-H3 sequence comprising, consisting of, or consisting essentially of SEQ ID NO: 544, and a Kabat CDR-H1 sequence comprising, consisting of, or consisting essentially of SEQ ID NOS: 532. In some embodiments, the Kabat CDR-H3 sequence and the Kabat CDR-H1 sequence are both from a single illustrative V_{H} sequence provided in this disclosure. For example, in some embodiments, the Kabat CDR-H3 and Kabat CDR-H1 are both from a single illustrative V_{H} sequence having SEQ ID NO: 560.

### Kabat CDR-H1+Kabat CDR-H2

In some embodiments, the additional binding domain capable of binding to an NKp46 epitope comprises a V_{H} sequence comprising a Kabat CDR-H1 sequence comprising, consisting of SEQ ID NO: 532 and a Kabat CDR-H2 sequence comprising, consisting of, or consisting essentially of SEQ ID NO: 540. In some embodiments, the Kabat CDR-H1 sequence and the Kabat CDR-H2 sequence are both from a single illustrative V_{H} sequence provided in this disclosure. For example, in some embodiments, the Kabat CDR-H1 and Kabat CDR-H2 are both from a single illustrative V_{H} sequence having SEQ ID NO: 560.

### Kabat CDR-H1+Kabat CDR-H2+Kabat CDR-H3

In some embodiments, the additional binding domain capable of binding to an NKp46 epitope comprises a V_{H} sequence comprising a Kabat CDR-H1 sequence comprising, consisting of, or consisting essentially of SEQ ID NO: 532, a Kabat CDR-H2 sequence comprising, consisting of, or consisting essentially of SEQ ID NO: 540, and a Kabat CDR-H3 sequence comprising, consisting of, or consisting essentially of SEQ ID NO: 544. In some embodiments, the Kabat CDR-H1 sequence, Kabat CDR-H2 sequence, and Kabat CDR-H3 sequence are all from a single illustrative V_{H} sequence provided in this disclosure. For example, in some embodiments, the Kabat CDR-H1, Kabat CDR-H2, and Kabat CDR-H3 are all from a single illustrative V_{H} sequence having SEQ ID NO: 560.

### Variants of V_{H} Sequences Comprising Illustrative Kabat CDRs

In some embodiments, the V_{H} sequences provided herein comprise a variant of an illustrative Kabat CDR-H3, CDR-H2, and/or CDR-H1 sequence provided in this disclosure.

In some embodiments, the Kabat CDR-H3 sequence comprises, consists of, or consists essentially of a variant of an illustrative Kabat CDR-H3 sequence provided in this disclosure. In some embodiments, the Kabat CDR-H3 sequence comprises, consists of, or consists essentially of a sequence having at least 70%, 75%, 80%, 85%, 90%, or 95% identity with any of the illustrative Kabat CDR-H3 sequences provided in this disclosure. In some embodiments, the Kabat CDR-H3 sequence comprises, consists of, or consists essentially of any of the illustrative Kabat CDR-H3 sequences provided in this disclosure, with 1, 2, or 3 amino acid substitutions. In some embodiments, the amino acid substitutions are conservative amino acid substitutions.

In some embodiments, the Kabat CDR-H2 sequence comprises, consists of, or consists essentially of a variant of an illustrative Kabat CDR-H2 sequence provided in this disclosure. In some embodiments, the Kabat CDR-H2 sequence comprises, consists of, or consists essentially of a sequence having at least 70%, 75%, 80%, 85%, 90%, or 95% identity with any of the illustrative Kabat CDR-H2 sequences provided in this disclosure. In some embodiments, the Kabat CDR-H2 sequence comprises, consists of, or consists essentially of any of the illustrative Kabat CDR-H2 sequences provided in this disclosure, with 1, 2, or 3 amino acid substitutions. In some embodiments, the amino acid substitutions are conservative amino acid substitutions.

In some embodiments, the Kabat CDR-H1 sequence comprises, consists of, or consists essentially of a variant of an illustrative Kabat CDR-H1 sequence provided in this disclosure. In some embodiments, the Kabat CDR-H1 sequence comprises, consists of, or consists essentially of a sequence having at least 70%, 75%, 80%, 85%, 90%, or 95% identity with any of the illustrative Kabat CDR-H1 sequences provided in this disclosure. In some embodiments, the Kabat CDR-H1 sequence comprises, consists of, or consists essentially of any of the illustrative Kabat CDR-H1 sequences provided in this disclosure, with 1, 2, or 3 amino acid substitutions. In some embodiments, the amino acid substitutions are conservative amino acid substitutions.

### V_{H} Sequences Comprising Illustrative Chothia CDRs

In some embodiments, the additional binding domain capable of binding to an NKp46 epitope comprises a V_{H} sequence comprising one or more Chothia CDR-H sequences comprising, consisting of, or consisting essentially of one or more illustrative Chothia CDR-H sequences provided in this disclosure, and variants thereof.

### Chothia CDR-H3

In some embodiments, the additional binding domain capable of binding to an NKp46 epitope comprises a V_{H} sequence comprising a Chothia CDR-H3 sequence comprising, consisting of, or consisting essentially of SEQ ID NO: 544.

### Chothia CDR-H2

In some embodiments, the additional binding domain capable of binding to an NKp46 epitope comprises a V_{H} sequence comprising a Chothia CDR-H2 sequence comprising, consisting of, or consisting essentially of SEQ ID NO: 536.

### Chothia CDR-H1

In some embodiments, the additional binding domain capable of binding to an NKp46 epitope comprises a V_{H} sequence comprising a Chothia CDR-H1 sequence comprising, consisting of, or consisting essentially of SEQ ID NO: 528.

### Chothia CDR-H3+Chothia CDR-H2

In some embodiments, the additional binding domain capable of binding to an NKp46 epitope comprises a V_{H} sequence comprising a Chothia CDR-H3 sequence comprising, consisting of, or consisting essentially of SEQ ID NO: 544, and a Chothia CDR-H2 sequence comprising, consisting of, or consisting essentially of a sequence SEQ ID NO: 536. In some embodiments, the Chothia CDR-H3 sequence and the Chothia CDR-H2 sequence are both from a single illustrative V_{H} sequence provided in this disclosure. For example, in some embodiments, the Chothia CDR-H3 and Chothia CDR-H2 are both from a single illustrative V_{H} sequence having SEQ ID NO: 560.

### Chothia CDR-H3+Chothia CDR-H1

In some embodiments, the additional binding domain capable of binding to an NKp46 epitope comprises a V_{H} sequence comprising a Chothia CDR-H3 sequence comprising, consisting of, or consisting essentially of SEQ ID NO: 544, and a Chothia CDR-H1 sequence comprising, consisting of, or consisting essentially of SEQ ID NO: 528. In some embodiments, the Chothia CDR-H3 sequence and the Chothia CDR-H1 sequence are both from a single illustrative V_{H} sequence provided in this disclosure. For example, in some embodiments, the Chothia CDR-H3 and Chothia CDR-H1 are both from a single illustrative V_{H} having SEQ ID NO: 560.

### Chothia CDR-H1+Chothia CDR-H2

In some embodiments, the additional binding domain capable of binding to an NKp46 comprises a V_{H} sequence comprising a Chothia CDR-H1 sequence comprising, consisting of, or consisting essentially of SEQ ID NO: 528 and a Chothia CDR-H2 sequence comprising, consisting of, or consisting essentially of SEQ ID NO: 536. In some embodiments, the Chothia CDR-H1 sequence and the Chothia CDR-H2 sequence are both from a single illustrative V_{H} sequence provided in this disclosure. For example, in some embodiments, the Chothia CDR-H1 and Chothia CDR-H2 are both from a single illustrative V_{H} sequence SEQ ID NO: 560.

### Chothia CDR-H1+Chothia CDR-H2+Chothia CDR-H3

In some embodiments, the additional binding domain capable of binding to an NKp46 epitope comprises a V_{H} sequence comprising a Chothia CDR-H1 sequence comprising SEQ ID NO: 528, a Chothia CDR-H2 sequence comprising SEQ ID NO: 536, and a Chothia CDR-H3 sequence comprising SEQ ID NO: 544. For example, in some embodiments, the Chothia CDR-H1, Chothia CDR-H2, and Chothia-H3 are all from a single illustrative V_{H} sequence having SEQ ID NO: 560.

### Variants of V_{H} Sequences Comprising Illustrative Chothia CDRs

In some embodiments, the V_{H} sequences provided herein comprise a variant of an illustrative Chothia CDR-H3, CDR-H2, and/or CDR-H1 sequence provided in this disclosure.

In some embodiments, the Chothia CDR-H3 sequence comprises, consists of, or consists essentially of a variant of an illustrative Chothia CDR-H3 sequence provided in this disclosure. In some embodiments, the Chothia CDR-H3 sequence comprises, consists of, or consists essentially of a sequence having at least 70%, 75%, 80%, 85%, 90%, or 95% identity with any of the illustrative Chothia CDR-H3 sequences provided in this disclosure. In some embodiments, the Chothia CDR-H3 sequence comprises, consists of, or consists essentially of any of the illustrative Chothia CDR-H3 sequences provided in this disclosure, with 1, 2, or 3 amino acid substitutions. In some embodiments, the amino acid substitutions are conservative amino acid substitutions.

In some embodiments, the Chothia CDR-H2 sequence comprises, consists of, or consists essentially of a variant of an illustrative Chothia CDR-H2 sequence provided in this disclosure. In some embodiments, the Chothia CDR-H2 sequence comprises, consists of, or consists essentially of a sequence having at least 70%, 75%, 80%, 85%, 90%, or 95% identity with any of the illustrative Chothia CDR-H2 sequences provided in this disclosure. In some embodiments, the Chothia CDR-H2 sequence comprises, consists of, or consists essentially of any of the illustrative Chothia CDR-H2 sequences provided in this disclosure, with 1, 2, or 3 amino acid substitutions. In some embodiments, the amino acid substitutions are conservative amino acid substitutions.

In some embodiments, the Chothia CDR-H1 sequence comprises, consists of, or consists essentially of a variant of an illustrative Chothia CDR-H1 sequence provided in this disclosure. In some embodiments, the Chothia CDR-H1 sequence comprises, consists of, or consists essentially of a sequence having at least 70%, 75%, 80%, 85%, 90%, or 95% identity with any of the illustrative Chothia CDR-H1 sequences provided in this disclosure. In some embodiments, the Chothia CDR-H1 sequence comprises, consists of, or consists essentially of any of the illustrative Chothia CDR-H1 sequences provided in this disclosure, with 1, 2, or 3 amino acid substitutions. In some embodiments, the amino acid substitutions are conservative amino acid substitutions.

### V_{H} Sequences

In some embodiments, the additional binding domain capable of binding to an NKp46 epitope comprises a V_{H} sequence comprising, consisting of, or consisting essentially of SEQ ID NO: 560.

In some embodiments, the binding domain capable of binding to an NKp46 epitope comprises three heavy chain CDRs each comprising, consisting of, or consisting essentially of a CDR sequence of a VH having the sequence set forth in one of SEQ ID NO: 560.

### Variants of V_{H} Sequences

In some embodiments, the V_{H} sequences provided herein comprise, consist of, or consist essentially of a variant of an illustrative V_{H} sequence provided in this disclosure.

In some embodiments, the V_{H} sequence comprises, consists of, or consists essentially of a variant of an illustrative V_{H} sequence provided in this disclosure. In some embodiments, the V_{H} sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

### CDR-L3 Sequences

In some embodiments, the additional binding domain capable of binding to an NKp46 epitope comprises a CDR-L3 sequence comprising, consisting of, or consisting essentially of SEQ ID NO: 556.

In some embodiments, the CDR-L3 sequence comprises, consists of, or consists essentially of a variant of an illustrative CDR-L3 sequence provided in this disclosure. In some embodiments, the CDR-L3 sequence comprises, consists of, or consists essentially of a sequence having at least 70%, 75%, 80%, 85%, 90%, or 95% identity with any of the illustrative CDR-L3 sequences provided in this disclosure. In some embodiments, the CDR-L3 sequence comprises, consists of, or consists essentially of any of the illustrative CDR-L3 sequences provided in this disclosure, with 1, 2, or 3 amino acid substitutions. In some embodiments, the amino acid substitutions are conservative amino acid substitutions.

### V_{L} Sequences Comprising Illustrative CDRs

In some embodiments, the additional binding domain capable of binding to an NKp46 epitope comprises a V_{L} sequence comprising one or more CDR-L sequences comprising, consisting of, or consisting essentially of one or more illustrative CDR-L sequences provided in this disclosure, and variants thereof.

### CDR-L3

In some embodiments, the additional binding domain capable of binding to an NKp46 epitope comprises a V_{L} sequence comprising a CDR-L3 sequence comprising, consisting of, or consisting essentially of SEQ ID NO: 556.

### CDR-L2

In some embodiments, the additional binding domain capable of binding to an NKp46 epitope comprises a V_{L} sequence comprising a CDR-L2 sequence comprising, consisting of, or consisting essentially of SEQ ID NO: 552.

### CDR-L1

In some embodiments, the additional binding domain capable of binding to an NKp46 epitope comprises a V_{L} sequence comprising a CDR-L1 sequence comprising, consisting of, or consisting essentially of SEQ ID NO: 548.

### CDR-L3+CDR-L2

In some embodiments, the additional binding domain capable of binding to an NKp46 epitope comprises a V_{L} sequence comprising a CDR-L3 sequence comprising, consisting of, or consisting essentially of SEQ ID NO: 556 and a CDR-L2 sequence comprising, consisting of, or consisting essentially of SEQ ID NO: 552. In some embodiments, the CDR-L3 sequence and the CDR-L2 sequence are both from a single illustrative V_{L} sequence provided in this disclosure. For example, in some embodiments, the CDR-L3 and CDR-L2 are both from a single illustrative V_{L} sequence having SEQ ID NOS: 564.

### CDR-L3+CDR-L1

In some embodiments, the additional binding domain capable of binding to an NKp46 epitope comprises a V_{L} sequence comprising a CDR-L3 sequence comprising, consisting of, or consisting essentially of SEQ ID NO: 556 and a CDR-L1 sequence comprising, consisting of, or consisting essentially of SEQ ID NO: 548. In some embodiments, the CDR-L3 sequence and the CDR-L1 sequence are both from a single illustrative V_{L} sequence provided in this disclosure. For example, in some embodiments, the CDR-L3 and CDR-L1 are both from a single illustrative V_{L} sequence having SEQ ID NO: 564.

### CDR-L1+CDR-L2

In some embodiments, the additional binding domain capable of binding to an NKp46 epitope comprises a V_{L} sequence comprising a CDR-L1 sequence comprising, consisting of, or consisting essentially of SEQ ID NO: 548 and a CDR-L2 sequence comprising, consisting of, or consisting essentially of SEQ ID NOS: 552. In some embodiments, the CDR-L1 sequence and the CDR-L2 sequence are both from a single illustrative V_{L} sequence provided in this disclosure. For example, in some embodiments, the CDR-L1 and CDR-L2 are both from a single illustrative V_{L} sequence having SEQ ID NO: 564.

### CDR-L1+CDR-L2+CDR-L3

In some embodiments, the additional binding domain capable of binding to an NKp46 epitope comprises a VL sequence comprising a CDR-L1 sequence comprising SEQ ID NO: 548, a CDR-L2 sequence comprising SEQ ID NO: 552, and a CDR-L3 sequence SEQ ID NO: 556. In some embodiments, the CDR-L1 sequence, CDR-L2 sequence, and CDR-L3 sequence are all from a single illustrative V_{L} sequence provided in this disclosure. For example, in some embodiments, the CDR-L1, CDR-L2, and CDR-L3 are all from a single illustrative V_{L} sequence having SEQ ID NOS: 564.

### Variants of V_{L} Sequences Comprising Illustrative CDR-Ls

In some embodiments, the V_{L} sequences provided herein comprise a variant of an illustrative CDR-L3, CDR-L2, and/or CDR-L1 sequence provided in this disclosure.

In some embodiments, the CDR-L3 sequence comprises, consists of, or consists essentially of a variant of an illustrative CDR-L3 sequence provided in this disclosure. In some embodiments, the CDR-L3 sequence comprises, consists of, or consists essentially of a sequence having at least 70%, 75%, 80%, 85%, 90%, or 95% identity with any of the illustrative CDR-L3 sequences provided in this disclosure. In some embodiments, the CDR-L3 sequence comprises, consists of, or consists essentially of any of the illustrative CDR-L3 sequences provided in this disclosure, with 1, 2, or 3 amino acid substitutions. In some embodiments, the amino acid substitutions are conservative amino acid substitutions.

In some embodiments, the CDR-L2 sequence comprises, consists of, or consists essentially of a variant of an illustrative CDR-L2 sequence provided in this disclosure. In some embodiments, the CDR-L2 sequence comprises, consists of, or consists essentially of a sequence having at least 70%, 75%, 80%, 85%, 90%, or 95% identity with any of the illustrative CDR-L2 sequences provided in this disclosure. In some embodiments, the CDR-L2 sequence comprises, consists of, or consists essentially of any of the illustrative CDR-L2 sequences provided in this disclosure, with 1, 2, or 3 amino acid substitutions. In some embodiments, the amino acid substitutions are conservative amino acid substitutions.

In some embodiments, the CDR-L1 sequence comprises, consists of, or consists essentially of a variant of an illustrative CDR-L1 sequence provided in this disclosure. In some embodiments, the CDR-L1 sequence comprises, consists of, or consists essentially of a sequence having at least 70%, 75%, 80%, 85%, 90%, or 95% identity with any of the illustrative CDR-L1 sequences provided in this disclosure. In some embodiments, the CDR-L1 sequence comprises, consists of, or consists essentially of any of the illustrative CDR-L1 sequences provided in this disclosure, with 1, 2, or 3 amino acid substitutions. In some embodiments, the amino acid substitutions are conservative amino acid substitutions.

### V_{L} Sequences

In some embodiments, the additional binding domain capable of binding to an NKp46 epitope comprises a V_{L} sequence comprising, consisting of, or consisting essentially of SEQ ID NO: 564.

In some embodiments, the binding domain capable of binding to an NKp46 epitope comprises three light chain CDRs each comprising, consisting of, or consisting essentially of a CDR sequence of a VL having the sequence set forth in one of SEQ ID NO: 564.

### Variants of V_{L} Sequences

In some embodiments, the V_{L} sequences provided herein comprise, consist of, or consist essentially of a variant of an illustrative V_{L} sequence provided in this disclosure.

In some embodiments, the V_{L} sequence comprises, consists of, or consists essentially of a variant of an illustrative V_{L} sequence provided in this disclosure. In some embodiments, the V_{L} sequence comprises, consists of, or consists essentially of a sequence having at least 85%, 90%, 95%, 96%, 97%, 98%, 99%, or 99.05% identity with any of the illustrative V_{L} sequences provided in this disclosure.

In some embodiments, the V_{L} sequence comprises, consists of, or consists essentially of any of the illustrative V_{L} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

### Pairs

### CDR-H3-CDR-L3 Pairs

In some embodiments, the additional binding domain capable of binding to an NKp46 epitope comprises a CDR-H3 sequence and a CDR-L3 sequence. In some embodiments, the CDR-H3 sequence is part of a V_{H} and the CDR-L3 sequence is part of a V_{L}. In some embodiments, the CDR-H3 sequence is SEQ ID NO: 544 and the CDR-L3 sequence is SEQ ID NO: 556.

### Variants of CDR-H3-CDR-L3 Pairs

In some embodiments, the CDR-H3-CDR-L3 pairs provided herein comprise a variant of an illustrative CDR-H3 and/or CDR-L1 sequence provided in this disclosure.

In some embodiments, the CDR-H3 sequence comprises, consists of, or consists essentially of a variant of an illustrative CDR-H3 sequence provided in this disclosure. In some embodiments, the CDR-H3 sequence comprises, consists of, or consists essentially of a sequence having at least 70%, 75%, 80%, 85%, 90%, or 95% identity with any of the illustrative CDR-H3 sequences provided in this disclosure. In some embodiments, the CDR-H3 sequence comprises, consists of, or consists essentially of any of the illustrative CDR-H3 sequences provided in this disclosure, with 1, 2, or 3 amino acid substitutions. In some embodiments, the amino acid substitutions are conservative amino acid substitutions.

In some embodiments, the CDR-L3 sequence comprises, consists of, or consists essentially of a variant of an illustrative CDR-L3 sequence provided in this disclosure. In some embodiments, the CDR-L3 sequence comprises, consists of, or consists essentially of a sequence having at least 70%, 75%, 80%, 85%, 90%, or 95% identity with any of the illustrative CDR-L3 sequences provided in this disclosure. In some embodiments, the CDR-L3 sequence comprises, consists of, or consists essentially of any of the illustrative CDR-L3 sequences provided in this disclosure, with 1, 2, or 3 amino acid substitutions. In some embodiments, the amino acid substitutions are conservative amino acid substitutions.

V_{H}-V_{L} Pairs

In some embodiments, the additional binding domain capable of binding to an NKp46 epitope comprises a V_{H} sequence and a V_{L} sequence. In some embodiments, the V_{H} sequence is SEQ ID NO: 560 and the V_{L} sequence is SEQ ID NO: 564.

In some embodiments, the binding domain capable of binding to an NKp46 epitope comprises three heavy chain CDRs and three light chain CDRs, each comprising, consisting of, or consisting essentially of a CDR sequence of a VH-VL pair set forth above. In some embodiments, the binding domain capable of binding to an HLA-G epitope comprises three heavy chain CDRs and three light chain CDRs, each comprising, consisting of, or consisting essentially of a CDR sequence of a VH-VL pair set forth in Table S.

### CDR-H1+CDR-H2+CDR-H3+CDR-L1+CDR-L2+CDR-L3

In some embodiments, the additional binding domain capable of binding to an NKp46 epitope comprises a V_{H} sequence comprising a Kabat CDR-H1 sequence comprising SEQ ID NO: 532, a Kabat CDR-H2 sequence comprising SEQ ID NO: 540, and a Kabat CDR-H3 sequence comprising SEQ ID NO: 544 and a VL sequence comprising a CDR-L1 sequence comprising SEQ ID NO: 548, a CDR-L2 sequence comprising SEQ ID NO: 552, and a CDR-L3 sequence SEQ ID NO: 556.

In some embodiments, the additional binding domain capable of binding to an NKp46 epitope comprises a V_{H} sequence comprising a Chothia CDR-H1 sequence comprising SEQ ID NO: 528, a Chothia CDR-H2 sequence comprising SEQ ID NO: 536, and a Chothia CDR-H3 sequence comprising SEQ ID NO: 544 and a VL sequence comprising a CDR-L1 sequence comprising SEQ ID NO: 548, a CDR-L2 sequence comprising SEQ ID NO: 552, and a CDR-L3 sequence SEQ ID NO: 556.

Variants of V_{H}-V_{L} Pairs

In some embodiments, the V_{H}-V_{L} pairs provided herein comprise a variant of an illustrative V_{H} and/or V_{L} sequence provided in this disclosure.

In some embodiments, the V_{H} sequence comprises, consists of, or consists essentially of a variant of an illustrative V_{H} sequence provided in this disclosure. In some embodiments, the V_{H} sequence comprises, consists of, or consists essentially of a sequence having at least 85%, 90%, 95%, 96%, 97%, 98%, 99%, or 99.1% identity with any of the illustrative V_{H} sequences provided in this disclosure.

In some embodiments, the V_{H} sequence comprises, consists of, or consists essentially of any of the illustrative V_{H} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

In some embodiments, the V_{L} sequence comprises, consists of, or consists essentially of a variant of an illustrative V_{L} sequence provided in this disclosure. In some embodiments, the V_{L} sequence comprises, consists of, or consists essentially of a sequence having at least 85%, 90%, 95%, 96%, 97%, 98%, 99%, or 99.05% identity with any of the illustrative V_{L} sequences provided in this disclosure.

In some embodiments, the V_{L} sequence comprises, consists of, or consists essentially of any of the illustrative V_{L} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

### NKp30

In some embodiments, the NK cell engager comprises an antibody for an NKp30 epitope. In some embodiments, the NKp30 epitope comprises one or more amino acids in SEQ ID NOS: 638-643. In some embodiments, the additional binding domain capable of binding to an NKp30 epitope comprises a CDR-H3 sequence comprising, consisting of, or consisting essentially of a sequence selected from SEQ ID NOS: 568-570.

NKp30 is a protein that is encoded by the NCR3 gene. NKp30, also known as natural cytotoxicity receptor 3 (NCR3), was identified as a 30 kDa protein that, similarly to NKp46, is expressed on all mature resting and activated NK cells. Molecular cloning of the NKp30 cDNA revealed an open reading frame predicted to encode one extracellular IgV domain and a hydrophobic TM domain with a charged arginine residue capable of associating with the ITAM adaptors, CD3ζ and/or FcRγ. The crystal structure of NKp30 reveals some structural similarity to CTLA-4 and PD-1 and NKp30 is thus considered a member of the CD28 family of receptors (83, 89). Both NKp30 and NKp46 have reduced surface expression on adaptive memory NK cells most likely due to the downregulated expression of the FcRγ signaling chain required for the surface expression of these receptors.

Six alternatively spliced transcripts are transcribed from the NKp30 gene, termed NKp30a-f. Whilst NKp30a and NKp30b evoke NK cell activation, the NKp30c isoform was shown to elicit secretion of the immunosuppressive cytokine, IL-10, from NK cells (93). NKp30 has also been shown to be expressed by γδ T cells (30), CD8⁺ T cells (94), and UCB T cells cultured in IL-15.

### V_{H} Sequences Comprising Illustrative CDRs

In some embodiments, the additional binding domain capable of binding to an NKp30 epitope comprises a V_{H} sequence comprising one or more CDR-H sequences comprising, consisting of, or consisting essentially of one or more illustrative CDR-H sequences provided in this disclosure, and variants thereof.

### V_{H} Sequences Comprising Illustrative Kabat CDRs

In some embodiments, the additional binding domain capable of binding to an NKp30 epitope comprises a V_{H} sequence comprising one or more Kabat CDR-H sequences comprising, consisting of, or consisting essentially of one or more illustrative Kabat CDR-H sequences provided in this disclosure, and variants thereof.

### Kabat CDR-H3

In some embodiments, the additional binding domain capable of binding to an NKp30 epitope comprises a V_{H} sequence comprising a Kabat CDR-H3 sequence comprising, consisting of, or consisting essentially of a sequence selected from SEQ ID NOS: 592-594.

### Kabat CDR-H2

In some embodiments, the additional binding domain capable of binding to an NKp30 epitope comprises a V_{H} sequence comprising a Kabat CDR-H2 sequence comprising, consisting of, or consisting essentially of a sequence selected from SEQ ID NOS: 586-588.

### Kabat CDR-H1

In some embodiments, the additional binding domain capable of binding to an NKp30 epitope comprises a V_{H} sequence comprising a Kabat CDR-H1 sequence comprising, consisting of, or consisting essentially of a sequence selected from SEQ ID NOS: 574-576.

### Kabat CDR-H3+Kabat CDR-H2

In some embodiments, the additional binding domain capable of binding to an NKp30 epitope comprises a V_{H} sequence comprising a Kabat CDR-H3 sequence comprising, consisting of, or consisting essentially of a sequence selected from SEQ ID NOS: 592-594, and a Kabat CDR-H2 sequence comprising, consisting of, or consisting essentially of a sequence selected from SEQ ID NOS: 586-588. In some embodiments, the Kabat CDR-H3 sequence and the Kabat CDR-H2 sequence are both from a single illustrative V_{H} sequence provided in this disclosure. For example, in some embodiments, the Kabat CDR-H3 and Kabat CDR-H2 are both from a single illustrative V_{H} sequence selected from SEQ ID NOS: 613-616.

### Kabat CDR-H3+Kabat CDR-H1

In some embodiments, the additional binding domain capable of binding to an NKp30 epitope comprises a V_{H} sequence comprising a Kabat CDR-H3 sequence comprising, consisting of, or consisting essentially of a sequence selected from SEQ ID NOS: 592-594, and a Kabat CDR-H1 sequence comprising, consisting of, or consisting essentially of a sequence selected from SEQ ID NOS: 574-576. In some embodiments, the Kabat CDR-H3 sequence and the Kabat CDR-H1 sequence are both from a single illustrative V_{H} sequence provided in this disclosure. For example, in some embodiments, the Kabat CDR-H3 and Kabat CDR-H1 are both from a single illustrative V_{H} sequence selected from SEQ ID NOS: 613-616.

### Kabat CDR-H1+Kabat CDR-H2

In some embodiments, the additional binding domain capable of binding to an NKp30 epitope comprises a V_{H} sequence comprising a Kabat CDR-H1 sequence comprising, consisting of, or consisting essentially of a sequence selected from SEQ ID NOS: 574-576 and a Kabat CDR-H2 sequence comprising, consisting of, or consisting essentially of a sequence selected from SEQ ID NOS: 586-588. In some embodiments, the Kabat CDR-H1 sequence and the Kabat CDR-H2 sequence are both from a single illustrative V_{H} sequence provided in this disclosure. For example, in some embodiments, the Kabat CDR-H1 and Kabat CDR-H2 are both from a single illustrative V_{H} sequence selected from SEQ ID NOS: 613-616.

### Kabat CDR-H1+Kabat CDR-H2+Kabat CDR-H3

In some embodiments, the additional binding domain capable of binding to an NKp30 epitope comprises a V_{H} sequence comprising a Kabat CDR-H1 sequence comprising, consisting of, or consisting essentially of a sequence selected from SEQ ID NOS: 574-576, a Kabat CDR-H2 sequence comprising, consisting of, or consisting essentially of a sequence selected from SEQ ID NOS: 586-588, and a Kabat CDR-H3 sequence comprising, consisting of, or consisting essentially of a sequence selected from SEQ ID NOS: 592-594. In some embodiments, the Kabat CDR-H1 sequence, Kabat CDR-H2 sequence, and Kabat CDR-H3 sequence are all from a single illustrative V_{H} sequence provided in this disclosure. For example, in some embodiments, the Kabat CDR-H1, Kabat CDR-H2, and Kabat CDR-H3 are all from a single illustrative V_{H} sequence selected from SEQ ID NOS: 613-616.

### Variants of V_{H} Sequences Comprising Illustrative Kabat CDRs

In some embodiments, the V_{H} sequences provided herein comprise a variant of an illustrative Kabat CDR-H3, CDR-H2, and/or CDR-H1 sequence provided in this disclosure.

In some embodiments, the Kabat CDR-H3 sequence comprises, consists of, or consists essentially of a variant of an illustrative Kabat CDR-H3 sequence provided in this disclosure. In some embodiments, the Kabat CDR-H3 sequence comprises, consists of, or consists essentially of a sequence having at least 70%, 75%, 80%, 85%, 90%, or 95% identity with any of the illustrative Kabat CDR-H3 sequences provided in this disclosure. In some embodiments, the Kabat CDR-H3 sequence comprises, consists of, or consists essentially of any of the illustrative Kabat CDR-H3 sequences provided in this disclosure, with 1, 2, or 3 amino acid substitutions. In some embodiments, the amino acid substitutions are conservative amino acid substitutions.

In some embodiments, the Kabat CDR-H2 sequence comprises, consists of, or consists essentially of a variant of an illustrative Kabat CDR-H2 sequence provided in this disclosure. In some embodiments, the Kabat CDR-H2 sequence comprises, consists of, or consists essentially of a sequence having at least 70%, 75%, 80%, 85%, 90%, or 95% identity with any of the illustrative Kabat CDR-H2 sequences provided in this disclosure. In some embodiments, the Kabat CDR-H2 sequence comprises, consists of, or consists essentially of any of the illustrative Kabat CDR-H2 sequences provided in this disclosure, with 1, 2, or 3 amino acid substitutions. In some embodiments, the amino acid substitutions are conservative amino acid substitutions.

In some embodiments, the Kabat CDR-H1 sequence comprises, consists of, or consists essentially of a variant of an illustrative Kabat CDR-H1 sequence provided in this disclosure. In some embodiments, the Kabat CDR-H1 sequence comprises, consists of, or consists essentially of a sequence having at least 70%, 75%, 80%, 85%, 90%, or 95% identity with any of the illustrative Kabat CDR-H1 sequences provided in this disclosure. In some embodiments, the Kabat CDR-H1 sequence comprises, consists of, or consists essentially of any of the illustrative Kabat CDR-H1 sequences provided in this disclosure, with 1, 2, or 3 amino acid substitutions. In some embodiments, the amino acid substitutions are conservative amino acid substitutions.

### V_{H} Sequences Comprising Illustrative Chothia CDRs

In some embodiments, the additional binding domain capable of binding to an NKp30 epitope comprises a V_{H} sequence comprising one or more Chothia CDR-H sequences comprising, consisting of, or consisting essentially of one or more illustrative Chothia CDR-H sequences provided in this disclosure, and variants thereof.

### Chothia CDR-H3

In some embodiments, the additional binding domain capable of binding to an NKp30 epitope comprises a V_{H} sequence comprising a Chothia CDR-H3 sequence comprising, consisting of, or consisting essentially of a sequence selected from SEQ ID NOS: 592-594.

### Chothia CDR-H2

In some embodiments, the additional binding domain capable of binding to an NKp30 epitope comprises a V_{H} sequence comprising a Chothia CDR-H2 sequence comprising, consisting of, or consisting essentially of a sequence selected from SEQ ID NOS: 580-582.

### Chothia CDR-H1

In some embodiments, the additional binding domain capable of binding to an NKp30 epitope comprises a V_{H} sequence comprising a Chothia CDR-H1 sequence comprising, consisting of, or consisting essentially of a sequence selected from SEQ ID NOS: 568-570.

### Chothia CDR-H3+Chothia CDR-H2

In some embodiments, the additional binding domain capable of binding to an NKp30 epitope comprises a V_{H} sequence comprising a Chothia CDR-H3 sequence comprising, consisting of, or consisting essentially of a sequence selected from SEQ ID NOS: 592-594, and a Chothia CDR-H2 sequence comprising, consisting of, or consisting essentially of a sequence selected from SEQ ID NOS: 580-582. In some embodiments, the Chothia CDR-H3 sequence and the Chothia CDR-H2 sequence are both from a single illustrative V_{H} sequence provided in this disclosure. For example, in some embodiments, the Chothia CDR-H3 and Chothia CDR-H2 are both from a single illustrative V_{H} sequence selected from SEQ ID NOS: 613-616.

### Chothia CDR-H3+Chothia CDR-H1

In some embodiments, the additional binding domain capable of binding to an NKp30 epitope comprises a V_{H} sequence comprising a Chothia CDR-H3 sequence comprising, consisting of, or consisting essentially of a sequence selected from SEQ ID NOS: 592-594, and a Chothia CDR-H1 sequence comprising, consisting of, or consisting essentially of a sequence selected from SEQ ID NOS: 568-570. In some embodiments, the Chothia CDR-H3 sequence and the Chothia CDR-H1 sequence are both from a single illustrative V_{H} sequence provided in this disclosure. For example, in some embodiments, the Chothia CDR-H3 and Chothia CDR-H1 are both from a single illustrative V_{H} sequence selected from SEQ ID NOS: 613-616.

### Chothia CDR-H1+Chothia CDR-H2

In some embodiments, the additional binding domain capable of binding to an NKp30 epitope comprises a V_{H} sequence comprising a Chothia CDR-H1 sequence comprising, consisting of, or consisting essentially of a sequence selected from SEQ ID NOS: 568-570 and a Chothia CDR-H2 sequence comprising, consisting of, or consisting essentially of a sequence selected from SEQ ID NOS: 580-582. In some embodiments, the Chothia CDR-H1 sequence and the Chothia CDR-H2 sequence are both from a single illustrative V_{H} sequence provided in this disclosure. For example, in some embodiments, the Chothia CDR-H1 and Chothia CDR-H2 are both from a single illustrative V_{H} sequence selected from SEQ ID NOS: 613-616.

### Chothia CDR-H1+Chothia CDR-H2+Chothia CDR-H3

In some embodiments, the additional binding domain capable of binding to an NKp30 epitope comprises a V_{H} sequence comprising a Chothia CDR-H1 sequence comprising, consisting of, or consisting essentially of a sequence selected from SEQ ID NOS: 568-570, a Chothia CDR-H2 sequence comprising, consisting of, or consisting essentially of a sequence selected from SEQ ID NOS: 580-582, and a Chothia CDR-H3 sequence comprising, consisting of, or consisting essentially of a sequence selected from SEQ ID NOS: 592-594. In some embodiments, the Chothia CDR-H1 sequence, Chothia CDR-H2 sequence, and Chothia CDR-H3 sequence are all from a single illustrative V_{H} sequence provided in this disclosure. For example, in some embodiments, the Chothia CDR-H1, Chothia CDR-H2, and Chothia CDR-H3 are all from a single illustrative V_{H} sequence selected from SEQ ID NOS: 613-616.

### Variants of V_{H} Sequences Comprising Illustrative Chothia CDRs

In some embodiments, the V_{H} sequences provided herein comprise a variant of an illustrative Chothia CDR-H3, CDR-H2, and/or CDR-H1 sequence provided in this disclosure.

In some embodiments, the Chothia CDR-H3 sequence comprises, consists of, or consists essentially of a variant of an illustrative Chothia CDR-H3 sequence provided in this disclosure. In some embodiments, the Chothia CDR-H3 sequence comprises, consists of, or consists essentially of a sequence having at least 70%, 75%, 80%, 85%, 90%, or 95% identity with any of the illustrative Chothia CDR-H3 sequences provided in this disclosure. In some embodiments, the Chothia CDR-H3 sequence comprises, consists of, or consists essentially of any of the illustrative Chothia CDR-H3 sequences provided in this disclosure, with 1, 2, or 3 amino acid substitutions. In some embodiments, the amino acid substitutions are conservative amino acid substitutions.

In some embodiments, the Chothia CDR-H2 sequence comprises, consists of, or consists essentially of a variant of an illustrative Chothia CDR-H2 sequence provided in this disclosure. In some embodiments, the Chothia CDR-H2 sequence comprises, consists of, or consists essentially of a sequence having at least 70%, 75%, 80%, 85%, 90%, or 95% identity with any of the illustrative Chothia CDR-H2 sequences provided in this disclosure. In some embodiments, the Chothia CDR-H2 sequence comprises, consists of, or consists essentially of any of the illustrative Chothia CDR-H2 sequences provided in this disclosure, with 1, 2, or 3 amino acid substitutions. In some embodiments, the amino acid substitutions are conservative amino acid substitutions.

In some embodiments, the Chothia CDR-H1 sequence comprises, consists of, or consists essentially of a variant of an illustrative Chothia CDR-H1 sequence provided in this disclosure. In some embodiments, the Chothia CDR-H1 sequence comprises, consists of, or consists essentially of a sequence having at least 70%, 75%, 80%, 85%, 90%, or 95% identity with any of the illustrative Chothia CDR-H1 sequences provided in this disclosure. In some embodiments, the Chothia CDR-H1 sequence comprises, consists of, or consists essentially of any of the illustrative Chothia CDR-H1 sequences provided in this disclosure, with 1, 2, or 3 amino acid substitutions. In some embodiments, the amino acid substitutions are conservative amino acid substitutions.

### V_{H} Sequences

In some embodiments, the additional binding domain capable of binding to an NKp30 epitope comprises a V_{H} sequence comprising, consisting of, or consisting essentially of a sequence selected from SEQ ID NOS: 613-616. In some embodiments, the additional binding domain capable of binding to an NKp30 epitope comprises a V_{H} sequence comprising, consisting of, or consisting essentially of SEQ ID NO: 613. In some embodiments, the additional binding domain capable of binding to an NKp30 epitope comprises a V_{H} sequence comprising, consisting of, or consisting essentially of SEQ ID NO: 614. In some embodiments, the additional binding domain capable of binding to an NKp30 epitope comprises a V_{H} sequence comprising, consisting of, or consisting essentially of SEQ ID NO: 615. In some embodiments, the additional binding domain capable of binding to an NKp30 epitope comprises a V_{H} sequence comprising, consisting of, or consisting essentially of SEQ ID NO: 616.

In some embodiments, the binding domain capable of binding to an NKp30 epitope comprises three heavy chain CDRs each comprising, consisting of, or consisting essentially of a CDR sequence of a VH having the sequence set forth in one of SEQ ID NOS: 613-616.

### Variants of V_{H} Sequences

In some embodiments, the V_{H} sequences provided herein comprise, consist of, or consist essentially of a variant of an illustrative V_{H} sequence provided in this disclosure.

In some embodiments, the V_{H} sequence comprises, consists of, or consists essentially of a variant of an illustrative V_{H} sequence provided in this disclosure. In some embodiments, the V_{H} sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

### CDR-L3 Sequences

In some embodiments, the additional binding domain capable of binding to an NKp30 epitope comprises a CDR-L3 sequence comprising, consisting of, or consisting essentially of a sequence selected from SEQ ID NOS: 608-609.

In some embodiments, the CDR-L3 sequence comprises, consists of, or consists essentially of a variant of an illustrative CDR-L3 sequence provided in this disclosure. In some embodiments, the CDR-L3 sequence comprises, consists of, or consists essentially of a sequence having at least 70%, 75%, 80%, 85%, 90%, or 95% identity with any of the illustrative CDR-L3 sequences provided in this disclosure. In some embodiments, the CDR-L3 sequence comprises, consists of, or consists essentially of any of the illustrative CDR-L3 sequences provided in this disclosure, with 1, 2, or 3 amino acid substitutions. In some embodiments, the amino acid substitutions are conservative amino acid substitutions.

### V_{L} Sequences Comprising Illustrative CDRs

In some embodiments, the additional binding domain capable of binding to an NKp30 epitope comprises a V_{L} sequence comprising one or more CDR-L sequences comprising, consisting of, or consisting essentially of one or more illustrative CDR-L sequences provided in this disclosure, and variants thereof.

### CDR-L3

In some embodiments, the additional binding domain capable of binding to an NKp30 epitope comprises a V_{L} sequence comprising a CDR-L3 sequence comprising, consisting of, or consisting essentially of a sequence selected from SEQ ID NOS: 608-609.

### CDR-L2

In some embodiments, the additional binding domain capable of binding to an NKp30 epitope comprises a V_{L} sequence comprising a CDR-L2 sequence comprising, consisting of, or consisting essentially of a sequence selected from SEQ ID NOS: 603-604.

### CDR-L1

In some embodiments, the additional binding domain capable of binding to an NKp30 epitope comprises a V_{L} sequence comprising a CDR-L1 sequence comprising, consisting of, or consisting essentially of a sequence selected from SEQ ID NOS: 598-599.

### CDR-L3+CDR-L2

In some embodiments, the additional binding domain capable of binding to an NKp30 epitope comprises a V_{L} sequence comprising a CDR-L3 sequence comprising, consisting of, or consisting essentially of a sequence selected from SEQ ID NOS: 608-609 and a CDR-L2 sequence comprising, consisting of, or consisting essentially of a sequence selected from SEQ ID NOS: 603-604. In some embodiments, the CDR-L3 sequence and the CDR-L2 sequence are both from a single illustrative V_{L} sequence provided in this disclosure. For example, in some embodiments, the CDR-L3 and CDR-L2 are both from a single illustrative V_{L} sequence selected from SEQ ID NOS: 624-625.

### CDR-L3+CDR-L1

In some embodiments, the additional binding domain capable of binding to an NKp30 epitope comprises a V_{L} sequence comprising a CDR-L3 sequence comprising, consisting of, or consisting essentially of a sequence selected from SEQ ID NOS: 608-609 and a CDR-L1 sequence comprising, consisting of, or consisting essentially of a sequence selected from SEQ ID NOS: 598-599. In some embodiments, the CDR-L3 sequence and the CDR-L1 sequence are both from a single illustrative V_{L} sequence provided in this disclosure. For example, in some embodiments, the CDR-L3 and CDR-L1 are both from a single illustrative V_{L} sequence selected from SEQ ID NOS: 624-625.

### CDR-L1+CDR-L2

In some embodiments, the additional binding domain capable of binding to an NKp30 epitope comprises a V_{L} sequence comprising a CDR-L1 sequence comprising, consisting of, or consisting essentially of a sequence selected from SEQ ID NOS: 598-599 and a CDR-L2 sequence comprising, consisting of, or consisting essentially of a sequence selected from SEQ ID NOS: 603-604. In some embodiments, the CDR-L1 sequence and the CDR-L2 sequence are both from a single illustrative V_{L} sequence provided in this disclosure. For example, in some embodiments, the CDR-L1 and CDR-L2 are both from a single illustrative V_{L} sequence selected from SEQ ID NOS: 624-625.

### CDR-L1+CDR-L2+CDR-L3

In some embodiments, the additional binding domain capable of binding to an NKp30 epitope comprises a V_{L} sequence comprising a CDR-L1 sequence comprising, consisting of, or consisting essentially of a sequence selected from SEQ ID NOS: 598-599, a CDR-L2 sequence comprising, consisting of, or consisting essentially of a sequence selected from SEQ ID NOS: 603-604, and a CDR-L3 sequence comprising, consisting of, or consisting essentially of a sequence selected from SEQ ID NOS: 608-609. In some embodiments, the CDR-L1 sequence, CDR-L2 sequence, and CDR-L3 sequence are all from a single illustrative V_{L} sequence provided in this disclosure. For example, in some embodiments, the CDR-L1, CDR-L2, and CDR-L3 are all from a single illustrative V_{L} sequence selected from SEQ ID NOS: 624-625.

### Variants of V_{L} Sequences Comprising Illustrative CDR-Ls

In some embodiments, the V_{L} sequences provided herein comprise a variant of an illustrative CDR-L3, CDR-L2, and/or CDR-L1 sequence provided in this disclosure.

In some embodiments, the CDR-L3 sequence comprises, consists of, or consists essentially of a variant of an illustrative CDR-L3 sequence provided in this disclosure. In some embodiments, the CDR-L3 sequence comprises, consists of, or consists essentially of a sequence having at least 70%, 75%, 80%, 85%, 90%, or 95% identity with any of the illustrative CDR-L3 sequences provided in this disclosure. In some embodiments, the CDR-L3 sequence comprises, consists of, or consists essentially of any of the illustrative CDR-L3 sequences provided in this disclosure, with 1, 2, or 3 amino acid substitutions. In some embodiments, the amino acid substitutions are conservative amino acid substitutions.

In some embodiments, the CDR-L2 sequence comprises, consists of, or consists essentially of a variant of an illustrative CDR-L2 sequence provided in this disclosure. In some embodiments, the CDR-L2 sequence comprises, consists of, or consists essentially of a sequence having at least 70%, 75%, 80%, 85%, 90%, or 95% identity with any of the illustrative CDR-L2 sequences provided in this disclosure. In some embodiments, the CDR-L2 sequence comprises, consists of, or consists essentially of any of the illustrative CDR-L2 sequences provided in this disclosure, with 1, 2, or 3 amino acid substitutions. In some embodiments, the amino acid substitutions are conservative amino acid substitutions.

In some embodiments, the CDR-L1 sequence comprises, consists of, or consists essentially of a variant of an illustrative CDR-L1 sequence provided in this disclosure. In some embodiments, the CDR-L1 sequence comprises, consists of, or consists essentially of a sequence having at least 70%, 75%, 80%, 85%, 90%, or 95% identity with any of the illustrative CDR-L1 sequences provided in this disclosure. In some embodiments, the CDR-L1 sequence comprises, consists of, or consists essentially of any of the illustrative CDR-L1 sequences provided in this disclosure, with 1, 2, or 3 amino acid substitutions. In some embodiments, the amino acid substitutions are conservative amino acid substitutions.

### V_{L} Sequences

In some embodiments, the additional binding domain capable of binding to an NKp30 epitope comprises a V_{L} sequence comprising, consisting of, or consisting essentially of a sequence selected from SEQ ID NOS: 624-625. In some embodiments, the additional binding domain capable of binding to an NKp30 epitope comprises a V_{L} sequence comprising, consisting of, or consisting essentially of SEQ ID NO: 624. In some embodiments, the additional binding domain capable of binding to an NKp30 epitope comprises a V_{L} sequence comprising, consisting of, or consisting essentially of SEQ ID NO: 625.

In some embodiments, the binding domain capable of binding to an NKp30 epitope comprises three light chain CDRs each comprising, consisting of, or consisting essentially of a CDR sequence of a VL having the sequence set forth in one of SEQ ID NOS: 624-625.

### Variants of V_{L} Sequences

In some embodiments, the V_{L} sequences provided herein comprise, consist of, or consist essentially of a variant of an illustrative V_{L} sequence provided in this disclosure.

In some embodiments, the V_{L} sequence comprises, consists of, or consists essentially of a variant of an illustrative V_{L} sequence provided in this disclosure. In some embodiments, the V_{L} sequence comprises, consists of, or consists essentially of a sequence having at least 85%, 90%, 95%, 96%, 97%, 98%, 99%, or 99.05% identity with any of the illustrative V_{L} sequences provided in this disclosure.

In some embodiments, the V_{L} sequence comprises, consists of, or consists essentially of any of the illustrative V_{L} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

### Pairs

### CDR-H3-CDR-L3 Pairs

In some embodiments, the additional binding domain capable of binding to an NKp30 epitope comprises a CDR-H3 sequence and a CDR-L3 sequence. In some embodiments, the CDR-H3 sequence is part of a V_{H} and the CDR-L3 sequence is part of a V_{L}.

In some embodiments, the CDR-H3 sequence is a CDR-H3 sequence comprising, consisting of, or consisting essentially of SEQ ID NOS: 592-594, and the CDR-L3 sequence is a CDR-L3 sequence comprising, consisting of, or consisting essentially of SEQ ID NOS: 608-609.

### Variants of CDR-H3-CDR-L3 Pairs

In some embodiments, the CDR-H3-CDR-L3 pairs provided herein comprise a variant of an illustrative CDR-H3 and/or CDR-L1 sequence provided in this disclosure.

In some embodiments, the CDR-H3 sequence comprises, consists of, or consists essentially of a variant of an illustrative CDR-H3 sequence provided in this disclosure. In some embodiments, the CDR-H3 sequence comprises, consists of, or consists essentially of a sequence having at least 70%, 75%, 80%, 85%, 90%, or 95% identity with any of the illustrative CDR-H3 sequences provided in this disclosure. In some embodiments, the CDR-H3 sequence comprises, consists of, or consists essentially of any of the illustrative CDR-H3 sequences provided in this disclosure, with 1, 2, or 3 amino acid substitutions. In some embodiments, the amino acid substitutions are conservative amino acid substitutions.

In some embodiments, the CDR-L3 sequence comprises, consists of, or consists essentially of a variant of an illustrative CDR-L3 sequence provided in this disclosure. In some embodiments, the CDR-L3 sequence comprises, consists of, or consists essentially of a sequence having at least 70%, 75%, 80%, 85%, 90%, or 95% identity with any of the illustrative CDR-L3 sequences provided in this disclosure. In some embodiments, the CDR-L3 sequence comprises, consists of, or consists essentially of any of the illustrative CDR-L3 sequences provided in this disclosure, with 1, 2, or 3 amino acid substitutions. In some embodiments, the amino acid substitutions are conservative amino acid substitutions.

V_{H}-V_{L} Pairs

In some embodiments, the additional binding domain capable of binding to an NKp30 epitope comprises a V_{H} sequence and a V_{L} sequence.

In some embodiments, the V_{H} sequence is a V_{H} sequence comprising, consisting of, or consisting essentially of SEQ ID NOS: 613-616 and the V_{L} sequence is a V_{L} sequence comprising, consisting of, or consisting essentially of SEQ ID NOS: 624-625.

In some embodiments, the binding domain capable of binding to an NKp30 epitope comprises three heavy chain CDRs and three light chain CDRs, each comprising, consisting of, or consisting essentially of a CDR sequence of a VH-VL pair set forth above. In some embodiments, the binding domain capable of binding to an HLA-G epitope comprises three heavy chain CDRs and three light chain CDRs, each comprising, consisting of, or consisting essentially of a CDR sequence of a VH-VL pair set forth in Table S.

Variants of V_{H}-V_{L} Pairs

In some embodiments, the V_{H}-V_{L} pairs provided herein comprise a variant of an illustrative V_{H} and/or V_{L} sequence provided in this disclosure.

In some embodiments, the V_{H} sequence comprises, consists of, or consists essentially of a variant of an illustrative V_{H} sequence provided in this disclosure. In some embodiments, the V_{H} sequence comprises, consists of, or consists essentially of a sequence having at least 85%, 90%, 95%, 96%, 97%, 98%, 99%, or 99.1% identity with any of the illustrative V_{H} sequences provided in this disclosure.

In some embodiments, the V_{H} sequence comprises, consists of, or consists essentially of any of the illustrative V_{H} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

In some embodiments, the V_{L} sequence comprises, consists of, or consists essentially of a variant of an illustrative V_{L} sequence provided in this disclosure. In some embodiments, the V_{L} sequence comprises, consists of, or consists essentially of a sequence having at least 85%, 90%, 95%, 96%, 97%, 98%, 99%, or 99.05% identity with any of the illustrative V_{L} sequences provided in this disclosure.

In some embodiments, the V_{L} sequence comprises, consists of, or consists essentially of any of the illustrative V_{L} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some embodiments, the amino acid substitutions are conservative amino acid substitutions.

### Pharmaceutical Compositions

Some embodiments provide a pharmaceutical composition comprising or consisting of any of the bispecific antigen binding constructs provided herein. Suitable routes of administration include, but are not limited to, the inhalation, intra-arterial, intradermal, intramuscular, intraperitoneal, intravenous, nasal, parenteral, pulmonary, and subcutaneous routes.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

Further encompassed herein are anhydrous pharmaceutical compositions and dosage forms comprising an antibody, since water can facilitate the degradation of some antibodies. Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

In some embodiments, the pharmaceutical composition further comprises one or both of an antibody to an immune inhibitory receptor or ligand and/or an antibody to an immune stimulatory receptor and/or ligand. In some embodiments, the pharmaceutical composition further comprises an effective amount of at least one of the following: an anti-ILT2 antibody or a small molecule inhibitor; an anti-ILT4 antibody or small molecule inhibitor; an anti-KIR2DL4 antibody or small molecule inhibitor; an anti-PD-L1 antibody or small molecule inhibitor; an anti-PD-1 antibody or small molecule inhibitor; an anti-CTLA4 antibody or small molecule inhibitor; an anti-CD38 antibody or small molecule inhibitor; an anti-CD73 antibody or small molecule inhibitor; an anti-A2A receptor antibody or small molecule inhibitor; an anti-A2B receptor antibody or small molecule inhibitor; an anti-A2A/A2B dual receptor antibody or small molecule inhibitor or a combination thereof; an anti-CD39 antibody or small molecule inhibitor; an anti-CD73 antibody or small molecule inhibitor; an anti-CD47 antibody or small molecule inhibitor; and/or a small molecule inhibitor. In some embodiments, the pharmaceutical composition further comprises an anti-ILT2 antibody or small molecule inhibitor, an anti-ILT4 antibody or small molecule inhibitor, an anti-PD-L1 antibody or small molecule inhibitor, and/or an anti-CD47 antibody or small molecule inhibitor.

In some embodiments, the pharmaceutical composition further comprises an effective amount of one or more of:
a) an anti-PD-L1 antibody or small molecule inhibitor;
b) an anti-PD-1 antibody or small molecule inhibitor;
c) an anti-CD38 antibody or small molecule inhibitor;
d) an anti-CD39 antibody or small molecule inhibitor;
e) an anti-CD73 antibody or small molecule inhibitor;
f) an anti-A2A receptor antibody or small molecule inhibitor;
g) an anti-A2B receptor antibody or small molecule inhibitor;
h) an anti-A2A/A2B dual receptor antibody or small molecule inhibitor;
i) an anti-CD47 antibody or small molecule inhibitor;
j) an anti-CTLA-4 antibody or small molecule inhibitor;
k) an anti-LAG-3 antibody or small molecule inhibitor;
l) an anti-TIM-3 antibody or small molecule inhibitor;
m) an anti-TIGIT antibody or small molecule inhibitor;
n) an anti-VISTA antibody or small molecule inhibitor;
o) an anti-CD94 antibody or small molecule inhibitor;
p) a small molecule inhibitor;
q) an oncolytic virus;
r) a chemotherapy;
s) ADCC capable therapies using effector competent antibodies such as anti-CD19, anti-CD20, anti-EGFR, anti-Her2, anti-SLAMF7, anti-CD52, anti-BCMA, anti-GD2, and/or anti-CCR4.

In some embodiments, chemotherapy comprises one or more receptor tyrosine kinase inhibitors. Examples of receptor tyrosine inhibitors include acalabrutinibafatinib, alecensa, alectinib, avapritinib, axitinib, bosulif, bosutinib, brukinsa cabozantinib, calquence, caprelsa, cometriq, crizotinib, dacomitinib, dasatinib, entrectinib, erlotinib, gilotrif, gilteritinib, gleevec, ibrutinib, iclusig, imatinib, imbruvica, inlyta, lapatinib, midostaurin, neratinib, nerlynx, nexavar, nilotinib, pacritinib, pazopanib, pexidartinib, ponatinib, quizartinib, regorafenib, Rozlytrek, rydapt, sorafenib, sprycel, stivarga, sunitinib, sutent, tarceva, tasigna, turalio, tykerb, vandetanib, vizimpro, votrient, xalkori, xospata, zaltrap, zanubrutinib, ziv-aflibercept.

In some embodiments, chemotherapy comprises one or more antimetabolites. Antimetabolities include, without limitation, zacitidine, 5-fluorouracil (5-FU), 6-mercaptopurine (6-MP), capecitabine (Xeloda), cladribine, clofarabine, cytarabine (Ara-C), decitabine, floxuridine, fludarabine, gemcitabine (Gemzar), hydroxyurea, methotrexate, nelarabine, pemetrexed (Alimta), pentostatin, pralatrexate, thioguanine, and/or trifluridine/tipiracil.

In some embodiments, chemotherapy comprises one or more alkylating agents. Alkylating agents include, with out limitation, altretamine, bendamustine, busulfan, carboplatin, carmustine, chlorambucil, cisplatin, cyclophosphamide, dacarbazin, iosfamide, lomustine, mechlorethamine, melphalan, oxaliplatin, temozolomide, thiotepa, and/or trabectedin.

In some embodiments, chemotherapy comprises one or more anthracyclines. Anthracyclines include, without limitation, daunorubicin, doxorubicin (Adriamycin), doxorubicin liposomal, epirubicin, idarubicin, and/or valrubicin.

In some embodiments, chemotherapy comprises one or more topoisomerase inhibitors such as, for example, without limitation, irinotecan, irinotecan liposomal, and/or topotecan.

In some embodiments, chemotherapy comprises one or more taxanes such as, for example, without limitation, cabazitaxel, docetaxel, nab-paclitaxel, and/or paclitaxel.

In some embodiments, chemotherapy comprises one or more, vinca alkaloids such, for example, without limitation, vinblastine, vincristine, vincristine liposomal, and/or vinorelbine.

In some embodiments, chemotherapy comprises one or more of all-trans-retinoic acid, arsenic trioxide, asparaginase, eribulin, hydroxyurea, ixabepilone, mitotane, omacetaxine, pegaspargase, procarbazine, romidepsin, and/or vorinostat.

In some embodiments, the pharmaceutical composition further comprises one or both of:
c) an antibody to an immune inhibitory receptor or ligand and/or
d) an antibody to an immune stimulatory receptor or ligand.

In some embodiments, the one or more immunomodulatory agents comprise an antagonist to an inhibitory receptor of an immune cell. In some embodiments, the inhibitory receptor is at least one of LILRB1, LILRB2, LILRB4, KIR2DL4, CTLA-4, PD-1, PD-L1, PD-L2, LAG-3, Tim3, TIGIT, B7-H3, B7-H4, neuritin, BTLA, CECAM-1, CECAM-5, VISTA, LAIR1, CD160, 2B4, TGF-B receptor, NKG2A, and/or a Killer-cell immunoglobulin-like receptor (KIR). In some embodiments, the one or more immunomodulatory agents comprise an agonist of a co-stimulatory receptor of an immune cell. In some embodiments, the co-stimulatory receptor is at least one of OX40, CD2, CD27, ICAM-1, LFA-1, ICOS (CD278), 4-1BB (CD137), GITR, CD28, CD30, CD40, BAFFR, HVEM, CD7, LIGHT, NKG2C, SLAMF7, NKp30, NKp46, NKp80, CD160, and/or CD83.

In some embodiments, the one or more immunomodulatory agents is one or more cytokines. In some embodiments, the one or more cytokines is at least one of G-CSF, GM-CSF, IFN-alpha, IFN-beta, IFN-gamma, FLt3 ligand, IL-1, IL-2, IL-5, IL-7, IL-10, IL-12, IL-15, IL-18, IL-21, and/or IL-27.

In some embodiments, the one or more immunomodulatory agents is one or more oncolytic viruses. In some embodiments, the one or more oncolytic viruses is a Herpes simplex virus, a Vesicular stomatitis virus, an adenovirus, a Newcastle disease virus, a vaccinia virus, and/or a maraba virus.

### Nucleic Acids, Vectors, Hosts, and Cell Lines

A second aspect provides a one or more nucleic acids encoding any of the bispecific antigen binding constructs provided herein. In some embodiments, the one or more nucleic acids comprises one or more vectors. Some embodiments provide a host transformed with the one or more vectors. Some embodiments provide a method for the production of one or more bispecific antigen binding construct comprising the steps of expressing any of the one or more nucleic acids provided herein in a prokaryotic or eukaryotic host cell and recovering the one or more bispecific antigen binding construct from the cell or the cell culture supernatant.

For recombinant production of the antibody or bispecific antigen binding construct, the one or more nucleic acids encoding it may be isolated and inserted into one or more replicable vectors for further cloning (i.e., amplification of the DNA) or expression. In some aspects, the one or more nucleic acids may be produced by homologous recombination, for example as described in U.S. Pat. No. 5,204,244, which is incorporated by reference in its entirety herein.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615, which is incorporated in its entirety herein.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W3110 are suitable.

In addition to prokaryotes, eukaryotic microbes such as filamentous fungi or yeast are also suitable cloning or expression hosts for antibody-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the bispecific antigen binding construct or antibody of this invention may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 may be used; each of the above-noted references are incorporated by reference herein in their entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the bispecific antigen binding construct or antibody can be produced intracellularly, in the periplasmic space, or directly secreted into the medium. If the antibody or bispecific antigen binding construct is produced intracellularly, as a first step, the particulate debris, either host cells or lysed fragments, is removed, for example, by centrifugation or ultrafiltration. For example, Carter et al. (BioTechnology, 1992, 10:163-167, which is incorporated by reference in its entirety herein) describes a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 minutes. Cell debris can be removed by centrifugation.

In some embodiments, the bispecific antigen binding construct or antibody is produced in a cell-free system. In some embodiments, the cell-free system is an in vitro transcription and translation system as described in Yin et al., mAbs, 2012, 4:217-225, incorporated by reference in its entirety. In some embodiments, the cell-free system utilizes a cell-free extract from a eukaryotic cell or from a prokaryotic cell. In some embodiments, the prokaryotic cell is E. coli. Cell-free expression of the antibody or bispecific antigen binding construct may be useful, for example, where the antibody or bispecific antigen binding construct accumulates in a cell as an insoluble aggregate, or where yields from periplasmic expression are low.

Where the bispecific antigen binding construct or antibody is secreted into the medium, supernatants from such expression systems are generally first concentrated using a commercially available protein concentration filter, for example, an Amicon® or Millipore® Pelicon® ultrafiltration unit. A protease inhibitor such as PMSF may be included in any of the foregoing steps to inhibit proteolysis and antibiotics may be included to prevent the growth of adventitious contaminants.

The bispecific antigen binding construct or antibody composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the antibody. Protein A can be used to purify antibodies that are based on human 71, y2, or y4 heavy chains (Lindmark et al., J. Immunol. Meth., 1983, 62:1-13). Protein G is useful for all mouse isotypes and for human y3 (Guss et al., EMBO J., 1986, 5:1567-1575).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the bispecific antigen binding construct or antibody comprises a CH3 domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the antibody or bispecific antigen binding construct of interest and contaminants may be subjected to low pH hydrophobic interaction chromatography using an elution buffer at a pH between about 2.5 to about 4.5, generally performed at low salt concentrations (e.g., from about 0 to about 0.25 M salt).

### Preparation of Anti-HLA-G Bispecific Antigen Binding Constructs

Methods for making bispecific antigen binding constructs or bispecific antibodies are known in the art (See, e.g., Suresh et al., Methods in Enzymology 121:210, 1986, which is incorporated by reference herein in its entirety). Traditionally, recombinant production of bispecific antigen binding constructs or bispecific antibodies was based on the coexpression of two IgG heavy chain-light chain pairs, with the two heavy chains having different specificities (See, Millstein and Cuello, Nature 305: 537-539, 1983, which is incorporated by reference in its entirety herein).

For example, in one approach, bispecific antigen binding constructs or bispecific antibodies may be composed of a hybrid IgG heavy chain with a first binding specificity in one arm and a hybrid IgG heavy chain-light chain pair (providing a second binding specificity) in the other arm. This asymmetric structure, with an IgG light chain in only one half of the bispecific molecule, facilitates the separation of the desired bispecific compound from unwanted IgG chain combinations. The approach is described in PCT Publication No. WO 94/04690, which is incorporated by reference in its entirety herein.

In another approach, for example, bispecific antigen binding constructs or bispecific antibodies are composed of amino acid modification by replacement of a small amino acid with a larger one in the CH3 domain in one arm to introduce a protuberance at the IgG Fc interface (‘knob’) and replacement of a large residue with a smaller one in the CH3 domain in another arm to introduce a cavity of the corresponding IgG Fc interface (‘hole’). The ‘knob’ can be positioned in the ‘hole’ so as to promote heteromultimer formation and hinder homomultimer formation. This ‘knobs-into-holes’ approach is described in Ridgway et al Protein Engineering 9: 617-621, 1996, and U.S. Pat. No. 8,679,785 B2, which is incorporated by reference herein in its entirety.

In another approach, bispecific antigen binding constructs or bispecific antibodies are composed of amino acid modification in the first hinge region in one arm and the substituted/replaced amino acid in the first hinge region has an opposite charge to the corresponding amino acid in a second hinge region in another arm. The formation of the bispecific antigen binding constructs or bispecific antibody is enhanced by altering or engineering an interface between a first and a second IgG Fc region (e.g., a hinge region and/or a CH3 region). In this approach, the bispecific antigen binding constructs or bispecific antibodies may be composed of a CH3 region, wherein the CH3 region comprises a first CH3 polypeptide and a second CH3 polypeptide which interact together to form a CH3 interface, wherein one or more amino acids within the CH3 interface destabilize homodimer formation and are not electrostatically unfavorable to homodimer formation. This approach is described in International Patent Application No. PCT/US2011/036419 (WO2011/143545) and Strop et al. JMB 420: 204-219, 2012, which is incorporated by reference in its entirety herein.

In some embodiments, the bispecific antigen binding construct comprises or consist of one or more diabodies. Diabodies are bivalent, bispecific antigen binding constructs or bispecific antibodies in which heavy chain variable (VH) and light chain variable (VL) domains are expressed on a single polypeptide chain, but using a linker that is too short to allow for pairing between the two domains on the same chain, thereby forcing the domains to pair with complementary domains of another chain and creating two antigen binding sites (See, e.g., Holliger, P., et al., Proc. Natl. Acad Sci. USA 90:6444-6448, 1993; Poljak, R. J., et al., Structure 2:1121-1123, 1994, which is incorporated by reference in its entirety herein). The short linking peptide bridges between the carboxy terminus of one variable region (e.g. of HLA-G) and the amino terminus of the other variable region (e.g. of CD3), producing a VL-VH sequence. The VL-VH sequence may be further linked to an IgG Fc region. In some embodiments, the IgG Fc can be either human or mouse or human/mouse IgG1, IgG2, IgG3, or IgG4 isotype.

In some embodiments, a bispecific diabody-Fc antibody comprises or consists of a diabody fused to an IgG Fc domain, wherein a first antibody variable domain of the diabody is capable of specifically binding to a target antigen (e.g. HLA-G), and wherein a second antibody variable domain of the diabody is capable of recruiting the activity of a human immune effector cell by specifically binding to an effector antigen located on a human immune effector cell. In some embodiments, the effector antigen is CD3. In some embodiments, the human immune effector cell is a CD8⁺ T cell.

In some embodiments, bispecific antigen binding fragments comprise or consist of one or more of an scFv, minibodies, or VHH single domain antibodies. For scFv, single chain variable region fragments are made by linking light and/or heavy chain variable regions by using a short linking peptide (See, Bird et al., Science 242:423-426, 1988, which is incorporated by reference in its entirety). An example of a linking peptide is (GGGGS)₄, which bridges between the carboxy terminus of one variable region and the amino terminus of the other variable region. Linkers of other sequences have been designed and used (See, Bird et al., Science 242:423-426, 1988). For example, two scFvs targeting two different antigens can be linked by another linker to form a bispecific scFv.

In some embodiments, the bispecific antigen binding construct comprises or consists of a minibody. A minibody is an antibody which features a smaller molecular weight that a traditional large antibody while still maintaining binding. Because of the smaller size, a minibody features faster clearance from a subject's system and could also feature enhanced penetration when targeting a tumor tissue.

A minibody may be composed of a pair of scFv fragments which are linked via CH3 domains, and Fvs with distinct specificity, which are paired to scFv fragments through heterodimerization process. To promote the heterodimerization efficiency, single-residue mutations can be introduced into each CH3 domain to achieve the knob and hole approach. Far more than that, additional cysteine residues can also be introduced into CH3 domains to stabilize the bispecific minibody structure.

Minibodies can also include the VL and VH domains of a native antibody fused to the hinge region and CH3 domain of the immunoglobulin molecule. See, e.g., U.S. Pat. No. 5,837,821, which is incorporated by reference in its entirety herein.

In some embodiments, the bispecific antigen binding construct comprises or consists of one or more VHH single domain antibodies. A VHH single domain antibody is engineered from VH domain found in camelids (See, Muyldermans and Lauwereys, Journal of Molecular Recognition, 1999, which is incorporated by reference herein), and can be fused to IgG Fc domain to form a bispecific antigen binding construct VHH antibody. Exemplary heterologous sequences include, but are not limited to, a “tag” such as a Avi tag or a 8His tag. Tags are well known in the art.

### Methods of Treatment

A third aspect provides a method for treating a subject with cancer comprising administering any of the bispecific antigen binding constructs or pharmaceutical compositions provided herein to the subject in a therapeutically effective amount.

In some embodiments, the cancer is a solid cancer. In some embodiments, the cancer is a hematological cancer. In some embodiments, the cancer is selected from the group consisting of a hematopeietic cancer, hepatocellular carcinoma, leukemia, colorectal cancer (CRC), breast cancer, gastric cancer, esophageal cancer, endometrial cancer, prostate cancer, bladder cancer, thyroid cancer, liver cancer, pancreatic cancer, triple negative breast cancer, cervical cancer, ovarian cancer, uterine cancer, vaginal cancer, vulvar cancer, lung cancer, head and neck cancer, melanoma, renal cell carcinoma, cutaneous squamous cell carcinoma, Hodgkin's lymphoma, a metastasis of the brain, a metastasis of the lung, a metastasis of the liver, and/or a metastasis of the bone, or an unresectable or metastatic solid tumor with DNA mismatch repair deficiencies or a microsatellite instability-high state. In some embodiments, the cancer is a cancer that expresses HLA-G.

In some embodiments, the method further comprises one or more of the following:
a) administering chemotherapy to the subject;
b) administering radiation therapy to the subject; and/or
c) administering one or more additional therapeutic agents to the subject.
   In some embodiments, the one or more additional therapeutic agents comprise one or more immunomodulatory agents.

### Dosage

Parenteral dosage forms of the bispecific antigen binding constructs are provided in some embodiments. Parenteral dosage forms can be administered to subjects by various routes including, but not limited to, subcutaneous, intravenous (including bolus injection), intramuscular, and intra-arterial. Because their administration typically bypasses subjects' natural defenses against contaminants, parenteral dosage forms are typically sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to, water for injection; aqueous vehicles such as for example including, but not limited to, sodium chloride injection, ringer's injection, dextrose injection, dextrose and sodium chloride injection, lactated ringer's injection; water miscible vehicles such as for example including, but not limited to, ethyl alcohol, polyethylene glycol, and polypropylene glycol; and non-aqueous vehicles such as for example including, but not limited to, corn oil, cottonseed oil, peanut oil, sesame oil, ethyl oleate, isopropyl myristate, and benzyl benzoate.

Excipients that increase the solubility of one or more of the bispecific antigen binding constructs disclosed herein can also be incorporated into the parenteral dosage forms.

In human therapeutics, the doctor will determine the dosology which she considers most appropriate according to a preventive or curative treatment and according to the age, weight, condition, and other factors specific to the subject to be treated.

The amount of the bispecific antigen binding construct or composition which will be effective in the prevention or treatment of a disorder or one or more symptoms thereof will vary with the nature and severity of the disease or condition, and the route by which the antibody is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

Bispecific antigen binding constructs or pharmaceutical compositions according to the invention may be administered according to any suitable schedule. Bispecific antigen binding constructs or pharmaceutical compositions may be administered multiple times and at the same of different dosages. Bispecific antigen binding constructs or pharmaceutical compositions may be administered in any order and at any dose with respect to any of the other agents. Some embodiments comprise or consist of administering to the subject a bispecific antigen binding constructs or pharmaceutical compositions simultaneously or sequentially with any other agents provided herein. In some embodiments, bispecific antigen binding constructs or pharmaceutical compositions are administered before other agents. In some embodiments, bispecific antigen binding constructs or pharmaceutical compositions are administered after other agents. In some embodiments, bispecific antigen binding constructs or pharmaceutical compositions are administered multiple times and at the same of different dosages, or a combination thereof, as other agents.

In certain embodiments, exemplary doses of a bispecific antigen binding construct or composition include milligram or microgram amounts of the antibody per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). In certain embodiment, the dosage of the bispecific antigen binding construct provided herein, based on weight, administered to prevent, treat, manage, or ameliorate a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the bispecific antigen binding construct or a composition provided herein administered to prevent, treat, manage, or ameliorate a disorder, or one or more symptoms thereof in a subject is 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.25 mg to 2.5 mg, 0.5 mg to 20 mg, 0.5 to 15 mg, 0.5 to 12 mg, 0.5 to 10 mg, 0.5 mg to 7.5 mg, 0.5 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

The dose can be administered according to a suitable schedule, for example, once, two times, three times, or four times weekly. It may be necessary to use dosages of the bispecific antigen binding construct outside the ranges disclosed herein in some cases, as will be apparent to those of ordinary skill in the art. Furthermore, it is noted that the clinician or treating physician will know how and when to interrupt, adjust, or terminate therapy in conjunction with subject response.

In certain embodiments, treatment or prevention can be initiated with one or more loading doses of a bispecific antigen binding construct or composition provided herein followed by one or more maintenance doses.

In certain embodiments, a dose of a bispecific antigen binding construct or composition provided herein can be administered to achieve a steady-state concentration of the bispecific antigen binding construct in blood or serum of the subject. The steady-state concentration can be determined by measurement according to techniques available to those of skill or can be based on the physical characteristics of the subject such as height, weight and age.

In certain embodiments, administration of the same bispecific antigen binding construct or composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least 1 day, 2 days, 3 days, 5 days, 10 days, 15 days, 30 days, 45 days, 2 months, 75 days, 3 months, or 6 months.

In some embodiments, a therapeutically effective amount comprises or consists of determining an amount used to achieve a response according to a clinical endpoint. In some embodiments, the clinical endpoint comprises Objective Response Rate (ORR), Progression Free Survival (PFS), and/or Response Evaluation Criteria in Solid Tumors (“RECIST”).

### EXAMPLES

### Example 1: Selection of HLA-G Antibodies

HLA-G antibodies were selected from a synthetic library of human antibodies presented on the surface of yeast cells in IgG format, as generally described, e.g., in WO2009036379; WO2010105256; WO2012009568; and Xu et al., Protein Eng. Des. Sel., 2013, 26:663-670 (each incorporated by reference in its entirety), and more specifically as provided below. The sequences and characteristics of the antibodies isolated from the recombinant library are provided in Table S.

Eight naïve human synthetic yeast libraries each of ^{˜}10E+09 diversity were propagated as described in WO2009036379; WO2010105256; WO2012009568; and Xu et al., Protein Eng. Des. Sel., 2013, 26:663-670; each incorporated by reference in its entirety. For the first two rounds of selection, a magnetic bead sorting technique utilizing the Miltenyi MACS® system was performed, as described in Siegel et al., J. Immunol. Meth., 2004, 286:141-153. The following rounds of selection were performed using flow cytometry-based sorting. For all rounds of selection, the antigen was biotinylated human HLA-G, decreasing concentrations of antigen were used in each subsequent round of selection. In addition to selection on antigen, some rounds of selection were employed to reduce the number of non-specific binders utilizing soluble membrane proteins from CHO cells (see WO2014179363 and Xu et al., Protein Eng. Des. Sel., 2013, 26:663-670, each incorporated by reference in its entirety). In addition to the CHO cell proteins, deselections against recombinant HLA-A/B/C proteins were performed to maintain specific binding to HLA-G. After the final round of sorting, yeast were plated and individual colonies were picked for characterization and for nomination of clones for affinity maturation.

Antibody variable domains of interest were synthesized, with codon optimization to maximize transient expression in host cells. The variable regions were cloned into expression vectors containing human immunoglobulin constant domains and their sequence confirmed. Antibody heavy and light chain vector pairings were transfected into Expi293 cells using the Expifectamine system (Invitrogen). Transient cultures were harvested on day 4 and clarified cell culture supernatant IgG titer was estimated using Bio-Layer Interferometry (BLI) using Octet (ForteBio) alongside standards. Antibodies were subsequently purified on a Protein A column and eluted using low pH glycine. Purified antibody samples were then buffer-exchanged or dialyzed into downstream assay-compatible buffers.

Antibody purity was assessed by running samples on SDS-PAGE and on an analytical size exclusion chromatography column.

Light Chain Shuffling: Heavy chain plasmids were extracted from naïve outputs (described herein) and transformed into a pre-made naïve light chain library with a diversity of 10E+06. Selections were performed as described above with one round of MACS sorting and three rounds of FACS sorting using decreasing amounts of biotinylated HLA-G antigen for respective rounds.

### Example 2: Affinity Maturation of HLA-G Antibodies

Optimization of naïve clones was carried out utilizing four maturation strategies; diversification of CDR-H1 and CDR-H2; diversification of CDR-H3; diversification of CDR-L1, L2 and L3; shuffling of diversified heavy and light chains.

CDR-H1 and CDR-H2 Selection: The CDR-H3s from clones selected from light chain batch diversification, light chain diversification, and naive discovery efforts were independently recombined into premade libraries with CDR-H1 and CDR-H2 variants of a diversity of >10E+8. Selections were performed using HLA-G antigen. Affinity pressures were applied by using decreasing concentrations of antigen and HLA-G specificity was maintained with deselections against HLA-A/B/C antigens.

CDR-H3 Selection: After characterization of CDR-H1 and CDR-H2 variants, clones with binding to HLA antigens outside of HLA-G were removed. Chemical liabilities were also removed from the variable regions when applicable. The remaining clones obtained from the CDR-H1 and CDR-H2 selection procedure were subject to additional rounds of affinity maturation via walking dimer mutagenesis of the CDR-H3. Selections were performed using HLA-G as antigen generally as described above, except for employing FACS sorting for all selection rounds.

CDR-L1, L2, L3 Selection: Clones obtained from the CDR-H1 and CDR-H2 selection procedure were subject to additional rounds of affinity maturation via mutagenesis of the light chain. The CDR-L1 and CDR-L2 diversity was derived from a pre-made library while CDR-L3 diversity was derived from walking monomer mutagenesis. Selections were performed using HLA-G as antigen, starting with one round of MACS followed by three rounds of FACS in the CDR-L1, L2, L3 process described here.

Diversified Heavy Chain and Light Chain Shuffling: Outputs from CDR-H3 diversification and CDR-L1, L2, L3 diversification described above were recombined and selections were performed using HLA-G as antigen generally as described above, except for employing FACS sorting for all selection rounds.

### Example 3: Generation of HLA-G×CD3 Bispecific Antibody

The HLA-G×CD3 bispecific antibody was prepared using two single-chain VL-VH-Fc constructs. Anti-human CD3 VL (SEQ ID NO: 422) domain was linked on its C-terminus via a 7-amino acid linker, GGGSGGG, to the N-terminus of the anti-human HLA-G VH domain (SEQ ID NO: 192). This VL-VH sequence was linked to the ‘effector-silent’ human Fc region containing the “EEE” mutations in the hinge region and the CH3 domain. The resultant construct was transiently co-transfected with the single-chain anti-HLA-G VL (SEQ ID NO: 220)-anti-CD3 VH (SEQ ID NO: 413)-Fc with the “RRR” mutations in Expi-293 cells to form the bispecific antibody-Fc fusion molecules. The bispecific antibody was then purified using protein A, size-exclusion and ion exchange chromatography. This bispecific antibody generation approach is described in International Patent Application No. PCT/US2011/036419 (WO2011/143545) and Strop et al. JMB 420: 204-219, 2012, which is incorporated by reference in its entirety herein.

### Example 4: HLA-G×CD3 Bispecific Antibody Binds to HLA-G Expressing Cancer Cell Lines and to Human CD4⁺ and CD8⁺ T Cells

To evaluate binding to HLA-G⁺ tumor cells, A549 lung adenocarcinoma cells engineered to express HLA-G or JEG-3 choriocarcinoma cells that endogenously express HLA-G were incubated with a titration of an HLA-G×CD3 bispecific antibody and the parental anti-HLA-G monoclonal antibody for 30 minutes at 4° C. in staining buffer (Phosphate Buffered Saline (PBS), 2% Fetal bovine serum (FBS), 6% mouse serum, 1:10 human Fc block (Becton Dickinson) and 2 mM EDTA). After incubation, cells were washed in wash buffer (PBS, 2% FBS, 2 mM EDTA) followed by a 30-minute incubation with a PE-conjugated anti-human Fc secondary antibody (Biolegend). A final wash was performed followed by sample acquisition using a BD Celesta flow cytometer (Becton Dickinson). Sample data was exported as FCS files and analyzed using FlowJo software v10 (Tree Star, Inc.).

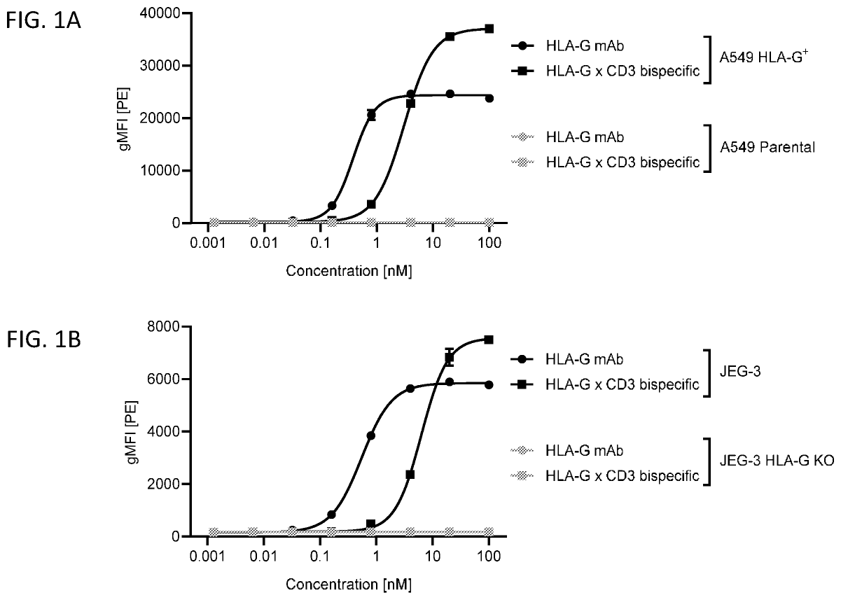

The results are shown in FIG. 1A and FIG. 1B. Negative controls included JEG-3 HLA-G KO cells generated by CRISPR-Cas9 gene editing and A549 parental cells.

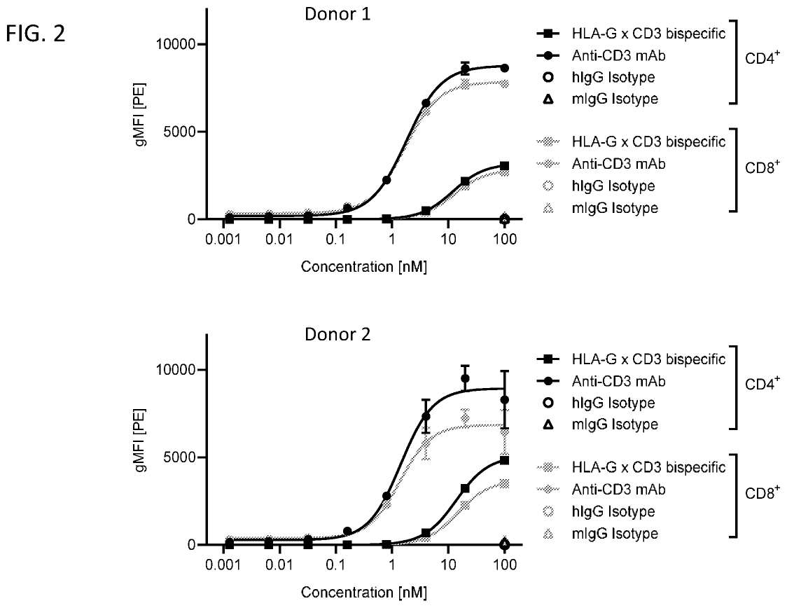

Binding of an HLA-G×CD3 bispecific antibody to CD3 was evaluated on primary human T cells. Briefly, pan T cells were isolated by negative selection using a pan T cell negative isolation kit (StemCell Technologies). Similar to the method used to evaluate tumor cell binding, T cells were incubated with a titration of an HLA-G×CD3 bispecific antibody followed by a 30-minute incubation with a PE-conjugated anti-human Fc secondary antibody (Biolegend). For the parental anti-CD3 antibody, a PE-conjugated anti-mouse Fc secondary antibody (Biolegend) was used. Cells were subsequently washed twice with wash buffer and then stained with fluorescently conjugated antibodies against CD4, CD8, and CD28 for 30 minutes at 4° C. A final wash was performed followed by sample acquisition using a BD Celesta flow cytometer (Becton Dickinson). Sample data was exported as FCS files and analyzed using FlowJo software v10 (Tree Star, Inc.). Gating was performed on CD28⁺CD4⁺ T cells and CD28⁺CD8⁺ T cells. Results from 2 representative donors are shown in FIG. 2.

### Example 5: HLA-G×CD3 Bispecific Antibody Induces T-Cell Mediated Cellular Cytotoxicity

To demonstrate that an HLA-G×CD3 bispecific antibody can induce killing of HLA-G⁺ cancer cells by human T cells, A549-HLA-G⁺ or JEG-3 cells were co-cultured with human peripheral blood mononuclear cells (PBMC). Negative controls included JEG-3 HLA-G KO cells and A549 parental cells, neither of which express detectable levels of HLA-G. Isolated PBMC (effector cells) were plated in complete RPMI (cRPMI) in a 96-well flat bottom plate at 400,000 cells/well. The effector cells were subsequently incubated with the HLA-G×CD3 bispecific antibody for 1 hour at 37° C., 5% CO₂. Target cells (A549 parental cells, A549-HLA-G⁺ cells, JEG-3 cells or JEG-3 HLA-G KO cells) were stained with 1:1000 CellTrace Violet at 37° C. in PBS, washed and plated in cRPMI at 10,000 cells/well. The mixture of effector cells and HLA-G×CD3 bispecific antibody was then combined with the target cells and incubated for 48 hours at 37° C., 5% CO₂. The final ratio of PBMC to target cells was 40:1.

After the 48-hour incubation, cells were washed once in 4° C. PBS, then resuspended in 50 μL TrypLE Express Enzyme (1×) with phenol red. The cells were incubated with TrypLE for 7 minutes at 37° C., 5% CO₂ before being quenched with 150 μL cRPMI. After quenching the enzyme, the cells were washed once in 4° C. wash buffer (Phosphate Buffered Saline, 2% FBS, 2 mM EDTA), and then stained with 1:2000 Fixable Viability Dye eFluor™ 780 (Invitrogen) for 30 minutes at 4° C. Cells were washed again in 4° C. wash buffer before being resuspended in wash buffer and analyzed for percent dead target cells by flow cytometry analysis on a BD LSRFortessa. Percent of dead target cells was determined by gating on CTV positive cells, then dividing the Fixable Viability Dye positive portion of these cells by the total CTV count.

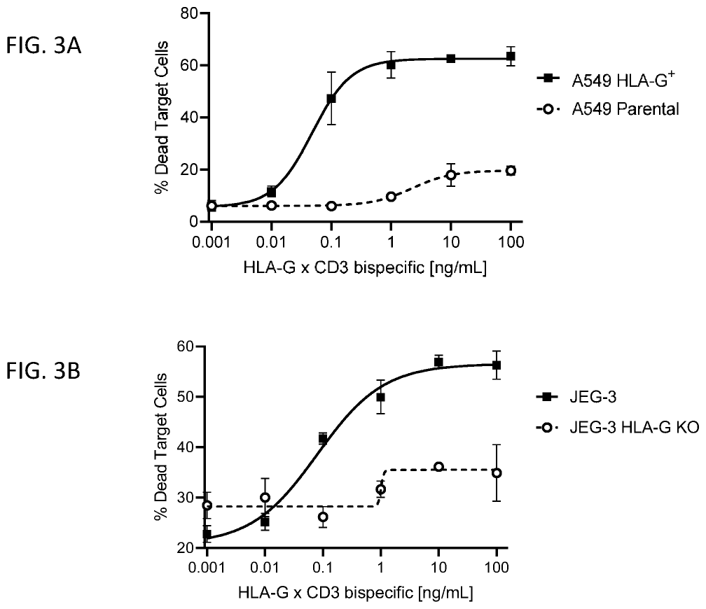

FIG. 3A and FIG. 3B demonstrate the titration of an HLA-G×CD3 bispecific antibody to induce T-cell mediated cytotoxicity of A549-HLA-G⁺ cells and JEG-3 cells, respectively.

TABLE S
Sequences.

| SEQ ID | | | |
|---|---|---|---|
| NO: | Region | Scheme/Clone | Sequence |
| 1 | CDR-H1 | Chothia | GGSISSSDY |
| 2 | CDR-H1 | Chothia | GGSISSSST |
| 3 | CDR-H1 | Chothia | GGSISSSDT |
| 4 | CDR-H1 | Chothia | GGSISSADN |
| 5 | CDR-H1 | Chothia | GGSVSSSST |
| 6 | CDR-H1 | Chothia | GYSISSGY |
| 7 | CDR-H1 | Chothia | GYSILSGY |
| 8 | CDR-H1 | Chothia | GYSISSGH |
| 9 | CDR-H1 | Chothia | GYSISSGF |
| 10 | CDR-H1 | Chothia | GFTFDNY |
| 11 | CDR-H1 | Chothia | GFTFSDY |
| 12 | CDR-H1 | Chothia | GFTFSSS |
| 13 | CDR-H1 | Chothia | GGSISSSN |
| 14 | CDR-H1 | Chothia | GGSISSSSY |
| 15 | | | |
| 16 | | | |
| 17 | | | |
| 18 | CDR-H1 | Kabat | SSDYYWG |
| 19 | CDR-H1 | Kabat | SSSTYWA |
| 20 | CDR-H1 | Kabat | SSSTYWG |
| 21 | CDR-H1 | Kabat | SSDTYWG |
| 22 | CDR-H1 | Kabat | SADNYWG |
| 23 | CDR-H1 | Kabat | SSSTYWS |
| 24 | CDR-H1 | Kabat | SGYYWG |
| 25 | CDR-H1 | Kabat | SGYYWF |
| 26 | CDR-H1 | Kabat | SGHYWI |
| 27 | CDR-H1 | Kabat | SGFYWT |
| 28 | CDR-H1 | Kabat | SGYYWL |
| 29 | CDR-H1 | Kabat | SGHYWT |
| 30 | CDR-H1 | Kabat | NYAMH |
| 31 | CDR-H1 | Kabat | DYYMS |
| 32 | CDR-H1 | Kabat | SSAMA |
| 33 | CDR-H1 | Kabat | SSNWWS |
| 34 | CDR-H1 | Kabat | SSSYYWG |
| 35 | | | |
| 36 | | | |
| 37 | | | |
| 38 | CDR-H2 | Chothia | YYSGS |
| 39 | CDR-H2 | Chothia | SSSGS |
| 40 | CDR-H2 | Chothia | HHSGA |
| 41 | CDR-H2 | Chothia | HYSGS |
| 42 | CDR-H2 | Chothia | AYSGS |
| 43 | CDR-H2 | Chothia | SYNAL |
| 44 | CDR-H2 | Chothia | YHSGS |
| 45 | CDR-H2 | Chothia | YHSAS |
| 46 | CDR-H2 | Chothia | SARAGI |
| 47 | CDR-H2 | Chothia | ASSGSV |
| 48 | CDR-H2 | Chothia | SGSGIT |
| 49 | CDR-H2 | Chothia | SYSGS |
| 50 | CDR-H2 | Chothia | SSSGST |
| 51 | | | |
| 52 | | | |
| 53 | | | |
| 54 | CDR-H2 | Kabat | SIYYSGSTYYNPSLKS |
| 55 | CDR-H2 | Kabat | SISSSGSTYYNPSLKS |
| 56 | CDR-H2 | Kabat | SIHHSGATYYNPSLKS |
| 57 | CDR-H2 | Kabat | SIHYSGSTLYNPSLKS |
| 58 | CDR-H2 | Kabat | SIHYSGSTYYNPSLKS |
| 59 | CDR-H2 | Kabat | GIAYSGSTYYNPSLKS |
| 60 | CDR-H2 | Kabat | SISYNALTYYNPSLKS |
| 61 | CDR-H2 | Kabat | SIYHSGSTYYNPSLKS |
| 62 | CDR-H2 | Kabat | GIYHSASTAYNPSLKS |
| 63 | CDR-H2 | Kabat | GIYHSGSTYYNPSLKS |
| 64 | CDR-H2 | Kabat | AIYHSGSTVYNPSLKS |
| 65 | CDR-H2 | Kabat | GIYHSGSTAYNPSLKS |
| 66 | CDR-H2 | Kabat | AISARAGITYYADSVKG |
| 67 | CDR-H2 | Kabat | YIASSGSVIYYADSVKG |
| 68 | CDR-H2 | Kabat | TISGSGITTWYADSVKG |
| 69 | CDR-H2 | Kabat | EIYHSGSTNYNPSLKS |
| 70 | CDR-H2 | Kabat | SISYSGSTYYNPSLKS |
| 71 | CDR-H2 | Kabat | YISSSGSTIYYADSVKG |
| 72 | | | |
| 73 | | | |
| 74 | | | |
| 75 | | | |
| 76 | CDR-H3 | | GVRRAVPFDY |
| 77 | CDR-H3 | | GIARAVPFDY |
| 78 | CDR-H3 | | GPKRAVPFDY |
| 79 | CDR-H3 | | GVRRAVPFVD |
| 80 | CDR-H3 | | GVRRAVPFQR |
| 81 | CDR-H3 | | GTRRAVPFDY |
| 82 | CDR-H3 | | GVRRAVPFAD |
| 83 | CDR-H3 | | GIRRAVPFDY |
| 84 | CDR-H3 | | GQFRAVPFDY |
| 85 | CDR-H3 | | GGTHTYSRGPMDV |
| 86 | CDR-H3 | | GGTHTYSRGPFDV |
| 87 | CDR-H3 | | GGTPIYSRGPLDV |
| 88 | CDR-H3 | | GGGQTYSRGPLDV |
| 89 | CDR-H3 | | GGGATYSRGPLDV |
| 90 | CDR-H3 | | GGTHTYSRGPLDV |
| 91 | CDR-H3 | | GGTVKYSRGPLDV |
| 92 | CDR-H3 | | GGQVTYSRGPLDV |
| 93 | CDR-H3 | | GGEVTYSRGPLDV |
| 94 | CDR-H3 | | RIGYSYGTAPPFDV |
| 95 | CDR-H3 | | HGTPRAFDI |
| 96 | CDR-H3 | | GSRHLNAFNR |
| 97 | CDR-H3 | | GVYHYDPYGMDV |
| 98 | CDR-H3 | | TELGKMHFDY |
| 99 | CDR-H3 | | GSPRYMQD |
| 100 | CDR-H3 | | HSSLGTHNWFDP |
| 101 | CDR-H3 | | EGALSYSWLAAFDI |
| 102 | | | |
| 103 | | | |
| 104 | | | |
| 105 | CDR-L1 | | RASQSVSSSYLA |
| 106 | CDR-L1 | | GASQSVSSDYLA |
| 107 | CDR-L1 | | QASQAVSSNYLA |
| 108 | CDR-L1 | | GASQSVSSAFLA |
| 109 | CDR-L1 | | RASQSVSSTYLA |
| 110 | CDR-L1 | | QASQSVSSSYLA |
| 111 | CDR-L1 | | KASQAVSSSYLA |
| 112 | CDR-L1 | | EASQSVSSSYLA |
| 113 | CDR-L1 | | EASQSVSASYLA |
| 114 | CDR-L1 | | EASQSVSSAYLA |
| 115 | CDR-L1 | | RVSQSVSDAYLA |
| 116 | CDR-L1 | | EVSQSVSASYLA |
| 117 | CDR-L1 | | RASQSVSSAYLA |
| 118 | CDR-L1 | | RASNAVSSSYLA |
| 119 | CDR-L1 | | RASQSINSWLA |
| 120 | CDR-L1 | | AASQGISSDLA |
| 121 | CDR-L1 | | RASQDISTYLN |
| 122 | CDR-L1 | | RSSQSLLHSNGYNYLD |
| 123 | CDR-L1 | | RASQSISSYLN |
| 124 | CDR-L1 | | RASQSVSSNLA |
| 125 | | | |
| 126 | | | |
| 127 | | | |
| 128 | CDR-L2 | | GASSRAT |
| 129 | CDR-L2 | | GAYSLAT |
| 130 | CDR-L2 | | GASARAT |
| 131 | CDR-L2 | | GASSREA |
| 132 | CDR-L2 | | GASNRAA |
| 133 | CDR-L2 | | GASSRQD |
| 134 | CDR-L2 | | GASNRAT |
| 135 | CDR-L2 | | DASSRAS |
| 136 | CDR-L2 | | DASTRAT |
| 137 | CDR-L2 | | GASDRAN |
| 138 | CDR-L2 | | GASYRAT |
| 139 | CDR-L2 | | DASSLES |
| 140 | CDR-L2 | | SASSTQS |
| 141 | CDR-L2 | | DAFNLET |
| 142 | CDR-L2 | | LGSNRAS |
| 143 | CDR-L2 | | GASRRAT |
| 144 | CDR-L2 | | AASSLQS |
| 145 | CDR-L2 | | GASTRAT |
| 146 | | | |
| 147 | | | |
| 148 | | | |
| 149 | CDR-L3 | | QQAVHSPYT |
| 150 | CDR-L3 | | QWAVHSPYT |
| 151 | CDR-L3 | | QQVVHSPYT |
| 152 | CDR-L3 | | QQTVHSPYT |
| 153 | CDR-L3 | | QQAIHSPYT |
| 154 | CDR-L3 | | QQHSSYPPT |
| 155 | CDR-L3 | | QQHSLYPPT |
| 156 | CDR-L3 | | QQFSSYPPT |
| 157 | CDR-L3 | | QQVSSYPPT |
| 158 | CDR-L3 | | QQHSIYPPT |
| 159 | CDR-L3 | | QQYDSHIT |
| 160 | CDR-L3 | | QQAYLYPIT |
| 161 | CDR-L3 | | QQLPFLPIT |
| 162 | CDR-L3 | | MQALGGPWT |
| 163 | CDR-L3 | | QQYVSDPIT |
| 164 | CDR-L3 | | QQVGSSPIT |
| 165 | CDR-L3 | | QQSHLVPRT |
| 166 | CDR-L3 | | QQANHHPPFT |
| 167 | | | |
| 168 | | | |
| 169 | | | |
| 170 | VH | | QLQLQESGPGLVKPSETLSLTCTVSGGSISSSD |
| | | | YYWGWIRQPPGKGLEWIGSIYYSGSTYYNPSLK |
| | | | SRVTISVDTSKNQFSLKLSSVTAADTAVYYCAR |
| | | | GVRRAVPFDYWGQGTLVTVSS |
| 171 | VH | | QLQLQESGPGLVKPSETLSLTCTVSGGSISSSS |
| | | | TYWAWIRQPPGKGLEWIGSISSSGSTYYNPSLK |
| | | | SRVTISVDTSKNQFSLKLSSVTAADTAVYYCAR |
| | | | GIARAVPFDYWGQGTLVTVSS |
| 172 | VH | | QLQLQESGPGLVKPSETLSLTCTVSGGSISSSS |
| | | | TYWGWIRQSPGKGLEWIGSIHHSGATYYNPSLK |
| | | | SRVTISVDTSKNQFSLKLSSVTAADTAVYYCAR |
| | | | GPKRAVPFDYWGQGTLVTVSS |
| 173 | VH | | QLQLQESGPGLVKPSETLSLTCTVSGGSISSSD |
| | | | TYWGWIRQPPGKGLEWIGSIHYSGSTLYNPSLK |
| | | | SRVTISVDTSKNQFSLKLSSVTAADTAVYYCAR |
| | | | GVRRAVPFVDWGQGTLVTVSS |
| 174 | VH | | QLQLQESGPGLVKPSETLSLTCTVSGGSISSAD |
| | | | NYWGWIRQPPGKGLEWIGSIHYSGSTYYNPSLK |
| | | | SRVTISVDTSKNQFSLKLSSVTAADTAVYYCAR |
| | | | GVRRAVPFQRWGQGTLVTVSS |
| 175 | VH | | QLQLQESGPGLVKPSETLSLTCTVSGGSISSSD |
| | | | TYWGWIRQPPGKGPEWIGSIHYSGSTLYNPSLK |
| | | | SRVTISVDTSKNQFSLKLSSVTAADTAVYYCAR |
| | | | GVRRAVPFDYWGQGTLVTVSS |
| 176 | VH | | QLQLQESGPGLVKPSETLSLTCTVSGGSISSSS |
| | | | TYWSWIRQPPGKGLEWIGGIAYSGSTYYNPSLK |
| | | | SRVTISVDTSKNQFSLKLSSVTAADTAVYYCAR |
| | | | GVRRAVPFDYWGQGTLVTVSS |
| 177 | VH | | QLQLQESGPGLVKPSETLSLTCTVSGGSISSSS |
| | | | TYWSWIRQPPGKGLEWIGSISYNALTYYNPSLK |
| | | | SRVTISVDTSKNQFSLKLSSVTAADTAVYYCAR |
| | | | GTRRAVPFDYWGQGTLVTVSS |
| 178 | VH | | QLQLQESGPGLVKPSETLSLTCTVSGGSISSSS |
| | | | TYWSWIRQPPGKGLEWIGGIAYSGSTYYNPSLK |
| | | | SRVTISVDTSKNQFSLKLSSVTAADTAVYYCAR |
| | | | GVRRAVPFADWGQGTLVTVSS |
| 179 | VH | | QLQLQESGPGLVKPSETLSLTCTVSGGSVSSSS |
| | | | TYWSWIRQPPGKGLEWIGGIAYSGSTYYNPSLK |
| | | | SRVTISVDTSKNQFSLKLSSVTAADTAVYYCAR |
| | | | GVRRAVPFDYWGQGTLVTVSS |
| 180 | VH | | QLQLQESGPGLVKPSETLSLTCTVSGGSISSSS |
| | | | TYWSWIRQPPGKGLEWIGGIAYSGSTYYNPSLK |
| | | | SRVTISVDTSKNQFSLKLSSVTAADTAVYYCAR |
| | | | GTRRAVPFDYWGQGTLVTVSS |
| 181 | VH | | QLQLQESGPGLVKPSETLSLTCTVSGGSISSSD |
| | | | TYWGWIRQPPGKGLEWIGSIHYSGSTLYNPSLK |
| | | | SRVTISVDTSKNQFSLKLSSVTAADTAVYYCAR |
| | | | GIRRAVPFDYWGQGTLVTVSS |
| 182 | VH | | QLQLQESGPGLVKPSETLSLTCTVSGGSISSSD |
| | | | TYWGWIRQPPGKGLEWIGSIHYSGSTLYNPSLK |
| | | | SRVTISVDTSKNQFSLKLSSVTAADTAVYYCAR |
| | | | GQFRAVPFDYWGQGTLVTVSS |
| 183 | VH | | QVQLQESGPGLVKPSETLSLTCAVSGYSISSGY |
| | | | YWGWIRQPPGKGLEWIGSIYHSGSTYYNPSLKS |
| | | | RVTISVDTSKNQFSLKLSSVTAADTAVYYCARG |
| | | | GTHTYSRGPMDVWGQGTTVTVSS |
| 184 | VH | | QLQLQESGPRLVKPSETLSLTCAVSGYSISSGY |
| | | | YWGWIRQPPGKGLEWIGSIYHSGSTYYNPSLKS |
| | | | RVTISVDTSKNQFSLKLSSVTAADTAVYYCARG |
| | | | GTHTYSRGPFDVWGQGTTVTVSS |
| 185 | VH | | LVQLQESGPGLVKPSETLSLTCAVSGYSILSGY |
| | | | YWFWIRQPPGKGLEWIGGIYHSASTAYNPSLKS |
| | | | RVTISVDTSKNQFSLKLSSVTAADTAVYYCARG |
| | | | GTPIYSRGPLDVWGQGTTVTVSS |
| 186 | VH | | QVQLQESGPGLVKPSETLSLTCAVSGYSISSGH |
| | | | YWIWIRQPPGKGLEWIGGIYHSGSTYYNPSLKS |
| | | | RVTISVDTSKDQFSLKLSSVTAADTAVYYCARG |
| | | | GGQTYSRGPLDVWGQGTTVTVSS |
| 187 | VH | | QVQLQESGPGLVKPPETLSLTCAVSGYSISSGH |
| | | | YWIWIRQPPGKGLEWIGGIYHSGSTYYNPSLKS |
| | | | RVTISVDTSKNQFSLKLSSVTAADTAVYYCARG |
| | | | GGATYSRGPLDVWGQGTTVTVSS |
| 188 | VH | | QVQLQESGPGLVKPSETLSLTCAVSGYSILSGY |
| | | | YWFWIRQPPGKGLEWIGGIYHSGSTYYNPSLKS |
| | | | RVTISVDTSKNQFSLKLSSVTAADTAVYYCARG |
| | | | GTHTYSRGPLDVWGQGTTVTVSS |
| 189 | VH | | QVQLQESGPGLVKPSETLSLTCAVSGYSISSGF |
| | | | YWTWIRQPPGKGLEWIGAIYHSGSTVYNPSLKS |
| | | | RVTISVDTSKNQFSLKLSSVTAADTAVYYCARG |
| | | | GTHTYSRGPLDVWGQGTTVTVSS |
| 190 | VH | | QVQLQESGPGLVKPSETLSLTCAVSGYSISSGY |
| | | | YWLWIRQPPGKGLEWIGGIYHSASTAYNPSLKS |
| | | | RVTISVDTSKNQFSLKLSSVTAADTAVYYCARG |
| | | | GTVKYSRGPLDVWGQGTTVTVSS |
| 191 | VH | | QVQLQESGPGLVKPSETLSLTCAVSGYSISSGH |
| | | | YWTWIRQPPGKGLEWIGAIYHSGSTVYNPSLKS |
| | | | RVTISVDTSKNQFSLKLSSVTAADTAVYYCARG |
| | | | GQVTYSRGPLDVWGQGTTVTVSS |
| 192 | VH | | QVQLQESGPGLVKPSETLSLTCAVSGYSILSGY |
| | | | YWFWIRQPPGKGLEWIGGIYHSGSTAYNPSLKS |
| | | | RVTISVDTSKNQFSLKLSSVTAADTAVYYCARG |
| | | | GEVTYSRGPLDVWGQGTTVTVSS |
| 193 | VH | | EVQLLESGGGLVQPGGSLRLSCAASGFTFDNYA |
| | | | MHWVRQAPGKGLEWVSAISARAGITYYADSVKG |
| | | | RFTISRDNSKNTLYLQMNSLRAEDTAVYYCARR |
| | | | IGYSYGTAPPFDVWGQGTTVTVSS |
| 194 | VH | | QVQLVESGGGLVQPGGSLRLSCAASGFTFSDYY |
| | | | MSWIRQAPGKGLEWVSYIASSGSVIYYADSVKG |
| | | | RFTISRDNAKNSLYLQMNSLRAEDTAVYYCARH |
| | | | GTPRAFDIWGQGTTVTVSS |
| 195 | VH | | EVQLLESGGGLVQPGGSLRLSCAASGFTFSSSA |
| | | | MAWVRQAPGKGLEWVSTISGSGITTWYADSVKG |
| | | | RFTISRDNSKNTLYLQMNSLRAEDTAVYYCAKG |
| | | | SRHLNAFNRWGQGTTVTVSS |
| 196 | VH | | QVQLQESGPGLVKPSGTLSLTCAVSGGSISSSN |
| | | | WWSWVRQPPGKGLEWIGEIYHSGSTNYNPSLKS |
| | | | RVTISVDKSKNQFSLKLSSVTAADTAVYYCARG |
| | | | VYHYDPYGMDVWGQGTTVTVSS |
| 197 | VH | | QLQLQESGPGLVKPSETLSLTCTVSGGSISSSS |
| | | | YYWGWIRQPPGKGLEWIGSISYSGSTYYNPSLK |
| | | | SRVTISVDTSKNQFSLKLSSVTAADTAVYYCAR |
| | | | TELGKMHFDYWGQGTLVTVSS |
| 198 | VH | | QLQLQESGPGLVKPSETLSLTCTVSGGSISSSD |
| | | | YYWGWIRQPPGKGLEWIGSIYYSGSTYYNPSLK |
| | | | SRVTISVDTSKNQFSLKLSSVTAADTAVYYCAR |
| | | | GSPRYMQDWGQGTLVTVSS |
| 199 | VH | | QVQLVESGGGLVKPGGSLRLSCAASGFTFSDYY |
| | | | MSWIRQAPGKGLEWVSYISSSGSTIYYADSVKG |
| | | | RFTISRDNAKNSLYLQMNSLRAEDTAVYYCARH |
| | | | SSLGTHNWFDPWGQGTLVTVSS |
| 200 | VH | | QVQLQESGPGLVKPSETLSLTCAVSGYSISSGY |
| | | | YWGWIRQPPGKGLEWIGSIYHSGSTYYNPSLKS |
| | | | RVTISVDTSKNQFSLKLSSVTAADTAVYYCARE |
| | | | GALSYSWLAAFDIWGQGTMVTVSS |
| 201 | | | |
| 202 | | | |
| 203 | | | |
| 204 | VL | | EIVLTQSPGTLSLSPGERATLSCRASQSVSSSY |
| | | | LAWYQQKPGQAPRLLIYGASSRATGIPDRFSGS |
| | | | GSGTDFTLTISRLEPEDFAVYYCQQAVHSPYTF |
| | | | GGGTKVEIK |
| 205 | VL | | EIVLTQSPGTLSLSPGERATLSCGASQSVSSDY |
| | | | LAWYQQKPGQAPRLLIYGAYSLATGIPDRFSGS |
| | | | GSGTDFTLTISRLEPEDFAVYYCQWAVHSPYTF |
| | | | GGGTKVEIK |
| 206 | VL | | EIVLTQSPGTLSLSPGERATLSCQASQAVSSNY |
| | | | LAWYQQKPGQAPRLLIYGASSRATGIPDRFSGS |
| | | | GSGTDFTLTISRLEPEDFAVYYCQQVVHSPYTF |
| | | | GGGTKVEIK |
| 207 | VL | | EIVLTQSPGTLSLSPGERATLSCGASQSVSSAF |
| | | | LAWYQQKPGQAPRLLIYGASARATGIPDRFSGS |
| | | | GSGTDFTLTISRLEPEDFAVYYCQQVVHSPYTF |
| | | | GGGTKVEIK |
| 208 | VL | | EIVLTQSPGTLSLSPGERATLSCRASQSVSSTY |
| | | | LAWYQQKPGQAPRLLIYGASSREAGIPDRFSGS |
| | | | GSGTDFTLTISRLEPEDFAVYYCQQTVHSPYTF |
| | | | GGGTKVEIK |
| 209 | VL | | EIVLTQSPGTLSLSPGERATLSCQASQAVSSNY |
| | | | LAWYQQKPGQAPRLLIYGASNRAAGIPDRFSGS |
| | | | GSGTDFTLTISRLEPEDFAVYYCQQAIHSPYTF |
| | | | GGGTKVEIK |
| 210 | VL | | EIVLTQSPGTLSLSPGERATLSCQASQSVSSSY |
| | | | LAWYQQKPGQAPRLLIYGASNRAAGIPDRFSGS |
| | | | GSGTDFTLTISRLEPEDFAVYYCQQVVHSPYTF |
| | | | GGGTKVEIK |
| 211 | VL | | EIVLTQSPGTLSLSPGERATLSCKASQAVSSSY |
| | | | LAWYQQKPGQAPRLLIYGASSRQDGIPDRFSGS |
| | | | GSGTDFTLTISRLEPEDFAVYYCQQVVHSPYTF |
| | | | GGGTKVEIK |
| 212 | VL | | EIVLTQSPGTLSLSPGERATLSCRASQSVSSSY |
| | | | LAWYQQKPGQAPRLLIYGASSRATGIPDRFSGS |
| | | | GSGTDFTLTISRLEPEDFAVYYCQQHSSYPPTF |
| | | | GGGTKVEIK |
| 213 | VL | | EIVLTQSPGTLSLSPGERATLSCEASQSVSSSY |
| | | | LAWYQQKPGQAPRLLIYGASNRATGIPDRFSGS |
| | | | GSGTDFTLTISRLEPEDFAVYYCQQHSLYPPTF |
| | | | GGGTKVEIK |
| 214 | VL | | EIVLTQSPGTLSLSPGERATLSCEASQSVSASY |
| | | | LAWYQQKPGQAPRLLIYDASSRASGIPDRFSGS |
| | | | GSGTDFTLTISRLEPEDFAVYYCQQFSSYPPTF |
| | | | GGGTKVEIK |
| 215 | VL | | EIVLTQSPGTLSLSPGERATLSCEASQSVSSAY |
| | | | LAWYQQKPGQAPRLLIYDASTRATGIPDRFSGS |
| | | | GSGTDFTLTISRLEPEDFAVYYCQQVSSYPPTF |
| | | | GGGTKVEIK |
| 216 | VL | | EIVLTQSPGTLSLSPGERAALSCRVSQSVSDAY |
| | | | LAWYQQKPGQAPRLLIYDASSRASGIPDRFSGS |
| | | | GSGTDFTLTISRLEPEDFAVYYCQQVSSYPPTF |
| | | | GGGTKVEIK |
| 217 | VL | | EIVLTQSPGTLSLSPGERATLSCEVSQSVSASY |
| | | | LAWYQQKPGQAPRLLIYGASSRATGIPDRFSGS |
| | | | GSGTDFTLTISRLEPEDFAVYYCQQHSLYPPTF |
| | | | GGGTKVEIK |
| 218 | VL | | EIVLTQSPGTLSLSPGERATLSCRASQSVSSAY |
| | | | LAWYQQKPGQAPRLLIYGASDRANGIPDRFSGS |
| | | | GSGTDFTLTISRLEPEDFAVYYCQQHSLYPPTF |
| | | | GGGTKVEIK |
| 219 | VL | | EIVLTQSPGTLSLSPGERATLSCRASNAVSSSY |
| | | | LAWYQQKPGQAPRLLIYGASDRANGIPDRFSGS |
| | | | GSGTDFTLTISRLEPEDFAVYYCQQHSIYPPTF |
| | | | GGGTKVEIK |
| 220 | VL | | EIVLTQSPGTLSLSPGERATLSCRASNAVSSSY |
| | | | LAWYQQKPGQAPRLLIYGASYRATGIPDRFSGS |
| | | | GSGTDFTLTISRLEPEDFAVYYCQQHSLYPPTF |
| | | | GGGTKVEIK |
| 221 | VL | | DIQMTQSPSTLSASVGDRVTITCRASQSINSWL |
| | | | AWYQQKPGKAPKLLISDASSLESGVPSRFSGSG |
| | | | SGTEFTLTISSLQPDDFATYYCQQYDSHITFGG |
| | | | GTKVEIK |
| 222 | VL | | DIQMTQSPSSVSASVGDRVTITCAASQGISSDL |
| | | | AWYQQKPGKAPKLLIYSASSTQSGVPSRFSGSG |
| | | | SGTDFTLTISSLQPEDFATYYCQQAYLYPITFG |
| | | | GGTKVEIK |
| 223 | VL | | GVQMTQSPSSLSASVGDRVTITCRASQDISTYL |
| | | | NWYQQKPGKAPKLLIYDAFNLETGVPSRFSGSG |
| | | | SGTDFTFTISSLQPEDIATYYCQQLPFLPITFG |
| | | | GGTKVEIK |
| 224 | VL | | DIVMTQSPLSLPVTPGEPASISCRSSQSLLHSN |
| | | | GYNYLDWYLQKPGQS PQLLIYLGSNRASGVPDR |
| | | | FSGSGSGTDFTLKISRVEAEDVGVYYCMQALGG |
| | | | PWTFGGGTKVEIK |
| 225 | VL | | EIVLTQSPGTLSLSPGERATLSCRASQSVSSSY |
| | | | LAWYQQKPGQAPRLLIYGASRRATGIPDRFSGS |
| | | | GSGTDFTLTISRLEPEDFAVYYCQQYVSDPITF |
| | | | GGGTKVEIK |
| 226 | VL | | EIVLTQSPGTLSLSPGERATLSCRASQSVSSSY |
| | | | LAWYQQKPGQAPRLLIYGASRRATGIPDRFSGS |
| | | | GSGTDFTLTISRLEPEDFAVYYCQQVGSSPITF |
| | | | GGGTKVEIK |
| 227 | VL | | DIQMTQSPSSLSASVGDRVTITCRASQSISSYL |
| | | | NWYQQKPGKAPKLLIYAASSLQSGVPSRFSGSG |
| | | | SGTDFTLTISSLQPEDFATYYCQQSHLVPRTFG |
| | | | GGTKVEIK |
| 228 | VL | | EIVMTQSPATLSVSPGERATLSCRASQSVSSNL |
| | | | AWYQQKPGQAPRLLIYGASTRATGIPARFSGSG |
| | | | SGTEFTLTISSLQSEDFAVYYCQQANHHPPFTF |
| | | | GGGTKVEIK |
| 229 | | | |
| 230 | | | |
| 231 | | | |
| 232 | IGG1 AAA HC | 33343 | QLQLQESGPGLVKPSETLSLTCTVSGGSISSSD |
| | | | YYWGWIRQPPGKGLEWIGSIYYSGSTYYNPSLK |
| | | | SRVTISVDTSKNQFSLKLSSVTAADTAVYYCAR |
| | | | GVRRAVPFDYWGQGTLVTVSSASTKGPSVFPLA |
| | | | PSSKSTSGGTAALGCLVKDYFPEPVTVSWNSGA |
| | | | LTSGVHTFPAVLQSSGLYSLSSVVTVPSSSLGT |
| | | | QTYICNVNHKPSNTKVDKKVEPKSCDKTHTCPP |
| | | | CPAPEAAGAPSVFLFPPKPKDTLMISRTPEVTC |
| | | | VVVDVSHEDPEVKFNWYVDGVEVHNAKTKPREE |
| | | | QYNSTYRVVSVLTVLHQDWLNGKEYKCKVSNKA |
| | | | LPAPIEKTISKAKGQPREPQVYTLPPSREEMTK |
| | | | NQVSLTCLVKGFYPSDIAVEWESNGQPENNYKT |
| | | | TPPVLDSDGSFFLYSKLTVDKSRWQQGNVFSCS |
| | | | VMHEALHNHYTQKSLSLSPGK |
| 233 | IGG1 AAA HC | 37268 | QLQLQESGPGLVKPSETLSLTCTVSGGSISSSS |
| | | | TYWAWIRQPPGKGLEWIGSISSSGSTYYNPSLK |
| | | | SRVT1SVDTSKNQFSLKLSSVTAADTAVYYCAR |
| | | | GIARAVPFDYWGQGTLVTVSSASTKGPSVFPLA |
| | | | PSSKSTSGGTAALGCLVKDYFPEPVTVSWNSGA |
| | | | LTSGVHTFPAVLQSSGLYSLSSVVTVPSSSLGT |
| | | | QTYICNVNHKPSNTKVDKKVEPKSCDKTHTCPP |
| | | | CPAPEAAGAPSVFLFPPKPKDTLMISRTPEVTC |
| | | | VVVDVSHEDPEVKFNWYVDGVEVHNAKTKPREE |
| | | | QYNSTYRVVSVLTVLHQDWLNGKEYKCKVSNKA |
| | | | LPAPIEKTISKAKGQPREPQVYTLPPSREEMTK |
| | | | NQVSLTCLVKGFYPSDIAVEWESNGQPENNYKT |
| | | | TPPVLDSDGSFFLYSKLTVDKSRWQQGNVFSCS |
| | | | VMHEALHNHYTQKSLSLSPGK |
| 234 | IGG1 AAA HC | 37269 | QLQLQESGPGLVKPSETLSLTCTVSGGSISSSS |
| | | | TYWGWIRQSPGKGLEWIGSIHHSGATYYNPSLK |
| | | | SRVTISVDTSKNQFSLKLSSVTAADTAVYYCAR |
| | | | GPKRAVPFDYWGQGTLVTVSSASTKGPSVFPLA |
| | | | PSSKSTSGGTAALGCLVKDYFPEPVTVSWNSGA |
| | | | LTSGVHTFPAVLQSSGLYSLSSVVTVPSSSLGT |
| | | | QTYICNVNHKPSNTKVDKKVEPKSCDKTHTCPP |
| | | | CPAPEAAGAPSVFLFPPKPKDTLMISRTPEVTC |
| | | | VVVDVSHEDPEVKFNWYVDGVEVHNAKTKPREE |
| | | | QYNSTYRVVSVLTVLHQDWLNGKEYKCKVSNKA |
| | | | LPAPIEKTISKAKGQPREPQVYTLPPSREEMTK |
| | | | NQVSLTCLVKGFYPSDIAVEWESNGQPENNYKT |
| | | | TPPVLDSDGSFFLYSKLTVDKSRWQQGNVFSCS |
| | | | VMHEALHNHYTQKSLSLSPGK |
| 235 | IGG1 AAA HC | 37271 | QLQLQESGPGLVKPSETLSLTCTVSGGSISSSD |
| | | | TYWGWIRQPPGKGLEWIGSIHYSGSTLYNPSLK |
| | | | SRVTISVDTSKNQFSLKLSSVTAADTAVYYCAR |
| | | | GVRRAVPFVDWGQGTLVTVSSASTKGPSVFPLA |
| | | | PSSKSTSGGTAALGCLVKDYFPEPVTVSWNSGA |
| | | | LTSGVHTFPAVLQSSGLYSLSSVVTVPSSSLGT |
| | | | QTYICNVNHKPSNTKVDKKVEPKSCDKTHTCPP |
| | | | CPAPEAAGAPSVFLFPPKPKDTLMISRTPEVTC |
| | | | VVVDVSHEDPEVKFNWYVDGVEVHNAKTKPREE |
| | | | QYNSTYRVVSVLTVLHQDWLNGKEYKCKVSNKA |
| | | | LPAPIEKTISKAKGQPREPQVYTLPPSREEMTK |
| | | | NQVSLTCLVKGFYPSDIAVEWESNGQPENNYKT |
| | | | TPPVLDSDGSFFLYSKLTVDKSRWQQGNVFSCS |
| | | | VMHEALHNHYTQKSLSLSPGK |
| 236 | IGG1 AAA HC | 37272 | QLQLQESGPGLVKPSETLSLTCTVSGGSISSAD |
| | | | NYWGWIRQPPGKGLEWIGSIHYSGSTYYNPSLK |
| | | | SRVTISVDTSKNQFSLKLSSVTAADTAVYYCAR |
| | | | GVRRAVPFQRWGQGTLVTVSSASTKGPSVFPLA |
| | | | PSSKSTSGGTAALGCLVKDYFPEPVTVSWNSGA |
| | | | LTSGVHTFPAVLQSSGLYSLSSVVTVPSSSLGT |
| | | | QTYICNVNHKPSNTKVDKKVEPKSCDKTHTCPP |
| | | | CPAPEAAGAPSVFLFPPKPKDTLMISRTPEVTC |
| | | | VVVDVSHEDPEVKFNWYVDGVEVHNAKTKPREE |
| | | | QYNSTYRVVSVLTVLHQDWLNGKEYKCKVSNKA |
| | | | LPAPIEKTISKAKGQPREPQVYTLPPSREEMTK |
| | | | NQVSLTCLVKGFYPSDIAVEWESNGQPENNYKT |
| | | | TPPVLDSDGSFFLYSKLTVDKSRWQQGNVFSCS |
| | | | VMHEALHNHYTQKSLSLSPGK |
| 237 | IGG1 AAA HC | 37277 | QLQLQESGPGLVKPSETLSLTCTVSGGSISSSD |
| | | | TYWGWIRQPPGKGPEWIGSIHYSGSTLYNPSLK |
| | | | SRVTISVDTSKNQFSLKLSSVTAADTAVYYCAR |
| | | | GVRRAVPFDYWGQGTLVTVSSASTKGPSVFPLA |
| | | | PSSKSTSGGTAALGCLVKDYFPEPVTVSWNSGA |
| | | | LTSGVHTFPAVLQSSGLYSLSSVVTVPSSSLGT |
| | | | QTYICNVNHKPSNTKVDKKVEPKSCDKTHTCPP |
| | | | CPAPEAAGAPSVFLFPPKPKDTLMISRTPEVTC |
| | | | VVVDVSHEDPEVKFNWYVDGVEVHNAKTKPREE |
| | | | QYNSTYRVVSVLTVLHQDWLNGKEYKCKVSNKA |
| | | | LPAPIEKTISKAKGQPREPQVYTLPPSREEMTK |
| | | | NQVSLTCLVKGFYPSDIAVEWESNGQPENNYKT |
| | | | TPPVLDSDGSFFLYSKLTVDKSRWQQGNVFSCS |
| | | | VMHEALHNHYTQKSLSLSPGK |
| 238 | IGG1 AAA HC | 38373 | QLQLQESGPGLVKPSETLSLTCTVSGGSISSSS |
| | | | TYWSWIRQPPGKGLEWIGGIAYSGSTYYNPSLK |
| | | | SRVTISVDTSKNQFSLKLSSVTAADTAVYYCAR |
| | | | GVRRAVPFDYWGQGTLVTVSSASTKGPSVFPLA |
| | | | PSSKSTSGGTAALGCLVKDYFPEPVTVSWNSGA |
| | | | LTSGVHTFPAVLQSSGLYSLSSVVTVPSSSLGT |
| | | | QTYICNVNHKPSNTKVDKKVEPKSCDKTHTCPP |
| | | | CPAPEAAGAPSVFLFPPKPKDTLMISRTPEVTC |
| | | | VVVDVSHEDPEVKFNWYVDGVEVHNAKTKPREE |
| | | | QYNSTYRVVSVLTVLHQDWLNGKEYKCKVSNKA |
| | | | LPAPIEKTISKAKGQPREPQVYTLPPSREEMTK |
| | | | NQVSLTCLVKGFYPSDIAVEWESNGQPENNYKT |
| | | | TPPVLDSDGSFFLYSKLTVDKSRWQQGNVFSCS |
| | | | VMHEALHNHYTQKSLSLSPGK |
| 239 | IGG1 AAA HC | 38375 | QLQLQESGPGLVKPSETLSLTCTVSGGSISSSS |
| | | | TYWSWIRQPPGKGLEWIGSISYNALTYYNPSLK |
| | | | SRVTISVDTSKNQFSLKLSSVTAADTAVYYCAR |
| | | | GTRRAVPFDYWGQGTLVTVSSASTKGPSVFPLA |
| | | | PSSKSTSGGTAALGCLVKDYFPEPVTVSWNSGA |
| | | | LTSGVHTFPAVLQSSGLYSLSSVVTVPSSSLGT |
| | | | QTYICNVNHKPSNTKVDKKVEPKSCDKTHTCPP |
| | | | CPAPEAAGAPSVFLFPPKPKDTLMISRTPEVTC |
| | | | VVVDVSHEDPEVKFNWYVDGVEVHNAKTKPREE |
| | | | QYNSTYRVVSVLTVLHQDWLNGKEYKCKVSNKA |
| | | | LPAPIEKTISKAKGQPREPQVYTLPPSREEMTK |
| | | | NQVSLTCLVKGFYPSDIAVEWESNGQPENNYKT |
| | | | TPPVLDSDGSFFLYSKLTVDKSRWQQGNVFSCS |
| | | | VMHEALHNHYTQKSLSLSPGK |
| 240 | IGG1 AAA HC | 38379 | QLQLQESGPGLVKPSETLSLTCTVSGGSISSSS |
| | | | TYWSWIRQPPGKGLEWIGGIAYSGSTYYNPSLK |
| | | | SRVTISVDTSKNQFSLKLSSVTAADTAVYYCAR |
| | | | GVRRAVPFADWGQGTLVTVSSASTKGPSVFPLA |
| | | | PSSKSTSGGTAALGCLVKDYFPEPVTVSWNSGA |
| | | | LTSGVHTFPAVLQSSGLYSLSSVVTVPSSSLGT |
| | | | QTYICNVNHKPSNTKVDKKVEPKSCDKTHTCPP |
| | | | CPAPEAAGAPSVFLFPPKPKDTLMISRTPEVTC |
| | | | VVVDVSHEDPEVKFNWYVDGVEVHNAKTKPREE |
| | | | QYNSTYRVVSVLTVLHQDWLNGKEYKCKVSNKA |
| | | | LPAPIEKTISKAKGQPREPQVYTLPPSREEMTK |
| | | | NQVSLTCLVKGFYPSDIAVEWESNGQPENNYKT |
| | | | TPPVLDSDGSFFLYSKLTVDKSRWQQGNVFSCS |
| | | | VMHEALHNHYTQKSLSLSPGK |
| 241 | IGG1 AAA HC | 38381 | QLQLQESGPGLVKPSETLSLTCTVSGGSVSSSS |
| | | | TYWSWIRQPPGKGLEWIGGIAYSGSTYYNPSLK |
| | | | SRVTISVDTSKNQFSLKLSSVTAADTAVYYCAR |
| | | | GVRRAVPFDYWGQGTLVTVSSASTKGPSVFPLA |
| | | | PSSKSTSGGTAALGCLVKDYFPEPVTVSWNSGA |
| | | | LTSGVHTFPAVLQSSGLYSLSSVVTVPSSSLGT |
| | | | QTYICNVNHKPSNTKVDKKVEPKSCDKTHTCPP |
| | | | CPAPEAAGAPSVFLFPPKPKDTLMISRTPEVTC |
| | | | VVVDVSHEDPEVKFNWYVDGVEVHNAKTKPREE |
| | | | QYNSTYRVVSVLTVLHQDWLNGKEYKCKVSNKA |
| | | | LPAPIEKTISKAKGQPREPQVYTLPPSREEMTK |
| | | | NQVSLTCLVKGFYPSDIAVEWESNGQPENNYKT |
| | | | TPPVLDSDGSFFLYSKLTVDKSRWQQGNVFSCS |
| | | | VMHEALHNHYTQKSLSLSPGK |
| 242 | IGG1 AAA HC | 38383 | QLQLQESGPGLVKPSETLSLTCTVSGGSISSSS |
| | | | TYWSWIRQPPGKGLEWIGGIAYSGSTYYNPSLK |
| | | | SRVTISVDTSKNQFSLKLSSVTAADTAVYYCAR |
| | | | GTRRAVPFDYWGQGTLVTVSSASTKGPSVFPLA |
| | | | PSSKSTSGGTAALGCLVKDYFPEPVTVSWNSGA |
| | | | LTSGVHTFPAVLQSSGLYSLSSVVTVPSSSLGT |
| | | | QTYICNVNHKPSNTKVDKKVEPKSCDKTHTCPP |
| | | | CPAPEAAGAPSVFLFPPKPKDTLMISRTPEVTC |
| | | | VVVDVSHEDPEVKFNWYVDGVEVHNAKTKPREE |
| | | | QYNSTYRVVSVLTVLHQDWLNGKEYKCKVSNKA |
| | | | LPAPIEKTISKAKGQPREPQVYTLPPSREEMTK |
| | | | NQVSLTCLVKGFYPSDIAVEWESNGQPENNYKT |
| | | | TPPVLDSDGSFFLYSKLTVDKSRWQQGNVFSCS |
| | | | VMHEALHNHYTQKSLSLSPGK |
| 243 | IGG1 AAA HC | 38386 | QLQLQESGPGLVKPSETLSLTCTVSGGSISSSD |
| | | | TYWGWIRQPPGKGLEWIGSIHYSGSTLYNPSLK |
| | | | SRVTISVDTSKNQFSLKLSSVTAADTAVYYCAR |
| | | | GIRRAVPFDYWGQGTLVTVSSASTKGPSVFPLA |
| | | | PSSKSTSGGTAALGCLVKDYFPEPVTVSWNSGA |
| | | | LTSGVHTFPAVLQSSGLYSLSSVVTVPSSSLGT |
| | | | QTYICNVNHKPSNTKVDKKVEPKSCDKTHTCPP |
| | | | CPAPEAAGAPSVFLFPPKPKDTLMISRTPEVTC |
| | | | VVVDVSHEDPEVKFNWYVDGVEVHNAKTKPREE |
| | | | QYNSTYRVVSVLTVLHQDWLNGKEYKCKVSNKA |
| | | | LPAPIEKTISKAKGQPREPQVYTLPPSREEMTK |
| | | | NQVSLTCLVKGFYPSDIAVEWESNGQPENNYKT |
| | | | TPPVLDSDGSFFLYSKLTVDKSRWQQGNVFSCS |
| | | | VMHEALHNHYTQKSLSLSPGK |
| 244 | IGG1 AAA HC | 37273 | QLQLQESGPGLVKPSETLSLTCTVSGGSISSSD |
| | | | TYWGWIRQPPGKGLEWIGSIHYSGSTLYNPSLK |
| | | | SRVTISVDTSKNQFSLKLSSVTAADTAVYYCAR |
| | | | GQFRAVPFDYWGQGTLVTVSSASTKGPSVFPLA |
| | | | PSSKSTSGGTAALGCLVKDYFPEPVTVSWNSGA |
| | | | LTSGVHTFPAVLQSSGLYSLSSVVTVPSSSLGT |
| | | | QTYICNVNHKPSNTKVDKKVEPKSCDKTHTCPP |
| | | | CPAPEAAGAPSVFLFPPKPKDTLMISRTPEVTC |
| | | | VVVDVSHEDPEVKFNWYVDGVEVHNAKTKPREE |
| | | | QYNSTYRVVSVLTVLHQDWLNGKEYKCKVSNKA |
| | | | LPAPIEKTISKAKGQPREPQVYTLPPSREEMTK |
| | | | NQVSLTCLVKGFYPSDIAVEWESNGQPENNYKT |
| | | | TPPVLDSDGSFFLYSKLTVDKSRWQQGNVFSCS |
| | | | VMHEALHNHYTQKSLSLSPGK |
| 245 | IGG1 AAA HC | 33361 | QVQLQESGPGLVKPSETLSLTCAVSGYSISSGY |
| | | | YWGWIRQPPGKGLEWIGSIYHSGSTYYNPSLKS |
| | | | RVTISVDTSKNQFSLKLSSVTAADTAVYYCARG |
| | | | GTHTYSRGPMDVWGQGTTVTVSSASTKGPSVFP |
| | | | LAPSSKSTSGGTAALGCLVKDYFPEPVTVSWNS |
| | | | GALTSGVHTFPAVLQSSGLYSLSSVVTVPSSSL |
| | | | GTQTYICNVNHKPSNTKVDKKVEPKSCDKTHTC |
| | | | PPCPAPEAAGAPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSREEM |
| | | | TKNQVSLTCLVKGFYPSDIAVEWESNGQPENNY |
| | | | KTTPPVLDSDGSFFLYSKLTVDKSRWQQGNVFS |
| | | | CSVMHEALHNHYTQKSLSLSPGK |
| 246 | IGG1 AAA HC | 35624 | QLQLQESGPRLVKPSETLSLTCAVSGYSISSGY |
| | | | YWGWIRQPPGKGLEWIGSIYHSGSTYYNPSLKS |
| | | | RVTISVDTSKNQFSLKLSSVTAADTAVYYCARG |
| | | | GTHTYSRGPFDVWGQGTTVTVSSASTKGPSVFP |
| | | | LAPSSKSTSGGTAALGCLVKDYFPEPVTVSWNS |
| | | | GALTSGVHTFPAVLQSSGLYSLSSVVTVPSSSL |
| | | | GTQTYICNVNHKPSNTKVDKKVEPKSCDKTHTC |
| | | | PPCPAPEAAGAPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSREEM |
| | | | TKNQVSLTCLVKGFYPSDIAVEWESNGQPENNY |
| | | | KTTPPVLDSDGSFFLYSKLTVDKSRWQQGNVFS |
| | | | CSVMHEALHNHYTQKSLSLSPGK |
| 247 | IGG1 AAA HC | 38410 | LVQLQESGPGLVKPSETLSLTCAVSGYSILSGY |
| | | | YWFWIRQPPGKGLEWIGGIYHSASTAYNPSLKS |
| | | | RVT1SVDTSKNQFSLKLSSVTAADTAVYYCARG |
| | | | GTPIYSRGPLDVWGQGTTVTVSSASTKGPSVFP |
| | | | LAPSSKSTSGGTAALGCLVKDYFPEPVTVSWNS |
| | | | GALTSGVHTFPAVLQSSGLYSLSSVVTVPSSSL |
| | | | GTQTYICNVNHKPSNTKVDKKVEPKSCDKTHTC |
| | | | PPCPAPEAAGAPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSREEM |
| | | | TKNQVSLTCLVKGFYPSDIAVEWESNGQPENNY |
| | | | KTTPPVLDSDGSFFLYSKLTVDKSRWQQGNVFS |
| | | | CSVMHEALHNHYTQKSLSLSPGK |
| 248 | IGG1 AAA HC | 38418 | QVQLQESGPGLVKPSETLSLTCAVSGYSISSGH |
| | | | YWIWIRQPPGKGLEWIGGIYHSGSTYYNPSLKS |
| | | | RVTISVDTSKDQFSLKLSSVTAADTAVYYCARG |
| | | | GGQTYSRGPLDVWGQGTTVTVSSASTKGPSVFP |
| | | | LAPSSKSTSGGTAALGCLVKDYFPEPVTVSWNS |
| | | | GALTSGVHTFPAVLQSSGLYSLSSVVTVPSSSL |
| | | | GTQTYICNVNHKPSNTKVDKKVEPKSCDKTHTC |
| | | | PPCPAPEAAGAPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSREEM |
| | | | TKNQVSLTCLVKGFYPSDIAVEWESNGQPENNY |
| | | | KTTPPVLDSDGSFFLYSKLTVDKSRWQQGNVFS |
| | | | CSVMHEALHNHYTQKSLSLSPGK |
| 249 | IGG1 AAA HC | 38420 | QVQLQESGPGLVKPPETLSLTCAVSGYSISSGH |
| | | | YWIWIRQPPGKGLEWIGGIYHSGSTYYNPSLKS |
| | | | RVTISVDTSKNQFSLKLSSVTAADTAVYYCARG |
| | | | GGATYSRGPLDVWGQGTTVTVSSASTKGPSVFP |
| | | | LAPSSKSTSGGTAALGCLVKDYFPEPVTVSWNS |
| | | | GALTSGVHTFPAVLQSSGLYSLSSVVTVPSSSL |
| | | | GTQTYICNVNHKPSNTKVDKKVEPKSCDKTHTC |
| | | | PPCPAPEAAGAPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSREEM |
| | | | TKNQVSLTCLVKGFYPSDIAVEWESNGQPENNY |
| | | | KTTPPVLDSDGSFFLYSKLTVDKSRWQQGNVFS |
| | | | CSVMHEALHNHYTQKSLSLSPGK |
| 250 | IGG1 AAA HC | 38421 | QVQLQESGPGLVKPSETLSLTCAVSGYSILSGY |
| | | | YWFWIRQPPGKGLEWIGGIYHSGSTYYNPSLKS |
| | | | RVT1SVDTSKNQFSLKLSSVTAADTAVYYCARG |
| | | | GTHTYSRGPLDVWGQGTTVTVSSASTKGPSVFP |
| | | | LAPSSKSTSGGTAALGCLVKDYFPEPVTVSWNS |
| | | | GALTSGVHTFPAVLQSSGLYSLSSVVTVPSSSL |
| | | | GTQTYICNVNHKPSNTKVDKKVEPKSCDKTHTC |
| | | | PPCPAPEAAGAPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSREEM |
| | | | TKNQVSLTCLVKGFYPSDIAVEWESNGQPENNY |
| | | | KTTPPVLDSDGSFFLYSKLTVDKSRWQQGNVFS |
| | | | CSVMHEALHNHYTQKSLSLSPGK |
| 251 | IGG1 AAA HC | 38422 | QVQLQESGPGLVKPSETLSLTCAVSGYSISSGF |
| | | | YWTWIRQPPGKGLEWIGAIYHSGSTVYNPSLKS |
| | | | RVTISVDTSKNQFSLKLSSVTAADTAVYYCARG |
| | | | GTHTYSRGPLDVWGQGTTVTVSSASTKGPSVFP |
| | | | LAPSSKSTSGGTAALGCLVKDYFPEPVTVSWNS |
| | | | GALTSGVHTFPAVLQSSGLYSLSSVVTVPSSSL |
| | | | GTQTYICNVNHKPSNTKVDKKVEPKSCDKTHTC |
| | | | PPCPAPEAAGAPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSREEM |
| | | | TKNQVSLTCLVKGFYPSDIAVEWESNGQPENNY |
| | | | KTTPPVLDSDGSFFLYSKLTVDKSRWQQGNVFS |
| | | | CSVMHEALHNHYTQKSLSLSPGK |
| 252 | IGG1 AAA HC | 38424 | QVQLQESGPGLVKPSETLSLTCAVSGYSISSGY |
| | | | YWLWIRQPPGKGLEWIGGIYHSASTAYNPSLKS |
| | | | RVTISVDTSKNQFSLKLSSVTAADTAVYYCARG |
| | | | GTVKYSRGPLDVWGQGTTVTVSSASTKGPSVFP |
| | | | LAPSSKSTSGGTAALGCLVKDYFPEPVTVSWNS |
| | | | GALTSGVHTFPAVLQSSGLYSLSSVVTVPSSSL |
| | | | GTQTYICNVNHKPSNTKVDKKVEPKSCDKTHTC |
| | | | PPCPAPEAAGAPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSREEM |
| | | | TKNQVSLTCLVKGFYPSDIAVEWESNGQPENNY |
| | | | KTTPPVLDSDGSFFLYSKLTVDKSRWQQGNVFS |
| | | | CSVMHEALHNHYTQKSLSLSPGK |
| 253 | IGG1 AAA HC | 38425 | QVQLQESGPGLVKPSETLSLTCAVSGYSISSGH |
| | | | YWTWIRQPPGKGLEWIGAIYHSGSTVYNPSLKS |
| | | | RVT1SVDTSKNQFSLKLSSVTAADTAVYYCARG |
| | | | GQVTYSRGPLDVWGQGTTVTVSSASTKGPSVFP |
| | | | LAPSSKSTSGGTAALGCLVKDYFPEPVTVSWNS |
| | | | GALTSGVHTFPAVLQSSGLYSLSSVVTVPSSSL |
| | | | GTQTYICNVNHKPSNTKVDKKVEPKSCDKTHTC |
| | | | PPCPAPEAAGAPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSREEM |
| | | | TKNQVSLTCLVKGFYPSDIAVEWESNGQPENNY |
| | | | KTTPPVLDSDGSFFLYSKLTVDKSRWQQGNVFS |
| | | | CSVMHEALHNHYTQKSLSLSPGK |
| 254 | IGG1 AAA HC | 38426 | QVQLQESGPGLVKPSETLSLTCAVSGYSILSGY |
| | | | YWFWIRQPPGKGLEWIGGIYHSGSTAYNPSLKS |
| | | | RVTISVDTSKNQFSLKLSSVTAADTAVYYCARG |
| | | | GEVTYSRGPLDVWGQGTTVTVSSASTKGPSVFP |
| | | | LAPSSKSTSGGTAALGCLVKDYFPEPVTVSWNS |
| | | | GALTSGVHTFPAVLQSSGLYSLSSVVTVPSSSL |
| | | | GTQTYICNVNHKPSNTKVDKKVEPKSCDKTHTC |
| | | | PPCPAPEAAGAPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSREEM |
| | | | TKNQVSLTCLVKGFYPSDIAVEWESNGQPENNY |
| | | | KTTPPVLDSDGSFFLYSKLTVDKSRWQQGNVFS |
| | | | CSVMHEALHNHYTQKSLSLSPGK |
| 255 | IGG1 AAA HC | 37323 | EVQLLESGGGLVQPGGSLRLSCAASGFTFDNYA |
| | | | MHWVRQAPGKGLEWVSAISARAGITYYADSVKG |
| | | | RFTISRDNSKNTLYLQMNSLRAEDTAVYYCARR |
| | | | IGYSYGTAPPFDVWGQGTTVTVSSASTKGPSVF |
| | | | PLAPSSKSTSGGTAALGCLVKDYFPEPVTVSWN |
| | | | SGALTSGVHTFPAVLQSSGLYSLSSVVTVPSSS |
| | | | LGTQTYICNVNHKPSNTKVDKKVEPKSCDKTHT |
| | | | CPPCPAPEAAGAPSVFLFPPKPKDTLMISRTPE |
| | | | VTCVVVDVSHEDPEVKFNWYVDGVEVHNAKTKP |
| | | | REEQYNSTYRVVSVLTVLHQDWLNGKEYKCKVS |
| | | | NKALPAPIEKTISKAKGQPREPQVYTLPPSREE |
| | | | MTKNQVSLTCLVKGFYPSDIAVEWESNGQPENN |
| | | | YKTTPPVLDSDGSFFLYSKLTVDKSRWQQGNVF |
| | | | SCSVMHEALHNHYTQKSLSLSPGK |
| 256 | IGG1 AAA HC | 38389 | QVQLVESGGGLVQPGGSLRLSCAASGFTFSDYY |
| | | | MSWIRQAPGKGLEWVSYIASSGSVIYYADSVKG |
| | | | RFTISRDNAKNSLYLQMNSLRAEDTAVYYCARH |
| | | | GTPRAFDIWGQGTTVTVSSASTKGPSVFPLAPS |
| | | | SKSTSGGTAALGCLVKDYFPEPVTVSWNSGALT |
| | | | SGVHTFPAVLQSSGLYSLSSVVTVPSSSLGTQT |
| | | | YICNVNHKPSNTKVDKKVEPKSCDKTHTCPPCP |
| | | | APEAAGAPSVFLFPPKPKDTLMISRTPEVTCVV |
| | | | VDVSHEDPEVKFNWYVDGVEVHNAKTKPREEQY |
| | | | NSTYRVVSVLTVLHQDWLNGKEYKCKVSNKALP |
| | | | APIEKTISKAKGQPREPQVYTLPPSREEMTKNQ |
| | | | VSLTCLVKGFYPSDIAVEWESNGQPENNYKTTP |
| | | | PVLDSDGSFFLYSKLTVDKSRWQQGNVFSCSVM |
| | | | HEALHNHYTQKSLSLSPGK |
| 257 | IGG1 AAA HC | 38358 | EVQLLESGGGLVQPGGSLRLSCAASGFTFSSSA |
| | | | MAWVRQAPGKGLEWVSTISGSGITTWYADSVKG |
| | | | RFTISRDNSKNTLYLQMNSLRAEDTAVYYCAKG |
| | | | SRHLNAFNRWGQGTTVTVSSASTKGPSVFPLAP |
| | | | SSKSTSGGTAALGCLVKDYFPEPVTVSWNSGAL |
| | | | TSGVHTFPAVLQSSGLYSLSSVVTVPSSSLGTQ |
| | | | TYICNVNHKPSNTKVDKKVEPKSCDKTHTCPPC |
| | | | PAPEAAGAPSVFLFPPKPKDTLMISRTPEVTCV |
| | | | VVDVSHEDPEVKFNWYVDGVEVHNAKTKPREEQ |
| | | | YNSTYRVVSVLTVLHQDWLNGKEYKCKVSNKAL |
| | | | PAPIEKTISKAKGQPREPQVYTLPPSREEMTKN |
| | | | QVSLTCLVKGFYPSDIAVEWESNGQPENNYKTT |
| | | | PPVLDSDGSFFLYSKLTVDKSRWQQGNVFSCSV |
| | | | MHEALHNHYTQKSLSLSPGK |
| 258 | IGG1 AAA HC | 33303 | QVQLQESGPGLVKPSGTLSLTCAVSGGSISSSN |
| | | | WWSWVRQPPGKGLEWIGEIYHSGSTNYNPSLKS |
| | | | RVTISVDKSKNQFSLKLSSVTAADTAVYYCARG |
| | | | VYHYDPYGMDVWGQGTTVTVSSASTKGPSVFPL |
| | | | APSSKSTSGGTAALGCLVKDYFPEPVTVSWNSG |
| | | | ALTSGVHTFPAVLQSSGLYSLSSVVTVPSSSLG |
| | | | TQTYICNVNHKPSNTKVDKKVEPKSCDKTHTCP |
| | | | PCPAPEAAGAPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSREEMT |
| | | | KNQVSLTCLVKGFYPSDIAVEWESNGQPENNYK |
| | | | TTPPVLDSDGSFFLYSKLTVDKSRWQQGNVFSC |
| | | | SVMHEALHNHYTQKSLSLSPGK |
| 259 | IGG1 AAA HC | 33342 | QLQLQESGPGLVKPSETLSLTCTVSGGSISSSS |
| | | | YYWGWIRQPPGKGLEWIGSISYSGSTYYNPSLK |
| | | | SRVTISVDTSKNQFSLKLSSVTAADTAVYYCAR |
| | | | TELGKMHFDYWGQGTLVTVSSASTKGPSVFPLA |
| | | | PSSKSTSGGTAALGCLVKDYFPEPVTVSWNSGA |
| | | | LTSGVHTFPAVLQSSGLYSLSSVVTVPSSSLGT |
| | | | QTYICNVNHKPSNTKVDKKVEPKSCDKTHTCPP |
| | | | CPAPEAAGAPSVFLFPPKPKDTLMISRTPEVTC |
| | | | VVVDVSHEDPEVKFNWYVDGVEVHNAKTKPREE |
| | | | QYNSTYRVVSVLTVLHQDWLNGKEYKCKVSNKA |
| | | | LPAPIEKTISKAKGQPREPQVYTLPPSREEMTK |
| | | | NQVSLTCLVKGFYPSDIAVEWESNGQPENNYKT |
| | | | TPPVLDSDGSFFLYSKLTVDKSRWQQGNVFSCS |
| | | | VMHEALHNHYTQKSLSLSPGK |
| 260 | IGG1 AAA HC | 33299 | QLQLQESGPGLVKPSETLSLTCTVSGGSISSSD |
| | | | YYWGWIRQPPGKGLEWIGSIYYSGSTYYNPSLK |
| | | | SRVTISVDTSKNQFSLKLSSVTAADTAVYYCAR |
| | | | GSPRYMQDWGQGTLVTVSSASTKGPSVFPLAPS |
| | | | SKSTSGGTAALGCLVKDYFPEPVTVSWNSGALT |
| | | | SGVHTFPAVLQSSGLYSLSSVVTVPSSSLGTQT |
| | | | YICNVNHKPSNTKVDKKVEPKSCDKTHTCPPCP |
| | | | APEAAGAPSVFLFPPKPKDTLMISRTPEVTCVV |
| | | | VDVSHEDPEVKFNWYVDGVEVHNAKTKPREEQY |
| | | | NSTYRVVSVLTVLHQDWLNGKEYKCKVSNKALP |
| | | | APIEKTISKAKGQPREPQVYTLPPSREEMTKNQ |
| | | | VSLTCLVKGFYPSDIAVEWESNGQPENNYKTTP |
| | | | PVLDSDGSFFLYSKLTVDKSRWQQGNVFSCSVM |
| | | | HEALHNHYTQKSLSLSPGK |
| 261 | IGG1 AAA HC | 33351 | QVQLVESGGGLVKPGGSLRLSCAASGFTFSDYY |
| | | | MSWIRQAPGKGLEWVSYISSSGSTIYYADSVKG |
| | | | RFTISRDNAKNSLYLQMNSLRAEDTAVYYCARH |
| | | | SSLGTHNWFDPWGQGTLVTVSSASTKGPSVFPL |
| | | | APSSKSTSGGTAALGCLVKDYFPEPVTVSWNSG |
| | | | ALTSGVHTFPAVLQSSGLYSLSSVVTVPSSSLG |
| | | | TQTYICNVNHKPSNTKVDKKVEPKSCDKTHTCP |
| | | | PCPAPEAAGAPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSREEMT |
| | | | KNQVSLTCLVKGFYPSDIAVEWESNGQPENNYK |
| | | | TTPPVLDSDGSFFLYSKLTVDKSRWQQGNVFSC |
| | | | SVMHEALHNHYTQKSLSLSPGK |
| 262 | IGG1 AAA HC | 33357 | QVQLQESGPGLVKPSETLSLTCAVSGYSISSGY |
| | | | YWGWIRQPPGKGLEWIGSIYHSGSTYYNPSLKS |
| | | | RVT1SVDTSKNQFSLKLSSVTAADTAVYYCARE |
| | | | GALSYSWLAAFDIWGQGTMVTVSSASTKGPSVF |
| | | | PLAPSSKSTSGGTAALGCLVKDYFPEPVTVSWN |
| | | | SGALTSGVHTFPAVLQSSGLYSLSSVVTVPSSS |
| | | | LGTQTYICNVNHKPSNTKVDKKVEPKSCDKTHT |
| | | | CPPCPAPEAAGAPSVFLFPPKPKDTLMISRTPE |
| | | | VTCVVVDVSHEDPEVKFNWYVDGVEVHNAKTKP |
| | | | REEQYNSTYRVVSVLTVLHQDWLNGKEYKCKVS |
| | | | NKALPAPIEKTISKAKGQPREPQVYTLPPSREE |
| | | | MTKNQVSLTCLVKGFYPSDIAVEWESNGQPENN |
| | | | YKTTPPVLDSDGSFFLYSKLTVDKSRWQQGNVF |
| | | | SCSVMHEALHNHYTQKSLSLSPGK |
| 263 | | | |
| 264 | | | |
| 265 | | | |
| 266 | IGG4 HC | 33343 | QLQLQESGPGLVKPSETLSLTCTVSGGSISSSD |
| | | | YYWGWIRQPPGKGLEWIGSIYYSGSTYYNPSLK |
| | | | SRVTISVDTSKNQFSLKLSSVTAADTAVYYCAR |
| | | | GVRRAVPFDYWGQGTLVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVKGFYPSDIAVEWESNGQPENNYKTTPP |
| | | | VLDSDGSFFLYSRLTVDKSRWQEGNVFSCSVMH |
| | | | EALHNHYTQKSLSLSPGK |
| 267 | IGG4 HC | 37268 | QLQLQESGPGLVKPSETLSLTCTVSGGSISSSS |
| | | | TYWAWIRQPPGKGLEWIGSISSSGSTYYNPSLK |
| | | | SRVTISVDTSKNQFSLKLSSVTAADTAVYYCAR |
| | | | GIARAVPFDYWGQGTLVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVKGFYPSDIAVEWESNGQPENNYKTTPP |
| | | | VLDSDGSFFLYSRLTVDKSRWQEGNVFSCSVMH |
| | | | EALHNHYTQKSLSLSPGK |
| 268 | IGG4 HC | 37269 | QLQLQESGPGLVKPSETLSLTCTVSGGSISSSS |
| | | | TYWGWIRQSPGKGLEWIGSIHHSGATYYNPSLK |
| | | | SRVTISVDTSKNQFSLKLSSVTAADTAVYYCAR |
| | | | GPKRAVPFDYWGQGTLVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVKGFYPSDIAVEWESNGQPENNYKTTPP |
| | | | VLDSDGSFFLYSRLTVDKSRWQEGNVFSCSVMH |
| | | | EALHNHYTQKSLSLSPGK |
| 269 | IGG4 HC | 37271 | QLQLQESGPGLVKPSETLSLTCTVSGGSISSSD |
| | | | TYWGWIRQPPGKGLEWIGSIHYSGSTLYNPSLK |
| | | | SRVTISVDTSKNQFSLKLSSVTAADTAVYYCAR |
| | | | GVRRAVPFVDWGQGTLVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVKGFYPSDIAVEWESNGQPENNYKTTPP |
| | | | VLDSDGSFFLYSRLTVDKSRWQEGNVFSCSVMH |
| | | | EALHNHYTQKSLSLSPGK |
| 270 | IGG4 HC | 37272 | QLQLQESGPGLVKPSETLSLTCTVSGGSISSAD |
| | | | NYWGWIRQPPGKGLEWIGSIHYSGSTYYNPSLK |
| | | | SRVTISVDTSKNQFSLKLSSVTAADTAVYYCAR |
| | | | GVRRAVPFQRWGQGTLVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVKGFYPSDIAVEWESNGQPENNYKTTPP |
| | | | VLDSDGSFFLYSRLTVDKSRWQEGNVFSCSVMH |
| | | | EALHNHYTQKSLSLSPGK |
| 271 | IGG4 HC | 37277 | QLQLQESGPGLVKPSETLSLTCTVSGGSISSSD |
| | | | TYWGWIRQPPGKGPEWIGSIHYSGSTLYNPSLK |
| | | | SRVTISVDTSKNQFSLKLSSVTAADTAVYYCAR |
| | | | GVRRAVPFDYWGQGTLVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVKGFYPSDIAVEWESNGQPENNYKTTPP |
| | | | VLDSDGSFFLYSRLTVDKSRWQEGNVFSCSVMH |
| | | | EALHNHYTQKSLSLSPGK |
| 272 | IGG4 HC | 38373 | QLQLQESGPGLVKPSETLSLTCTVSGGSISSSS |
| | | | TYWSWIRQPPGKGLEWIGGIAYSGSTYYNPSLK |
| | | | SRVTISVDTSKNQFSLKLSSVTAADTAVYYCAR |
| | | | GVRRAVPFDYWGQGTLVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVKGFYPSDIAVEWESNGQPENNYKTTPP |
| | | | VLDSDGSFFLYSRLTVDKSRWQEGNVFSCSVMH |
| | | | EALHNHYTQKSLSLSPGK |
| 273 | IGG4 HC | 38375 | QLQLQESGPGLVKPSETLSLTCTVSGGSISSSS |
| | | | TYWSWIRQPPGKGLEWIGSISYNALTYYNPSLK |
| | | | SRVTISVDTSKNQFSLKLSSVTAADTAVYYCAR |
| | | | GTRRAVPFDYWGQGTLVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVKGFYPSDIAVEWESNGQPENNYKTTPP |
| | | | VLDSDGSFFLYSRLTVDKSRWQEGNVFSCSVMH |
| | | | EALHNHYTQKSLSLSPGK |
| 274 | IGG4 HC | 38379 | QLQLQESGPGLVKPSETLSLTCTVSGGSISSSS |
| | | | TYWSWIRQPPGKGLEWIGGIAYSGSTYYNPSLK |
| | | | SRVTISVDTSKNQFSLKLSSVTAADTAVYYCAR |
| | | | GVRRAVPFADWGQGTLVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVKGFYPSDIAVEWESNGQPENNYKTTPP |
| | | | VLDSDGSFFLYSRLTVDKSRWQEGNVFSCSVMH |
| | | | EALHNHYTQKSLSLSPGK |
| 275 | IGG4 HC | 38381 | QLQLQESGPGLVKPSETLSLTCTVSGGSVSSSS |
| | | | TYWSWIRQPPGKGLEWIGGIAYSGSTYYNPSLK |
| | | | SRVTISVDTSKNQFSLKLSSVTAADTAVYYCAR |
| | | | GVRRAVPFDYWGQGTLVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVKGFYPSDIAVEWESNGQPENNYKTTPP |
| | | | VLDSDGSFFLYSRLTVDKSRWQEGNVFSCSVMH |
| | | | EALHNHYTQKSLSLSPGK |
| 276 | IGG4 HC | 38383 | QLQLQESGPGLVKPSETLSLTCTVSGGSISSSS |
| | | | TYWSWIRQPPGKGLEWIGGIAYSGSTYYNPSLK |
| | | | SRVTISVDTSKNQFSLKLSSVTAADTAVYYCAR |
| | | | GTRRAVPFDYWGQGTLVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVKGFYPSDIAVEWESNGQPENNYKTTPP |
| | | | VLDSDGSFFLYSRLTVDKSRWQEGNVFSCSVMH |
| | | | EALHNHYTQKSLSLSPGK |
| 277 | IGG4 HC | 38386 | QLQLQESGPGLVKPSETLSLTCTVSGGSISSSD |
| | | | TYWGWIRQPPGKGLEWIGSIHYSGSTLYNPSLK |
| | | | SRVTISVDTSKNQFSLKLSSVTAADTAVYYCAR |
| | | | GIRRAVPFDYWGQGTLVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVKGFYPSDIAVEWESNGQPENNYKTTPP |
| | | | VLDSDGSFFLYSRLTVDKSRWQEGNVFSCSVMH |
| | | | EALHNHYTQKSLSLSPGK |
| 278 | IGG4 HC | 37273 | QLQLQESGPGLVKPSETLSLTCTVSGGSISSSD |
| | | | TYWGWIRQPPGKGLEWIGSIHYSGSTLYNPSLK |
| | | | SRVTISVDTSKNQFSLKLSSVTAADTAVYYCAR |
| | | | GQFRAVPFDYWGQGTLVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVKGFYPSDIAVEWESNGQPENNYKTTPP |
| | | | VLDSDGSFFLYSRLTVDKSRWQEGNVFSCSVMH |
| | | | EALHNHYTQKSLSLSPGK |
| 279 | IGG4 HC | 33361 | QVQLQESGPGLVKPSETLSLTCAVSGYSISSGY |
| | | | YWGWIRQPPGKGLEWIGSIYHSGSTYYNPSLKS |
| | | | RVTISVDTSKNQFSLKLSSVTAADTAVYYCARG |
| | | | GTHTYSRGPMDVWGQGTTVTVSSASTKGPSVFP |
| | | | LAPCSRSTSESTAALGCLVKDYFPEPVTVSWNS |
| | | | GALTSGVHTFPAVLQSSGLYSLSSVVTVPSSSL |
| | | | GTKTYTCNVDHKPSNTKVDKRVESKYGPPCPPC |
| | | | PAPEFLGGPSVFLFPPKPKDTLMISRTPEVTCV |
| | | | VVDVSQEDPEVQFNWYVDGVEVHNAKTKPREEQ |
| | | | FNSTYRVVSVLTVLHQDWLNGKEYKCKVSNKGL |
| | | | PSSIEKTISKAKGQPREPQVYTLPPSQEEMTKN |
| | | | QVSLTCLVKGFYPSDIAVEWESNGQPENNYKTT |
| | | | PPVLDSDGSFFLYSRLTVDKSRWQEGNVFSCSV |
| | | | MHEALHNHYTQKSLSLSPGK |
| 280 | IGG4 HC | 35624 | QLQLQESGPRLVKPSETLSLTCAVSGYSISSGY |
| | | | YWGWIRQPPGKGLEWIGSIYHSGSTYYNPSLKS |
| | | | RVTISVDTSKNQFSLKLSSVTAADTAVYYCARG |
| | | | GTHTYSRGPFDVWGQGTTVTVSSASTKGPSVFP |
| | | | LAPCSRSTSESTAALGCLVKDYFPEPVTVSWNS |
| | | | GALTSGVHTFPAVLQSSGLYSLSSVVTVPSSSL |
| | | | GTKTYTCNVDHKPSNTKVDKRVESKYGPPCPPC |
| | | | PAPEFLGGPSVFLFPPKPKDTLMISRTPEVTCV |
| | | | VVDVSQEDPEVQFNWYVDGVEVHNAKTKPREEQ |
| | | | FNSTYRVVSVLTVLHQDWLNGKEYKCKVSNKGL |
| | | | PSSIEKTISKAKGQPREPQVYTLPPSQEEMTKN |
| | | | QVSLTCLVKGFYPSDIAVEWESNGQPENNYKTT |
| | | | PPVLDSDGSFFLYSRLTVDKSRWQEGNVFSCSV |
| | | | MHEALHNHYTQKSLSLSPGK |
| 281 | IGG4 HC | 38410 | LVQLQESGPGLVKPSETLSLTCAVSGYSILSGY |
| | | | YWFWIRQPPGKGLEWIGGIYHSASTAYNPSLKS |
| | | | RVTISVDTSKNQFSLKLSSVTAADTAVYYCARG |
| | | | GTPIYSRGPLDVWGQGTTVTVSSASTKGPSVFP |
| | | | LAPCSRSTSESTAALGCLVKDYFPEPVTVSWNS |
| | | | GALTSGVHTFPAVLQSSGLYSLSSVVTVPSSSL |
| | | | GTKTYTCNVDHKPSNTKVDKRVESKYGPPCPPC |
| | | | PAPEFLGGPSVFLFPPKPKDTLMISRTPEVTCV |
| | | | VVDVSQEDPEVQFNWYVDGVEVHNAKTKPREEQ |
| | | | FNSTYRVVSVLTVLHQDWLNGKEYKCKVSNKGL |
| | | | PSSIEKTISKAKGQPREPQVYTLPPSQEEMTKN |
| | | | QVSLTCLVKGFYPSDIAVEWESNGQPENNYKTT |
| | | | PPVLDSDGSFFLYSRLTVDKSRWQEGNVFSCSV |
| | | | MHEALHNHYTQKSLSLSPGK |
| 282 | IGG4 HC | 38418 | QVQLQESGPGLVKPSETLSLTCAVSGYSISSGH |
| | | | YWIWIRQPPGKGLEWIGGIYHSGSTYYNPSLKS |
| | | | RVTISVDTSKDQFSLKLSSVTAADTAVYYCARG |
| | | | GGQTYSRGPLDVWGQGTTVTVSSASTKGPSVFP |
| | | | LAPCSRSTSESTAALGCLVKDYFPEPVTVSWNS |
| | | | GALTSGVHTFPAVLQSSGLYSLSSVVTVPSSSL |
| | | | GTKTYTCNVDHKPSNTKVDKRVESKYGPPCPPC |
| | | | PAPEFLGGPSVFLFPPKPKDTLMISRTPEVTCV |
| | | | VVDVSQEDPEVQFNWYVDGVEVHNAKTKPREEQ |
| | | | FNSTYRVVSVLTVLHQDWLNGKEYKCKVSNKGL |
| | | | PSSIEKTISKAKGQPREPQVYTLPPSQEEMTKN |
| | | | QVSLTCLVKGFYPSDIAVEWESNGQPENNYKTT |
| | | | PPVLDSDGSFFLYSRLTVDKSRWQEGNVFSCSV |
| | | | MHEALHNHYTQKSLSLSPGK |
| 283 | IGG4 HC | 38420 | QVQLQESGPGLVKPPETLSLTCAVSGYSISSGH |
| | | | YWIWIRQPPGKGLEWIGGIYHSGSTYYNPSLKS |
| | | | RVTISVDTSKNQFSLKLSSVTAADTAVYYCARG |
| | | | GGATYSRGPLDVWGQGTTVTVSSASTKGPSVFP |
| | | | LAPCSRSTSESTAALGCLVKDYFPEPVTVSWNS |
| | | | GALTSGVHTFPAVLQSSGLYSLSSVVTVPSSSL |
| | | | GTKTYTCNVDHKPSNTKVDKRVESKYGPPCPPC |
| | | | PAPEFLGGPSVFLFPPKPKDTLMISRTPEVTCV |
| | | | VVDVSQEDPEVQFNWYVDGVEVHNAKTKPREEQ |
| | | | FNSTYRVVSVLTVLHQDWLNGKEYKCKVSNKGL |
| | | | PSSIEKTISKAKGQPREPQVYTLPPSQEEMTKN |
| | | | QVSLTCLVKGFYPSDIAVEWESNGQPENNYKTT |
| | | | PPVLDSDGSFFLYSRLTVDKSRWQEGNVFSCSV |
| | | | MHEALHNHYTQKSLSLSPGK |
| 284 | IGG4 HC | 38421 | QVQLQESGPGLVKPSETLSLTCAVSGYSILSGY |
| | | | YWFWIRQPPGKGLEWIGGIYHSGSTYYNPSLKS |
| | | | RVTISVDTSKNQFSLKLSSVTAADTAVYYCARG |
| | | | GTHTYSRGPLDVWGQGTTVTVSSASTKGPSVFP |
| | | | LAPCSRSTSESTAALGCLVKDYFPEPVTVSWNS |
| | | | GALTSGVHTFPAVLQSSGLYSLSSVVTVPSSSL |
| | | | GTKTYTCNVDHKPSNTKVDKRVESKYGPPCPPC |
| | | | PAPEFLGGPSVFLFPPKPKDTLMISRTPEVTCV |
| | | | VVDVSQEDPEVQFNWYVDGVEVHNAKTKPREEQ |
| | | | FNSTYRVVSVLTVLHQDWLNGKEYKCKVSNKGL |
| | | | PSSIEKTISKAKGQPREPQVYTLPPSQEEMTKN |
| | | | QVSLTCLVKGFYPSDIAVEWESNGQPENNYKTT |
| | | | PPVLDSDGSFFLYSRLTVDKSRWQEGNVFSCSV |
| | | | MHEALHNHYTQKSLSLSPGK |
| 285 | IGG4 HC | 38422 | QVQLQESGPGLVKPSETLSLTCAVSGYSISSGF |
| | | | YWTWIRQPPGKGLEWIGAIYHSGSTVYNPSLKS |
| | | | RVTISVDTSKNQFSLKLSSVTAADTAVYYCARG |
| | | | GTHTYSRGPLDVWGQGTTVTVSSASTKGPSVFP |
| | | | LAPCSRSTSESTAALGCLVKDYFPEPVTVSWNS |
| | | | GALTSGVHTFPAVLQSSGLYSLSSVVTVPSSSL |
| | | | GTKTYTCNVDHKPSNTKVDKRVESKYGPPCPPC |
| | | | PAPEFLGGPSVFLFPPKPKDTLMISRTPEVTCV |
| | | | VVDVSQEDPEVQFNWYVDGVEVHNAKTKPREEQ |
| | | | FNSTYRVVSVLTVLHQDWLNGKEYKCKVSNKGL |
| | | | PSSIEKTISKAKGQPREPQVYTLPPSQEEMTKN |
| | | | QVSLTCLVKGFYPSDIAVEWESNGQPENNYKTT |
| | | | PPVLDSDGSFFLYSRLTVDKSRWQEGNVFSCSV |
| | | | MHEALHNHYTQKSLSLSPGK |
| 286 | IGG4 HC | 38424 | QVQLQESGPGLVKPSETLSLTCAVSGYSISSGY |
| | | | YWLWIRQPPGKGLEWIGGIYHSASTAYNPSLKS |
| | | | RVTISVDTSKNQFSLKLSSVTAADTAVYYCARG |
| | | | GTVKYSRGPLDVWGQGTTVTVSSASTKGPSVFP |
| | | | LAPCSRSTSESTAALGCLVKDYFPEPVTVSWNS |
| | | | GALTSGVHTFPAVLQSSGLYSLSSVVTVPSSSL |
| | | | GTKTYTCNVDHKPSNTKVDKRVESKYGPPCPPC |
| | | | PAPEFLGGPSVFLFPPKPKDTLMISRTPEVTCV |
| | | | VVDVSQEDPEVQFNWYVDGVEVHNAKTKPREEQ |
| | | | FNSTYRVVSVLTVLHQDWLNGKEYKCKVSNKGL |
| | | | PSSIEKTISKAKGQPREPQVYTLPPSQEEMTKN |
| | | | QVSLTCLVKGFYPSDIAVEWESNGQPENNYKTT |
| | | | PPVLDSDGSFFLYSRLTVDKSRWQEGNVFSCSV |
| | | | MHEALHNHYTQKSLSLSPGK |
| 287 | IGG4 HC | 38425 | QVQLQESGPGLVKPSETLSLTCAVSGYSISSGH |
| | | | YWTWIRQPPGKGLEWIGAIYHSGSTVYNPSLKS |
| | | | RVTISVDTSKNQFSLKLSSVTAADTAVYYCARG |
| | | | GQVTYSRGPLDVWGQGTTVTVSSASTKGPSVFP |
| | | | LAPCSRSTSESTAALGCLVKDYFPEPVTVSWNS |
| | | | GALTSGVHTFPAVLQSSGLYSLSSVVTVPSSSL |
| | | | GTKTYTCNVDHKPSNTKVDKRVESKYGPPCPPC |
| | | | PAPEFLGGPSVFLFPPKPKDTLMISRTPEVTCV |
| | | | VVDVSQEDPEVQFNWYVDGVEVHNAKTKPREEQ |
| | | | FNSTYRVVSVLTVLHQDWLNGKEYKCKVSNKGL |
| | | | PSSIEKTISKAKGQPREPQVYTLPPSQEEMTKN |
| | | | QVSLTCLVKGFYPSDIAVEWESNGQPENNYKTT |
| | | | PPVLDSDGSFFLYSRLTVDKSRWQEGNVFSCSV |
| | | | MHEALHNHYTQKSLSLSPGK |
| 288 | IGG4 HC | 38426 | QVQLQESGPGLVKPSETLSLTCAVSGYSILSGY |
| | | | YWFWIRQPPGKGLEWIGGIYHSGSTAYNPSLKS |
| | | | RVTISVDTSKNQFSLKLSSVTAADTAVYYCARG |
| | | | GEVTYSRGPLDVWGQGTTVTVSSASTKGPSVFP |
| | | | LAPCSRSTSESTAALGCLVKDYFPEPVTVSWNS |
| | | | GALTSGVHTFPAVLQSSGLYSLSSVVTVPSSSL |
| | | | GTKTYTCNVDHKPSNTKVDKRVESKYGPPCPPC |
| | | | PAPEFLGGPSVFLFPPKPKDTLMISRTPEVTCV |
| | | | VVDVSQEDPEVQFNWYVDGVEVHNAKTKPREEQ |
| | | | FNSTYRVVSVLTVLHQDWLNGKEYKCKVSNKGL |
| | | | PSSIEKTISKAKGQPREPQVYTLPPSQEEMTKN |
| | | | QVSLTCLVKGFYPSDIAVEWESNGQPENNYKTT |
| | | | PPVLDSDGSFFLYSRLTVDKSRWQEGNVFSCSV |
| | | | MHEALHNHYTQKSLSLSPGK |
| 289 | IGG4 HC | 37323 | EVQLLESGGGLVQPGGSLRLSCAASGFTFDNYA |
| | | | MHWVRQAPGKGLEWVSAISARAGITYYADSVKG |
| | | | RFTISRDNSKNTLYLQMNSLRAEDTAVYYCARR |
| | | | IGYSYGTAPPFDVWGQGTTVTVSSASTKGPSVF |
| | | | PLAPCSRSTSESTAALGCLVKDYFPEPVTVSWN |
| | | | SGALTSGVHTFPAVLQSSGLYSLSSVVTVPSSS |
| | | | LGTKTYTCNVDHKPSNTKVDKRVESKYGPPCPP |
| | | | CPAPEFLGGPSVFLFPPKPKDTLMISRTPEVTC |
| | | | VVVDVSQEDPEVQFNWYVDGVEVHNAKTKPREE |
| | | | QFNSTYRVVSVLTVLHQDWLNGKEYKCKVSNKG |
| | | | LPSSIEKTISKAKGQPREPQVYTLPPSQEEMTK |
| | | | NQVSLTCLVKGFYPSDIAVEWESNGQPENNYKT |
| | | | TPPVLDSDGSFFLYSRLTVDKSRWQEGNVFSCS |
| | | | VMHEALHNHYTQKSLSLSPGK |
| 290 | IGG4 HC | 38389 | QVQLVESGGGLVQPGGSLRLSCAASGFTFSDYY |
| | | | MSWIRQAPGKGLEWVSYIASSGSVIYYADSVKG |
| | | | RFTISRDNAKNSLYLQMNSLRAEDTAVYYCARH |
| | | | GTPRAFDIWGQGTTVTVSSASTKGPSVFPLAPC |
| | | | SRSTSESTAALGCLVKDYFPEPVTVSWNSGALT |
| | | | SGVHTFPAVLQSSGLYSLSSVVTVPSSSLGTKT |
| | | | YTCNVDHKPSNTKVDKRVESKYGPPCPPCPAPE |
| | | | FLGGPSVFLFPPKPKDTLMISRTPEVTCVVVDV |
| | | | SQEDPEVQFNWYVDGVEVHNAKTKPREEQFNST |
| | | | YRVVSVLTVLHQDWLNGKEYKCKVSNKGLPSSI |
| | | | EKTISKAKGQPREPQVYTLPPSQEEMTKNQVSL |
| | | | TCLVKGFYPSDIAVEWESNGQPENNYKTTPPVL |
| | | | DSDGSFFLYSRLTVDKSRWQEGNVFSCSVMHEA |
| | | | LHNHYTQKSLSLSPGK |
| 291 | IGG4 HC | 38358 | EVQLLESGGGLVQPGGSLRLSCAASGFTFSSSA |
| | | | MAWVRQAPGKGLEWVSTISGSGITTWYADSVKG |
| | | | RFTISRDNSKNTLYLQMNSLRAEDTAVYYCAKG |
| | | | SRHLNAFNRWGQGTTVTVSSASTKGPSVFPLAP |
| | | | CSRSTSESTAALGCLVKDYFPEPVTVSWNSGAL |
| | | | TSGVHTFPAVLQSSGLYSLSSVVTVPSSSLGTK |
| | | | TYTCNVDHKPSNTKVDKRVESKYGPPCPPCPAP |
| | | | EFLGGPSVFLFPPKPKDTLMISRTPEVTCVVVD |
| | | | VSQEDPEVQFNWYVDGVEVHNAKTKPREEQFNS |
| | | | TYRVVSVLTVLHQDWLNGKEYKCKVSNKGLPSS |
| | | | IEKTISKAKGQPREPQVYTLPPSQEEMTKNQVS |
| | | | LTCLVKGFYPSDIAVEWESNGQPENNYKTTPPV |
| | | | LDSDGSFFLYSRLTVDKSRWQEGNVFSCSVMHE |
| | | | ALHNHYTQKSLSLSPGK |
| 292 | IGG4 HC | 33303 | QVQLQESGPGLVKPSGTLSLTCAVSGGSISSSN |
| | | | WWSWVRQPPGKGLEWIGEIYHSGSTNYNPSLKS |
| | | | RVTISVDKSKNQFSLKLSSVTAADTAVYYCARG |
| | | | VYHYDPYGMDVWGQGTTVTVSSASTKGPSVFPL |
| | | | APCSRSTSESTAALGCLVKDYFPEPVTVSWNSG |
| | | | ALTSGVHTFPAVLQSSGLYSLSSVVTVPSSSLG |
| | | | TKTYTCNVDHKPSNTKVDKRVESKYGPPCPPCP |
| | | | APEFLGGPSVFLFPPKPKDTLMISRTPEVTCVV |
| | | | VDVSQEDPEVQFNWYVDGVEVHNAKTKPREEQF |
| | | | NSTYRVVSVLTVLHQDWLNGKEYKCKVSNKGLP |
| | | | SSIEKTISKAKGQPREPQVYTLPPSQEEMTKNQ |
| | | | VSLTCLVKGFYPSDIAVEWESNGQPENNYKTTP |
| | | | PVLDSDGSFFLYSRLTVDKSRWQEGNVFSCSVM |
| | | | HEALHNHYTQKSLSLSPGK |
| 293 | IGG4 HC | 33342 | QLQLQESGPGLVKPSETLSLTCTVSGGSISSSS |
| | | | YYWGWIRQPPGKGLEWIGSISYSGSTYYNPSLK |
| | | | SRVTISVDTSKNQFSLKLSSVTAADTAVYYCAR |
| | | | TELGKMHFDYWGQGTLVTVSSASTKGPSVFPLA |
| | | | PCSRSTSESTAALGCLVKDYFPEPVTVSWNSGA |
| | | | LTSGVHTFPAVLQSSGLYSLSSVVTVPSSSLGT |
| | | | KTYTCNVDHKPSNTKVDKRVESKYGPPCPPCPA |
| | | | PEFLGGPSVFLFPPKPKDTLMISRTPEVTCVVV |
| | | | DVSQEDPEVQFNWYVDGVEVHNAKTKPREEQFN |
| | | | STYRVVSVLTVLHQDWLNGKEYKCKVSNKGLPS |
| | | | SIEKTISKAKGQPREPQVYTLPPSQEEMTKNQV |
| | | | SLTCLVKGFYPSDIAVEWESNGQPENNYKTTPP |
| | | | VLDSDGSFFLYSRLTVDKSRWQEGNVFSCSVMH |
| | | | EALHNHYTQKSLSLSPGK |
| 294 | IGG4 HC | 33329 | QLQLQESGPGLVKPSETLSLTCTVSGGSISSSD |
| | | | YYWGWIRQPPGKGLEWIGSIYYSGSTYYNPSLK |
| | | | SRVTISVDTSKNQFSLKLSSVTAADTAVYYCAR |
| | | | GSPRYMQDWGQGTLVTVSSASTKGPSVFPLAPC |
| | | | SRSTSESTAALGCLVKDYFPEPVTVSWNSGALT |
| | | | SGVHTFPAVLQSSGLYSLSSVVTVPSSSLGTKT |
| | | | YTCNVDHKPSNTKVDKRVESKYGPPCPPCPAPE |
| | | | FLGGPSVFLFPPKPKDTLMISRTPEVTCVVVDV |
| | | | SQEDPEVQFNWYVDGVEVHNAKTKPREEQFNST |
| | | | YRVVSVLTVLHQDWLNGKEYKCKVSNKGLPSSI |
| | | | EKTISKAKGQPREPQVYTLPPSQEEMTKNQVSL |
| | | | TCLVKGFYPSDIAVEWESNGQPENNYKTTPPVL |
| | | | DSDGSFFLYSRLTVDKSRWQEGNVFSCSVMHEA |
| | | | LHNHYTQKSLSLSPGK |
| 295 | IGG4 HC | 33351 | QVQLVESGGGLVKPGGSLRLSCAASGFTFSDYY |
| | | | MSWIRQAPGKGLEWVSYISSSGSTIYYADSVKG |
| | | | RFTISRDNAKNSLYLQMNSLRAEDTAVYYCARH |
| | | | SSLGTHNWFDPWGQGTLVTVSSASTKGPSVFPL |
| | | | APCSRSTSESTAALGCLVKDYFPEPVTVSWNSG |
| | | | ALTSGVHTFPAVLQSSGLYSLSSVVTVPSSSLG |
| | | | TKTYTCNVDHKPSNTKVDKRVESKYGPPCPPCP |
| | | | APEFLGGPSVFLFPPKPKDTLMISRTPEVTCVV |
| | | | VDVSQEDPEVQFNWYVDGVEVHNAKTKPREEQF |
| | | | NSTYRVVSVLTVLHQDWLNGKEYKCKVSNKGLP |
| | | | SSIEKTISKAKGQPREPQVYTLPPSQEEMTKNQ |
| | | | VSLTCLVKGFYPSDIAVEWESNGQPENNYKTTP |
| | | | PVLDSDGSFFLYSRLTVDKSRWQEGNVFSCSVM |
| | | | HEALHNHYTQKSLSLSPGK |
| 296 | IGG4 HC | 33357 | QVQLQESGPGLVKPSETLSLTCAVSGYSISSGY |
| | | | YWGWIRQPPGKGLEWIGSIYHSGSTYYNPSLKS |
| | | | RVTISVDTSKNQFSLKLSSVTAADTAVYYCARE |
| | | | GALSYSWLAAFDIWGQGTMVTVSSASTKGPSVF |
| | | | PLAPCSRSTSESTAALGCLVKDYFPEPVTVSWN |
| | | | SGALTSGVHTFPAVLQSSGLYSLSSVVTVPSSS |
| | | | LGTKTYTCNVDHKPSNTKVDKRVESKYGPPCPP |
| | | | CPAPEFLGGPSVFLFPPKPKDTLMISRTPEVTC |
| | | | VVVDVSQEDPEVQFNWYVDGVEVHNAKTKPREE |
| | | | QFNSTYRVVSVLTVLHQDWLNGKEYKCKVSNKG |
| | | | LPSSIEKTISKAKGQPREPQVYTLPPSQEEMTK |
| | | | NQVSLTCLVKGFYPSDIAVEWESNGQPENNYKT |
| | | | TPPVLDSDGSFFLYSRLTVDKSRWQEGNVFSCS |
| | | | VMHEALHNHYTQKSLSLSPGK |
| 297 | | | |
| 298 | | | |
| 299 | | | |
| 300 | LC | 33343 | EIVLTQSPGTLSLSPGERATLSCRASQSVSSSY |
| | | | LAWYQQKPGQAPRLLIYGASSRATGIPDRFSGS |
| | | | GSGTDFTLTISRLEPEDFAVYYCQQAVHSPYTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 301 | LC | 37268 | EIVLTQSPGTLSLSPGERATLSCRASQSVSSSY |
| | | | LAWYQQKPGQAPRLLIYGASSRATGIPDRFSGS |
| | | | GSGTDFTLTISRLEPEDFAVYYCQQAVHSPYTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 302 | LC | 37269 | EIVLTQSPGTLSLSPGERATLSCRASQSVSSSY |
| | | | LAWYQQKPGQAPRLLIYGASSRATGIPDRFSGS |
| | | | GSGTDFTLTISRLEPEDFAVYYCQQAVHSPYTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 303 | LC | 37271 | EIVLTQSPGTLSLSPGERATLSCRASQSVSSSY |
| | | | LAWYQQKPGQAPRLLIYGASSRATGIPDRFSGS |
| | | | GSGTDFTLTISRLEPEDFAVYYCQQAVHSPYTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 304 | LC | 37272 | EIVLTQSPGTLSLSPGERATLSCRASQSVSSSY |
| | | | LAWYQQKPGQAPRLLIYGASSRATGIPDRFSGS |
| | | | GSGTDFTLTISRLEPEDFAVYYCQQAVHSPYTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 305 | LC | 37277 | EIVLTQSPGTLSLSPGERATLSCGASQSVSSDY |
| | | | LAWYQQKPGQAPRLLIYGAYSLATGIPDRFSGS |
| | | | GSGTDFTLTISRLEPEDFAVYYCQWAVHSPYTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 306 | LC | 38373 | EIVLTQSPGTLSLSPGERATLSCQASQAVSSNY |
| | | | LAWYQQKPGQAPRLLIYGASSRATGIPDRFSGS |
| | | | GSGTDFTLTISRLEPEDFAVYYCQQVVHSPYTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 307 | LC | 38375 | EIVLTQSPGTLSLSPGERATLSCGASQSVSSAF |
| | | | LAWYQQKPGQAPRLLIYGASARATGIPDRFSGS |
| | | | GSGTDFTLTISRLEPEDFAVYYCQQVVHSPYTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 308 | LC | 38379 | EIVLTQSPGTLSLSPGERATLSCRASQSVSSTY |
| | | | LAWYQQKPGQAPRLLIYGASSREAGIPDRFSGS |
| | | | GSGTDFTLTISRLEPEDFAVYYCQQTVHSPYTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 309 | LC | 38381 | EIVLTQSPGTLSLSPGERATLSCQASQAVSSNY |
| | | | LAWYQQKPGQAPRLLIYGASNRAAGIPDRFSGS |
| | | | GSGTDFTLTISRLEPEDFAVYYCQQAIHSPYTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 310 | LC | 38383 | EIVLTQSPGTLSLSPGERATLSCQASQSVSSSY |
| | | | LAWYQQKPGQAPRLLIYGASNRAAGIPDRFSGS |
| | | | GSGTDFTLTISRLEPEDFAVYYCQQVVHSPYTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 311 | LC | 38386 | EIVLTQSPGTLSLSPGERATLSCKASQAVSSSY |
| | | | LAWYQQKPGQAPRLLIYGASSRQDGIPDRFSGS |
| | | | GSGTDFTLTISRLEPEDFAVYYCQQVVHSPYTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 312 | LC | 37273 | EIVLTQSPGTLSLSPGERATLSCRASQSVSSSY |
| | | | LAWYQQKPGQAPRLLIYGASSRATGIPDRFSGS |
| | | | GSGTDFTLTISRLEPEDFAVYYCQQAVHSPYTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 313 | LC | 33361 | EIVLTQSPGTLSLSPGERATLSCRASQSVSSSY |
| | | | LAWYQQKPGQAPRLLIYGASSRATGIPDRFSGS |
| | | | GSGTDFTLTISRLEPEDFAVYYCQQHSSYPPTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 314 | LC | 35624 | EIVLTQSPGTLSLSPGERATLSCRASQSVSSSY |
| | | | LAWYQQKPGQAPRLLIYGASSRATGIPDRFSGS |
| | | | GSGTDFTLTISRLEPEDFAVYYCQQHSSYPPTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 315 | LC | 38410 | EIVLTQSPGTLSLSPGERATLSCEASQSVSSSY |
| | | | LAWYQQKPGQAPRLLIYGASNRATGIPDRFSGS |
| | | | GSGTDFTLTISRLEPEDFAVYYCQQHSLYPPTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 316 | LC | 38418 | EIVLTQSPGTLSLSPGERATLSCEASQSVSASY |
| | | | LAWYQQKPGQAPRLLIYDASSRASGIPDRFSGS |
| | | | GSGTDFTLTISRLEPEDFAVYYCQQFSSYPPTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 317 | LC | 38420 | EIVLTQSPGTLSLSPGERATLSCEASQSVSSAY |
| | | | LAWYQQKPGQAPRLLIYDASTRATGIPDRFSGS |
| | | | GSGTDFTLTISRLEPEDFAVYYCQQVSSYPPTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 318 | LC | 38421 | EIVLTQSPGTLSLSPGERAALSCRVSQSVSDAY |
| | | | LAWYQQKPGQAPRLLIYDASSRASGIPDRFSGS |
| | | | GSGTDFTLTISRLEPEDFAVYYCQQVSSYPPTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 319 | LC | 38422 | EIVLTQSPGTLSLSPGERATLSCEVSQSVSASY |
| | | | LAWYQQKPGQAPRLLIYGASSRATGIPDRFSGS |
| | | | GSGTDFTLTISRLEPEDFAVYYCQQHSLYPPTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 320 | LC | 38424 | EIVLTQSPGTLSLSPGERATLSCRASQSVSSAY |
| | | | LAWYQQKPGQAPRLLIYGASDRANGIPDRFSGS |
| | | | GSGTDFTLTISRLEPEDFAVYYCQQHSLYPPTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 321 | LC | 38425 | EIVLTQSPGTLSLSPGERATLSCRASNAVSSSY |
| | | | LAWYQQKPGQAPRLLIYGASDRANGIPDRFSGS |
| | | | GSGTDFTLTISRLEPEDFAVYYCQQHSIYPPTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 322 | LC | 38426 | EIVLTQSPGTLSLSPGERATLSCRASNAVSSSY |
| | | | LAWYQQKPGQAPRLLIYGASYRATGIPDRFSGS |
| | | | GSGTDFTLTISRLEPEDFAVYYCQQHSLYPPTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 323 | LC | 37323 | DIQMTQSPSTLSASVGDRVTITCRASQSINSWL |
| | | | AWYQQKPGKAPKLLISDASSLESGVPSRFSGSG |
| | | | SGTEFTLTISSLQPDDFATYYCQQYDSHITFGG |
| | | | GTKVEIKRTVAAPSVFIFPPSDEQLKSGTASVV |
| | | | CLLNNFYPREAKVQWKVDNALQSGNSQESVTEQ |
| | | | DSKDSTYSLSSTLTLSKADYEKHKVYACEVTHQ |
| | | | GLSSPVTKSFNRGEC |
| 324 | LC | 38389 | DIQMTQSPSSVSASVGDRVTITCAASQGISSDL |
| | | | AWYQQKPGKAPKLLIYSASSTQSGVPSRFSGSG |
| | | | SGTDFTLTISSLQPEDFATYYCQQAYLYPITFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 325 | LC | 38358 | GVQMTQSPSSLSASVGDRVTITCRASQDISTYL |
| | | | NWYQQKPGKAPKLLIYDAFNLETGVPSRFSGSG |
| | | | SGTDFTFTISSLQPEDIATYYCQQLPFLPITFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 326 | LC | 33303 | DIVMTQSPLSLPVTPGEPASISCRSSQSLLHSN |
| | | | GYNYLDWYLQKPGQSPQLLIYLGSNRASGVPDR |
| | | | FSGSGSGTDFTLKISRVEAEDVGVYYCMQALGG |
| | | | PWTFGGGTKVEIKRTVAAPSVFIFPPSDEQLKS |
| | | | GTASVVCLLNNFYPREAKVQWKVDNALQSGNSQ |
| | | | ESVTEQDSKDSTYSLSSTLTLSKADYEKHKVYA |
| | | | CEVTHQGLSSPVTKSFNRGEC |
| 327 | LC | 33342 | EIVLTQSPGTLSLSPGERATLSCRASQSVSSSY |
| | | | LAWYQQKPGQAPRLLIYGASRRATGIPDRFSGS |
| | | | GSGTDFTLTISRLEPEDFAVYYCQQYVSDPITF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 328 | LC | 33299 | EIVLTQSPGTLSLSPGERATLSCRASQSVSSSY |
| | | | LAWYQQKPGQAPRLLIYGASRRATGIPDRFSGS |
| | | | GSGTDFTLTISRLEPEDFAVYYCQQVGSSPITF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 329 | LC | 33351 | DIQMTQSPSSLSASVGDRVTITCRASQSISSYL |
| | | | NWYQQKPGKAPKLLIYAASSLQSGVPSRFSGSG |
| | | | SGTDFTLTISSLQPEDFATYYCQQSHLVPRTFG |
| | | | GGTKVEIKRTVAAPSVFIFPPSDEQLKSGTASV |
| | | | VCLLNNFYPREAKVQWKVDNALQSGNSQESVTE |
| | | | QDSKDSTYSLSSTLTLSKADYEKHKVYACEVTH |
| | | | QGLSSPVTKSFNRGEC |
| 330 | LC | 33357 | EIVMTQSPATLSVSPGERATLSCRASQSVSSNL |
| | | | AWYQQKPGQAPRLLIYGASTRATGIPARFSGSG |
| | | | SGTEFTLTISSLQSEDFAVYYCQQANHHPPFTF |
| | | | GGGTKVEIKRTVAAPSVFIFPPSDEQLKSGTAS |
| | | | VVCLLNNFYPREAKVQWKVDNALQSGNSQESVT |
| | | | EQDSKDSTYSLSSTLTLSKADYEKHKVYACEVT |
| | | | HQGLSSPVTKSFNRGEC |
| 331 | | | |
| 332 | | | |
| 333 | | | |
| 334 | Fc for IGG1 | | ASTKGPSVFPLAPSSKSTSGGTAALGCLVKDYF |
| | | | PEPVTVSWNSGALTSGVHTFPAVLQSSGLYSLS |
| | | | SVVTVPSSSLGTQTYICNVNHKPSNTKVDKKVE |
| | | | PKSCDKTHTCPPCPAPEAAGAPSVFLFPPKPKD |
| | | | TLMISRTPEVTCVVVDVSHEDPEVKFNWYVDGV |
| | | | EVHNAKTKPREEQYNSTYRVVSVLTVLHQDWLN |
| | | | GKEYKCKVSNKALPAPIEKTISKAKGQPREPQV |
| | | | YTLPPSREEMTKNQVSLTCLVKGFYPSDIAVEW |
| | | | ESNGQPENNYKTTPPVLDSDGSFFLYSKLTVDK |
| | | | SRWQQGNVFSCSVMHEALHNHYTQKSLSLSPGK |
| 335 | Fc region for | | ASTKGPSVFPLAPCSRSTSESTAALGCLVKDYF |
| | IGG4 | | PEPVTVSWNSGALTSGVHTFPAVLQSSGLYSLS |
| | | | SVVTVPSSSLGTKTYTCNVDHKPSNTKVDKRVE |
| | | | SKYGPPCPPCPAPEFLGGPSVFLFPPKPKDTLM |
| | | | ISRTPEVTCVVVDVSQEDPEVQFNWYVDGVEVH |
| | | | NAKTKPREEQFNSTYRVVSVLTVLHQDWLNGKE |
| | | | YKCKVSNKGLPSSIEKTISKAKGQPREPQVYTL |
| | | | PPSQEEMTKNQVSLTCLVKGFYPSDIAVEWESN |
| | | | GQPENNYKTTPPVLDSDGSFFLYSRLTVDKSRW |
| | | | QEGNVFSCSVMHEALHNHYTQKSLSLSPGK |
| 336 | Kappa region | | RTVAAPSVFIFPPSDEQLKSGTASVVCLLNNFY |
| | for LC | | PREAKVQWKVDNALQSGNSQESVTEQDSKDSTY |
| | | | SLSSTLTLSKADYEKHKVYACEVTHQGLSSPVT |
| | | | KSFNRGEC |
| 337 | | hHLA-Gl | MVVMAPRTLFLLLSGALTLTETWAGSHSMRYFS |
| | | | AAVSRPGRGEPRFIAMGYVDDTQFVRFDSDSAC |
| | | | PRMEPRAPWVEQEGPEYWEEETRNTKAHAQTDR |
| | | | MNLQTLRGYYNQSEASSHTLQWMIGCDLGSDGR |
| | | | LLRGYEQYAYDGKDYLALNEDLRSWTAADTAAQ |
| | | | ISKRKCEAANVAEQRRAYLEGTCVEWLHRYLEN |
| | | | GKEMLQRADPPKTHVTHHPVFDYEATLRCWALG |
| | | | FYPAEIILTWQRDGEDQTQDVELVETRPAGDGT |
| | | | FQKWAAVVVPSGEEQRYTCHVQHEGLPEPLMLR |
| | | | WKQSSLPTIPIMGIVAGLVVLAAVVTGAAVAAV |
| | | | LWRKKSSD |
| 338 | | hHLA-G5 | MVVMAPRTLFLLLSGALTLTETWAGSHSMRYFS |
| | | | AAVSRPGRGEPRFIAMGYVDDTQFVRFDSDSAC |
| | | | PRMEPRAPWVEQEGPEYWEEETRNTKAHAQTDR |
| | | | MNLQTLRGYYNQSEASSHTLQWMIGCDLGSDGR |
| | | | LLRGYEQYAYDGKDYLALNEDLRSWTAADTAAQ |
| | | | ISKRKCEAANVAEQRRAYLEGTCVEWLHRYLEN |
| | | | GKEMLQRADPPKTHVTHHPVFDYEATLRCWALG |
| | | | FYPAEIILTWQRDGEDQTQDVELVETRPAGDGT |
| | | | FQKWAAVVVPSGEEQRYTCHVQHEGLPEPLMLR |
| | | | WSKEGDGGIMSVRESRSLSEDL |
| 339 | | Cyno MHC-AG | MAVMAPRTLLLVLSGVLALTQPRAGSHSMRYFY |
| | | | TAVSRPGRGQPRFIAVGYVDDTQFVRFDSDAES |
| | | | PRMEPRAPWVEQEGPEYWDRETQNMKTATQTYQ |
| | | | ANLRTLLRYYNQSEAGSHTFQKMYGCDLGPDGR |
| | | | LLRGYEQFAYDGRDYIILNEDLRSWTAADMAAQ |
| | | | NTQRKWEAAGAAEQHRTYLEGECLEWLRRYLEN |
| | | | GKETLQRADPPKTNVTHHPVSDYEATLRCWALG |
| | | | FYPAEITLTWQRDGEEQTEDTELVETRPTGDGT |
| | | | FQKWAAVVVPSGEEQRYTCHVQHEGLPKPLTLR |
| | | | WEPSSQSTILIVGIIAGLVLLGTVVTGAVVAAV |
| | | | MWRRKS |
| 340 | | Rhesus MHC-AG | LLLVLSGVLALTQTRAGSHSMRYFYTSMSRPGR |
| | | | GQPRFIAVGYVDDTQFVRFDSDAESPRMEPRAP |
| | | | WVEQEGPEYWDRETQNMKTATQTYRENLRTLLR |
| | | | YYNQSEAGSHTIQKMYGCDLGPDGRLLRGYEQF |
| | | | AYDGRDYIALNEDLRSWTAADMAAQFTQRKWEA |
| | | | AGAAEQHRTYLEGECLEWLRRYLENGKETLQRA |
| | | | DPPKTNVTHHPVSDYEATLRCWALGFYPAEITL |
| | | | TWQRDGEEQTEDTELVETRPTGDGTFQKWAAVV |
| | | | VPSGEEQRYTCHVQHEGLPEPLTLRWEPSSQST |
| | | | ILIVGIIAGLVLLGTWTGAVVAAVMWRRKSSD |
| | | | R |
| 341 | | hHLA-G ECD | MVVMAPRTLFLLLSGALTLTETWAGSHSMRYFS |
| | | with signal | AAVSRPGRGEPRFIAMGYVDDTQFVRFDSDSAC |
| | | peptide | PRMEPRAPWVEQEGPEYWEEETRNTKAHAQTDR |
| | | | MNLQTLRGYYNQSEASSHTLQWMIGCDLGSDGR |
| | | | LLRGYEQYAYDGKDYLALNEDLRSWTAADTAAQ |
| | | | 1SKRKCEAANVAEQRRAYLEGTCVEWLHRYLEN |
| | | | GKEMLQRADPPKTHVTHHPVFDYEATLRCWALG |
| | | | FYPAEIILTWQRDGEDQTQDVELVETRPAGDGT |
| | | | FQKWAAVVVPSGEEQRYTCHVQHEGLPEPLMLR |
| | | | W |
| 342 | | hHLA-G ECD | GSHSMRYFSAAVSRPGRGEPRFIAMGYVDDTQF |
| | | without signal | VRFDSDSACPRMEPRAPWVEQEGPEYWEEETRN |
| | | peptide | TKAHAQTDRMNLQTLRGYYNQSEASSHTLQWMI |
| | | | GCDLGSDGRLLRGYEQYAYDGKDYLALNEDLRS |
| | | | WTAADTAAQISKRKCEAANVAEQRRAYLEGTCV |
| | | | EWLHRYLENGKEMLQRADPPKTHVTHHPVFDYE |
| | | | ATLRCWALGFYPAEIILTWQRDGEDQTQDVELV |
| | | | ETRPAGDGTFQKWAAVVVPSGEEQRYTCHVQHE |
| | | | GLPEPLMLRW |
| 343 | | | |
| 344 | | | |
| 345 | | | |
| 346 | CDR-H1 | Chothia | GYSFTGY |
| | CD3 | | |
| 347 | CDR-H1 | Chothia | GYTFTRY |
| | CD3 | | |
| 348 | CDR-H1 | Chothia | GFKFSGY |
| | CD3 | | |
| 349 | CDRH-1 | Chothia | GYTFTNY |
| | CD3 | | |
| 350 | | | |
| 351 | | | |
| 352 | | | |
| 353 | | | |
| 354 | CDR-H1 | Kabat | GYTMN |
| | CD3 | | |
| 355 | CDR-H1 | Kabat | RYTMH |
| | CD3 | | |
| 356 | CDR-H1 | Kabat | GYGMH |
| | CD3 | | |
| 357 | CDR-H1 | Kabat | NYYIH |
| | CD3 | | |
| 358 | | | |
| 359 | | | |
| 360 | | | |
| 361 | | | |
| 362 | CDR-H2 | Chothia | NPYKGV |
| | CD3 | | |
| 363 | CDR-H2 | Chothia | WYDGSK |
| | CD3 | | |
| 364 | CDR-H2 | Chothia | NPSRGY |
| | CD3 | | |
| 365 | CDR-H2 | Chothia | YPGDGN |
| | CD3 | | |
| 366 | | | |
| 367 | | | |
| 368 | | | |
| 369 | | | |
| 370 | | | |
| 371 | CDR-H2 | Kabat | LINPYKGVSTYNQKFKD |
| | CD3 | | |
| 372 | CDR-H2 | Kabat | YINPSRGYTNYNQKFKD |
| | CD3 | | |
| 373 | CDR-H2 | Kabat | YINPSRGYTNYNQKVKD |
| | CD3 | | |
| 374 | CDR-H2 | Kabat | WIYPGDGNTKYNEKEKG |
| | CD3 | | |
| 375 | CDR-H2 | Kabat | VIWYDGSKKYYVDSVKG |
| | CD3 | | |
| 376 | | | |
| 377 | | | |
| 378 | | | |
| 379 | CDR-H3 | | SGYYGDSDWYFDV |
| | CD3 | | |
| 380 | CDR-H3 | | DSYSNYYFDY |
| | CD3 | | |
| 381 | CDR-H3 | | QMGYWHFDL |
| | CD3 | | |
| 382 | CDR-H3 | | YYDDHYCLDY |
| | CD3 | | |
| 383 | | | |
| 384 | | | |
| 385 | | | |
| 386 | | | |
| 387 | | | |
| 388 | CDR-L1 | | RASQDIRNYLN |
| | CD3 | | |
| 389 | CDR-L1 | | SASSVSYMS |
| | CD3 | | |
| 390 | CDR-L1 | | RASSSVSYMN |
| | CD3 | | |
| 391 | CDR-L1 | | KSSQSLLNSRTRKNYLA |
| | CD3 | | |
| 392 | CDR-L1 | | RASQSVSSYLA |
| | CD3 | | |
| 393 | | | |
| 394 | | | |
| 395 | | | |
| 396 | CDR-L2 | | YTSRLHS |
| | CD3 | | |
| 397 | CDR-L2 | | DTSKLAS |
| | CD3 | | |
| 398 | CDR-L2 | | DTSKVAS |
| | CD3 | | |
| 399 | CDR-L2 | | WASTRES |
| | CD3 | | |
| 400 | CDR-L2 | | DASNRAT |
| | CD3 | | |
| 401 | | | |
| 402 | | | |
| 403 | | | |
| 404 | CDR-L3 | | QQGNTLPWT |
| | CD3 | | |
| 405 | CDR-L3 | | QQWSSNPFT |
| | CD3 | | |
| 406 | CDR-L3 | | QQWSSNPLT |
| | CD3 | | |
| 407 | CDR-L3 | | TQSFILRT |
| | CD3 | | |
| 408 | CDR-L3 | | QQRSNWPPLT |
| | CD3 | | |
| 409 | | | |
| 410 | | | |
| 411 | | | |
| 412 | | | |
| 413 | VH | | EVQLQQSGPELVKPGASMKISCKASGYSFTGYT |
| | CD3 | | MNWVKQSHGKNLEWMGLINPYKGVSTYNQKFKD |
| | | | KATLTVDKSSSTAYMELLSLTSEDSAVYYCARS |
| | | | GYYGDSDWYFDVWGQGTTVTVSS |
| 414 | VH | | QVQLQQSGAELARPGASVKMSCKASGYTFTRYT |
| | CD3 | | MHWVKQRPGQGLEWIGYINPSRGYTNYNQKFKD |
| | | | KATLTTDKSSSTAYMQLSSLTSEDSAVYYCARY |
| | | | YDDHYCLDYWGQGTTLTVSS |
| 415 | ScFV | | DIKLQQSGAELARPGASVKMSCKTSGYTFTRYT |
| | CD3 | | MHWVKQRPGQGLEWIGYINPSRGYTNYNQKFKD |
| | | | KATLTTDKSSSTAYMQLSSLTSEDSAVYYCARY |
| | | | YDDHYCLDYWGQGTTLTVSS |
| 416 | VH | | EVQLVQSGAEVKKPGASVKVSCKASGYTFTNYY |
| | CD3 | | IHWVRQAPGQGLEWIGWIYPGDGNTKYNEKEKG |
| | | | RATLTADTSTSTAYLELSSLRSEDTAVYYCARD |
| | | | SYSNYYFDYWGQGTLVTVSS |
| 417 | VH | | QVQLVESGGGVVQPGRSLRLSCAASGFKFSGYG |
| | CD3 | | MHWVRQAPGKGLEWVAVIWYDGSKKYYVDSVKG |
| | | | RFTISRDNSKNTLYLQMNSLRAEDTAVYYCARQ |
| | | | MGYWHFDLWGRGTLVTVSS |
| 418 | VH | | QVQLVQSGGGVVQPGRSLRLSCKASGYTFTRYT |
| | CD3 | | MHWVRQAPGKGLEWIGYINPSRGYTNYNQKVKD |
| | | | RFTISRDNSKNTAFLQMDSLRPEDTGVYFCARY |
| | | | YDDHYCLDYWGQGTPVTVSS |
| 419 | | | |
| 420 | | | |
| 421 | | | |
| 422 | VL | | DIQMTQTTSSLSASLGDRVTISCRASQDIRNYL |
| | CD3 | | NWYQQKPDGTVKLLIYYTSRLHSGVPSKFSGSG |
| | | | SGTDYSLTISNLEQEDIATYFCQQGNTLPWTFA |
| | | | GGTKLEIK |
| 423 | VL | | QIVLTQSPAIMSASPGEKVTMTCSASSSVSYMN |
| | CD3 | | WYQQKSGTSPKRWIYDTSKLASGVPAHFRGSGS |
| | | | GTSYSLTISGMEAEDAATYYCQQWSSNPFTFGS |
| | | | GTKLEIK |
| 424 | ScFV LCVR | | DIQLTQSPAIMSASPGEKVTMTCRASSSVSYMN |
| | CD3 | | WYQQKSGTSPKRWIYDTSKVASGVPYRFSGSGS |
| | | | GTSYSLTISSMEAEDAATYYCQQWSSNPLTFGA |
| | | | GTKLELK |
| 425 | VL | | DIVMTQSPDSLAVSLGERATINCKSSQSLLNSR |
| | CD3 | | TRKNYLAWYQQKPGQPPKLLIYWASTRESGVPD |
| | | | RESGSGSGTDETLTISSLQAEDVAVYYCTQSFI |
| | | | LRTFGQGTKVEIK |
| 426 | VL | | EIVLTQSPATLSLSPGERATLSCRASQSVSSYL |
| | CD3 | | AWYQQKPGQAPRLLIYDASNRATGIPARFSGSG |
| | | | SGTDFTLTISSLEPEDFAVYYCQQRSNWPPLTF |
| | | | GGGTKVEIK |
| 427 | VL | | DIQMTQSPSSLSASVGDRVTITCSASSSVSYMN |
| | CD3 | | WYQQTPGKAPKRWIYDTSKLASGVPSRFSGSGS |
| | | | GTDYTFTISSLQPEDIATYYCQQWSSNPFTFGQ |
| | | | GTKLQIT |
| 428 | | | |
| 429 | | | |
| 430 | | | |
| 431 | CDR-H1 | Chothia | GFSLSTSGM |
| | CD16 | | |
| 432 | CDR-H1 | Chothia | GFTFNNY |
| | CD16 | | |
| 433 | CDR-H1 | Chothia | GFTFSNY |
| | CD16 | | |
| 434 | CDR-H1 | Chothia | GYTFTSS |
| | CD16 | | |
| 435 | CDR-H1 | Chothia | GFTFSNYG |
| | CD16 | | |
| 436 | CDR-H1 | Chothia | GFTFSSYG |
| | CD16 | | |
| 437 | CDR-H1 | Chothia | GYTFTSYY |
| | CD16 | | |
| 438 | | | |
| 439 | | | |
| 440 | | | |
| 441 | CDR-H1 | Kabat | TSGMGVG |
| | CD16 | | |
| 442 | CDR-H1 | Kabat | TSGVGVG |
| | CD16 | | |
| 443 | CDR-H1 | Kabat | NYGMS |
| | CD16 | | |
| 444 | CDR-H1 | Chothia | SSAMH |
| | CD16 | | |
| 445 | | | |
| 446 | | | |
| 447 | | | |
| 448 | CDR-H2 | Chothia | WWDDD |
| | CD16 | | |
| 449 | CDR-H2 | Chothia | SGGGSY |
| | CD16 | | |
| 450 | CDR-H2 | Chothia | NHYNDG |
| | CD16 | | |
| 451 | CDR-H2 | Chothia | IYYSGGST |
| | CD16 | | |
| 452 | CDR-H2 | Chothia | VNHSGGST |
| | CD16 | | |
| 453 | CDR-H2 | Chothia | IEPMYGST |
| | CD16 | | |
| 454 | CDR-H2 | Chothia | INPSGGST |
| | CD16 | | |
| 455 | | | |
| 456 | | | |
| 457 | | | |
| 458 | CDR-H2 | Kabat | YINHYNDGIKYNERFKG |
| | CD16 | | |
| 459 | CDR-H2 | Kabat | HIWWDDDKRYNPALKS |
| | CD16 | | |
| 460 | CDR-H2 | Kabat | HIWWDDDKRYSPSLKS |
| | CD16 | | |
| 461 | CDR-H2 | Kabat | TISGGGSYTFYPDSVKG |
| | CD16 | | |
| 462 | | | |
| 463 | | | |
| 464 | | | |
| 465 | CDR-H3 | | TNPAWFAY |
| | CD16 | | |
| 466 | CDR-H3 | | INPAYFAY |
| | CD16 | | |
| 467 | CDR-H3 | | QSARAPEPY |
| | CD16 | | |
| 468 | CDR-H3 | | QSANSPVPY |
| | CD16 | | |
| 469 | CDR-H3 | | GYRYASWFAS |
| | CD16 | | |
| 470 | CDR-H3 | | ARESIDY |
| | CD16 | | |
| 471 | CDR-H3 | | ARVGSFDF |
| | CD16 | | |
| 472 | CDR-H3 | | ARGSAYYYDFADY |
| | CD16 | | |
| 473 | | | |
| 474 | | | |
| 475 | | | |
| 476 | CDR-L1 | | KASQSVDFDGDSFMN |
| | CD16 | | |
| 477 | CDR-L1 | | KASQNVGTHVA |
| | CD16 | | |
| 478 | CDR-L1 | | RASVNVGTHVA |
| | CD16 | | |
| 479 | CDR-L1 | | RASQNIGTSIH |
| | CD16 | | |
| 480 | CDR-L1 | | NIGSKN |
| | CD16 | | |
| 481 | | | |
| 482 | | | |
| 483 | | | |
| 484 | CDR-L2 | | TTSNLES |
| | CD16 | | |
| 485 | CDR-L2 | | TTSSLES |
| | CD16 | | |
| 486 | CDR-L2 | | SASYRYS |
| | CD16 | | |
| 487 | CDR-L2 | | SVSESIS |
| | CD16 | | |
| 488 | CDR-L2 | | QDN |
| | CD16 | | |
| 489 | | | |
| 490 | | | |
| 491 | | | |
| 492 | CDR-L3 | | QQSNEDPYT |
| | CD16 | | |
| 493 | CDR-L3 | | QQSNSDPYT |
| | CD16 | | |
| 494 | CDR-L3 | | QQYKSYPLT |
| | CD16 | | |
| 495 | CDR-L3 | | QDYTNYPLT |
| | CD16 | | |
| 496 | CDR-L3 | | QQSNSWPLT |
| | CD16 | | |
| 497 | CDR-L3 | | QVWDNYSVL |
| | CD16 | | |
| 498 | | | |
| 499 | | | |
| 500 | | | |
| 501 | VH | | QVTLRESGPALVKPTQTLTLTCTFSGFSLSTSG |
| | CD16 | | MGVGWIRQPPGKALEWLAHIWWDDDKRYNPALK |
| | | | SRLTISKDTSKNQVVLTMTNMDPVDTATYYCAR |
| | | | TNPAWFAYWGQGTLVTVSS |
| 502 | VH | | QVTLRESGPALVKPTQTLTLTCTFSGFSLSTSG |
| | CD16 | | MGVGWIRQPPGKALEWLAHIWWDDDKRYNPALK |
| | | | SRLTTSKDTSKNQWLTNTNMDPVDTATYYCAQ |
| | | | TNPAWFAYWGQGTLVTVSS |
| 503 | VH | | QVTLRESGPALVKPTQTLTLTCTFSGFSLSTSG |
| | CD16 | | VGVGWIRQPPGKALEWLAHIWWDDDKRYSPSLK |
| | | | SRLTISKDTSKNQVVLTMTNMDPVDTATYYCAR |
| | | | INPAYFAYWGQGTLVTVS |
| 504 | VH | | EVKLVESGGTLVKPGGSLKLSCAASGFTFNNYG |
| | CD16 | | MSWVRQTPEKRLEWVATISGGGSYTFYPDSVKG |
| | | | RFTISRDNAKNSLYLQMSSLRSEDTALYYCIRQ |
| | | | SARAPEPYWGQGTLVTVSS |
| 505 | VH | | EVQLVESGGGLVKPGGSLRLSCAASGFTFSNYG |
| | CD16 | | MSWVRQAPGKGLEWVATISGGGSYTFYPDSVKG |
| | | | RFTISRDNAKNSLYLQMNSLRTEDTALYYCVRQ |
| | | | SARAPEPYWGQGTLVTVSS |
| 506 | VH | | EVQLVESGGGLVKPGGSLRLSCAASGFTFSNYG |
| | CD16 | | MSWVRQAPGKGLEWVATISGGGSYTFYPDSVKG |
| | | | RFTISRDNAKNSLYLQMNSLRTEDTALYYCVRQ |
| | | | SANSPVPYWGQGTLVTVSS |
| 507 | VH | | EVQLQQSGPELVKPGASVKMSCKASGYTFTSSA |
| | CD16 | | MHWVKKNPGQGLEWIGYINHYNDGIKYNERFKG |
| | | | KATLTSDKSSSTAYMELSSLTSEDSAVYYCATG |
| | | | YRYASWFASWGQGTLVTVSS |
| 508 | VH | | QVQLVQSGAEVKKPGASVEVSCKASGYTFTSSA |
| | CD16 | | MHWVRQAPGQGLEWMGYINHYNDGIKYNERFKG |
| | | | RVTITALKSTSTAYMELSSLRSEDTAVYYCATG |
| | | | YRYASWFASWGQGTLVTVSS |
| 509 | VH | | QVQLVQSGAEVKKPGASVKVSCKASGYTFTSSA |
| | CD16 | | MHWVRQAPGQGLEWMGYINHYNDGIKYNERFKG |
| | | | RVTITADKSTSTAYMELSSLRSEDTAVYYCARG |
| | | | YRYASWFASWGQGTLVTVSS |
| 510 | VH | | EVQLVESGGGLVQPGGSLRLSCAASGFTFSNYG |
| | CD16 | | MSWVRQAPGKGLEWIGSLYYSGGSTNYNPSLKG |
| | | | SLVISRDNSKNTLYLQMNSLAEDTATYYC |
| | | | ARESIDYWGQGTLVTVSS |
| 511 | VH | | EVQLVESGGGLVQPGGSLRLSCAASGFTFSSYG |
| | CD16 | | MSWVRQAPGKGLEWIGEVNHSGGSTNYNPSLKG |
| | | | SFVISRDNSKNTLYLQMNSLAEDTAVYYCARVG |
| | | | SFDFWGQGTLVTVSS |
| 512 | VH | | QVQLVQSGAEVKKPGESLKVSCKASGYTFTSYY |
| | CD16 | | MHWVRQAPGQGLEWMGAIEPMYGSTSYAQKFQG |
| | | | RVTMTRDTSTSTVYMELSSLRSEDTAVYYCARG |
| | | | SAYYYDFADYWGQGTLVTVSS |
| 513 | VH | | QVQLVQSGAEVEKPGBSLEVSCKASGYTFTSYY |
| | CD16 | | MHWVRQAPGQGLEWMGIINPSGGSTSYAQKFQG |
| | | | RVTMTRDTSTSTVYMELSSLRSEDTAVYYCARG |
| | | | SAYYYDFADYWGQGTLVTVSS |
| 514 | | | |
| 515 | | | |
| 516 | | | |
| 517 | VL | | DIVMTQSPDSLAVSLGERATINCKASQSVDFDG |
| | CD16 | | DSFMNWYQQKPGQPPKLLIYTTSNLESGVPDRF |
| | | | SGSGSGTDFTLTISSLQAEDVAVYYCQQSNEDP |
| | | | YTFGQGTKLEIK |
| 518 | VL | | DIVMTQSPDSLAVSLGERATINCKSSQSVDFDG |
| | CD16 | | DSFMNWYQQKPGQPPKLLIYTTSSLESGVPDRF |
| | | | SGSGSGTDFTLTISSLQAEDVAVYYCQQSNSDP |
| | | | YTFGQGTKLEIK |
| 519 | VL | | DIVMTQSQKFMSTSVGDRVSVTCKASQNVGTHV |
| | CD16 | | AWYQQKSGQSPKSLLYSASYRYSGVPDRFSGSG |
| | | | SGTDFTLTISNVQSBDLAEYFCQQYKSYPLTFG |
| | | | AGTKLELK |
| 520 | VL | | DIQMTOSPSFLSASVGDRVTITCRASQNVGTHV |
| | CD16 | | AWYQQKPGKAPESLLYSASYRYSGVPSRFSGSG |
| | | | SGTDFTLTISSLQSEDIATYYCQQYKSYPLTFG |
| | | | QGTKLEIK |
| 521 | VL | | DIQMTQSPSFLSASVGDRVTITCRASQNVGTHV |
| | CD16 | | AWYQQKPGKAPKSLLYSASYRYSGVPSRFSGSG |
| | | | SGTDFTLTISSLQSEDIATYYCQDYTNYPLTFG |
| | | | QGTKLEIK |
| 522 | VL | | DILLTQSPAILSVSPGERVSFSCRASQNIGTSI |
| | CD16 | | HWYQQRTDGSPRLLIKSVSESISGIPSRFSGSG |
| | | | SGTDFTLTINGVESGDISDYYCQQSNSWPLTFG |
| | | | AGTKLELK |
| 523 | VL | | EIVLTQSPATLSVSPGERATLSCRASQNIGTSI |
| | CD16 | | HWYQQKPDQSPKLLIKSVSESISGVPSRFSGSG |
| | | | SGTDFTLTINSLEAEDFATYYCQQSNSWPLTFG |
| | | | QGTKLEIK |
| 524 | VL | | SYVLTQPSSVSVAPGQTATISCGGHNIGSKNVH |
| | CD16 | | WYQQRPGQSPVLVIYQDNKRPSGIPERFSGSNS |
| | | | GNTATLTISGTQAMDEADYYCQVWDNYSVLFGG |
| | | | GTKLTVL |
| 525 | | | |
| 526 | | | |
| 527 | | | |
| 528 | CDR-H1 | Chothia | GFSLSTYGI |
| | NKp46 | | |
| 529 | | | |
| 530 | | | |
| 531 | | | |
| 532 | CDR-H1 | Kabat | TYGIGVG |
| | NKp46 | | |
| 533 | | | |
| 534 | | | |
| 535 | | | |
| 536 | CDR-H2 | Chothia | WWNDN |
| | NKp46 | | |
| 537 | | | |
| 538 | | | |
| 539 | | | |
| 540 | CDR-H2 | Kabat | HIWWNDNEYYNIDLKS |
| | NKp46 | | |
| 541 | | | |
| 542 | | | |
| 543 | | | |
| 544 | CDR-H3 | | GNYRYARGYVMDY |
| | NKp46 | | |
| 545 | | | |
| 546 | | | |
| 547 | | | |
| 548 | CDR-L1 | | RASESVEYYGTSLMQ |
| | NKp46 | | |
| 549 | | | |
| 550 | | | |
| 551 | | | |
| 552 | CDR-L2 | | AASNVES |
| | NKp46 | | |
| 553 | | | |
| 554 | | | |
| 555 | | | |
| 556 | CDR-L3 | | QQNRKVPWT |
| | NKp46 | | |
| 557 | | | |
| 558 | | | |
| 559 | | | |
| 560 | VH | | QVTLKESGPGILQPSQTLSLTCSFSGFSLSTYG |
| | NKp46 | | IGVGWNRQPSGKGLEWLAHIWWNDNEYYNIDLK |
| | | | SRLTISKDTSNNQVFLKIASVDTADTATYYCVR |
| | | | GNYRYARGYVMDYWGQGTSVTVSS |
| 561 | | | |
| 562 | | | |
| 563 | | | |
| 564 | VL | | DIVLTQSPASLAVSLGQRATISCRASESVEYYG |
| | NKp46 | | TSLMQWYQQKPGQPPKLLIYAASNVESGVPARF |
| | | | SGSGSGTDFSLNIHPVEEDDFAMYFCQQNRKVP |
| | | | WTFGGGTKLEIK |
| 565 | | | |
| 566 | | | |
| 567 | | | |
| 568 | CDR-H1 | Chothia | GHTFTSY |
| | NKp30 | | |
| 569 | CDR-H1 | Chothia | GGTFTSY |
| | NKp30 | | |
| 570 | CDR-H1 | Chothia | GHTFTNY |
| | NKp30 | | |
| 571 | | | |
| 572 | | | |
| 573 | | | |
| 574 | CDR-H1 | Kabat | SYFMH |
| | NKp30 | | |
| 575 | CDR-H1 | Kabat | SYSVS |
| | NKp30 | | |
| 576 | CDR-H1 | Kabat | NYYMH |
| | NKp30 | | |
| 577 | | | |
| 578 | | | |
| 579 | | | |
| 580 | CDR-H2 | Chothia | NPSDDY |
| | NKp30 | | |
| 581 | CDR-H2 | Chothia | VPIFGT |
| | NKp30 | | |
| 582 | CDR-H2 | Chothia | NPNGGD |
| | NKp30 | | |
| 583 | | | |
| 584 | | | |
| 585 | | | |
| 586 | CDR-H2 | Kabat | IINPSDDYANYAQKFQG |
| | NKp30 | | |
| 587 | CDR-H2 | Kabat | GIVPIFGTADYAQKFQG |
| | NKp30 | | |
| 588 | CDR-H2 | Kabat | VINPNGGDTSYAQKFQG |
| | NKp30 | | |
| 589 | | | |
| 590 | | | |
| 591 | | | |
| 592 | CDR-H3 | | AIFDY |
| | NKp30 | | |
| 593 | CDR-H3 | | GYSYGQTFDY |
| | NKp30 | | |
| 594 | CDR-H3 | | DRAWYGGNVRAEDI |
| | NKp30 | | |
| 595 | | | |
| 596 | | | |
| 597 | | | |
| 598 | CDR-L1 | | RASQSISSYLN |
| | NKp30 | | |
| 599 | CDR-L1 | | RSSQSLLHSNGYNYLD |
| | NKp30 | | |
| 600 | | | |
| 601 | | | |
| 602 | | | |
| 603 | CDR-L2 | | AASSLQS |
| | NKp30 | | |
| 604 | CDR-L2 | | LGSNRAS |
| | NKp30 | | |
| 605 | | | |
| 606 | | | |
| 607 | | | |
| 608 | CDR-L3 | | QQSYSTPLT |
| | NKp30 | | |
| 609 | CDR-L3 | | MQALQTPYT |
| | NKp30 | | |
| 610 | | | |
| 611 | | | |
| 612 | | | |
| 613 | VH | | QVQLVQSGAEVKKPGASVKVSCKASGHTFTSYF |
| | NKp30 | | MHWVRQAPGQGLEWMGIINPSDDYANYAQKFQG |
| | | | RVTMTRDTSTSTVYMELSSLRSEDTAVYYCATA |
| | | | IFDYWGQGTLVTVSS |
| 614 | VH | | QVQLVQSGAEVKKPGASVKVSCKASGHTFTSYF |
| | NKp30 | | MHWVRQAPGQGLEWMGIINPSDDYANYAQKFQG |
| | | | RVTMTRDTSTSTVYMELSSLRSEDTAVYYCATA |
| | | | IFDYWGQGTPVTVSS |
| 615 | VH | | QVQLVQSGAEVKKPGASVKVSCKASGHTFTNYY |
| | NKp30 | | MHWVRQAPGQGLEWMGVINPNGGDTSYAQKFQG |
| | | | RVTMTRDTSTSTVYMELSSLRSEDTAVYYCARD |
| | | | RAWDYGGNVRAFDIWGQGTMVTVSS |
| 616 | VH | | QVQLVQSGAEVKKPGSSVKVSCKASGGTFTSYS |
| | NKp30 | | VSWVRQAPGQGLEWMGGIVPIFGTADYAQKFQG |
| | | | RVTITADESTSTAYMELSSLRSEDTAVYYCARG |
| | | | YSYGQTFDYWGQGTLVTVSS |
| 617 | | | |
| 618 | | | |
| 619 | | | |
| 620 | | | |
| 621 | | | |
| 622 | | | |
| 623 | | | |
| 624 | VL | | DIQMTQSPSSLSASVGDRVTITCRASQSISSYL |
| | NKp30 | | NWYQQKPGKAPKLLIYAASSLQSGVPSRFSGSG |
| | | | SGTDFTLTISSLQPEDFATYYCQQSYSTPLTFG |
| | | | GGTKVEIK |
| 625 | VL | | DIVMTQSPLSLPVTPGEPASISCRSSQSLLHSN |
| | NKp30 | | GYNYLDWYLQKPGQSPQLLIYLGSNRASGVPDR |
| | | | FSGSGSGTDFTLKISRVEAEDVGVYYCMQALQT |
| | | | PYTFGGGTKVEIK |
| 626 | | | |
| 627 | | | |
| 628 | | | |
| 629 | CD3ϵ | | MQSGTHWRVLGLCLLSVGVWGQDGNEEMGGITQ |
| | Sequence | | TPYKVSISGTTVILTCPQYPGSEILWQHNDKNI |
| | | | GGDEDDKNIGSDEDHLSLKEFSELEQSGYYVCY |
| | | | PRGSKPEDANFYLYLRARVCENCMEMDVMSVAT |
| | | | IVIVDICITGGLLLLVYYWSKNRKAKAKPVTRG |
| | | | AGAGGRQRGQNKERPPPVPNPDYEPIRKGQRDL |
| | | | YSGLNQRRI |
| 630 | CD16A | | MWQLLLPTALLLLVSAGMRTEDLPKAVVFLEPQ |
| | Sequence | | WYRVLEKDSVTLKCQGAYSPEDNSTQWFHNESL |
| | | | ISSQASSYFIDAATVDDSGEYRCQTNLSTLSDP |
| | | | VQLEVHIGWLLLQAPRWVFKEEDPIHLRCHSWK |
| | | | NTALHKVTYLQNGKGRKYFHHNSDFYIPKATLK |
| | | | DSGSYFCRGLFGSKNVSSETVNITITQGLAVST |
| | | | ISSFFPPGYQVSFCLVMVLLFAVDTGLYFSVKT |
| | | | NIRSSTRDWKDHKFKWRKDPQDK |
| 631 | CD16B | | MWQLLLPTALLLLVSAGMRTEDLPKAVVFLEPQ |
| | Sequence | | WYSVLEKDSVTLKCQGAYSPEDNSTQWFHNESL |
| | | | ISSQASSYFIDAATVNDSGEYRCQTNLSTLSDP |
| | | | VQLEVHIGWLLLQAPRWVFKEEDPIHLRCHSWK |
| | | | NTALHKVTYLQNGKDRKYFHHNSDFHIPKATLK |
| | | | DSGSYFCRGLVGSKNVSSETVNITITQGLAVST |
| | | | ISSFSPPGYQVSFCLVMVLLFAVDTGLYFSVKT |
| | | | NI |
| 632 | NKp46 | | MSSTLPALLCVGLCLSQRISAQQQTLPKPFIWA |
| | Sequence | | EPHFMVPKEKQVTICCQGNYGAVEYQLHFEGSL |
| | | | FAVDRPKPPERINKVKFYIPDMNSRMAGQYSCI |
| | | | YRVGELWSEPSNLLDLVVTEMYDTPTLSVHPGP |
| | | | EVISGEKVTFYCRLDTATSMFLLLKEGRSSHVQ |
| | | | RGYGKVQAEFPLGPVTTAHRGTYRCFGSYNNHA |
| | | | WSFPSEPVKLLVTGDIENTSLAPEDPTFPADTW |
| | | | GTYLLTTETGLQKDHALWDHTAQNLLRMGLAFL |
| | | | VLVALVWFLVEDWLSRKRTRERASRASTWEGRR |
| | | | RLNTQTL |
| 633 | NKp46 | | MSSTLPALLCVGLCLSQRISAQQQTLPKPFIWA |
| | Sequence | | EPHFMVPKEKQVTICCQGNYGAVEYQLHFEGSL |
| | | | FAVDRPKPPERINKVKFYIPDMNSRMAGQYSCI |
| | | | YRVGELWSEPSNLLDLVVTEMYDTPTLSVHPGP |
| | | | EVISGEKVTFYCRLDTATSMFLLLKEGRSSHVQ |
| | | | RGYGKVQAEFPLGPVTTAHRGTYRCFGSYNNHA |
| | | | WSFPSEPVKLLVTGDIENTSLAPEDPTFPDHAL |
| | | | WDHTAQNLLRMGLAFLVLVALVWFLVEDWLSRK |
| | | | RTRERASRASTWEGRRRLNTQTL |
| 634 | NKp4 6 | | MSSTLPALLCVGLCLSQRISAQQQMYDTPTLSV |
| | Sequence | | HPGPEVISGEKVTFYCRLDTATSMFLLLKEGRS |
| | | | SHVQRGYGKVQAEFPLGPVTTAHRGTYRCFGSY |
| | | | NNHAWSFPSEPVKLLVTGDIENTSLAPEDPTFP |
| | | | ADTWGTYLLTTETGLQKDHALWDHTAQNLLRMG |
| | | | LAFLVLVALVWFLVEDWLSRKRTRERASRASTW |
| | | | EGRRRLNTQTL |
| 635 | NKp46 | | MSSTLPALLCVGLCLSQRISAQQQMYDTPTLSV |
| | Sequence | | HPGPEVISGEKVTFYCRLDTATSMFLLLKEGRS |
| | | | SHVQRGYGKVQAEFPLGPVTTAHRGTYRCFGSY |
| | | | NNHAWSFPSEPVKLLVTGDIENTSLAPEDPTFP |
| | | | DHALWDHTAQNLLRMGLAFLVLVALVWFLVEDW |
| | | | LSRKRTRERASRASTWEGRRRLNTQTL |
| 636 | NKp46 | | MSSTLPALLCVEMYDTPTLSVHPGPEVISGEKV |
| | Sequence | | TFYCRLDTATSMFLLLKEGRSSHVQRGYGKVQA |
| | | | EFPLGPVTTAHRGTYRCFGSYNNHAWSFPSEPV |
| | | | KLLVTGDIENTSLAPEDPTFPADTWGTYLLTTE |
| | | | TGLQKDHALWDHTAQNLLRMGLAFLVLVALVWF |
| | | | LVEDWLSRKRTRERASRASTWEGRRRLNTQTL |
| 637 | NKp46 | | MSSTLPALLCVGLCLSQRISAQQQTLPKPFIWA |
| | | | EPHFMVPKEKQVTICCQGNYGAVEYQLHFEGSL |
| | | | FAVDRPKPPERINKVKFYIPDMNSRMAGQYSCI |
| | | | YRVGELWSEPSNLLDLVVTEMYDTPTLSVHPGP |
| | | | EVISGEKVTFYCRLDTATSMFLLLKEGRSSHVQ |
| | | | RGYGKVQAEFPLGPVTTAHRGTYRCFGSYNNHA |
| | | | WSFPSEPVKLLVTGDIENTSLAPEDPTFPDTWG |
| | | | TYLLTTETGLQKDHALWDHTAQNLLRMGLAFLV |
| | | | LVALVWFLVEDWLSRKRTRERASRASTWEGRRR |
| | | | LNTQTL |
| 638 | NKp30 | | MAWMLLLILIMVHPGSCALWVSQPPEIRTLEGS |
| | Sequence | | SAFLPCSFNASQGRLAIGSVTWFRDEVVPGKEV |
| | | | RNGTPEFRGRLAPLASSRFLHDHQAELHIRDVR |
| | | | GHDASIYVCRVEVLGLGVGTGNGTRLVVEKEHP |
| | | | QLGAGTVLLLRAGFYAVSFLSVAVGSTVYYQGK |
| | | | CLTWKGPRRQLPAVVPAPLPPPCGSSAHLLPPV |
| | | | PGG |
| 639 | NKp30 | | MAWMLLLILIMVHPGSCALWVSQPPEIRTLEGS |
| | Sequence | | SAFLPCSFNASQGRLAIGSVTWFRDEVVPGKEV |
| | | | RNGTPEFRGRLAPLASSRFLHDHQAELHIRDVR |
| | | | GHDASIYVCRVEVLGLGVGTGNGTRLVVEKEHP |
| | | | QLGAGTVLLLRAGFYAVSFLSVAVGSTVYYQGK |
| | | | CHCHMGTHCHSSDGPRGVIPEPRCP |
| 640 | NKp30 | | MAWMLLLILIMVHPGSCALWVSQPPEIRTLEGS |
| | Sequence | | SAFLPCSFNASQGRLAIGSVTWFRDEVVPGKEV |
| | | | RNGTPEFRGRLAPLASSRFLHDHQAELHIRDVR |
| | | | GHDASIYVCRVEVLGLGVGTGNGTRLVVEKEHP |
| | | | QLGAGTVLLLRAGFYAVSFLSVAVGSTVYYQGK |
| | | | YAKSTLSGFPQL |
| 641 | NKp30 | | MAWMLLLILIMVHPGSCALWVSQPPEIRTLEGS |
| | | | SAFLPCSFNASQGRLAIGSVTWFRDEVVPGKEA |
| | | | ELHIRDVRGHDASIYVCRVEVLGLGVGTGNGTR |
| | | | LVVEKEHPQLGAGTVLLLRAGFYAVSFLSVAVG |
| | | | STVYYQGKCLTWKGPRRQLPAVVPAPLPPPCGS |
| | | | SAHLLPPVPGG |
| 642 | NKp30 | | MAWMLLLILIMVHPGSCALWVSQPPEIRTLEGS |
| | | | SAFLPCSFNASQGRLAIGSVTWFRDEVVPGKEA |
| | | | ELHIRDVRGHDASIYVCRVEVLGLGVGTGNGTR |
| | | | LVVEKEHPQLGAGTVLLLRAGFYAVSFLSVAVG |
| | | | STVYYQGKCHCHMGTHCHSSDGPRGVIPEPRCP |
| 643 | NKp30 | | MAWMLLLILIMVHPGSCALWVSQPPEIRTLEGS |
| | | | SAFLPCSFNASQGRLAIGSVTWFRDEVVPGKEA |
| | | | ELHIRDVRGHDASIYVCRVEVLGLGVGTGNGTR |
| | | | LVVEKEHPQLGAGTVLLLRAGFYAVSFLSVAVG |
| | | | STVYYQGKYAKSTLSGFPQL |

1. A bispecific antigen binding construct comprising a binding domain capable of binding to an HLA-G epitope and an additional binding domain capable of binding to a second epitope.

2. The bispecific antigen binding construct according to claim 1, wherein the second epitope comprises a CD3ε epitope.

3. The bispecific antigen binding construct according to claim 2, wherein the CD3ε epitope comprises or consists of an amino acid sequence set forth in SEQ ID NO: 629.

4. The bispecific antigen binding construct according to claim 2, wherein the additional binding domain capable of binding to a CD3ε epitope comprises or consists of a heavy chain variable region (VH) and a light chain variable region (VL), with VH and/or VL comprising 1, 2, 3, 4, 5, or 6 of the following:
a) a VHCDR1 having the sequence set forth in any one of SEQ ID NOS: 346-349 or 354-357,
b) a VHCDR2 having the sequence set forth in any one of SEQ ID NOS: 362-365 or 371-375,
c) a VHCDR3 having the sequence set forth in any one of SEQ ID NOS: 379-382,
d) a VLCDR1 having the sequence set forth in any one of SEQ ID NOS: 388-392,
e) a VLCDR2 having the sequence set forth in any one of SEQ ID NOS: 396-400, and
f) a VLCDR3 having the sequence set forth in any one of SEQ ID NOS: 404-408.

5. The bispecific antigen binding construct according to claim 1, wherein the additional binding domain comprises an NK cell engager, dendritic cell engager, monocyte, or macrophage engager.

6. The bispecific antigen binding construct according to claim 5, wherein the NK cell engager comprises an antibody for a CD16 epitope.

7. The bispecific antigen binding construct according to claim 5, wherein the NK cell engager comprises an antibody for NKp46.

8. The bispecific antigen binding construct according to claim 5, wherein the NK cell engager comprises an antibody for NKp30.

9. The bispecific antigen binding construct according to claim 5, wherein the monocyte or macrophage engager comprises an antibody for a CD16 epitope.

10. The bispecific antigen binding construct according to claim 1, wherein the HLA-G epitope comprises or consists of an amino acid sequence set forth in SEQ ID NO: 342.

11. The bispecific antigen binding construct according to claim 1, wherein the binding domain capable of binding to an HLA-G epitope comprises or consists of a heavy chain variable region (VH) and a light chain variable region (VL), with VH and/or VL comprising 1, 2, 3, 4, 5, or 6 of the following:
a) a VHCDR1 having the sequence set forth in any one of SEQ ID NOS: 1-14 or 18-34,
b) a VHCDR2 having the sequence set forth in any one of SEQ ID NOS: 38-50 or 54-71,
c) a VHCDR3 having the sequence set forth in any one of SEQ ID NOS: 76-101,
d) a VLCDR1 having the sequence set forth in any one of SEQ ID NOS: 105-124,
e) a VLCDR2 having the sequence set forth in any one of SEQ ID NOS: 128-145, and
f) a VLCDR3 having the sequence set forth in any one of SEQ ID NOS: 149-166.

12. The bispecific antigen binding construct according to claim 1, wherein the binding domain capable of binding to an HLA-G epitope comprises a heavy chain variable region (VH) and a light chain variable region (VL), with VH comprising, consisting of, or consisting essentially of a VH having the sequence set forth in SEQ ID NOS: 170-200 and with the VL comprising, consisting of, or consisting essentially of a VL having the sequence set forth in SEQ ID NO: 204-228 and the additional binding domain capable of binding to a CD3ε epitope comprises a heavy chain variable region (VH) and a light chain variable region (VL), with VH comprising, consisting of, or consisting essentially of a VH having the sequence set forth in SEQ ID NOS: 413-418 and with the VL comprising, consisting of, or consisting essentially of a VL having the sequence set forth in SEQ ID NOS: 422-427.

13. A pharmaceutical composition comprising or consisting of a bispecific antigen binding construct according to claim 1.

14. The pharmaceutical composition according to claim 13, further comprising an effective amount of one or more of:
a) an anti-PD-L1 antibody or small molecule inhibitor;
b) an anti-PD-1 antibody or small molecule inhibitor;
c) an anti-CD38 antibody or small molecule inhibitor;
d) an anti-CD39 antibody or small molecule inhibitor;
e) an anti-CD73 antibody or small molecule inhibitor;
f) an anti-A2A receptor antibody or small molecule inhibitor;
g) an anti-A2B receptor antibody or small molecule inhibitor;
h) an anti-A2A/A2B dual receptor antibody or small molecule inhibitor;
i) an anti-CD47 antibody or small molecule inhibitor;
j) an anti-CTLA-4 antibody or small molecule inhibitor;
k) an anti-LAG-3 antibody or small molecule inhibitor;
l) an anti-TIM-3 antibody or small molecule inhibitor;
m) an anti-TIGIT antibody or small molecule inhibitor;
n) an anti-VISTA antibody or small molecule inhibitor;
o) an anti-CD94 antibody or small molecule inhibitor;
p) a small molecule inhibitor;
q) an oncolytic virus;
r) a chemotherapy;
s) an adoptive cell therapy; and/or
t) ADCC capable therapies using effector competent antibodies such as anti-CD19, anti-CD20, anti-EGFR, anti-Her2, anti-SLAMF7, anti-CD52, anti-BCMA, anti-GD2, and/or anti-CCR4.

15. The pharmaceutical composition according to claim 13, further comprising one or both of:
a) an antibody to an immune inhibitory receptor or ligand and/or
b) an antibody to an immune stimulatory receptor or ligand.

16-34. (canceled)


SEQUENCE LISTING (643 sequences)

SEQ ID NO: 1 - protein, 9 aa, Artificial Sequence
  Description of Artificial Sequence: Synthetic peptide
GGSISSSDY

SEQ ID NO: 2 - protein, 9 aa, Artificial Sequence
  Description of Artificial Sequence: Synthetic peptide
GGSISSSST

SEQ ID NO: 3 - protein, 9 aa, Artificial Sequence
  Description of Artificial Sequence: Synthetic peptide
GGSISSSDT

SEQ ID NO: 4 - protein, 9 aa, Artificial Sequence
  Description of Artificial Sequence: Synthetic peptide
GGSISSADN

SEQ ID NO: 5 - protein, 9 aa, Artificial Sequence
  Description of Artificial Sequence: Synthetic peptide
GGSVSSSST

SEQ ID NO: 6 - protein, 8 aa, Artificial Sequence
  Description of Artificial Sequence: Synthetic peptide
GYSISSGY

SEQ ID NO: 7 - protein, 8 aa, Artificial Sequence
  Description of Artificial Sequence: Synthetic peptide
GYSILSGY

SEQ ID NO: 8 - protein, 8 aa, Artificial Sequence
  Description of Artificial Sequence: Synthetic peptide
GYSISSGH

SEQ ID NO: 9 - protein, 8 aa, Artificial Sequence
  Description of Artificial Sequence: Synthetic peptide
GYSISSGF

SEQ ID NO: 10 - protein, 7 aa, Artificial Sequence
  Description of Artificial Sequence: Synthetic peptide
GFTFDNY

SEQ ID NO: 11 - protein, 7 aa, Artificial Sequence
  Description of Artificial Sequence: Synthetic peptide
GFTFSDY

SEQ ID NO: 12 - protein, 7 aa, Artificial Sequence
  Description of Artificial Sequence: Synthetic peptide
GFTFSSS

SEQ ID NO: 13 - protein, 8 aa, Artificial Sequence
  Description of Artificial Sequence: Synthetic peptide
GGSISSSN

SEQ ID NO: 14 - protein, 9 aa, Artificial Sequence
  Description of Artificial Sequence: Synthetic peptide
GGSISSSSY

SEQ ID NO: 15-17 - skipped (3 entries)

SEQ ID NO: 18 - protein, 7 aa, Artificial Sequence
  Description of Artificial Sequence: Synthetic peptide
SSDYYWG

SEQ ID NO: 19 - protein, 7 aa, Artificial Sequence
  Description of Artificial Sequence: Synthetic peptide
SSSTYWA

SEQ ID NO: 20 - protein, 7 aa, Artificial Sequence
  Description of Artificial Sequence: Synthetic peptide
SSSTYWG

SEQ ID NO: 21 - protein, 7 aa, Artificial Sequence
  Description of Artificial Sequence: Synthetic peptide
SSDTYWG

SEQ ID NO: 22 - protein, 7 aa, Artificial Sequence
  Description of Artificial Sequence: Synthetic peptide
SADNYWG

SEQ ID NO: 23 - protein, 7 aa, Artificial Sequence
  Description of Artificial Sequence: Synthetic peptide
SSSTYWS

SEQ ID NO: 24 - protein, 6 aa, Artificial Sequence
  Description of Artificial Sequence: Synthetic peptide
SGYYWG

SEQ ID NO: 25 - protein, 6 aa, Artificial Sequence
  Description of Artificial Sequence: Synthetic peptide
SGYYWF

SEQ ID NO: 26 - protein, 6 aa, Artificial Sequence
  Description of Artificial Sequence: Synthetic peptide
SGHYWI

SEQ ID NO: 27 - protein, 6 aa, Artificial Sequence
  Description of Artificial Sequence: Synthetic peptide
SGFYWT

SEQ ID NO: 28 - protein, 6 aa, Artificial Sequence
  Description of Artificial Sequence: Synthetic peptide
SGYYWL

SEQ ID NO: 29 - protein, 6 aa, Artificial Sequence
  Description of Artificial Sequence: Synthetic peptide
SGHYWT

SEQ ID NO: 30 - protein, 5 aa, Artificial Sequence
  Description of Artificial Sequence: Synthetic peptide
NYAMH

SEQ ID NO: 31 - protein, 5 aa, Artificial Sequence
  Description of Artificial Sequence: Synthetic peptide
DYYMS

SEQ ID NO: 32 - protein, 5 aa, Artificial Sequence
  Description of Artificial Sequence: Synthetic peptide
SSAMA

SEQ ID NO: 33 - protein, 6 aa, Artificial Sequence
  Description of Artificial Sequence: Synthetic peptide
SSNWWS

SEQ ID NO: 34 - protein, 7 aa, Artificial Sequence
  Description of Artificial Sequence: Synthetic peptide
SSSYYWG

SEQ ID NO: 35-37 - skipped (3 entries)

SEQ ID NO: 38 - protein, 5 aa, Artificial Sequence
  Description of Artificial Sequence: Synthetic peptide
YYSGS

SEQ ID NO: 39 - protein, 5 aa, Artificial Sequence
  Description of Artificial Sequence: Synthetic peptide
SSSGS

SEQ ID NO: 40 - protein, 5 aa, Artificial Sequence
  Description of Artificial Sequence: Synthetic peptide
HHSGA

SEQ ID NO: 41 - protein, 5 aa, Artificial Sequence
  Description of Artificial Sequence: Synthetic peptide
HYSGS

SEQ ID NO: 42 - protein, 5 aa, Artificial Sequence
  Description of Artificial Sequence: Synthetic peptide
AYSGS

SEQ ID NO: 43 - protein, 5 aa, Artificial Sequence
  Description of Artificial Sequence: Synthetic peptide
SYNAL

SEQ ID NO: 44 - protein, 5 aa, Artificial Sequence
  Description of Artificial Sequence: Synthetic peptide
YHSGS

SEQ ID NO: 45 - protein, 5 aa, Artificial Sequence
  Description of Artificial Sequence: Synthetic peptide
YHSAS

SEQ ID NO: 46 - protein, 6 aa, Artificial Sequence
  Description of Artificial Sequence: Synthetic peptide
SARAGI

SEQ ID NO: 47 - protein, 6 aa, Artificial Sequence
  Description of Artificial Sequence: Synthetic peptide
ASSGSV

SEQ ID NO: 48 - protein, 6 aa, Artificial Sequence
  Description of Artificial Sequence: Synthetic peptide
SGSGIT

SEQ ID NO: 49 - protein, 5 aa, Artificial Sequence
  Description of Artificial Sequence: Synthetic peptide
SYSGS

SEQ ID NO: 50 - protein, 6 aa, Artificial Sequence
  Description of Artificial Sequence: Synthetic peptide
SSSGST

SEQ ID NO: 51-53 - skipped (3 entries)

SEQ ID NO: 54 - protein, 16 aa, Artificial Sequence
  Description of Artificial Sequence: Synthetic peptide
SIYYSGSTYYNPSLKS

SEQ ID NO: 55 - protein, 16 aa, Artificial Sequence
  Description of Artificial Sequence: Synthetic peptide
SISSSGSTYYNPSLKS

SEQ ID NO: 56 - protein, 16 aa, Artificial Sequence
  Description of Artificial Sequence: Synthetic peptide
SIHHSGATYYNPSLKS

SEQ ID NO: 57 - protein, 16 aa, Artificial Sequence
  Description of Artificial Sequence: Synthetic peptide
SIHYSGSTLYNPSLKS

SEQ ID NO: 58 - protein, 16 aa, Artificial Sequence
  Description of Artificial Sequence: Synthetic peptide
SIHYSGSTYYNPSLKS

SEQ ID NO: 59 - protein, 16 aa, Artificial Sequence
  Description of Artificial Sequence: Synthetic peptide
GIAYSGSTYYNPSLKS

SEQ ID NO: 60 - protein, 16 aa, Artificial Sequence
  Description of Artificial Sequence: Synthetic peptide
SISYNALTYYNPSLKS

SEQ ID NO: 61 - protein, 16 aa, Artificial Sequence
  Description of Artificial Sequence: Synthetic peptide
SIYHSGSTYYNPSLKS

SEQ ID NO: 62 - protein, 16 aa, Artificial Sequence
  Description of Artificial Sequence: Synthetic peptide
GIYHSASTAYNPSLKS

SEQ ID NO: 63 - protein, 16 aa, Artificial Sequence
  Description of Artificial Sequence: Synthetic peptide
GIYHSGSTYYNPSLKS

SEQ ID NO: 64 - protein, 16 aa, Artificial Sequence
  Description of Artificial Sequence: Synthetic peptide
AIYHSGSTVYNPSLKS

SEQ ID NO: 65 - protein, 16 aa, Artificial Sequence
  Description of Artificial Sequence: Synthetic peptide
GIYHSGSTAYNPSLKS

SEQ ID NO: 66 - protein, 17 aa, Artificial Sequence
  Description of Artificial Sequence: Synthetic peptide
AISARAGITYYADSVKG

SEQ ID NO: 67 - protein, 17 aa, Artificial Sequence
  Description of Artificial Sequence: Synthetic peptide
YIASSGSVIYYADSVKG

SEQ ID NO: 68 - protein, 17 aa, Artificial Sequence
  Description of Artificial Sequence: Synthetic peptide
TISGSGITTWYADSVKG

SEQ ID NO: 69 - protein, 16 aa, Artificial Sequence
  Description of Artificial Sequence: Synthetic peptide
EIYHSGSTNYNPSLKS

SEQ ID NO: 70 - protein, 16 aa, Artificial Sequence
  Description of Artificial Sequence: Synthetic peptide
SISYSGSTYYNPSLKS

SEQ ID NO: 71 - protein, 17 aa, Artificial Sequence
  Description of Artificial Sequence: Synthetic peptide
YISSSGSTIYYADSVKG

SEQ ID NO: 72-75 - skipped (4 entries)

SEQ ID NO: 76 - protein, 10 aa, Artificial Sequence
  Description of Artificial Sequence: Synthetic peptide
GVRRAVPFDY

SEQ ID NO: 77 - protein, 10 aa, Artificial Sequence
  Description of Artificial Sequence: Synthetic peptide
GIARAVPFDY

SEQ ID NO: 78 - protein, 10 aa, Artificial Sequence
  Description of Artificial Sequence: Synthetic peptide
GPKRAVPFDY

SEQ ID NO: 79 - protein, 10 aa, Artificial Sequence
  Description of Artificial Sequence: Synthetic peptide
GVRRAVPFVD

SEQ ID NO: 80 - protein, 10 aa, Artificial Sequence
  Description of Artificial Sequence: Synthetic peptide
GVRRAVPFQR

SEQ ID NO: 81 - protein, 10 aa, Artificial Sequence
  Description of Artificial Sequence: Synthetic peptide
GTRRAVPFDY

SEQ ID NO: 82 - protein, 10 aa, Artificial Sequence
  Description of Artificial Sequence: Synthetic peptide
GVRRAVPFAD

SEQ ID NO: 83 - protein, 10 aa, Artificial Sequence
  Description of Artificial Sequence: Synthetic peptide
GIRRAVPFDY

SEQ ID NO: 84 - protein, 10 aa, Artificial Sequence
  Description of Artificial Sequence: Synthetic peptide
GQFRAVPFDY

SEQ ID NO: 85 - protein, 13 aa, Artificial Sequence
  Description of Artificial Sequence: Synthetic peptide
GGTHTYSRGPMDV

SEQ ID NO: 86 - protein, 13 aa, Artificial Sequence
  Description of Artificial Sequence: Synthetic peptide
GGTHTYSRGPFDV

SEQ ID NO: 87 - protein, 13 aa, Artificial Sequence
  Description of Artificial Sequence: Synthetic peptide
GGTPIYSRGPLDV

SEQ ID NO: 88 - protein, 13 aa, Artificial Sequence
  Description of Artificial Sequence: Synthetic peptide
GGGQTYSRGPLDV

SEQ ID NO: 89 - protein, 13 aa, Artificial Sequence
  Description of Artificial Sequence: Synthetic peptide
GGGATYSRGPLDV

SEQ ID NO: 90 - protein, 13 aa, Artificial Sequence
  Description of Artificial Sequence: Synthetic peptide
GGTHTYSRGPLDV

SEQ ID NO: 91 - protein, 13 aa, Artificial Sequence
  Description of Artificial Sequence: Synthetic peptide
GGTVKYSRGPLDV

SEQ ID NO: 92 - protein, 13 aa, Artificial Sequence
  Description of Artificial Sequence: Synthetic peptide
GGQVTYSRGPLDV

SEQ ID NO: 93 - protein, 13 aa, Artificial Sequence
  Description of Artificial Sequence: Synthetic peptide
GGEVTYSRGPLDV

SEQ ID NO: 94 - protein, 14 aa, Artificial Sequence
  Description of Artificial Sequence: Synthetic peptide
RIGYSYGTAPPFDV

SEQ ID NO: 95 - protein, 9 aa, Artificial Sequence
  Description of Artificial Sequence: Synthetic peptide
HGTPRAFDI

SEQ ID NO: 96 - protein, 10 aa, Artificial Sequence
  Description of Artificial Sequence: Synthetic peptide
GSRHLNAFNR

SEQ ID NO: 97 - protein, 12 aa, Artificial Sequence
  Description of Artificial Sequence: Synthetic peptide
GVYHYDPYGMDV

SEQ ID NO: 98 - protein, 10 aa, Artificial Sequence
  Description of Artificial Sequence: Synthetic peptide
TELGKMHFDY

SEQ ID NO: 99 - protein, 8 aa, Artificial Sequence
  Description of Artificial Sequence: Synthetic peptide
GSPRYMQD

SEQ ID NO: 100 - protein, 12 aa, Artificial Sequence
  Description of Artificial Sequence: Synthetic peptide
HSSLGTHNWFDP

SEQ ID NO: 101 - protein, 14 aa, Artificial Sequence
  Description of Artificial Sequence: Synthetic peptide
EGALSYSWLAAFDI

SEQ ID NO: 102-104 - skipped (3 entries)

SEQ ID NO: 105 - protein, 12 aa, Artificial Sequence
  Description of Artificial Sequence: Synthetic peptide
RASQSVSSSYLA

SEQ ID NO: 106 - protein, 12 aa, Artificial Sequence
  Description of Artificial Sequence: Synthetic peptide
GASQSVSSDYLA

SEQ ID NO: 107 - protein, 12 aa, Artificial Sequence
  Description of Artificial Sequence: Synthetic peptide
QASQAVSSNYLA

SEQ ID NO: 108 - protein, 12 aa, Artificial Sequence
  Description of Artificial Sequence: Synthetic peptide
GASQSVSSAFLA

SEQ ID NO: 109 - protein, 12 aa, Artificial Sequence
  Description of Artificial Sequence: Synthetic peptide
RASQSVSSTYLA

SEQ ID NO: 110 - protein, 12 aa, Artificial Sequence
  Description of Artificial Sequence: Synthetic peptide
QASQSVSSSYLA

SEQ ID NO: 111 - protein, 12 aa, Artificial Sequence
  Description of Artificial Sequence: Synthetic peptide
KASQAVSSSYLA

SEQ ID NO: 112 - protein, 12 aa, Artificial Sequence
  Description of Artificial Sequence: Synthetic peptide
EASQSVSSSYLA

SEQ ID NO: 113 - protein, 12 aa, Artificial Sequence
  Description of Artificial Sequence: Synthetic peptide
EASQSVSASYLA

SEQ ID NO: 114 - protein, 12 aa, Artificial Sequence
  Description of Artificial Sequence: Synthetic peptide
EASQSVSSAYLA

SEQ ID NO: 115 - protein, 12 aa, Artificial Sequence
  Description of Artificial Sequence: Synthetic peptide
RVSQSVSDAYLA

SEQ ID NO: 116 - protein, 12 aa, Artificial Sequence
  Description of Artificial Sequence: Synthetic peptide
EVSQSVSASYLA

SEQ ID NO: 117 - protein, 12 aa, Artificial Sequence
  Description of Artificial Sequence: Synthetic peptide
RASQSVSSAYLA

SEQ ID NO: 118 - protein, 12 aa, Artificial Sequence
  Description of Artificial Sequence: Synthetic peptide
RASNAVSSSYLA

SEQ ID NO: 119 - protein, 11 aa, Artificial Sequence
  Description of Artificial Sequence: Synthetic peptide
RASQSINSWLA

SEQ ID NO: 120 - protein, 11 aa, Artificial Sequence
  Description of Artificial Sequence: Synthetic peptide
AASQGISSDLA

SEQ ID NO: 121 - protein, 11 aa, Artificial Sequence
  Description of Artificial Sequence: Synthetic peptide
RASQDISTYLN

SEQ ID NO: 122 - protein, 16 aa, Artificial Sequence
  Description of Artificial Sequence: Synthetic peptide
RSSQSLLHSNGYNYLD

SEQ ID NO: 123 - protein, 11 aa, Artificial Sequence
  Description of Artificial Sequence: Synthetic peptide
RASQSISSYLN

SEQ ID NO: 124 - protein, 11 aa, Artificial Sequence
  Description of Artificial Sequence: Synthetic peptide
RASQSVSSNLA

SEQ ID NO: 125-127 - skipped (3 entries)

SEQ ID NO: 128 - protein, 7 aa, Artificial Sequence
  Description of Artificial Sequence: Synthetic peptide
GASSRAT

SEQ ID NO: 129 - protein, 7 aa, Artificial Sequence
  Description of Artificial Sequence: Synthetic peptide
GAYSLAT

SEQ ID NO: 130 - protein, 7 aa, Artificial Sequence
  Description of Artificial Sequence: Synthetic peptide
GASARAT

SEQ ID NO: 131 - protein, 7 aa, Artificial Sequence
  Description of Artificial Sequence: Synthetic peptide
GASSREA

SEQ ID NO: 132 - protein, 7 aa, Artificial Sequence
  Description of Artificial Sequence: Synthetic peptide
GASNRAA

SEQ ID NO: 133 - protein, 7 aa, Artificial Sequence
  Description of Artificial Sequence: Synthetic peptide
GASSRQD

SEQ ID NO: 134 - protein, 7 aa, Artificial Sequence
  Description of Artificial Sequence: Synthetic peptide
GASNRAT

SEQ ID NO: 135 - protein, 7 aa, Artificial Sequence
  Description of Artificial Sequence: Synthetic peptide
DASSRAS

SEQ ID NO: 136 - protein, 7 aa, Artificial Sequence
  Description of Artificial Sequence: Synthetic peptide
DASTRAT

SEQ ID NO: 137 - protein, 7 aa, Artificial Sequence
  Description of Artificial Sequence: Synthetic peptide
GASDRAN

SEQ ID NO: 138 - protein, 7 aa, Artificial Sequence
  Description of Artificial Sequence: Synthetic peptide
GASYRAT

SEQ ID NO: 139 - protein, 7 aa, Artificial Sequence
  Description of Artificial Sequence: Synthetic peptide
DASSLES

SEQ ID NO: 140 - protein, 7 aa, Artificial Sequence
  Description of Artificial Sequence: Synthetic peptide
SASSTQS

SEQ ID NO: 141 - protein, 7 aa, Artificial Sequence
  Description of Artificial Sequence: Synthetic peptide
DAFNLET

SEQ ID NO: 142 - protein, 7 aa, Artificial Sequence
  Description of Artificial Sequence: Synthetic peptide
LGSNRAS

SEQ ID NO: 143 - protein, 7 aa, Artificial Sequence
  Description of Artificial Sequence: Synthetic peptide
GASRRAT

SEQ ID NO: 144 - protein, 7 aa, Artificial Sequence
  Description of Artificial Sequence: Synthetic peptide
AASSLQS

SEQ ID NO: 145 - protein, 7 aa, Artificial Sequence
  Description of Artificial Sequence: Synthetic peptide
GASTRAT

SEQ ID NO: 146-148 - skipped (3 entries)

SEQ ID NO: 149 - protein, 9 aa, Artificial Sequence
  Description of Artificial Sequence: Synthetic peptide
QQAVHSPYT

SEQ ID NO: 150 - protein, 9 aa, Artificial Sequence
  Description of Artificial Sequence: Synthetic peptide
QWAVHSPYT

SEQ ID NO: 151 - protein, 9 aa, Artificial Sequence
  Description of Artificial Sequence: Synthetic peptide
QQVVHSPYT

SEQ ID NO: 152 - protein, 9 aa, Artificial Sequence
  Description of Artificial Sequence: Synthetic peptide
QQTVHSPYT

SEQ ID NO: 153 - protein, 9 aa, Artificial Sequence
  Description of Artificial Sequence: Synthetic peptide
QQAIHSPYT

SEQ ID NO: 154 - protein, 9 aa, Artificial Sequence
  Description of Artificial Sequence: Synthetic peptide
QQHSSYPPT

SEQ ID NO: 155 - protein, 9 aa, Artificial Sequence
  Description of Artificial Sequence: Synthetic peptide
QQHSLYPPT

SEQ ID NO: 156 - protein, 9 aa, Artificial Sequence
  Description of Artificial Sequence: Synthetic peptide
QQFSSYPPT

SEQ ID NO: 157 - protein, 9 aa, Artificial Sequence
  Description of Artificial Sequence: Synthetic peptide
QQVSSYPPT

SEQ ID NO: 158 - protein, 9 aa, Artificial Sequence
  Description of Artificial Sequence: Synthetic peptide
QQHSIYPPT

SEQ ID NO: 159 - protein, 8 aa, Artificial Sequence
  Description of Artificial Sequence: Synthetic peptide
QQYDSHIT

SEQ ID NO: 160 - protein, 9 aa, Artificial Sequence
  Description of Artificial Sequence: Synthetic peptide
QQAYLYPIT

SEQ ID NO: 161 - protein, 9 aa, Artificial Sequence
  Description of Artificial Sequence: Synthetic peptide
QQLPFLPIT

SEQ ID NO: 162 - protein, 9 aa, Artificial Sequence
  Description of Artificial Sequence: Synthetic peptide
MQALGGPWT

SEQ ID NO: 163 - protein, 9 aa, Artificial Sequence
  Description of Artificial Sequence: Synthetic peptide
QQYVSDPIT

SEQ ID NO: 164 - protein, 9 aa, Artificial Sequence
  Description of Artificial Sequence: Synthetic peptide
QQVGSSPIT

SEQ ID NO: 165 - protein, 9 aa, Artificial Sequence
  Description of Artificial Sequence: Synthetic peptide
QQSHLVPRT

SEQ ID NO: 166 - protein, 10 aa, Artificial Sequence
  Description of Artificial Sequence: Synthetic peptide
QQANHHPPFT

SEQ ID NO: 167-169 - skipped (3 entries)

SEQ ID NO: 170 - protein, 120 aa, Artificial Sequence
  Description of Artificial Sequence: Synthetic polypeptide
QLQLQESGPGLVKPSETLSLTCTVSGGSISSSDYYWGWIRQPPGKGLEWIGSIYYSGSTYYNPSLKSRVTISVDTSKNQFSLKLSSVTAADTAVYYCARGVRRAVPFDYWGQGTLVTVSS

SEQ ID NO: 171 - protein, 120 aa, Artificial Sequence
  Description of Artificial Sequence: Synthetic polypeptide
QLQLQESGPGLVKPSETLSLTCTVSGGSISSSSTYWAWIRQPPGKGLEWIGSISSSGSTYYNPSLKSRVTISVDTSKNQFSLKLSSVTAADTAVYYCARGIARAVPFDYWGQGTLVTVSS

SEQ ID NO: 172 - protein, 120 aa, Artificial Sequence
  Description of Artificial Sequence: Synthetic polypeptide
QLQLQESGPGLVKPSETLSLTCTVSGGSISSSSTYWGWIRQSPGKGLEWIGSIHHSGATYYNPSLKSRVTISVDTSKNQFSLKLSSVTAADTAVYYCARGPKRAVPFDYWGQGTLVTVSS

SEQ ID NO: 173 - protein, 120 aa, Artificial Sequence
  Description of Artificial Sequence: Synthetic polypeptide
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGVRRAVPFVDWGQGTLVTVSS

SEQ ID NO: 174 - protein, 120 aa, Artificial Sequence
  Description of Artificial Sequence: Synthetic polypeptide
QLQLQESGPGLVKPSETLSLTCTVSGGSISSADNYWGWIRQPPGKGLEWIGSIHYSGSTYYNPSLKSRVTISVDTSKNQFSLKLSSVTAADTAVYYCARGVRRAVPFQRWGQGTLVTVSS

SEQ ID NO: 175 - protein, 120 aa, Artificial Sequence
  Description of Artificial Sequence: Synthetic polypeptide
QLQLQESGPGLVKPSETLSLTCTVSGGSISSSDTYWGWIRQPPGKGPEWIGSIHYSGSTLYNPSLKSRVTISVDTSKNQFSLKLSSVTAADTAVYYCARGVRRAVPFDYWGQGTLVTVSS

SEQ ID NO: 176 - protein, 120 aa, Artificial Sequence
  Description of Artificial Sequence: Synthetic polypeptide
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGVRRAVPFDYWGQGTLVTVSS

SEQ ID NO: 177 - protein, 120 aa, Artificial Sequence
  Description of Artificial Sequence: Synthetic polypeptide
QLQLQESGPGLVKPSETLSLTCTVSGGSISSSSTYWSWIRQPPGKGLEWIGSISYNALTYYNPSLKSRVTISVDTSKNQFSLKLSSVTAADTAVYYCARGTRRAVPFDYWGQGTLVTVSS

SEQ ID NO: 178 - protein, 120 aa, Artificial Sequence
  Description of Artificial Sequence: Synthetic polypeptide
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGVRRAVPFADWGQGTLVTVSS

SEQ ID NO: 179 - protein, 120 aa, Artificial Sequence
  Description of Artificial Sequence: Synthetic polypeptide
QLQLQESGPGLVKPSETLSLTCTVSGGSVSSSSTYWSWIRQPPGKGLEWIGGIAYSGSTYYNPSLKSRVTISVDTSKNQFSLKLSSVTAADTAVYYCARGVRRAVPFDYWGQGTLVTVSS

SEQ ID NO: 180 - protein, 120 aa, Artificial Sequence
  Description of Artificial Sequence: Synthetic polypeptide
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGTRRAVPFDYWGQGTLVTVSS

SEQ ID NO: 181 - protein, 120 aa, Artificial Sequence
  Description of Artificial Sequence: Synthetic polypeptide
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGIRRAVPFDYWGQGTLVTVSS

SEQ ID NO: 182 - protein, 120 aa, Artificial Sequence
  Description of Artificial Sequence: Synthetic polypeptide
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGQFRAVPFDYWGQGTLVTVSS

SEQ ID NO: 183 - protein, 122 aa, Artificial Sequence
  Description of Artificial Sequence: Synthetic polypeptide
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGGTHTYSRGPMDVWGQGTTVTVSS

SEQ ID NO: 184 - protein, 122 aa, Artificial Sequence
  Description of Artificial Sequence: Synthetic polypeptide
QLQLQESGPRLVKPSETLSLTCAVSGYSISSGYYWGWIRQPPGKGLEWIGSIYHSGSTYYNPSLKSRVTISVDTSKNQFSLKLSSVTAADTAVYYCARGGTHTYSRGPFDVWGQGTTVTVSS

SEQ ID NO: 185 - protein, 122 aa, Artificial Sequence
  Description of Artificial Sequence: Synthetic polypeptide
LVQLQESGPGLVKPSETLSLTCAVSGYSILSGYYWFWIRQPPGKGLEWIGGIYHSASTAYNPSLKSRVTISVDTSKNQFSLKLSSVTAADTAVYYCARGGTPIYSRGPLDVWGQGTTVTVSS

SEQ ID NO: 186 - protein, 122 aa, Artificial Sequence
  Description of Artificial Sequence: Synthetic polypeptide
QVQLQESGPGLVKPSETLSLTCAVSGYSISSGHYWIWIRQPPGKGLEWIGGIYHSGSTYYNPSLKSRVTISVDTSKDQFSLKLSSVTAADTAVYYCARGGGQTYSRGPLDVWGQGTTVTVSS

SEQ ID NO: 187 - protein, 122 aa, Artificial Sequence
  Description of Artificial Sequence: Synthetic polypeptide
QVQLQESGPGLVKPPETLSLTCAVSGYSISSGHYWIWIRQPPGKGLEWIGGIYHSGSTYYNPSLKSRVTISVDTSKNQFSLKLSSVTAADTAVYYCARGGGATYSRGPLDVWGQGTTVTVSS

SEQ ID NO: 188 - protein, 122 aa, Artificial Sequence
  Description of Artificial Sequence: Synthetic polypeptide
QVQLQESGPGLVKPSETLSLTCAVSGYSILSGYYWFWIRQPPGKGLEWIGGIYHSGSTYYNPSLKSRVTISVDTSKNQFSLKLSSVTAADTAVYYCARGGTHTYSRGPLDVWGQGTTVTVSS

SEQ ID NO: 189 - protein, 122 aa, Artificial Sequence
  Description of Artificial Sequence: Synthetic polypeptide
QVQLQESGPGLVKPSETLSLTCAVSGYSISSGFYWTWIRQPPGKGLEWIGAIYHSGSTVYNPSLKSRVTISVDTSKNQFSLKLSSVTAADTAVYYCARGGTHTYSRGPLDVWGQGTTVTVSS

SEQ ID NO: 190 - protein, 122 aa, Artificial Sequence
  Description of Artificial Sequence: Synthetic polypeptide
QVQLQESGPGLVKPSETLSLTCAVSGYSISSGYYWLWIRQPPGKGLEWIGGIYHSASTAYNPSLKSRVTISVDTSKNQFSLKLSSVTAADTAVYYCARGGTVKYSRGPLDVWGQGTTVTVSS

SEQ ID NO: 191 - protein, 122 aa, Artificial Sequence
  Description of Artificial Sequence: Synthetic polypeptide
QVQLQESGPGLVKPSETLSLTCAVSGYSISSGHYWTWIRQPPGKGLEWIGAIYHSGSTVYNPSLKSRVTISVDTSKNQFSLKLSSVTAADTAVYYCARGGQVTYSRGPLDVWGQGTTVTVSS

SEQ ID NO: 192 - protein, 122 aa, Artificial Sequence
  Description of Artificial Sequence: Synthetic polypeptide
QVQLQESGPGLVKPSETLSLTCAVSGYSILSGYYWFWIRQPPGKGLEWIGGIYHSGSTAYNPSLKSRVTISVDTSKNQFSLKLSSVTAADTAVYYCARGGEVTYSRGPLDVWGQGTTVTVSS

SEQ ID NO: 193 - protein, 123 aa, Artificial Sequence
  Description of Artificial Sequence: Synthetic polypeptide
EVQLLESGGGLVQPGGSLRLSCAASGFTFDNYAMHWVRQAPGKGLEWVSAISARAGITYYADSVKGRFTISRDNSKNTLYLQMNSLRAEDTAVYYCARRIGYSYGTAPPFDVWGQGTTVTVSS

SEQ ID NO: 194 - protein, 118 aa, Artificial Sequence
  Description of Artificial Sequence: Synthetic polypeptide
QVQLVESGGGLVQPGGSLRLSCAASGFTFSDYYMSWIRQAPGKGLEWVSYIASSGSVIYYADSVKGRFTISRDNAKNSLYLQMNSLRAEDTAVYYCARHGTPRAFDIWGQGTTVTVSS

SEQ ID NO: 195 - protein, 119 aa, Artificial Sequence
  Description of Artificial Sequence: Synthetic polypeptide
EVQLLESGGGLVQPGGSLRLSCAASGFTFSSSAMAWVRQAPGKGLEWVSTISGSGITTWYADSVKGRFTISRDNSKNTLYLQMNSLRAEDTAVYYCAKGSRHLNAFNRWGQGTTVTVSS

SEQ ID NO: 196 - protein, 121 aa, Artificial Sequence
  Description of Artificial Sequence: Synthetic polypeptide
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARGVYHYDPYGMDVWGQGTTVTVSS

SEQ ID NO: 197 - protein, 120 aa, Artificial Sequence
  Description of Artificial Sequence: Synthetic polypeptide
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARTELGKMHFDYWGQGTLVTVSS

SEQ ID NO: 198 - protein, 118 aa, Artificial Sequence
  Description of Artificial Sequence: Synthetic polypeptide
QLQLQESGPGLVKPSETLSLTCTVSGGSISSSDYYWGWIRQPPGKGLEWIGSIYYSGSTYYNPSLKSRVTISVDTSKNQFSLKLSSVTAADTAVYYCARGSPRYMQDWGQGTLVTVSS

SEQ ID NO: 199 - protein, 121 aa, Artificial Sequence
  Description of Artificial Sequence: Synthetic polypeptide
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARHSSLGTHNWFDPWGQGTLVTVSS

SEQ ID NO: 200 - protein, 123 aa, Artificial Sequence
  Description of Artificial Sequence: Synthetic polypeptide
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCAREGALSYSWLAAFDIWGQGTMVTVSS

SEQ ID NO: 201-203 - skipped (3 entries)

SEQ ID NO: 204 - protein, 108 aa, Artificial Sequence
  Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQAVHSPYTFGGGTKVEIK

SEQ ID NO: 205 - protein, 108 aa, Artificial Sequence
  Description of Artificial Sequence: Synthetic polypeptide
EIVLTQSPGTLSLSPGERATLSCGASQSVSSDYLAWYQQKPGQAPRLLIYGAYSLATGIPDRFSGSGSGTDFTLTISRLEPEDFAVYYCQWAVHSPYTFGGGTKVEIK

SEQ ID NO: 206 - protein, 108 aa, Artificial Sequence
  Description of Artificial Sequence: Synthetic polypeptide
EIVLTQSPGTLSLSPGERATLSCQASQAVSSNYLAWYQQKPGQAPRLLIYGASSRATGIPDRFSGSGSGTDFTLTISRLEPEDFAVYYCQQVVHSPYTFGGGTKVEIK

SEQ ID NO: 207 - protein, 108 aa, Artificial Sequence
  Description of Artificial Sequence: Synthetic polypeptide
EIVLTQSPGTLSLSPGERATLSCGASQSVSSAFLAWYQQKPGQAPRLLIYGASARATGIPDRFSGSGSGTDFTLTISRLEPEDFAVYYCQQVVHSPYTFGGGTKVEIK

SEQ ID NO: 208 - protein, 108 aa, Artificial Sequence
  Description of Artificial Sequence: Synthetic polypeptide
EIVLTQSPGTLSLSPGERATLSCRASQSVSSTYLAWYQQKPGQAPRLLIYGASSREAGIPDRFSGSGSGTDFTLTISRLEPEDFAVYYCQQTVHSPYTFGGGTKVEIK

SEQ ID NO: 209 - protein, 108 aa, Artificial Sequence
  Description of Artificial Sequence: Synthetic polypeptide
EIVLTQSPGTLSLSPGERATLSCQASQAVSSNYLAWYQQKPGQAPRLLIYGASNRAAGIPDRFSGSGSGTDFTLTISRLEPEDFAVYYCQQAIHSPYTFGGGTKVEIK

SEQ ID NO: 210 - protein, 108 aa, Artificial Sequence
  Description of Artificial Sequence: Synthetic polypeptide
EIVLTQSPGTLSLSPGERATLSCQASQSVSSSYLAWYQQKPGQAPRLLIYGASNRAAGIPDRFSGSGSGTDFTLTISRLEPEDFAVYYCQQVVHSPYTFGGGTKVEIK

SEQ ID NO: 211 - protein, 108 aa, Artificial Sequence
  Description of Artificial Sequence: Synthetic polypeptide
EIVLTQSPGTLSLSPGERATLSCKASQAVSSSYLAWYQQKPGQAPRLLIYGASSRQDGIPDRFSGSGSGTDFTLTISRLEPEDFAVYYCQQVVHSPYTFGGGTKVEIK

SEQ ID NO: 212 - protein, 108 aa, Artificial Sequence
  Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQHSSYPPTFGGGTKVEIK

SEQ ID NO: 213 - protein, 108 aa, Artificial Sequence
  Description of Artificial Sequence: Synthetic polypeptide
EIVLTQSPGTLSLSPGERATLSCEASQSVSSSYLAWYQQKPGQAPRLLIYGASNRATGIPDRFSGSGSGTDFTLTISRLEPEDFAVYYCQQHSLYPPTFGGGTKVEIK

SEQ ID NO: 214 - protein, 108 aa, Artificial Sequence
  Description of Artificial Sequence: Synthetic polypeptide
EIVLTQSPGTLSLSPGERATLSCEASQSVSASYLAWYQQKPGQAPRLLIYDASSRASGIPDRFSGSGSGTDFTLTISRLEPEDFAVYYCQQFSSYPPTFGGGTKVEIK

SEQ ID NO: 215 - protein, 108 aa, Artificial Sequence
  Description of Artificial Sequence: Synthetic polypeptide
EIVLTQSPGTLSLSPGERATLSCEASQSVSSAYLAWYQQKPGQAPRLLIYDASTRATGIPDRFSGSGSGTDFTLTISRLEPEDFAVYYCQQVSSYPPTFGGGTKVEIK

SEQ ID NO: 216 - protein, 108 aa, Artificial Sequence
  Description of Artificial Sequence: Synthetic polypeptide
EIVLTQSPGTLSLSPGERAALSCRVSQSVSDAYLAWYQQKPGQAPRLLIYDASSRASGIPDRFSGSGSGTDFTLTISRLEPEDFAVYYCQQVSSYPPTFGGGTKVEIK

SEQ ID NO: 217 - protein, 108 aa, Artificial Sequence
  Description of Artificial Sequence: Synthetic polypeptide
EIVLTQSPGTLSLSPGERATLSCEVSQSVSASYLAWYQQKPGQAPRLLIYGASSRATGIPDRFSGSGSGTDFTLTISRLEPEDFAVYYCQQHSLYPPTFGGGTKVEIK

SEQ ID NO: 218 - protein, 108 aa, Artificial Sequence
  Description of Artificial Sequence: Synthetic polypeptide
EIVLTQSPGTLSLSPGERATLSCRASQSVSSAYLAWYQQKPGQAPRLLIYGASDRANGIPDRFSGSGSGTDFTLTISRLEPEDFAVYYCQQHSLYPPTFGGGTKVEIK

SEQ ID NO: 219 - protein, 108 aa, Artificial Sequence
  Description of Artificial Sequence: Synthetic polypeptide
EIVLTQSPGTLSLSPGERATLSCRASNAVSSSYLAWYQQKPGQAPRLLIYGASDRANGIPDRFSGSGSGTDFTLTISRLEPEDFAVYYCQQHSIYPPTFGGGTKVEIK

SEQ ID NO: 220 - protein, 108 aa, Artificial Sequence
  Description of Artificial Sequence: Synthetic polypeptide
EIVLTQSPGTLSLSPGERATLSCRASNAVSSSYLAWYQQKPGQAPRLLIYGASYRATGIPDRFSGSGSGTDFTLTISRLEPEDFAVYYCQQHSLYPPTFGGGTKVEIK

SEQ ID NO: 221 - protein, 106 aa, Artificial Sequence
  Description of Artificial Sequence: Synthetic polypeptide
DIQMTQSPSTLSASVGDRVTITCRASQSINSWLAWYQQKPGKAPKLLISDASSLESGVPSRFSGSGSGTEFTLTISSLQPDDFATYYCQQYDSHITFGGGTKVEIK

SEQ ID NO: 222 - protein, 107 aa, Artificial Sequence
  Description of Artificial Sequence: Synthetic polypeptide
DIQMTQSPSSVSASVGDRVTITCAASQGISSDLAWYQQKPGKAPKLLIYSASSTQSGVPSRFSGSGSGTDFTLTISSLQPEDFATYYCQQAYLYPITFGGGTKVEIK

SEQ ID NO: 223 - protein, 107 aa, Artificial Sequence
  Description of Artificial Sequence: Synthetic polypeptide
GVQMTQSPSSLSASVGDRVTITCRASQDISTYLNWYQQKPGKAPKLLIYDAFNLETGVPSRFSGSGSGTDFTFTISSLQPEDIATYYCQQLPFLPITFGGGTKVEIK

SEQ ID NO: 224 - protein, 112 aa, Artificial Sequence
  Description of Artificial Sequence: Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALGGPWTFGGGTKVEIK

SEQ ID NO: 225 - protein, 108 aa, Artificial Sequence
  Description of Artificial Sequence: Synthetic polypeptide
EIVLTQSPGTLSLSPGERATLSCRASQSVSSSYLAWYQQKPGQAPRLLIYGASRRATGIPDRFSGSGSGTDFTLTISRLEPEDFAVYYCQQYVSDPITFGGGTKVEIK

SEQ ID NO: 226 - protein, 108 aa, Artificial Sequence
  Description of Artificial Sequence: Synthetic polypeptide
EIVLTQSPGTLSLSPGERATLSCRASQSVSSSYLAWYQQKPGQAPRLLIYGASRRATGIPDRFSGSGSGTDFTLTISRLEPEDFAVYYCQQVGSSPITFGGGTKVEIK

SEQ ID NO: 227 - protein, 107 aa, Artificial Sequence
  Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSHLVPRTFGGGTKVEIK

SEQ ID NO: 228 - protein, 108 aa, Artificial Sequence
  Description of Artificial Sequence: Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQANHHPPFTFGGGTKVEIK

SEQ ID NO: 229-231 - skipped (3 entries)

SEQ ID NO: 232 - protein, 450 aa, Artificial Sequence
  Description of Artificial Sequence: Synthetic polypeptide
QLQLQESGPGLVKPSETLSLTCTVSGGSISSSDYYWGWIRQPPGKGLEWIGSIYYSGSTYYNPSLKSRVTISVDTSKNQFSLKLSSVTAADTAVYYCARGVRRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 450 aa, Artificial Sequence
  Description of Artificial Sequence: Synthetic polypeptide
QLQLQESGPGLVKPSETLSLTCTVSGGSISSSSTYWAWIRQPPGKGLEWIGSISSSGSTYYNPSLKSRVTISVDTSKNQFSLKLSSVTAADTAVYYCARGIARAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 234 - protein, 450 aa, Artificial Sequence
  Description of Artificial Sequence: Synthetic polypeptide
QLQLQESGPGLVKPSETLSLTCTVSGGSISSSSTYWGWIRQSPGKGLEWIGSIHHSGATYYNPSLKSRVTISVDTSKNQFSLKLSSVTAADTAVYYCARGPKRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 450 aa, Artificial Sequence
  Description of Artificial Sequence: Synthetic polypeptide
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGVRRAVPFVDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 450 aa, Artificial Sequence
  Description of Artificial Sequence: Synthetic polypeptide
QLQLQESGPGLVKPSETLSLTCTVSGGSISSADNYWGWIRQPPGKGLEWIGSIHYSGSTYYNPSLKSRVTISVDTSKNQFSLKLSSVTAADTAVYYCARGVRRAVPFQRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 450 aa, Artificial Sequence
  Description of Artificial Sequence: Synthetic polypeptide
QLQLQESGPGLVKPSETLSLTCTVSGGSISSSDTYWGWIRQPPGKGPEWIGSIHYSGSTLYNPSLKSRVTISVDTSKNQFSLKLSSVTAADTAVYYCARGVRRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - protein, 450 aa, Artificial Sequence
  Description of Artificial Sequence: Synthetic polypeptide
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGVRRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 450 aa, Artificial Sequence
  Description of Artificial Sequence: Synthetic polypeptide
QLQLQESGPGLVKPSETLSLTCTVSGGSISSSSTYWSWIRQPPGKGLEWIGSISYNALTYYNPSLKSRVTISVDTSKNQFSLKLSSVTAADTAVYYCARGTRRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 240 - protein, 450 aa, Artificial Sequence
  Description of Artificial Sequence: Synthetic polypeptide
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGVRRAVPFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - protein, 450 aa, Artificial Sequence
  Description of Artificial Sequence: Synthetic polypeptide
QLQLQESGPGLVKPSETLSLTCTVSGGSVSSSSTYWSWIRQPPGKGLEWIGGIAYSGSTYYNPSLKSRVTISVDTSKNQFSLKLSSVTAADTAVYYCARGVRRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 450 aa, Artificial Sequence
  Description of Artificial Sequence: Synthetic polypeptide
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGTRRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - protein, 450 aa, Artificial Sequence
  Description of Artificial Sequence: Synthetic polypeptide
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGIRRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 450 aa, Artificial Sequence
  Description of Artificial Sequence: Synthetic polypeptide
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGQFRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 245 - protein, 452 aa, Artificial Sequence
  Description of Artificial Sequence: Synthetic polypeptide
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGGTHTYSRGPMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 246 - protein, 452 aa, Artificial Sequence
  Description of Artificial Sequence: Synthetic polypeptide
QLQLQESGPRLVKPSETLSLTCAVSGYSISSGYYWGWIRQPPGKGLEWIGSIYHSGSTYYNPSLKSRVTISVDTSKNQFSLKLSSVTAADTAVYYCARGGTHTYSRGPFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - protein, 452 aa, Artificial Sequence
  Description of Artificial Sequence: Synthetic polypeptide
LVQLQESGPGLVKPSETLSLTCAVSGYSILSGYYWFWIRQPPGKGLEWIGGIYHSASTAYNPSLKSRVTISVDTSKNQFSLKLSSVTAADTAVYYCARGGTPIYSRGPLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 248 - protein, 452 aa, Artificial Sequence
  Description of Artificial Sequence: Synthetic polypeptide
QVQLQESGPGLVKPSETLSLTCAVSGYSISSGHYWIWIRQPPGKGLEWIGGIYHSGSTYYNPSLKSRVTISVDTSKDQFSLKLSSVTAADTAVYYCARGGGQTYSRGPLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 249 - protein, 452 aa, Artificial Sequence
  Description of Artificial Sequence: Synthetic polypeptide
QVQLQESGPGLVKPPETLSLTCAVSGYSISSGHYWIWIRQPPGKGLEWIGGIYHSGSTYYNPSLKSRVTISVDTSKNQFSLKLSSVTAADTAVYYCARGGGATYSRGPLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - protein, 452 aa, Artificial Sequence
  Description of Artificial Sequence: Synthetic polypeptide
QVQLQESGPGLVKPSETLSLTCAVSGYSILSGYYWFWIRQPPGKGLEWIGGIYHSGSTYYNPSLKSRVTISVDTSKNQFSLKLSSVTAADTAVYYCARGGTHTYSRGPLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 251 - protein, 452 aa, Artificial Sequence
  Description of Artificial Sequence: Synthetic polypeptide
QVQLQESGPGLVKPSETLSLTCAVSGYSISSGFYWTWIRQPPGKGLEWIGAIYHSGSTVYNPSLKSRVTISVDTSKNQFSLKLSSVTAADTAVYYCARGGTHTYSRGPLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 252 - protein, 452 aa, Artificial Sequence
  Description of Artificial Sequence: Synthetic polypeptide
QVQLQESGPGLVKPSETLSLTCAVSGYSISSGYYWLWIRQPPGKGLEWIGGIYHSASTAYNPSLKSRVTISVDTSKNQFSLKLSSVTAADTAVYYCARGGTVKYSRGPLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 253 - protein, 452 aa, Artificial Sequence
  Description of Artificial Sequence: Synthetic polypeptide
QVQLQESGPGLVKPSETLSLTCAVSGYSISSGHYWTWIRQPPGKGLEWIGAIYHSGSTVYNPSLKSRVTISVDTSKNQFSLKLSSVTAADTAVYYCARGGQVTYSRGPLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 254 - protein, 451 aa, Artificial Sequence
  Description of Artificial Sequence: Synthetic polypeptide
QVQLQESGPGLVKPSETLSLTCAVSGYSILSGYYWFWIRQPPGKGLEWIGGIYHSGSTAYNPSLKSRVTISVDTSKNQFSLKLSSVTAADTAVYYCARGGEVTYSRGPLDVWGQGTTVTVSSASTKGPSVFPLAPSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 255 - protein, 453 aa, Artificial Sequence
  Description of Artificial Sequence: Synthetic polypeptide
EVQLLESGGGLVQPGGSLRLSCAASGFTFDNYAMHWVRQAPGKGLEWVSAISARAGITYYADSVKGRFTISRDNSKNTLYLQMNSLRAEDTAVYYCARRIGYSYGTAPPFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 256 - protein, 448 aa, Artificial Sequence
  Description of Artificial Sequence: Synthetic polypeptide
QVQLVESGGGLVQPGGSLRLSCAASGFTFSDYYMSWIRQAPGKGLEWVSYIASSGSVIYYADSVKGRFTISRDNAKNSLYLQMNSLRAEDTAVYYCARHGTPRAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 257 - protein, 449 aa, Artificial Sequence
  Description of Artificial Sequence: Synthetic polypeptide
EVQLLESGGGLVQPGGSLRLSCAASGFTFSSSAMAWVRQAPGKGLEWVSTISGSGITTWYADSVKGRFTISRDNSKNTLYLQMNSLRAEDTAVYYCAKGSRHLNAFNRWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - protein, 451 aa, Artificial Sequence
  Description of Artificial Sequence: Synthetic polypeptide
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARGVYHYDPYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 259 - protein, 450 aa, Artificial Sequence
  Description of Artificial Sequence: Synthetic polypeptide
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARTELGKMHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 448 aa, Artificial Sequence
  Description of Artificial Sequence: Synthetic polypeptide
QLQLQESGPGLVKPSETLSLTCTVSGGSISSSDYYWGWIRQPPGKGLEWIGSIYYSGSTYYNPSLKSRVTISVDTSKNQFSLKLSSVTAADTAVYYCARGSPRYMQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 261 - protein, 451 aa, Artificial Sequence
  Description of Artificial Sequence: Synthetic polypeptide
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARHSSLGTHNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 262 - protein, 453 aa, Artificial Sequence
  Description of Artificial Sequence: Synthetic polypeptide
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCAREGALSYSWLAAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 263-265 - skipped (3 entries)

SEQ ID NO: 266 - protein, 447 aa, Artificial Sequence
  Description of Artificial Sequence: Synthetic polypeptide
QLQLQESGPGLVKPSETLSLTCTVSGGSISSSDYYWGWIRQPPGKGLEWIGSIYYSGSTYYNPSLKSRVTISVDTSKNQFSLKLSSVTAADTAVYYCARGVRRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 267 - protein, 447 aa, Artificial Sequence
  Description of Artificial Sequence: Synthetic polypeptide
QLQLQESGPGLVKPSETLSLTCTVSGGSISSSSTYWAWIRQPPGKGLEWIGSISSSGSTYYNPSLKSRVTISVDTSKNQFSLKLSSVTAADTAVYYCARGIARAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 268 - protein, 447 aa, Artificial Sequence
  Description of Artificial Sequence: Synthetic polypeptide
QLQLQESGPGLVKPSETLSLTCTVSGGSISSSSTYWGWIRQSPGKGLEWIGSIHHSGATYYNPSLKSRVTISVDTSKNQFSLKLSSVTAADTAVYYCARGPKRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 269 - protein, 447 aa, Artificial Sequence
  Description of Artificial Sequence: Synthetic polypeptide
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGVRRAVPFVDWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 270 - protein, 447 aa, Artificial Sequence
  Description of Artificial Sequence: Synthetic polypeptide
QLQLQESGPGLVKPSETLSLTCTVSGGSISSADNYWGWIRQPPGKGLEWIGSIHYSGSTYYNPSLKSRVTISVDTSKNQFSLKLSSVTAADTAVYYCARGVRRAVPFQRWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 271 - protein, 447 aa, Artificial Sequence
  Description of Artificial Sequence: Synthetic polypeptide
QLQLQESGPGLVKPSETLSLTCTVSGGSISSSDTYWGWIRQPPGKGPEWIGSIHYSGSTLYNPSLKSRVTISVDTSKNQFSLKLSSVTAADTAVYYCARGVRRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 272 - protein, 447 aa, Artificial Sequence
  Description of Artificial Sequence: Synthetic polypeptide
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGVRRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 273 - protein, 447 aa, Artificial Sequence
  Description of Artificial Sequence: Synthetic polypeptide
QLQLQESGPGLVKPSETLSLTCTVSGGSISSSSTYWSWIRQPPGKGLEWIGSISYNALTYYNPSLKSRVTISVDTSKNQFSLKLSSVTAADTAVYYCARGTRRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 274 - protein, 447 aa, Artificial Sequence
  Description of Artificial Sequence: Synthetic polypeptide
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGVRRAVPFADWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 275 - protein, 447 aa, Artificial Sequence
  Description of Artificial Sequence: Synthetic polypeptide
QLQLQESGPGLVKPSETLSLTCTVSGGSVSSSSTYWSWIRQPPGKGLEWIGGIAYSGSTYYNPSLKSRVTISVDTSKNQFSLKLSSVTAADTAVYYCARGVRRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 276 - protein, 447 aa, Artificial Sequence
  Description of Artificial Sequence: Synthetic polypeptide
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGTRRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 277 - protein, 447 aa, Artificial Sequence
  Description of Artificial Sequence: Synthetic polypeptide
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGIRRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 278 - protein, 447 aa, Artificial Sequence
  Description of Artificial Sequence: Synthetic polypeptide
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGQFRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 279 - protein, 449 aa, Artificial Sequence
  Description of Artificial Sequence: Synthetic polypeptide
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGGTHTYSRGPMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 280 - protein, 449 aa, Artificial Sequence
  Description of Artificial Sequence: Synthetic polypeptide
QLQLQESGPRLVKPSETLSLTCAVSGYSISSGYYWGWIRQPPGKGLEWIGSIYHSGSTYYNPSLKSRVTISVDTSKNQFSLKLSSVTAADTAVYYCARGGTHTYSRGPFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 281 - protein, 449 aa, Artificial Sequence
  Description of Artificial Sequence: Synthetic polypeptide
LVQLQESGPGLVKPSETLSLTCAVSGYSILSGYYWFWIRQPPGKGLEWIGGIYHSASTAYNPSLKSRVTISVDTSKNQFSLKLSSVTAADTAVYYCARGGTPIYSRGPLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 282 - protein, 449 aa, Artificial Sequence
  Description of Artificial Sequence: Synthetic polypeptide
QVQLQESGPGLVKPSETLSLTCAVSGYSISSGHYWIWIRQPPGKGLEWIGGIYHSGSTYYNPSLKSRVTISVDTSKDQFSLKLSSVTAADTAVYYCARGGGQTYSRGPLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 283 - protein, 449 aa, Artificial Sequence
  Description of Artificial Sequence: Synthetic polypeptide
QVQLQESGPGLVKPPETLSLTCAVSGYSISSGHYWIWIRQPPGKGLEWIGGIYHSGSTYYNPSLKSRVTISVDTSKNQFSLKLSSVTAADTAVYYCARGGGATYSRGPLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 284 - protein, 449 aa, Artificial Sequence
  Description of Artificial Sequence: Synthetic polypeptide
QVQLQESGPGLVKPSETLSLTCAVSGYSILSGYYWFWIRQPPGKGLEWIGGIYHSGSTYYNPSLKSRVTISVDTSKNQFSLKLSSVTAADTAVYYCARGGTHTYSRGPLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 285 - protein, 449 aa, Artificial Sequence
  Description of Artificial Sequence: Synthetic polypeptide
QVQLQESGPGLVKPSETLSLTCAVSGYSISSGFYWTWIRQPPGKGLEWIGAIYHSGSTVYNPSLKSRVTISVDTSKNQFSLKLSSVTAADTAVYYCARGGTHTYSRGPLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 286 - protein, 449 aa, Artificial Sequence
  Description of Artificial Sequence: Synthetic polypeptide
QVQLQESGPGLVKPSETLSLTCAVSGYSISSGYYWLWIRQPPGKGLEWIGGIYHSASTAYNPSLKSRVTISVDTSKNQFSLKLSSVTAADTAVYYCARGGTVKYSRGPLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 287 - protein, 449 aa, Artificial Sequence
  Description of Artificial Sequence: Synthetic polypeptide
QVQLQESGPGLVKPSETLSLTCAVSGYSISSGHYWTWIRQPPGKGLEWIGAIYHSGSTVYNPSLKSRVTISVDTSKNQFSLKLSSVTAADTAVYYCARGGQVTYSRGPLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 288 - protein, 449 aa, Artificial Sequence
  Description of Artificial Sequence: Synthetic polypeptide
QVQLQESGPGLVKPSETLSLTCAVSGYSILSGYYWFWIRQPPGKGLEWIGGIYHSGSTAYNPSLKSRVTISVDTSKNQFSLKLSSVTAADTAVYYCARGGEVTYSRGPLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 289 - protein, 450 aa, Artificial Sequence
  Description of Artificial Sequence: Synthetic polypeptide
EVQLLESGGGLVQPGGSLRLSCAASGFTFDNYAMHWVRQAPGKGLEWVSAISARAGITYYADSVKGRFTISRDNSKNTLYLQMNSLRAEDTAVYYCARRIGYSYGTAPPFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 290 - protein, 445 aa, Artificial Sequence
  Description of Artificial Sequence: Synthetic polypeptide
QVQLVESGGGLVQPGGSLRLSCAASGFTFSDYYMSWIRQAPGKGLEWVSYIASSGSVIYYADSVKGRFTISRDNAKNSLYLQMNSLRAEDTAVYYCARHGTPRAFDIWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 291 - protein, 446 aa, Artificial Sequence
  Description of Artificial Sequence: Synthetic polypeptide
EVQLLESGGGLVQPGGSLRLSCAASGFTFSSSAMAWVRQAPGKGLEWVSTISGSGITTWYADSVKGRFTISRDNSKNTLYLQMNSLRAEDTAVYYCAKGSRHLNAFNRWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 292 - protein, 448 aa, Artificial Sequence
  Description of Artificial Sequence: Synthetic polypeptide
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARGVYHYDPYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 293 - protein, 447 aa, Artificial Sequence
  Description of Artificial Sequence: Synthetic polypeptide
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARTELGKMHFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 294 - protein, 445 aa, Artificial Sequence
  Description of Artificial Sequence: Synthetic polypeptide
QLQLQESGPGLVKPSETLSLTCTVSGGSISSSDYYWGWIRQPPGKGLEWIGSIYYSGSTYYNPSLKSRVTISVDTSKNQFSLKLSSVTAADTAVYYCARGSPRYMQDWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 295 - protein, 448 aa, Artificial Sequence
  Description of Artificial Sequence: Synthetic polypeptide
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARHSSLGTHNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 296 - protein, 450 aa, Artificial Sequence
  Description of Artificial Sequence: Synthetic polypeptide
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCAREGALSYSWLAAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 297-299 - skipped (3 entries)

SEQ ID NO: 300 - protein, 215 aa, Artificial Sequence
  Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQAVHSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 301 - protein, 215 aa, Artificial Sequence; identical to SEQ ID NO: 300
  Description of Artificial Sequence: Synthetic polypeptide

SEQ ID NO: 302 - protein, 215 aa, Artificial Sequence; identical to SEQ ID NO: 300
  Description of Artificial Sequence: Synthetic polypeptide

SEQ ID NO: 303 - protein, 215 aa, Artificial Sequence; identical to SEQ ID NO: 300
  Description of Artificial Sequence: Synthetic polypeptide

SEQ ID NO: 304 - protein, 215 aa, Artificial Sequence; identical to SEQ ID NO: 300
  Description of Artificial Sequence: Synthetic polypeptide

SEQ ID NO: 305 - protein, 215 aa, Artificial Sequence
  Description of Artificial Sequence: Synthetic polypeptide
EIVLTQSPGTLSLSPGERATLSCGASQSVSSDYLAWYQQKPGQAPRLLIYGAYSLATGIPDRFSGSGSGTDFTLTISRLEPEDFAVYYCQWAVHSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 306 - protein, 215 aa, Artificial Sequence
  Description of Artificial Sequence: Synthetic polypeptide
EIVLTQSPGTLSLSPGERATLSCQASQAVSSNYLAWYQQKPGQAPRLLIYGASSRATGIPDRFSGSGSGTDFTLTISRLEPEDFAVYYCQQVVHSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 307 - protein, 215 aa, Artificial Sequence
  Description of Artificial Sequence: Synthetic polypeptide
EIVLTQSPGTLSLSPGERATLSCGASQSVSSAFLAWYQQKPGQAPRLLIYGASARATGIPDRFSGSGSGTDFTLTISRLEPEDFAVYYCQQVVHSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 308 - protein, 215 aa, Artificial Sequence
  Description of Artificial Sequence: Synthetic polypeptide
EIVLTQSPGTLSLSPGERATLSCRASQSVSSTYLAWYQQKPGQAPRLLIYGASSREAGIPDRFSGSGSGTDFTLTISRLEPEDFAVYYCQQTVHSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 309 - protein, 215 aa, Artificial Sequence
  Description of Artificial Sequence: Synthetic polypeptide
EIVLTQSPGTLSLSPGERATLSCQASQAVSSNYLAWYQQKPGQAPRLLIYGASNRAAGIPDRFSGSGSGTDFTLTISRLEPEDFAVYYCQQAIHSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 310 - protein, 215 aa, Artificial Sequence
  Description of Artificial Sequence: Synthetic polypeptide
EIVLTQSPGTLSLSPGERATLSCQASQSVSSSYLAWYQQKPGQAPRLLIYGASNRAAGIPDRFSGSGSGTDFTLTISRLEPEDFAVYYCQQVVHSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 311 - protein, 215 aa, Artificial Sequence
  Description of Artificial Sequence: Synthetic polypeptide
EIVLTQSPGTLSLSPGERATLSCKASQAVSSSYLAWYQQKPGQAPRLLIYGASSRQDGIPDRFSGSGSGTDFTLTISRLEPEDFAVYYCQQVVHSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 312 - protein, 215 aa, Artificial Sequence; identical to SEQ ID NO: 300
  Description of Artificial Sequence: Synthetic polypeptide

SEQ ID NO: 313 - protein, 215 aa, Artificial Sequence
  Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQHSSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 314 - protein, 215 aa, Artificial Sequence; identical to SEQ ID NO: 313
  Description of Artificial Sequence: Synthetic polypeptide

SEQ ID NO: 315 - protein, 215 aa, Artificial Sequence
  Description of Artificial Sequence: Synthetic polypeptide
EIVLTQSPGTLSLSPGERATLSCEASQSVSSSYLAWYQQKPGQAPRLLIYGASNRATGIPDRFSGSGSGTDFTLTISRLEPEDFAVYYCQQHSLYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 316 - protein, 215 aa, Artificial Sequence
  Description of Artificial Sequence: Synthetic polypeptide
EIVLTQSPGTLSLSPGERATLSCEASQSVSASYLAWYQQKPGQAPRLLIYDASSRASGIPDRFSGSGSGTDFTLTISRLEPEDFAVYYCQQFSSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 317 - protein, 215 aa, Artificial Sequence
  Description of Artificial Sequence: Synthetic polypeptide
EIVLTQSPGTLSLSPGERATLSCEASQSVSSAYLAWYQQKPGQAPRLLIYDASTRATGIPDRFSGSGSGTDFTLTISRLEPEDFAVYYCQQVSSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 318 - protein, 215 aa, Artificial Sequence
  Description of Artificial Sequence: Synthetic polypeptide
EIVLTQSPGTLSLSPGERAALSCRVSQSVSDAYLAWYQQKPGQAPRLLIYDASSRASGIPDRFSGSGSGTDFTLTISRLEPEDFAVYYCQQVSSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 319 - protein, 215 aa, Artificial Sequence
  Description of Artificial Sequence: Synthetic polypeptide
EIVLTQSPGTLSLSPGERATLSCEVSQSVSASYLAWYQQKPGQAPRLLIYGASSRATGIPDRFSGSGSGTDFTLTISRLEPEDFAVYYCQQHSLYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 320 - protein, 215 aa, Artificial Sequence
  Description of Artificial Sequence: Synthetic polypeptide
EIVLTQSPGTLSLSPGERATLSCRASQSVSSAYLAWYQQKPGQAPRLLIYGASDRANGIPDRFSGSGSGTDFTLTISRLEPEDFAVYYCQQHSLYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 321 - protein, 215 aa, Artificial Sequence
  Description of Artificial Sequence: Synthetic polypeptide
EIVLTQSPGTLSLSPGERATLSCRASNAVSSSYLAWYQQKPGQAPRLLIYGASDRANGIPDRFSGSGSGTDFTLTISRLEPEDFAVYYCQQHSIYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 322 - protein, 215 aa, Artificial Sequence
  Description of Artificial Sequence: Synthetic polypeptide
EIVLTQSPGTLSLSPGERATLSCRASNAVSSSYLAWYQQKPGQAPRLLIYGASYRATGIPDRFSGSGSGTDFTLTISRLEPEDFAVYYCQQHSLYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 323 - protein, 213 aa, Artificial Sequence
  Description of Artificial Sequence: Synthetic polypeptide
DIQMTQSPSTLSASVGDRVTITCRASQSINSWLAWYQQKPGKAPKLLISDASSLESGVPSRFSGSGSGTEFTLTISSLQPDDFATYYCQQYDSHITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 324 - protein, 214 aa, Artificial Sequence
  Description of Artificial Sequence: Synthetic polypeptide
DIQMTQSPSSVSASVGDRVTITCAASQGISSDLAWYQQKPGKAPKLLIYSASSTQSGVPSRFSGSGSGTDFTLTISSLQPEDFATYYCQQAYLYPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 325 - protein, 214 aa, Artificial Sequence
  Description of Artificial Sequence: Synthetic polypeptide
GVQMTQSPSSLSASVGDRVTITCRASQDISTYLNWYQQKPGKAPKLLIYDAFNLETGVPSRFSGSGSGTDFTFTISSLQPEDIATYYCQQLPFLPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 326 - protein, 219 aa, Artificial Sequence
  Description of Artificial Sequence: Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALGGPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 327 - protein, 215 aa, Artificial Sequence
  Description of Artificial Sequence: Synthetic polypeptide
EIVLTQSPGTLSLSPGERATLSCRASQSVSSSYLAWYQQKPGQAPRLLIYGASRRATGIPDRFSGSGSGTDFTLTISRLEPEDFAVYYCQQYVSDPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 328 - protein, 215 aa, Artificial Sequence
  Description of Artificial Sequence: Synthetic polypeptide
EIVLTQSPGTLSLSPGERATLSCRASQSVSSSYLAWYQQKPGQAPRLLIYGASRRATGIPDRFSGSGSGTDFTLTISRLEPEDFAVYYCQQVGSSPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 329 - protein, 214 aa, Artificial Sequence
  Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSHLVPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 330 - protein, 215 aa, Artificial Sequence
  Description of Artificial Sequence: Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQANHHPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 331-333 - skipped (3 entries)

SEQ ID NO: 334 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 335 - protein, 327 aa, Artificial Sequence
  Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 336 - protein, 107 aa, Artificial Sequence [sequence omitted]

SEQ ID NO: 337 - protein, 338 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 338 - protein, 319 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 339 - protein, 336 aa, Macaca fascicularis
MAVMAPRTLLLVLSGVLALTQPRAGSHSMRYFYTAVSRPGRGQPRFIAVGYVDDTQFVRFDSDAESPRMEPRAPWVEQEGPEYWDRETQNMKTATQTYQANLRTLLRYYNQSEAGSHTFQKMYGCDLGPDGRLLRGYEQFAYDGRDYIILNEDLRSWTAADMAAQNTQRKWEAAGAAEQHRTYLEGECLEWLRRYLENGKETLQRADPPKTNVTHHPVSDYEATLRCWALGFYPAEITLTWQRDGEEQTEDTELVETRPTGDGTFQKWAAVVVPSGEEQRYTCHVQHEGLPKPLTLRWEPSSQSTILIVGIIAGLVLLGTVVTGAVVAAVMWRRKS

SEQ ID NO: 340 - protein, 331 aa, Macaca mulatta
LLLVLSGVLALTQTRAGSHSMRYFYTSMSRPGRGQPRFIAVGYVDDTQFVRFDSDAESPRMEPRAPWVEQEGPEYWDRETQNMKTATQTYRENLRTLLRYYNQSEAGSHTIQKMYGCDLGPDGRLLRGYEQFAYDGRDYIALNEDLRSWTAADMAAQFTQRKWEAAGAAEQHRTYLEGECLEWLRRYLENGKETLQRADPPKTNVTHHPVSDYEATLRCWALGFYPAEITLTWQRDGEEQTEDTELVETRPTGDGTFQKWAAVVVPSGEEQRYTCHVQHEGLPEPLTLRWEPSSQSTILIVGIIAGLVLLGTVVTGAVVAAVMWRRKSSDR

SEQ ID NO: 341 - protein, 297 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLR

SEQ ID NO: 342 - protein, 274 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRW

SEQ ID NO: 343-345 - skipped (3 entries)

SEQ ID NO: 346 - protein, 7 aa, Artificial Sequence
  Description of Artificial Sequence: Synthetic peptide
GYSFTGY

SEQ ID NO: 347 - protein, 7 aa, Artificial Sequence
  Description of Artificial Sequence: Synthetic peptide
GYTFTRY

SEQ ID NO: 348 - protein, 7 aa, Artificial Sequence
  Description of Artificial Sequence: Synthetic peptide
GFKFSGY

SEQ ID NO: 349 - protein, 7 aa, Artificial Sequence
  Description of Artificial Sequence: Synthetic peptide
GYTFTNY

SEQ ID NO: 350-353 - skipped (4 entries)

SEQ ID NO: 354 - protein, 5 aa, Artificial Sequence
  Description of Artificial Sequence: Synthetic peptide
GYTMN

SEQ ID NO: 355 - protein, 5 aa, Artificial Sequence
  Description of Artificial Sequence: Synthetic peptide
RYTMH

SEQ ID NO: 356 - protein, 5 aa, Artificial Sequence
  Description of Artificial Sequence: Synthetic peptide
GYGMH

SEQ ID NO: 357 - protein, 5 aa, Artificial Sequence
  Description of Artificial Sequence: Synthetic peptide
NYYIH

SEQ ID NO: 358-361 - skipped (4 entries)

SEQ ID NO: 362 - protein, 6 aa, Artificial Sequence
  Description of Artificial Sequence: Synthetic peptide
NPYKGV

SEQ ID NO: 363 - protein, 6 aa, Artificial Sequence
  Description of Artificial Sequence: Synthetic peptide
WYDGSK

SEQ ID NO: 364 - protein, 6 aa, Artificial Sequence
  Description of Artificial Sequence: Synthetic peptide
NPSRGY

SEQ ID NO: 365 - protein, 6 aa, Artificial Sequence
  Description of Artificial Sequence: Synthetic peptide
YPGDGN

SEQ ID NO: 366-370 - skipped (5 entries)

SEQ ID NO: 371 - protein, 17 aa, Artificial Sequence
  Description of Artificial Sequence: Synthetic peptide
LINPYKGVSTYNQKFKD

SEQ ID NO: 372 - protein, 17 aa, Artificial Sequence
  Description of Artificial Sequence: Synthetic peptide
YINPSRGYTNYNQKFKD

SEQ ID NO: 373 - protein, 17 aa, Artificial Sequence
  Description of Artificial Sequence: Synthetic peptide
YINPSRGYTNYNQKVKD

SEQ ID NO: 374 - protein, 17 aa, Artificial Sequence
  Description of Artificial Sequence: Synthetic peptide
WIYPGDGNTKYNEKEKG

SEQ ID NO: 375 - protein, 17 aa, Artificial Sequence
  Description of Artificial Sequence: Synthetic peptide
VIWYDGSKKYYVDSVKG

SEQ ID NO: 376-378 - skipped (3 entries)

SEQ ID NO: 379 - protein, 13 aa, Artificial Sequence
  Description of Artificial Sequence: Synthetic peptide
SGYYGDSDWYFDV

SEQ ID NO: 380 - protein, 10 aa, Artificial Sequence
  Description of Artificial Sequence: Synthetic peptide
DSYSNYYFDY

SEQ ID NO: 381 - protein, 9 aa, Artificial Sequence
  Description of Artificial Sequence: Synthetic peptide
QMGYWHFDL

SEQ ID NO: 382 - protein, 10 aa, Artificial Sequence
  Description of Artificial Sequence: Synthetic peptide
YYDDHYCLDY

SEQ ID NO: 383-387 - skipped (5 entries)

SEQ ID NO: 388 - protein, 11 aa, Artificial Sequence
  Description of Artificial Sequence: Synthetic peptide
RASQDIRNYLN

SEQ ID NO: 389 - protein, 10 aa, Artificial Sequence
  Description of Artificial Sequence: Synthetic peptide
SASSSVSYMN

SEQ ID NO: 390 - protein, 10 aa, Artificial Sequence
  Description of Artificial Sequence: Synthetic peptide
RASSSVSYMN

SEQ ID NO: 391 - protein, 17 aa, Artificial Sequence
  Description of Artificial Sequence: Synthetic peptide
KSSQSLLNSRTRKNYLA

SEQ ID NO: 392 - protein, 11 aa, Artificial Sequence
  Description of Artificial Sequence: Synthetic peptide
RASQSVSSYLA

SEQ ID NO: 393-395 - skipped (3 entries)

SEQ ID NO: 396 - protein, 7 aa, Artificial Sequence
  Description of Artificial Sequence: Synthetic peptide
YTSRLHS

SEQ ID NO: 397 - protein, 7 aa, Artificial Sequence
  Description of Artificial Sequence: Synthetic peptide
DTSKLAS

SEQ ID NO: 398 - protein, 7 aa, Artificial Sequence
  Description of Artificial Sequence: Synthetic peptide
DTSKVAS

SEQ ID NO: 399 - protein, 7 aa, Artificial Sequence
  Description of Artificial Sequence: Synthetic peptide
WASTRES

SEQ ID NO: 400 - protein, 7 aa, Artificial Sequence
  Description of Artificial Sequence: Synthetic peptide
DASNRAT

SEQ ID NO: 401-403 - skipped (3 entries)

SEQ ID NO: 404 - protein, 9 aa, Artificial Sequence
  Description of Artificial Sequence: Synthetic peptide
QQGNTLPWT

SEQ ID NO: 405 - protein, 9 aa, Artificial Sequence
  Description of Artificial Sequence: Synthetic peptide
QQWSSNPFT

SEQ ID NO: 406 - protein, 9 aa, Artificial Sequence
  Description of Artificial Sequence: Synthetic peptide
QQWSSNPLT

SEQ ID NO: 407 - protein, 8 aa, Artificial Sequence
  Description of Artificial Sequence: Synthetic peptide
TQSFILRT

SEQ ID NO: 408 - protein, 10 aa, Artificial Sequence
  Description of Artificial Sequence: Synthetic peptide
QQRSNWPPLT

SEQ ID NO: 409-412 - skipped (4 entries)

SEQ ID NO: 413 - protein, 122 aa, Artificial Sequence
  Description of Artificial Sequence: Synthetic polypeptide
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTVTVSS

SEQ ID NO: 414 - protein, 119 aa, Artificial Sequence
  Description of Artificial Sequence: Synthetic polypeptide
QVQLQQSGAELARPGASVKMSCKASGYTFTRYTMHWVKQRPGQGLEWIGYINPSRGYTNYNQKFKDKATLTTDKESSTAYMQLSELTSEDSAVYYCARYYDDHYCLDYWGQGTTLTVSS

SEQ ID NO: 415 - protein, 119 aa, Artificial Sequence
  Description of Artificial Sequence: Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 416 - protein, 119 aa, Artificial Sequence
  Description of Artificial Sequence: Synthetic polypeptide
EVQLVQSGAEVKKPGASVKVSCKASGYTFTNYYIHWVRQAPGQGLEWIGWIYPGDGNTKYNEKEKGRATLTADTSTSTAYLELSSLRSEDTAVYYCARDSYSNYYFDYWGQGTLVTVSS

SEQ ID NO: 417 - protein, 118 aa, Artificial Sequence [sequence omitted]

SEQ ID NO: 418 - protein, 119 aa, Artificial Sequence [sequence omitted]

SEQ ID NO: 419-421 - skipped (3 entries)

SEQ ID NO: 422 - protein, 107 aa, Artificial Sequence
  Description of Artificial Sequence: Synthetic polypeptide
DIQMTQTTSSLSASLGDRVTISCRASQDIRNYLNWYQQKPDGTVKLLIYYTERLHSGVPSKFSGSGSGTDYSLTISNLEQEDIATYFCQQGNTLPWTFAGGTKLEIK

SEQ ID NO: 423 - protein, 106 aa, Artificial Sequence [sequence omitted]

SEQ ID NO: 424 - protein, 106 aa, Artificial Sequence
  Description of Artificial Sequence: Synthetic polypeptide
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 425 - protein, 112 aa, Artificial Sequence
  Description of Artificial Sequence: Synthetic polypeptide
DIVMTQSPDSLAVSLGERATINCKSSQSLLNSRTRKNYLAWYQQKPGQPPKLLIYWASTRESGVPDRESGSGSGTDETLTISSLQAEDVAVYYCTQSFILRTFGQGTKVEIK

SEQ ID NO: 426 - protein, 108 aa, Artificial Sequence [sequence omitted]

SEQ ID NO: 427 - protein, 106 aa, Artificial Sequence [sequence omitted]

SEQ ID NO: 428-430 - skipped (3 entries)

SEQ ID NO: 431 - protein, 9 aa, Artificial Sequence
  Description of Artificial Sequence: Synthetic peptide
GFSLSTSGM

SEQ ID NO: 432 - protein, 7 aa, Artificial Sequence
  Description of Artificial Sequence: Synthetic peptide
GFTFNNY

SEQ ID NO: 433 - protein, 7 aa, Artificial Sequence
  Description of Artificial Sequence: Synthetic peptide
GFTFSNY

SEQ ID NO: 434 - protein, 7 aa, Artificial Sequence
  Description of Artificial Sequence: Synthetic peptide
GYTFTSS

SEQ ID NO: 435 - protein, 8 aa, Artificial Sequence
  Description of Artificial Sequence: Synthetic peptide
GFTFSNYG

SEQ ID NO: 436 - protein, 8 aa, Artificial Sequence
  Description of Artificial Sequence: Synthetic peptide
GFTFSSYG

SEQ ID NO: 437 - protein, 8 aa, Artificial Sequence
  Description of Artificial Sequence: Synthetic peptide
GYTFTSYY

SEQ ID NO: 438-440 - skipped (3 entries)

SEQ ID NO: 441 - protein, 7 aa, Artificial Sequence
  Description of Artificial Sequence: Synthetic peptide
TSGMGVG

SEQ ID NO: 442 - protein, 7 aa, Artificial Sequence
  Description of Artificial Sequence: Synthetic peptide
TSGVGVG

SEQ ID NO: 443 - protein, 5 aa, Artificial Sequence
  Description of Artificial Sequence: Synthetic peptide
NYGMS

SEQ ID NO: 444 - protein, 5 aa, Artificial Sequence
  Description of Artificial Sequence: Synthetic peptide
SSAMH

SEQ ID NO: 445-447 - skipped (3 entries)

SEQ ID NO: 448 - protein, 5 aa, Artificial Sequence
  Description of Artificial Sequence: Synthetic peptide
WWDDD

SEQ ID NO: 449 - protein, 6 aa, Artificial Sequence
  Description of Artificial Sequence: Synthetic peptide
SGGGSY

SEQ ID NO: 450 - protein, 6 aa, Artificial Sequence
  Description of Artificial Sequence: Synthetic peptide
NHYNDG

SEQ ID NO: 451 - protein, 8 aa, Artificial Sequence
  Description of Artificial Sequence: Synthetic peptide
IYYSGGST

SEQ ID NO: 452 - protein, 8 aa, Artificial Sequence
  Description of Artificial Sequence: Synthetic peptide
VNHSGGST

SEQ ID NO: 453 - protein, 8 aa, Artificial Sequence
  Description of Artificial Sequence: Synthetic peptide
IEPMYGST

SEQ ID NO: 454 - protein, 8 aa, Artificial Sequence
  Description of Artificial Sequence: Synthetic peptide
INPSGGST

SEQ ID NO: 455-457 - skipped (3 entries)

SEQ ID NO: 458 - protein, 17 aa, Artificial Sequence
  Description of Artificial Sequence: Synthetic peptide
YINHYNDGIKYNERFKG

SEQ ID NO: 459 - protein, 15 aa, Artificial Sequence
  Description of Artificial Sequence: Synthetic peptide
HIWDDDKRYNPALKS

SEQ ID NO: 460 - protein, 15 aa, Artificial Sequence
  Description of Artificial Sequence: Synthetic peptide
HIWDDDKRYNPSLKS

SEQ ID NO: 461 - protein, 17 aa, Artificial Sequence
  Description of Artificial Sequence: Synthetic peptide
TISGGGSYTFYPDSVKG

SEQ ID NO: 462-464 - skipped (3 entries)

SEQ ID NO: 465 - protein, 8 aa, Artificial Sequence
  Description of Artificial Sequence: Synthetic peptide
TNPAWFAY

SEQ ID NO: 466 - protein, 8 aa, Artificial Sequence
  Description of Artificial Sequence: Synthetic peptide
INPAYFAY

SEQ ID NO: 467 - protein, 9 aa, Artificial Sequence
  Description of Artificial Sequence: Synthetic peptide
QSARAPEPY

SEQ ID NO: 468 - protein, 9 aa, Artificial Sequence
  Description of Artificial Sequence: Synthetic peptide
QSANSPVPY

SEQ ID NO: 469 - protein, 10 aa, Artificial Sequence
  Description of Artificial Sequence: Synthetic peptide
GYRYASWFAS

SEQ ID NO: 470 - protein, 7 aa, Artificial Sequence
  Description of Artificial Sequence: Synthetic peptide
ARESIDY

SEQ ID NO: 471 - protein, 8 aa, Artificial Sequence
  Description of Artificial Sequence: Synthetic peptide
ARVGSFDF

SEQ ID NO: 472 - protein, 13 aa, Artificial Sequence
  Description of Artificial Sequence: Synthetic peptide
ARGSAYYYDFADY

SEQ ID NO: 473-475 - skipped (3 entries)

SEQ ID NO: 476 - protein, 15 aa, Artificial Sequence
  Description of Artificial Sequence: Synthetic peptide
KASQSVDFDGDSFMN

SEQ ID NO: 477 - protein, 11 aa, Artificial Sequence
  Description of Artificial Sequence: Synthetic peptide
KASQNVGTHVA

SEQ ID NO: 478 - protein, 11 aa, Artificial Sequence
  Description of Artificial Sequence: Synthetic peptide
RASQNVGTHVA

SEQ ID NO: 479 - protein, 11 aa, Artificial Sequence
  Description of Artificial Sequence: Synthetic peptide
RASQNIGTSIH

SEQ ID NO: 480 - protein, 6 aa, Artificial Sequence
  Description of Artificial Sequence: Synthetic peptide
NIGSKN

SEQ ID NO: 481-483 - skipped (3 entries)

SEQ ID NO: 484 - protein, 7 aa, Artificial Sequence
  Description of Artificial Sequence: Synthetic peptide
TTSNLES

SEQ ID NO: 485 - protein, 7 aa, Artificial Sequence
  Description of Artificial Sequence: Synthetic peptide
TTSSLES

SEQ ID NO: 486 - protein, 7 aa, Artificial Sequence
  Description of Artificial Sequence: Synthetic peptide
SASYRYS

SEQ ID NO: 487 - protein, 7 aa, Artificial Sequence
  Description of Artificial Sequence: Synthetic peptide
SVSESIS

SEQ ID NO: 488 - protein, 3 aa, Artificial Sequence
  Description of Artificial Sequence: Synthetic peptide
QDN

SEQ ID NO: 489-491 - skipped (3 entries)

SEQ ID NO: 492 - protein, 9 aa, Artificial Sequence
  Description of Artificial Sequence: Synthetic peptide
QQSNEDPYT

SEQ ID NO: 493 - protein, 9 aa, Artificial Sequence
  Description of Artificial Sequence: Synthetic peptide
QQSNSDPYT

SEQ ID NO: 494 - protein, 9 aa, Artificial Sequence
  Description of Artificial Sequence: Synthetic peptide
QQYKSYPLT

SEQ ID NO: 495 - protein, 9 aa, Artificial Sequence
  Description of Artificial Sequence: Synthetic peptide
QDYTNYPLT

SEQ ID NO: 496 - protein, 9 aa, Artificial Sequence
  Description of Artificial Sequence: Synthetic peptide
QQSNSWPLT

SEQ ID NO: 497 - protein, 9 aa, Artificial Sequence
  Description of Artificial Sequence: Synthetic peptide
QVWDNYSVL

SEQ ID NO: 498-500 - skipped (3 entries)

SEQ ID NO: 501 - protein, 118 aa, Artificial Sequence
  Description of Artificial Sequence: Synthetic polypeptide
QVTLRESGPALVKPTQTLTLTCTFSGFSLSTSGMGVGWIRQPPGKALEWLAHIWWDDDKRYNPALKSRLTISKDTSKNQVVLTMTNMDPVDTATYYCARTNPAWFAYWGQGTLVTVSS

SEQ ID NO: 502 - protein, 118 aa, Artificial Sequence
  Description of Artificial Sequence: Synthetic polypeptide
QVTLRESGPALVKPTQTLTLTCTFSGFSLSTSGMGVGWIRQPPGKALEWLAHIWWDDDKRYNPALKSRLTTSKDTSKNQVVLTNTNMDPVDTATYYCAQTNPAWFAYWGQGTLVTVSS

SEQ ID NO: 503 - protein, 117 aa, Artificial Sequence
  Description of Artificial Sequence: Synthetic polypeptide
QVTLRESGPALVKPTQTLTLTCTFSGFSLSTSGVGVGWIRQPPGKALEWLAHIWWDDDKRYSPSLKSRLTISKDTSKNQVVLTMTNMDPVDTATYYCARINPAYFAYWGQGTLVTVS

SEQ ID NO: 504 - protein, 118 aa, Artificial Sequence
  Description of Artificial Sequence: Synthetic polypeptide
EVKLVESGGTLVKPGGSLKLSCAASGFTFNNYGMSWVRQTPEKRLEWVATISGGGSYTFVPDSVKGRFTISRDNAKNSLYLQMSSLRSEDTALYYCIRQSARAPEPYWGQGTLVTVSS

SEQ ID NO: 505 - protein, 118 aa, Artificial Sequence
  Description of Artificial Sequence: Synthetic polypeptide
EVQLVESGGGLVKPGGSLRLSCAASGFTFSNYGMSWVRQAPGKGLEWVATISGGGSYTFYPDSVKGRFTISRDNAKNSLYLQMNSLRTEDTALYYCVRQSARAPEPYWGQGTLVTVSS

SEQ ID NO: 506 - protein, 118 aa, Artificial Sequence
  Description of Artificial Sequence: Synthetic polypeptide
EVQLVESGGGLVKPGGSLRLSCAASGFTFSNYGMSWVRQAPGKGLEWVATISGGGSYTFYPDSVKGRFTISRDNAKNSLYLQMNSLRTEDTALYYCVRQSANSPVPYWGQGTLVTVSS

SEQ ID NO: 507 - protein, 119 aa, Artificial Sequence
  Description of Artificial Sequence: Synthetic polypeptide
EVQLQQSGPELVKPGASVKMSCRASGYTFTSSAMHWVKKNPGQGLEWIGYINHYNDGIKYNERFKGKATLTSDKSSSTAYMELSSLTSEDSAVYYCATGYRYASWFASWGQGTLVTVSS

SEQ ID NO: 508 - protein, 119 aa, Artificial Sequence
  Description of Artificial Sequence: Synthetic polypeptide
QVQLVQSGAEVKKPGASVKVSCKASGYTFTSSAMHWVRQAPGQGLEWMGYINHYNDGIKYNERFKGRVTITADKSTSTAYMELSSLRSEDTAVYYCATGYRYASWFASWGQGTLVTVSS

SEQ ID NO: 509 - protein, 119 aa, Artificial Sequence
  Description of Artificial Sequence: Synthetic polypeptide
QVQLVQSGAEVKKPGASVKVSCKASGYTFTSSAMHWVRQAPGQGLEWMGYINHYNDGIKYNERFKGRVTITADKSTSTAYMELSSLRSEDTAVYYCARGYRYASWFASWGQGTLVTVSS

SEQ ID NO: 510 - protein, 112 aa, Artificial Sequence
  Description of Artificial Sequence: Synthetic polypeptide
EVQLVESGGGLVQPGGSLRLSCAASGFTFSNYGMSWVRQAPGKGLEWIGSLYYSGGSTNYNPSLKGSLVISRDNSKNTLYLQMSLAEDTATYYCARESIDYWGQGTLVTVSS

SEQ ID NO: 511 - protein, 114 aa, Artificial Sequence
  Description of Artificial Sequence: Synthetic polypeptide
EVQLVESGGGLVQPGGSLRLSCAASGFTFSSYGMSWVRQAPGKGLEWIGEVNHSGGSTNYNPSLKGSFVISRDNSKNTLYLQMNSLAEDTAVYYCARVGSFDFWGQGTLVTVSS

SEQ ID NO: 512 - protein, 120 aa, Artificial Sequence
  Description of Artificial Sequence: Synthetic polypeptide
QVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSS

SEQ ID NO: 513 - protein, 120 aa, Artificial Sequence
  Description of Artificial Sequence: Synthetic polypeptide
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 514-516 - skipped (3 entries)

SEQ ID NO: 517 - protein, 111 aa, Artificial Sequence
  Description of Artificial Sequence: Synthetic polypeptide
DIVMTQSPDSLAVSLGERATINCKASQSVDFDGDSFMNWYQQKPGQPPKLLIYTTSNLESGVPDRFSGSGSGTDFTLTISSLQAEDVAVYYCQQSNEDPYTFGQGTKLEIK

SEQ ID NO: 518 - protein, 111 aa, Artificial Sequence
  Description of Artificial Sequence: Synthetic polypeptide
DIVMTQSPDSLAVSLGERATINCKSSQSVDFDGDSFMNWYQQKPGQPPKLLIYTTSSLESGVPDRFSGSGSGTDFTLTISSLQAEDVAVYYCQQSNSDPYTFGQGTKLEIK

SEQ ID NO: 519 - protein, 107 aa, Artificial Sequence
  Description of Artificial Sequence: Synthetic polypeptide
DIVMTQSQKFMSTSVGDRVSVTCKASQNVGTHVAWYQQKSGQSPKSLLYSASYRYSGVPDRFSGSGSGTDFTLTISNVQSEDLAEYFCQQYKSYPLTFGAGTKLELK

SEQ ID NO: 520 - protein, 107 aa, Artificial Sequence
  Description of Artificial Sequence: Synthetic polypeptide
DIQMTQSPSFLSASVGDRVTITCRASQNVGTHVAWYQQKPGKAPKSLLYSASYRYSGVPSRFSGSGSGTDFTLTISSLQSEDIATYYCQQYKSYPLTFGQGTKLEIK

SEQ ID NO: 521 - protein, 107 aa, Artificial Sequence
  Description of Artificial Sequence: Synthetic polypeptide
DIQMTQSPSFLSASVGDRVTITCRASQNVGTHVAWYQQKPGKAPKSLLYSASYRYSGVPSRFSGSGSGTDFTLTISSLQSEDIATYYCQDYTNYPLTFGQGTKLEIK

SEQ ID NO: 522 - protein, 105 aa, Artificial Sequence
  Description of Artificial Sequence: Synthetic polypeptide
DILLTQSPAILSVSPGERVSFSCRASQNIGTSIHWYQQRTDGSPRLLIKSVSESISGIPSRFSGSGTDFTLTINGVESGDISDYYCQQSNSWPLTFGAGTKLELK

SEQ ID NO: 523 - protein, 107 aa, Artificial Sequence
  Description of Artificial Sequence: Synthetic polypeptide
EIVLTQSPATLSVSPGERATLSCRASQNIGTSIHWYQQKPDQSPKLLIKSVSESISGVPSRFSGSGSGTDFTLTINSLEAEDFATYYCQQSNSWPLTFGQGTKLEIK

SEQ ID NO: 524 - protein, 106 aa, Artificial Sequence
  Description of Artificial Sequence: Synthetic polypeptide
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 525-527 - skipped (3 entries)

SEQ ID NO: 528 - protein, 9 aa, Artificial Sequence
  Description of Artificial Sequence: Synthetic peptide
GFSLSTYGI

SEQ ID NO: 529-531 - skipped (3 entries)

SEQ ID NO: 532 - protein, 7 aa, Artificial Sequence
  Description of Artificial Sequence: Synthetic peptide
TYGIGVG

SEQ ID NO: 533-535 - skipped (3 entries)

SEQ ID NO: 536 - protein, 5 aa, Artificial Sequence
  Description of Artificial Sequence: Synthetic peptide
WWNDN

SEQ ID NO: 537-539 - skipped (3 entries)

SEQ ID NO: 540 - protein, 16 aa, Artificial Sequence
  Description of Artificial Sequence: Synthetic peptide
HIWWNDNEYYNIDLKS

SEQ ID NO: 541-543 - skipped (3 entries)

SEQ ID NO: 544 - protein, 13 aa, Artificial Sequence
  Description of Artificial Sequence: Synthetic peptide
GNYRYARGYVMDY

SEQ ID NO: 545-547 - skipped (3 entries)

SEQ ID NO: 548 - protein, 15 aa, Artificial Sequence
  Description of Artificial Sequence: Synthetic peptide
RASESVEYYGTSLMQ

SEQ ID NO: 549-551 - skipped (3 entries)

SEQ ID NO: 552 - protein, 7 aa, Artificial Sequence
  Description of Artificial Sequence: Synthetic peptide
AASNVES

SEQ ID NO: 553-555 - skipped (3 entries)

SEQ ID NO: 556 - protein, 9 aa, Artificial Sequence
  Description of Artificial Sequence: Synthetic peptide
QQNRKVPWT

SEQ ID NO: 557-559 - skipped (3 entries)

SEQ ID NO: 560 - protein, 123 aa, Artificial Sequence
  Description of Artificial Sequence: Synthetic polypeptide
QVTLKESGPGILQPSQTLSLTCSFSGFSLSTYGIGVGWNRQPSGKGLEWLAHIWWNDNEYYNIDLKSRLTISKDTSNNQVFLKIASVDTADTATYYCVRGNYRYARGYVMDYWGQGTSVTVSS

SEQ ID NO: 561-563 - skipped (3 entries)

SEQ ID NO: 564 - protein, 111 aa, Artificial Sequence
  Description of Artificial Sequence: Synthetic polypeptide
DIVLTQSPASLAVSLGQRATISCRASESVEYYGTSLMQWYQQKPGQPPKLLIYAASNVESGVPARFSGSGSGTDFSLNIHPVEEDDFAMYFCQQNRKVPWTFGGGTKLEIK

SEQ ID NO: 565-567 - skipped (3 entries)

SEQ ID NO: 568 - protein, 7 aa, Artificial Sequence
  Description of Artificial Sequence: Synthetic peptide
GHTFTSY

SEQ ID NO: 569 - protein, 7 aa, Artificial Sequence
  Description of Artificial Sequence: Synthetic peptide
GGTFTSY

SEQ ID NO: 570 - protein, 7 aa, Artificial Sequence
  Description of Artificial Sequence: Synthetic peptide
GHTFTNY

SEQ ID NO: 571-573 - skipped (3 entries)

SEQ ID NO: 574 - protein, 5 aa, Artificial Sequence
  Description of Artificial Sequence: Synthetic peptide
SYFMH

SEQ ID NO: 575 - protein, 5 aa, Artificial Sequence
  Description of Artificial Sequence: Synthetic peptide
SYSVS

SEQ ID NO: 576 - protein, 5 aa, Artificial Sequence
  Description of Artificial Sequence: Synthetic peptide
NYYMH

SEQ ID NO: 577-579 - skipped (3 entries)

SEQ ID NO: 580 - protein, 6 aa, Artificial Sequence
  Description of Artificial Sequence: Synthetic peptide
NPSDDY

SEQ ID NO: 581 - protein, 6 aa, Artificial Sequence
  Description of Artificial Sequence: Synthetic peptide
VPIFGT

SEQ ID NO: 582 - protein, 5 aa, Artificial Sequence
  Description of Artificial Sequence: Synthetic peptide
NPNGD

SEQ ID NO: 583-585 - skipped (3 entries)

SEQ ID NO: 586 - protein, 17 aa, Artificial Sequence
  Description of Artificial Sequence: Synthetic peptide
IINPSDDYANYAQKFQG

SEQ ID NO: 587 - protein, 17 aa, Artificial Sequence
  Description of Artificial Sequence: Synthetic peptide
GIVPIFGTADYAQKFQG

SEQ ID NO: 588 - protein, 17 aa, Artificial Sequence
  Description of Artificial Sequence: Synthetic peptide
VINPNGGDTSYAQKFQG

SEQ ID NO: 589-591 - skipped (3 entries)

SEQ ID NO: 592 - protein, 5 aa, Artificial Sequence
  Description of Artificial Sequence: Synthetic peptide
AIFDY

SEQ ID NO: 593 - protein, 10 aa, Artificial Sequence
  Description of Artificial Sequence: Synthetic peptide
GYSYGQTFDY

SEQ ID NO: 594 - protein, 15 aa, Artificial Sequence
  Description of Artificial Sequence: Synthetic peptide
DRAWDYGGNVRAFDI

SEQ ID NO: 595-597 - skipped (3 entries)

SEQ ID NO: 598 - protein, 11 aa, Artificial Sequence
  Description of Artificial Sequence: Synthetic peptide
RASQSISSYLN

SEQ ID NO: 599 - protein, 16 aa, Artificial Sequence
  Description of Artificial Sequence: Synthetic peptide
RSSQSLLHSNGYNYLD

SEQ ID NO: 600-602 - skipped (3 entries)

SEQ ID NO: 603 - protein, 7 aa, Artificial Sequence
  Description of Artificial Sequence: Synthetic peptide
AASSLQS

SEQ ID NO: 604 - protein, 7 aa, Artificial Sequence
  Description of Artificial Sequence: Synthetic peptide
LGSNRAS

SEQ ID NO: 605-607 - skipped (3 entries)

SEQ ID NO: 608 - protein, 9 aa, Artificial Sequence
  Description of Artificial Sequence: Synthetic peptide
QQSYSTPLT

SEQ ID NO: 609 - protein, 9 aa, Artificial Sequence
  Description of Artificial Sequence: Synthetic peptide
MQALQTPYT

SEQ ID NO: 610-612 - skipped (3 entries)

SEQ ID NO: 613 - protein, 114 aa, Artificial Sequence
  Description of Artificial Sequence: Synthetic polypeptide
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTLVTVSS

SEQ ID NO: 614 - protein, 114 aa, Artificial Sequence
  Description of Artificial Sequence: Synthetic polypeptide
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTPVTVSS

SEQ ID NO: 615 - protein, 124 aa, Artificial Sequence
  Description of Artificial Sequence: Synthetic polypeptide
QVQLVQSGAEVKKPGASVKVSCKASGHTFTNYYMHWVRQAPGQGLEWMGVINPNGGDTSYAQKFQGRVTMTRDTSTSTVYMELSSLRSEDTAVYYCARDRAWDYGGNVRAFDIWGQGTMVTVSS

SEQ ID NO: 616 - protein, 119 aa, Artificial Sequence
  Description of Artificial Sequence: Synthetic polypeptide
QVQLVQSGAEVKKPGSSVKVSCKASGGTFTSYSVSWVRQAPGQGLEWMGGIVPIFGTADYAQKFQGRVTITADESTSTAYMELSSLRSEDTAVYYCARGYSYGQTFDYWGQGTLVTVSS

SEQ ID NO: 617-623 - skipped (7 entries)

SEQ ID NO: 624 - protein, 107 aa, Artificial Sequence [sequence omitted]

SEQ ID NO: 625 - protein, 112 aa, Artificial Sequence
  Description of Artificial Sequence: Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPYTFGGGTKVEIK

SEQ ID NO: 626-628 - skipped (3 entries)

SEQ ID NO: 629 - protein, 207 aa, Artificial Sequence
  Description of Artificial Sequence: Synthetic polypeptide
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 630 - protein, 254 aa, Artificial Sequence
  Description of Artificial Sequence: Synthetic polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 631 - protein, 233 aa, Artificial Sequence
  Description of Artificial Sequence: Synthetic polypeptide
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 632 - protein, 304 aa, Homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 633 - protein, 287 aa, Homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPDHALWDHTAQNLLRMGLAFLVLVALVWFLVEDWLSRKRTRERASRASTWEGRRRLNTQTL

SEQ ID NO: 634 - protein, 209 aa, Homo sapiens
MSSTLPALLCVGLCLSQRISAQQQMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 635 - protein, 192 aa, Homo sapiens
MSSTLPALLCVGLCLSQRISAQQQMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPDHALWDHTAQNLLRMGLAFLVLVALVWFLVEDWLSRKRTRERASRASTWEGRRRLNTQTL

SEQ ID NO: 636 - protein, 197 aa, Homo sapiens
MSSTLPALLCVEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 637 - protein, 303 aa, Homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPDTWGTYLLTTETGLQKDHALWDHTAQNLLRMGLAFLVLVALVWFLVEDWLSRKRTRERASRASTWEGRRRLNTQTL

SEQ ID NO: 638 - protein, 200 aa, Homo sapiens
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAWPAPLPPPCGSSAHLLPPVPGG

SEQ ID NO: 639 - protein, 190 aa, Homo sapiens
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCHCHMGTHCHSSDGPRGVIPEPRCP

SEQ ID NO: 640 - protein, 177 aa, Homo sapiens
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKYAKSTLSGFPQL

SEQ ID NO: 641 - protein, 174 aa, Homo sapiens
MAWMLLLILIMVHPGSCALWVSQPPEIRTLEGSSAFLPCSFNASQGRLAIGSVTWFRDEVVPGKEAELHIRDVRGHDASIYVCRVEVLGLGVGTGNGTRLWEKEHPQLGAGTVLLLRAGFYAVSFLSVAVGSTVYYQGKCLTWKGPRRQLPAWPAPLPPPCGSSAHLLPPVPGG

SEQ ID NO: 642 - protein, 164 aa, Homo sapiens
MAWMLLLILIMVHPGSCALWVSQPPEIRTLEGSSAFLPCSFNASQGRLAIGSVTWFRDEVVPGKEAELHIRDVRGHDASIYVCRVEVLGLGVGTGNGTRLWEKEHPQLGAGTVLLLRAGFYAVSFLSVAVGSTVYYQGKCHCHMGTHCHSSDGPRGVIPEPRCP

SEQ ID NO: 643 - protein, 151 aa, Homo sapiens
MAWMLLLILIMVHPGSCALWVSQPPEIRTLEGSSAFLPCSFNASQGRLAIGSVTWFRDEVVPGKEAELHIRDVRGHDASIYVCRVEVLGLGVGTGNGTRLWEKEHPQLGAGTVLLLRAGFYAVSFLSVAVGSTVYYQGKYAKSTLSGFPQL